(19) United States
(12) Patent Application Publication
(10) Pub. No.: US 2025/0154510 A1
(43) Pub. Date: May 15, 2025

(54) METHODS FOR TREATING PROSTATE AND LUNG CANCER

(71) Applicant: THE REGENTS OF THE UNIVERSITY OF CALIFORNIA, Oakland, CA (US)

(72) Inventors: Thomas G. GRAEBER, Pacific Palisades, CA (US); Chia-Chin CHEN, Los Angeles, CA (US); Owen WITTE, Los Angeles, CA (US); Jung Wook PARK, Los Angeles, CA (US)

(73) Assignee: THE REGENTS OF THE UNIVERSITY OF CALIFORNIA, Oakland, CA (US)

(21) Appl. No.: 18/949,317

(22) Filed: Nov. 15, 2024

Related U.S. Application Data
(60) Provisional application No. 63/599,350, filed on Nov. 15, 2023.

(51) Int. Cl. C12N 15/113; A61K 45/06; A61N 5/10

(52) U.S. Cl. CPC C12N 15/1135; A61K 45/06; A61N 5/10; C12N 2310/531; C12N 2320/31

(57) ABSTRACT

The current disclosure is centered on the hypothesis that a better understanding of the neuroendocrine transdifferentiation (NEtD) process will provide therapeutic targets for inhibiting this transition and thus improving standard of care therapies. Accordingly, the current disclosure provides for a method for treating cancer in a subject, the method comprising administering an inhibitor of a gene, wherein the gene comprises one or more of tenascin C (TNC), advillin (AVIL), S100A7, PPARG, LOX lysyl oxidase, KLF5, APOBEC2, FOSL1, FOXM1, hedgehog, RELA, p65, IKK complex, JAK, STAT, TFAP4, geminin, OCA-1, OCA-2, Nf-kB, Nf-kB family and associated regulators, angiogenesis regulators such as BMX kinase, TEK kinase, periostin (POSTN), and VEGF family, stress genes, such as the JUN/FOS family, a gene from the S100 family, and cell state regulators such as ASCL1, POU2F3, NEUROD1, ASCL2, and YAP1.

### BACKGROUND OF THE INVENTION

This application claims priority of U.S. Provisional Patent Application No. 63/599,350, filed Nov. 15, 2023, which is hereby incorporated by reference in its entirety.

This invention was made with government support under W81XWH-21-1-0806 awarded by the Medical Research and Development Command, and CA092131, GM008042, CA222877, and CA009056 awarded by the National Institutes of Health. The government has certain rights in the invention.

### I. FIELD OF THE INVENTION

This invention relates to the field of

### II. BACKGROUND

Transdifferentiation is an emerging resistance mechanism for otherwise effective targeted therapies. Next generation androgen axis inhibitors (e.g. enzalutamide, abiraterone) have been shown to extend survival in some groups of men with castration-resistant prostate cancer (CRPC). Nevertheless, the plasticity of cancer can break through these targeted therapy advances and result in therapy-resistance. Transdifferentiation to neuroendocrine prostate cancer represents a reprogramming resistance mechanism that results in a cell type no longer dependent on the originally targeted pathway (change in cell identity). A very analogous transdifferentiation mechanism also results in resistance to lung adenocarcinoma targeted therapies (e.g. anti-EGFR, anti-ALK), resulting in conversion to small cell lung cancer (SCLC). Additional examples of transdifferentiation and a change in cell identity to escape an effective targeting of a critical oncogene are found in multiple cancers, such as hedgehog targeting in basal cell carcinoma. Transdifferentiation is also observed in resistance mechanisms of hematopoietic cancers. In sum, the list of ‘change of cell identity’ therapy escape mechanisms is growing, and can be expected to expand as targeted therapies are improved. There is a need in the art for therapies that target the transdifferentiation process to prevent drug resistance and therapy escape.

### SUMMARY OF THE INVENTION

The current disclosure is centered on the hypothesis that a better understanding of the neuroendocrine transdifferentiation (NEtD) process will provide therapeutic targets for inhibiting this transition and thus improving standard of care therapies. Accordingly, the current disclosure provides for a method for treating cancer in a subject, the method comprising administering an inhibitor of a gene, wherein the gene comprises one or more of tenascin C (TNC), advillin (AVIL), S100A7, PPARG, LOX lysyl oxidase, KLF5, APOBEC2, FOSL1, FOXM1, hedgehog, RELA, p65, IKK complex, JAK, STAT, TFAP4, geminin, OCA-1, OCA-2, Nf-kB, Nf-kB family and associated regulators, angiogenesis regulators such as BMX kinase, TEK kinase, periostin (POSTN), and VEGF family, stress genes, such as the JUN/FOS family, a gene from the S100 family, and cell state regulators such as ASCL1, POU2F3, NEUROD1, ASCL2, and YAP1. Also described is a method for treating cancer prostate cancer or lung cancer in a subject, the method comprising administering an inhibitor of a gene, wherein the gene comprises one or more genes from Tables 1-6. Further methods relate to a method for inhibiting cancer transdifferentiation in a subject having cancer, the method comprising administering an inhibitor of a gene, wherein the gene comprises one or more of tenascin C (TNC), advillin (AVIL), S100A7, PPARG, LOX lysyl oxidase, KLF5, APOBEC2, FOSL1, FOXM1, hedgehog, RELA, p65, IKK complex, JAK, STAT, TFAP4, geminin, OCA-1, OCA-2, Nf-kB, Nf-kB family and associated regulators, angiogenesis regulators such as BMX kinase, TEK kinase, periostin (POSTN), and VEGF family, stress genes, such as the JUN/FOS family, a gene from the S100 family, and cell state regulators such as ASCL1, POU2F3, NEUROD1, ASCL2, and YAP1. Also provided is a method for inhibiting cancer transdifferentiation in a subject having cancer, the method comprising administering an inhibitor of a gene, wherein the gene comprises one or more genes from Tables 1-6.

The cancer may include or exclude lung, prostate, basal cell carcinoma, hematopoietic cancer, ovarian cancer, epithelial cancer, sarcomas, small round cell-cancers of childhood, or neuroblastoma. Inhibiting transdifferentiation comprises inhibiting neuroendocrine or small cell transdifferentiation. The prostate cancer may include or exclude prostate adenocarcinoma, castration-resistant prostate cancer, castration-sensitive prostate cancer, or hormone-refractory prostate cancer. The lung cancer may include or exclude non-small cell lung cancer, adenocarcinoma, adenocarcinoma in situ, squamous cell carcinoma, large cell carcinoma, large cell neuroendocrine carcinoma, adenosquamous carcinoma, sarcomatoid carcinoma, or small cell lung cancer. The hematopoietic cancer may include or exclude leukemia or lymphoma. The cancer may include or exclude acute lymphoblastic leukemia, acute myeloid leukemia, chronic lymphocytic leukemia, acute monocytic leukemia, Hodgkin's lymphoma, or Non-Hodgkin's lymphoma.

The method may comprise or exclude administration of an additional agent. The subject may include or exclude one that has been prescribed or is being treated with an additional agent or therapy. The subject may include or exclude on that is being treated or has been treated with an additional agent or therapy and wherein the subject has been determined to be resistant to the additional agent or therapy. The subject may be one that has not been treated with an additional agent or therapy.

The cancer may comprise prostate cancer. The additional agent may include or exclude one or more of androgen suppression therapy, chemotherapy, immunotherapy, targeted therapy, radiation, and surgery. The androgen suppression therapy may include or exclude one or more of leuprolide, goserelin, triptorelin, leuprolide mesylate, degarelix, relugolix, abiraterone, ketoconazole, flutamide, bicalutamide, nilutamide, enzalutamide, apalutamide, or darolutamid. The immunotherapy may include or exclude pembrolizumab. The targeted therapy may include or exclude rucaparib and/or olaparib.

The cancer may comprise lung cancer. The additional agent may include or exclude one or more of chemotherapy, immunotherapy, radiation therapy, targeted therapy, and surgery. The chemotherapy may include or exclude cisplatin, carboplatin, paclitaxel, albumin-bound paclitaxel, docetaxel, gemcitabine, vinorelbine, etoposide, pemetrexed, and combinations thereof. The immunotherapy may include or exclude nivolumab, atezolizumab, durvalumab, ipilimumab, tremelimumab, and combinations thereof. The targeted therapy may include or exclude bevacizumab, ramucirumab, sotorasib, adagrasib, erlotinib, afatinib, gefitinib, osimertinib, dacomitinib, amivantamab, mobocertinib, necitumumab, crizotinib, ceritinib, alectinib, brigatinib, lorlatinib, entrectinib, dabrafenib, trametinib, selpercatinib, pralsetinib, capmatinib, tepotinib, trastuzumab deruxtecan, larotrectinib, and combinations thereof.

The inhibitor may include or exclude an inhibitor nucleic acid, inhibitory protein, or inhibitory small molecule. The inhibitor may include or exclude an siRNA, a double stranded RNA, a short hairpin RNA, and an antisense oligonucleotide. The inhibitor may be an antibody. The inhibitor may be one known in the art, for example, the inhibitor may include or exclude an inhibitor described in Gamble C et al., Br J Pharmacol. 2012; 165 (4): 802-819, Midwood KS et al., J Cell Commun Signal. 2009 December; 3 (3-4): 287-310, Bariwal J, et al., Med Res Rev. 2019 May; 39 (3): 1137-1204, Jamieson C. et al., Blood Cancer Discov. 2020 Sep. 1;1 (2): 134-145, Chen S, et al., Cancer Res. 2018 Sep. 15;78 (18): 5203-5215, Jarboe J S et al., Recent Patents Anticancer Drug Discov. 2013 September; 8 (3): 228-238, Saharinen P. et al., Nat Rev Drug Discov. Nature Publishing Group; 2017 September; 16 (9): 635-661, and Pasparakis M. et al., Cell Death Differ. Nature Publishing Group; 2006 May; 13 (5): 861-872, all of which are incorporated by reference.

The cancer may include or exclude a stage I, II, III, or IV cancer. The cancer may comprise or exclude metastatic cancer. The cancer may comprise non-metastatic cancer.

The subject may be a human. The subject may be a laboratory animal such as a rat, mouse, rabbit, monkey, goat, pig, or horse. The subject may be a mammalian subject. The subject may be a non-human primate.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments and aspects described herein in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments and aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment or aspect discussed with respect to one aspect of the disclosure applies to other aspects of the disclosure as well and vice versa.

Other objects, features and advantages of the present invention will become apparent from the following detailed description. It should be understood, however, that the detailed description and the specific examples, while indicating specific embodiments or aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1F. Temporal gene expression programs of the PARCB transformation model reveal SCNPC trans-differentiation pathways. See also FIG. 7. (A) Schematic summary of PARCB time course study and representative Hematoxylin and eosin (H&E) staining and immunohistochemistry (IHC) staining of neuroendocrine markers (SYP and NCAM1) on sequential tumors from the tissue microarray. Time point (TP1-6) samples were sequenced using bulk RNA sequencing (green circle), bulk ATAC-sequencing (red circle) and/or single cell RNA sequencing (blue circle, tumors only). (B) Projection of the PARCB time course samples onto the principal component analysis (PCA) framework defined by pan-cancer clinical tumor datasets 4,10,32-36). LUAD: Lung adenocarcinoma. LUAD norm: lung adenocarcinoma adjacent normal tissue. SCLC: small cell lung cancer. PRAD: prostate adenocarcinoma. PRAD norm: prostate adenocarcinoma adjacent normal tissue. CRPC: castration resistant prostate cancer. SCNPC: small cell neuroendocrine prostate cancer. (C) Average gene expression of selected SCNPC-associated proteins and markers. (D) Heatmap of hierarchical clusters (HC) of samples (columns) and corresponding differentially upregulated gene modules (rows). Differential expression defined by one HC vs all other HCs). (E) PCA analysis of the PARCB time course samples and trans-differentiation trajectories including primary arc and secondary bifurcation. (F) Selected enriched GO terms across HC.

FIG. 2A-2F. Sequential transcription regulators modulate reprogramming and neuroendocrine programs through a highly entropic and accessible chromatin state. See also FIG. 8. (A) Overall differential chromatin accessibility across HC. (B) PCA analysis of chromatin accessibility of PARCB time course samples with entropy analysis using ATAC sequencing. (C) Overall mean accessible peaks near TSS of each HC in PARCB time course study. (D) Enriched motifs from suites of transcription factors in each HC using ATAC-sequencing. Top 5 motif suites for each comparison are shown, with additional analysis in FIG. 8B, and full results in Table S4. (E) Top ranked transcription factors and known neuroendocrine transcription factors across PARCB time course using bulk RNA sequencing. HOXC TFs avg: Average expression of HOXC4, HOXC5, HOXC6, HOXC8, HOXC9, HOXC10, HOXC11, HOXC12 and HOXC13. (F) Expression of ASCL1, ASCL2, NEURDO1 and POU2F3 in each HC.

FIG. 3A-3F. Transcription factor-defined cell populations contribute to lineage divergence and tumor heterogeneity. See also FIG. 9. (A) Dimension reduction UMAP analysis of four patient series (P2, P5, P6 and P7) over time (TP3-6) using single cell RNA sequencing. (B) Temporal UMAP analysis of all the samples. (C) Expression of selected markers and transcription factors. KRT5 marks basal cells. KRT15 marks luminal cells. The expression is presented in log normalized counts. (D) Top enriched inferred cell types from the Human Cell Type Database using SingleR (48). (E) Projection of single cell RNA-seq samples on PCA framework by bulk RNA-seq samples (top panel) and the expression of selected markers and transcription factors (bottom panel). Each data point is a single cell colored by their corresponding HC. (F) Expression of ASCL1 (top) and ASCL2 (middle) and percentage of ASCL1/2 positive cells (cells with expression value >0) (bottom) in human biopsy and GEMM model tumors from five single cell RNA-seq datasets (31,49-51). Other: prostatic intraepithelial neoplasia. NMYC_RB_M: Pten^{f/f}; Rb1^{f/f}; MYCN+ (PRN) and RB_M: Pten^{f/f}; Rb1^{f/f} (PR) mouse model in Brady et al.

FIG. 4A-4G. ASCL1 and ASCL2 specify independent transcriptional programs and sub-lineages in SCNPC. See also FIG. 10. (A) Inferred clonal tracing analysis of the PARCB time course samples using Monocle 2 (52). (B) Relative expression of KRT5, ASCL1 and ASCL2 in the inferred clonal tracing analysis (pseudo-time). (C) Percentages of ASCL1 or ASCL2 positive, double positive and double negative cell populations over time. (D) Volcano plot of differential gene expression in high ASCL1+vs high ASCL2+ cell populations. (E) Representative genes from the predicted transcriptional programs of ASCL1 and ASCL2 trained on data from patient and model prostate cancer tumors (6,10,33) including TCGA), as determined by the ARACNE algorithm (81). (F) Western blot of panel of genes in the PARCB tumor derived cell lines from different tissue of origin (prostate, bladder and lung) (6,7). (G) Representative images of in situ hybridization of ASCL1 and ASCL2 mRNA analysis on transitional tumors (P7-TP5 and P9-TP4).

FIG. 5A-5F. ASCL1 and ASCL2 as pan-cancer classifiers. See also FIG. 11. (A) Projection of the PARCB time course samples on the PCA framework defined by the CRPC subtypes using RNA sequencing (left) and ATAC-sequencing (right) (57). SCL: stem-cell like. NEPC: Neuroendocrine prostate cancer. (B) Projection of the PARCB time course samples on the PCA framework defined by the SCLC subtypes (32,46). (C) mRNA expression of ASCL1 and ASCL2 in the PARCB time course samples and multiple sets of clinical CRPC-PRAD and SCNPC samples including TCGA and different research groups (10,33-36). (D) Representative images of in situ RNA hybridization of ASCL1 and ASCL2 in clinical SCNPC tissues. (E) mRNA expression of ASCL1 and ASCL2 in pan cancer cell lines (CCLE). (F) mRNA expression of ASCL1 and ASCL2 in pan cancer tumors from TCGA.

FIG. 6A-6G. Alternating ASCL1 and ASCL2 expression through reciprocal interaction and TFAP4 epigenetic regulation. See also FIG. 12. (A) Western blot analysis of exogenously expressing either V5 tagged ASCL2 in ASCL1+ cell lines (6) (left) or V5-tagged ASCL1 in ASCL2+ cell lines (right). (B) Schematic of putative cis regulatory elements (CREs) of ASCL1 and ASCL2 (top) and the heatmap of open chromatin accessibility across CREs of ASCL1 and ASCL2 using the PARCB time course ATAC-seq (bottom). Red box: CREs containing predicted TFAP4 binding sites by HOMER motif enrichment analysis (58). (C) Top 8 ranked transcription factor motifs in ASCL1 promoter and ASCL2 enhancer regions, ranked by p-values. (D) Western blot analysis of doxycycline-inducible knockout of TFAP4 and proteins of interest in P7-TP6 (ASCL1+) and P3-TP5 (ASCL2+) cell lines. DOX: doxycycline. (E) Cell proliferation analysis of P7-TP6 (ASCL1+) and P3-TP5 (ASCL2+) cell lines with doxycycline-inducible knockout of TFAP4. Ctrl: no addition of doxycycline. TFAP4: with addition of doxycycline. (G) Schematic summary of the PARCB time course study.

FIG. 7A-7G. Temporal gene expression programs of the PARCB transformation model reveal SCNPC trans-differentiation pathways. Related to FIG. 1. (A) Representative H&E staining of squamous cell carcinoma, adenocarcinoma, and small cell carcinoma in PARCB temporal tumor tissue microarray. (B) Distribution of mixed squamous/adenocarcinoma, adenocarcinoma, and mixed small cell/adenocarcinoma histology in PARCB temporal tumors. (C) Representative H&E staining images of tumors at transitional stages in PARCB temporal tumors. (D) Representative images of Immunohistochemistry staining of HLA, P63 and AR in PARCB temporal tumors. (E) PCA analysis of individual PARCB patient series (P1-P10). Note, each patient series time point is a tumor derived from the same starting material: a particular patient sample transformed by PARCB, then grown independently in different mice and harvested at the indicated time point. Thus, late time-point tumors from the same patient material, but with distinct ASCL2+ (HC5) or ASCL1+ (HC6) status do not necessarily represent late jumps between these states (i.e., P10-TP4 and P10-TP5). In other words, it is possible that the tumor in each of these individual mouse cases had at even earlier, non-sampled, time points began to commit to the ASCL2+ or ASCL1+ trajectory. (F) Two-dimensional visualization of PCA analysis of PARCB time course series using bulk RNA-sequencing. Left: PC1 vs-PC3. Right: PC1 vs PC2.

FIG. 8A-8D. Sequential transcription regulators modulate reprogramming and neuroendocrine programs through a highly entropic and accessible chromatin state. Related to FIG. 2. (A) PCA analysis of PARCB temporal samples masked with corresponding entropy scores using bulk RNA sequencing. (B) Motif enrichment analysis of HC5 (a) or HC6 (b) vs. HC1-4 (A) PCA analysis of PARCB temporal samples colored by their entropy scores based on bulk RNA sequencing. (B) Motif enrichment analysis of HC5 (a) or HC6 (b) vs. HC1-4. Note that in the GimmeMotif enrichment analysis, transcription factors are culled to minimize redundancy, and this step is impacted by the exact input data and sample group comparison indicated. Thus, each motif suite may contain slightly different enriched transcription factors. However the transcription factors set remain highly consistent between each case. Top 10 motif suites for each comparison are shown, with full results in Table S1D. (C) Adult stem cell signature 1 and SCNPC scores 2-4 of each HC. (D) PCA loading visualization of top ranked transcription factors in PC1 vs PC2 using bulk RNA sequencing.

FIG. 9A-9F. Transcription factor-defined cell populations contribute to lineage divergence and tumor heterogeneity. Related to FIG. 3. (A) UMAP analysis of single cell RNA-seq samples labeled by the time of collection (TP3-TP6) and HC (HC3-6). (B) SCNPC scores2 of each single cell. (C) Density analysis of the distribution of ASCL1 and ASCL2 expression in PARCB temporal tumors using single cell RNA sequencing. (D) PCA analysis of PARCB temporal tumor samples and corresponding expression of KRT5, ASCL1, ASCL2 and NEUROD1 using single cell RNA sequencing. (E) Single cell-based heterogeneity of top ranked transcription factors defined previously by bulk RNA sequencing (FIG. 2E), across the identified HCs. 4000 cells were randomly selected for the plot. (F) Percentage distribution of neuroendocrine marker expressing cells in the ASCL1- or ASCL2-positive population in PARCB temporal study using single cell RNA sequencing. Low/medium/high expression of CHGA/NCAM1/SYP expression are defined by the following cut-offs: low expression: [0, 1), medium: [1, 2), high: >=2.

FIG. 10A-10D. ASCL1 and ASCL2 specify independent transcriptional programs and sub-lineages in SCNPC. Related to FIG. 4. (A) Inferred clonal tracing analysis and expression of KRT5 (basal marker), ASCL1, ASCL2 and NEUROD1 by RNA Velocity 5. UMAP analysis includes the top 20 PCA dimensions. (B) Expression of previously defined ASCL2 direct targets in intestinal stem cells 6 vs. ASCL1 or ASCL2 in PARCB time course study. (C) RT-qPCR analysis of ASCL2 in 293T alone, 293T with exogenous expression of ASCL2 (ASCL2 OE) and multiple PARCB end point tumor derived cell lines. (D) Signature scores (left) and heatmaps (right) of ASCL1 and ASCL2 transcriptional programs/gene sets from HC5/6 genes (FIG. 1D) (top) and ASCL1/2 ARACNE genes (FIG. 4E) (bottom) in PARCB tumor derived cell lines with respective exogenous expression of ASCL1 or ASCL2.

FIG. 11A-11C. ASCL1 and ASCL2 as pan-cancer classifiers. Related to FIG. 5. (A) In situ RNA hybridization of ASCL1 and ASCL2 in clinical CRPC-PRAD and SCNPC tissues. (B) In situ RNA hybridization of ASCL1 and ASCL2 in the FHPCX20-01A and FHPCX20-01B CRPC PDX models. (C) Western blot analysis of selected cell lines from CCLE including lung squamous carcinoma, subtypes of SCLC, and SCNPC cell lines.

FIG. 12A-12E. Alternating ASCL1 and ASCL2 expressions through reciprocal interaction and TFAP4 epigenetic regulation. Related to FIG. 6. (A) RT-PCR of ASCL1 (left) and ASCL2 (right) in PARCB tumor derived cell lines with or without exogenous expression of V5 tagged ASCL1. (B) Western blot analysis of TFAP4 expression in multiple PARCB tumor derived cell lines. (C) Differential binding analysis of TFAP4 on ASCL1 and ASCL2 promoter and enhancer regions by CUT&RUN using TFAP4 antibody7. (D) Western blot analysis of doxycycline-inducible knockout of TFAP4 and proteins of interest in multiple SCNPC cell lines including NCI-H660 and PARCB tumors derived cell lines8. DOX: doxycycline. (E) mRNA expression of TFAP4 in normal tissue (GTEx) and pan cancer tumors (TCGA).

FIG. 13A-13C. ASCL1 and ASCL2 demonstrate mutually exclusive expression in hormonally treated, CRPC patient therapy-resistant tumor cells. Transdifferentiation to a NEPC state is a documented resistance mechanism in both prostate and lung adenocarcinomas treated with targeted therapies (anti- androgen in prostate, anti-EGFR, anti-ALK, and others in lung). Well-documented prostate cancer cases involve expression of the SCNE critical transcription factor (TF) ASCL1. Cases involving alternate subtypes and expression of alternate TFs are also observed. This includes ASCL2+, POU2F3+, and NEUROD1+ cases. These alternative critical TFs have strong parallels in the molecular pathology definitions of SCLC subtypes (Rudin et al., 2019; Huang et al., 2018). Here the inventors focus on examples of ASCL2+ therapy-resistant prostate cancer cases reported in the literature. (A-B) Bulk RNAseq data from hormonally treated and resistant prostate cancer patient tumors demonstrating a generally mutually exclusive expression pattern for ASCL1 and ASCL2 (A, Abida et al., 2019, SU2C; B, Labrecque et al., 2019 and Sharp et al., 2019). (C) Single cell RNAseq (sc-RNAseq) data from hormonally treated and resistant prostate cancer patient tumors demonstrating a generally mutually exclusive expression pattern for ASCL1 and ASCL2 (He et al., 2021). Similar mutually exclusive expression is detected in the PARCB NEPC model. Overall the single cell results are more definitive, since bulk tumors can have mixed cell populations.

FIG. 14A-14C. Candidate genes from the NEtD transition states, i.e., from the apex of the arc trajectory. Genes were ranked based on the strength of their PC3 loading from Example 1, identifying genes expressed more in transitional stages compared to early and end point stages. (A) Gene set enrichment analysis (GSEA) of the genes yielded pathways that are further described in the main text. (B-C) Examples of highly ranked individual genes matching either the enrichment analysis, or literature implicating them in functions such as inducing differentiation, inflammation or angiogenesis.

FIG. 15A-15F. Preliminary data supporting further pursuit of candidate AVIL. A-C non-prostate cancer data from Hui Li. D-F prostate cancer data. (A) Two rhabdosarcoma lines are sensitive to the AVIL inhibitor C1 (RH30 and RD), compared with more resistant mesenchymal stem cells (MSC). (B) MSC's exogenously expressing AVIL demonstrate differentiation changes and become sensitive to AVIL inhibition by drug C1. (C) Drug C1 and its derivative drug have anti-tumor efficacy against RD cell line sub-cutaneous xenografts. Drugs were intraperitoneal (IP) injected after one week of tumor inoculation, every three days. (D) Exogenous expression of AVIL in LNCaP C4-2B prostate adenocarcinoma cell line (with p53 and RB1 disrupted by genetic engineering), results in upregulation of the NEPC-associated TF FOXM1. Experiments underway to clarify the double AVIL isoform bands. (E-F) AVIL inhibitor C1 sensitivity of three PARCB model-derived NEPC cell lines. As hypothesized from experiments in other cancer types, increased AVIL expression correlates with more sensitivity to the AVIL inhibitor. Note, PARCB lines have similar sensitivity to AVIL inhibition as rhabdosarcoma lines, where AVIL's oncogenic driver properties were first discovered (Xie et al., PNAS, 2016) . . .

FIG. 16A-16B. Alternating ASCL1 and ASCL2 expression through reciprocal interaction. (A-B) Immunoblot analysis of ASCL1 or ASCL2 knockdown at the organoid stage of the PARCB model (A), or overexpression of either V5 tagged ASCL2 in ASCL1+ PARCB model-derived cell lines (B, left) or V5-tagged ASCL1 in ASCL2+ PARCB model-derived cell lines (B, right). Increased (decreased) ASCL1 protein expression leads to increased ASCL2 expression, and increased (decreased) ASCL2 expression leads to decreased (increased) ASCL1 expression. Thus, in the inventors' model cells ASCL1 and ASCL2 mutually regulate each other at the protein level, but each in the opposite manner.

FIG. 17A-17C. ASCL1 and ASCL2 have a shared positive regulatory factor, TFAP4. The ATAC-seq results combined with an extensive literature search of all the factors whose motifs were found in both ASCL1 and ASCL2 regulatory regions, revealed that TFAP4 (a.k.a. AP-4) was reported to form different transcription complex to either activate or repress target genes and thus mediate cell fate decisions. (A) Subsequent differential binding analysis of TFAP4 on ASCL1 and ASCL2 promoter and enhancer regions by CUT&RUN. (B) Immunoblot analysis of ASCL1+ and ASCL2+ cell lines containing inducible CRISPR sgRNA targeting TFAP4. DOX: Doxycycline. (C) Cell proliferation analysis of ASCL1+ and ASCL2+ cell lines containing inducible CRISPR sgRNA targeting TFAP4. Ctrl: No added doxycycline. TFAP4 KO: knockout of TFAP4 with the addition of doxycycline.

### DETAILED DESCRIPTION OF THE INVENTION

### I. Inhibitors

### A. Inhibitory Oligonucleotides

The disclosure provides for inhibitory oligonucleotides that inhibit the gene expression of a target gene. Examples of an inhibitory oligonucleotides include but are not limited to siRNA (small interfering RNA), short hairpin RNA (shRNA), double-stranded RNA, an antisense oligonucleotide, a ribozyme and a oligonucleotide encoding thereof. An inhibitory oligonucleotide may inhibit the transcription of a gene or prevent the translation of a gene transcript in a cell. An inhibitory oligonucleotide acid may be from 16 to 1000 nucleotides long or from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of the protein by at least 10%, 20%, 30%, or 40%, more particularly by at least 50%, 60%, or 70%, and most particularly by at least 75%, 80%, 90%, 95%, 99%, or 100% more or any range or value in between the foregoing.

Also described are synthetic oligonucleotides that are inhibitors. An inhibitor may be between 17 to 25 nucleotides in length and comprises a 5′ to 3′ sequence that is at least 90% complementary to the 5′ to 3′ sequence of a mature mRNA. An inhibitor molecule may be 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

The inhibitory oligonucleotide may be an analog and my include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. The oligonucleotide may be further defined as having one or more locked nucleotides, ethylene bridged nucleotides, peptide nucleic acids, or a 5′ (E)-vinyl-phosphonate (VP) modification. The oligonucleotides may have one or more phosphorothioated DNA or RNA bases.

### B. Antibodies

An antibody or a fragment thereof may be one that binds to at least a portion of a target gene and modulates it's activity, such as its binding activity, enzymatic activity, or binding specificity.

Also described are antibodies comprising a heavy or light chain, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (Δ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the—COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)₂, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following: The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. Bispecific antibodies can be biparatopic, wherein a bispecific antibody may specifically recognize a different epitope from the same antigen. Bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

The antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

Multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

Affinity matured antibodies may be enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

Portions of the heavy and/or light chain may be identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

Minimizing the antibody polypeptide sequence from the non-human species may optimize chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Also provided are antibody fragments, such as antibody fragments that bind to and modulate activity. The term functional antibody fragment includes antigen-binding fragments of an antibody that retain the ability to specifically bind to an antigen. These fragments are constituted of various arrangements of the variable region heavy chain (VH) and/or light chain (VL); and include constant region heavy chain 1 (CHI) and light chain (CL). They may lack the Fc region constituted of heavy chain 2 (CH2) and 3 (CH3) domains. Antigen binding fragments and the modifications thereof may include: (i) the Fab fragment type constituted with the VL, VH, CL, and CHI domains; (ii) the Fd fragment type constituted with the VH and CHI domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)₂ fragment means a bivalent antigen-binding fragment of a monoclonal antibody comprising two Fab′ fragments linked by a disulfide bridge at the hinge region. An F(ab′)₂ fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### Fragment Crystallizable Region, Fc

A fragment crystallizable region (Fc region) contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

Antigen-binding peptide scaffolds, such as complementarity-determining regions (CDRs), are used to generate protein-binding molecules. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

II. Additional Agents

### A. Immunostimulators

The method may further comprise administration of an additional agent. The additional agent may be an immunostimulator. The term “immunostimulator” as used herein refers to a compound that can stimulate an immune response in a subject, and may include an adjuvant. An immunostimulator may be an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

The additional agent may comprise an agonist for pattern recognition receptors (PRR), including, but not limited to Toll-Like Receptors (TLRs), specifically TLRs 2, 3, 4, 5, 7, 8, 9 and/or combinations thereof. Additional agents may comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. The additional agents also may comprise immunostimulatory RNA molecules, such as but not limited to dsRNA, poly I:C or poly I:poly C12U (available as Ampligen.RTM., both poly I:C and poly I:polyC12U being known as TLR3 stimulants), and/or those disclosed in F. Heil et al., “Species-Specific Recognition of Single-Stranded RNA via Toll-like Receptor 7 and 8” Science 303 (5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. An additional agent may be a TLR-4 agonist, such as bacterial lipopolysaccharide (LPS), VSV-G, and/or HMGB-1. Additional agents may comprise TLR-5 agonists, such as flagellin, or portions or derivatives thereof, including but not limited to those disclosed in U.S. Pat. Nos. 6,130,082, 6,585,980, and 7,192,725.

Additional agents may be proinflammatory stimuli released from necrotic cells (e.g., urate crystals). Additional agents may be activated components of the complement cascade (e.g., CD21, CD35, etc.). Additional agents may be activated components of immune complexes. Additional agents also include complement receptor agonists, such as a molecule that binds to CD21 or CD35. The complement receptor agonist may induce endogenous complement opsonization of the synthetic nanocarrier. Immunostimulators may be cytokines, which are small proteins or biological factors (in the range of 5 kD-20 kD) that are released by cells and have specific effects on cell-cell interaction, communication and behavior of other cells. The cytokine receptor agonist may be a small molecule, antibody, fusion protein, or aptamer.

### B. Immunotherapies

The additional therapy may comprise a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

The immunotherapy may comprise an inhibitor of a co-stimulatory molecule. The inhibitor may comprise an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta).

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

The additional therapy may comprise immune checkpoint inhibitors. Certain aspects are further described below.

### a. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be further defined as human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DClg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHlgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

### b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

The inhibitor may comprise the heavy and light chain CDRs or VRs of tremelimumab or ipilimumab. Accordingly, the inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may be one that competes for binding with and/or binds to the same epitope on PD-1, B7-1, or B7-2 as the above-mentioned antibodies. The antibody may be one that has at least about 70, 75, 80, 85, 90, 95, 97, or 99% (or any derivable range therein) variable region amino acid sequence identity with the above-mentioned antibodies.

### C. Oncolytic Virus

The additional therapy may comprise an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### D. Polysaccharides

The additional therapy may comprise polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

The additional therapy may comprise neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### F. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated. The amount of cisplatin delivered to the cell and/or subject, when used in combination with the inhibitors described herein, may be less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. Appropriate intravenous doses for an adult may include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

The additional therapy or prior therapy may comprise radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

The amount of ionizing radiation may be greater than 20 Gy and is administered in one dose. The amount of ionizing radiation may be 18 Gy and may be administered in three doses. The amount of ionizing radiation may be at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of IR may be presented as a total dose of IR, which is then administered in fractionated doses. For example, the total dose is 50 Gy administered in 10 fractionated doses of 5 Gy each. The total dose may be 50-90 Gy, administered in 20-60 fractionated doses of 2-3 Gy each. The total dose of IR may be at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the inhibitors of the disclosure, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Other Agents

It is contemplated that other agents may be used in combination with certain aspects of the disclosure to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. Cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present disclosure to improve the treatment efficacy.

### III. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, the first cancer therapy and the second cancer therapy are administered substantially simultaneously. In some embodiments, the first cancer therapy and the second cancer therapy are administered sequentially. In some embodiments, the first cancer therapy, the second cancer therapy, and a third therapy are administered sequentially. In some embodiments, the first cancer therapy is administered before administering the second cancer therapy. In some embodiments, the first cancer therapy is administered after administering the second cancer therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the first cancer therapy comprises a first cancer protein, a nucleic acid encoding for the first cancer protein, a vector comprising the nucleic acid encoding for the first cancer protein, or a cell comprising the first cancer protein, a nucleic acid encoding for the first cancer protein, or a vector comprising the nucleic acid encoding for the first cancer protein. In some embodiments, a single dose of the first cancer protein therapy is administered. In some embodiments, multiple doses of the first cancer protein are administered. In some embodiments, the first cancer protein is administered at a dose of between 1 mg/kg and 5000 mg/kg. In some embodiments, the first cancer protein is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some embodiments, a single dose of the second cancer therapy is administered. In some embodiments, multiple doses of the second cancer therapy are administered. In some embodiments, the second cancer therapy is administered at a dose of between 1 mg/kg and 100 mg/kg. In some embodiments, the second cancer therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 M; or about 1 μM to 50 μM; or about 1 μM to 40 M; or about 1 μM to 30 M; or about 1 μM to 20 μM; or about 1 μM to 10 μM; or about 10 μM to 150 μM; or about 10 M to 100 μM; or about 10 μM to 50 μM; or about 25 μM to 150 μM; or about 25 μM to 100 μM; or about 25 μM to 50 M; or about 50 μM to 150 M; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above . . .

### IV. Pharmaceutical Compositions

In some embodiments, pharmaceutical compositions are administered to a subject. Different aspects may involve administering an effective amount of a composition to a subject. In some embodiments, an antibody or antigen binding fragment capable of binding to [protein of interest] may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Temporal Evolution Reveals Bifurcated Lineages in Aggressive Neuroendocrine Small Cell Prostate Cancer Trans-Differentiation

Trans-differentiation from an adenocarcinoma to a small cell neuroendocrine state is associated with therapy escape in multiple cancer types. To gain insight into the molecular events that promote resistance via cancer trans-differentiation, the inventors performed a multi-omics time course analysis of a pan-small cell neuroendocrine cancer model (termed PARCB), a forward genetic transformation using human prostate basal cells. With integrative analyses of RNA sequencing and ATAC sequencing, a shared developmental arc-like trajectory is identified among all transformed patient samples. Further mapping with single cell resolution reveals two distinct lineages defined by mutually exclusive expression of ASCL1 or ASCL2. Temporal regulation by groups of transcription factors across developmental stages reveals that cellular reprogramming precedes the induction of neuronal programs. Lastly, TFAP4 and ASCL1/2 feedback are identified as potential regulators of ASCL1 and ASCL2 expression. This study provides temporal transcriptional patterns and uncovers pan-tissue parallels between prostate and lung cancers, as well as connections to normal neuroendocrine cell states. As additional successful targeted therapies come to the clinic, resistance mechanisms involving changes in cell identity stand to further expand.

### A. Introduction

Small cell neuroendocrine (SCN) cancer is an aggressive variant that arises from multiple tissues such as the lung and prostate (1,2). SCN is characterized by its histologically defined small cell morphology of densely packed cells with scant cytoplasm, poor differentiation, and aggressive tumor growth, as well as expression of canonical neuroendocrine markers including SYP, CHGA and NCAM1 (3). In addition to their phenotypic resemblance, SCN cancers across multiple tissues show a striking transcriptional and epigenetic convergence in clinically annotated tumors (4,5). This molecular signature convergence is recapitulated by the inventors' established SCN transformation model that utilizes either normal lung epithelial cells, patient-derived benign prostate epithelial or bladder urothelial cells as the cells of origin (6,7).

Small cell neuroendocrine prostate cancer (SCNPC) occurs either de novo (<1% of untreated prostate cancer cases), or through therapy-mediated transversion of castration resistant prostate cancer (CRPC) (˜20% of the resistance cases). The terminology SCNPC is canonical for the prostate cancer field, while the SCN terminology has been adopted to reflect the shared pan-tissue aspects of multiple SCN tumors, such as small cell lung cancer (SCLC). CRPC is a resistant variant of prostate adenocarcinoma (PRAD), which often responds to androgen deprivation therapy (8,9). Trans-differentiation from PRAD to the SCNPC state entails complicated epigenetic reprogramming at the chromatin level, resulting in transcriptional changes driven by a number of key master regulators (10,11). For example, methylation modulated by EZH2 and activation of transcriptional programs by SOX2 are required in TP53 and RB1 loss-mediated neuroendocrine differentiation in mouse transgenic models of SCNPC (12,13). Oncogenic mutation of FOXA1 potentiate pioneering activity and differentiation status of prostate cancer (14,15). Lastly, knockdown of transcription factors such as ONECUT2 has been shown to inhibit SCN differentiation (16,17). While the importance of these factors has been demonstrated, the chronological sequence of the associated epigenetic and transcriptional changes remains uncharacterized during the progression to SCNPC. Examination of the temporal evolution of lung cancer revealed a connection between transcription factor defined subtypes and cell plasticity (18,19). In this study, the inventors sought to answer the following questions: 1) when do SCN-associated transcription factors emerge during SCNPC progression, 2) how do they coordinate SCN differentiation, and 3) can one identify a transition state defined by transcription factors that can be targeted?

Leveraging the inventors' previously developed human pan-small cell neuroendocrine cancer model, the PARCB forward genetics transformation model (driven by knockdown of RB1, alongside exogenous expression of dominant negative TP53, cMYC, BCL2 and myristoylated AKT1 via three lentiviral vectors) (6,7), tumor samples were harvested at different time points for multi-omics analyses. The transcriptional and epigenetic status of each time point was determined using integrative bulk RNA sequencing, ATAC sequencing, and single cell RNA sequencing. This longitudinal study provides insight into the temporal evolution of the epigenetic and transcriptional landscape during trans-differentiation and small cell cancer progression. The inventors found consistent transcriptional patterns and differentiation trajectories across samples generated from independent patient tissues, as well as a bifurcation of end-stage neuroendocrine lineages, defined by ASCL1 and ASCL2 and their associated programs.

### B. RESULTS

### 1. Temporal Gene Expression Programs of the PARCB Transformation Model Reveal SCNPC Trans-Differentiation Pathways

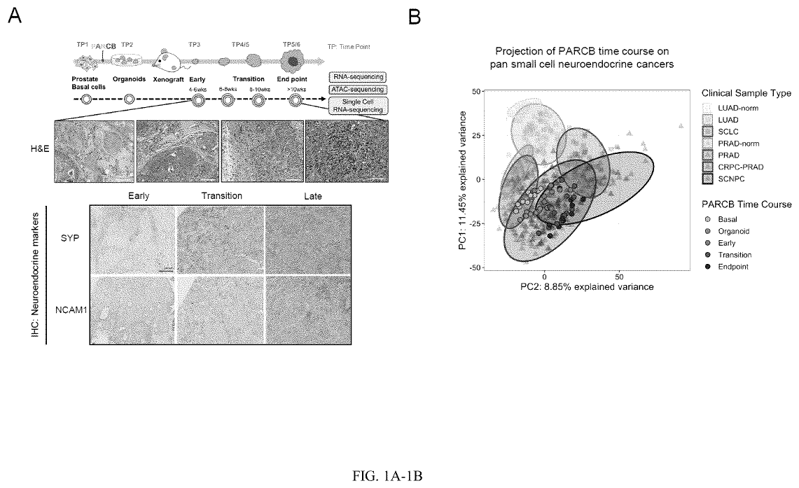

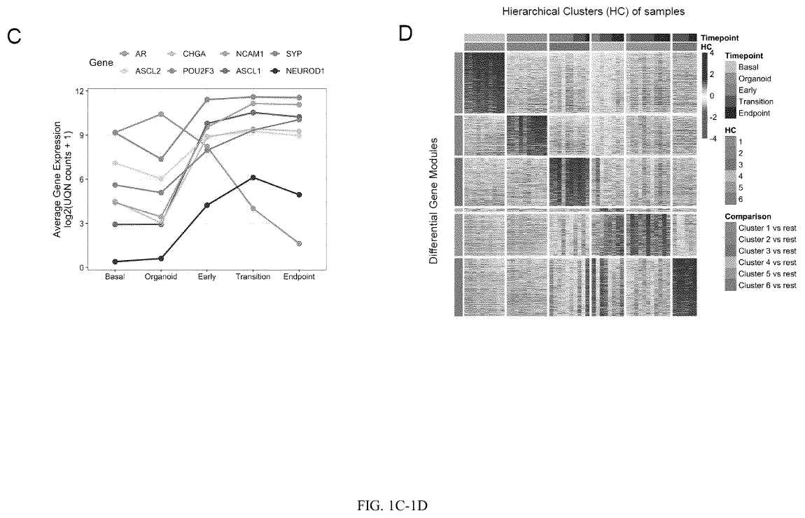

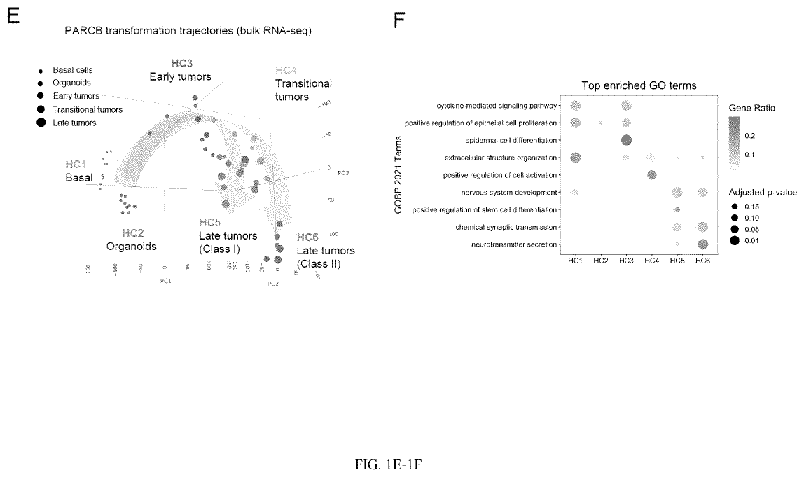

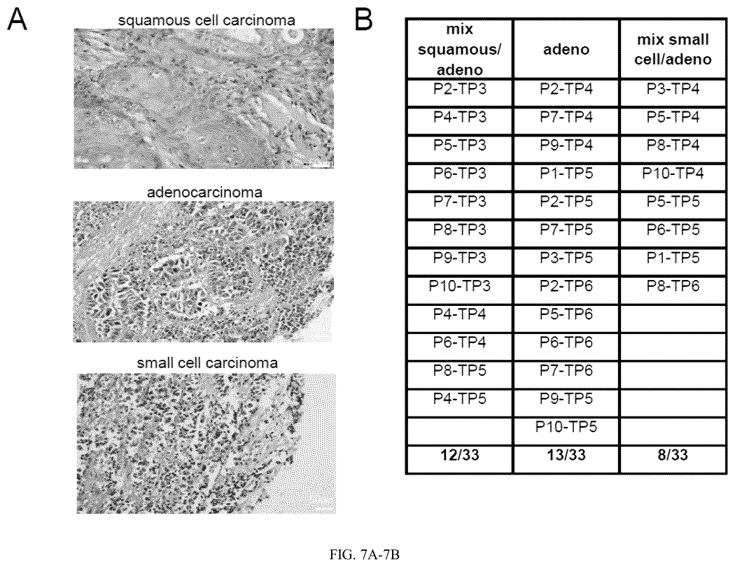

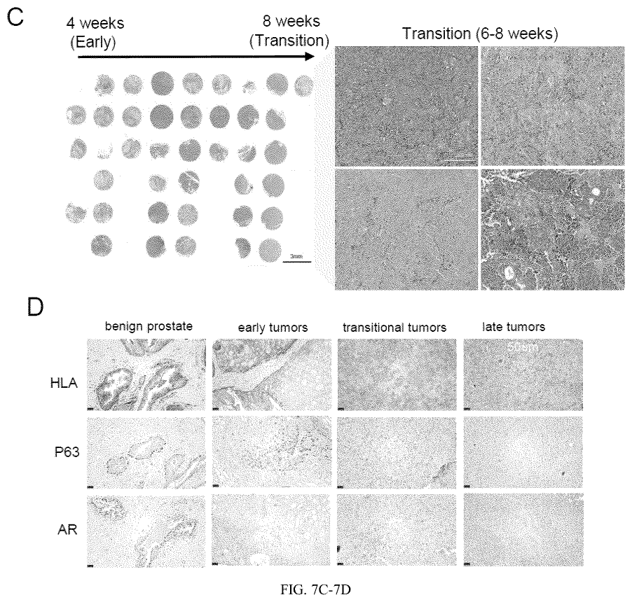

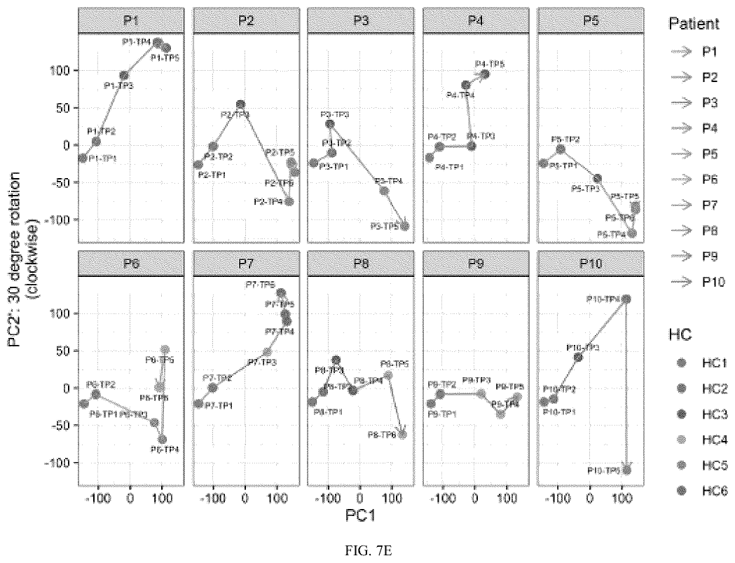

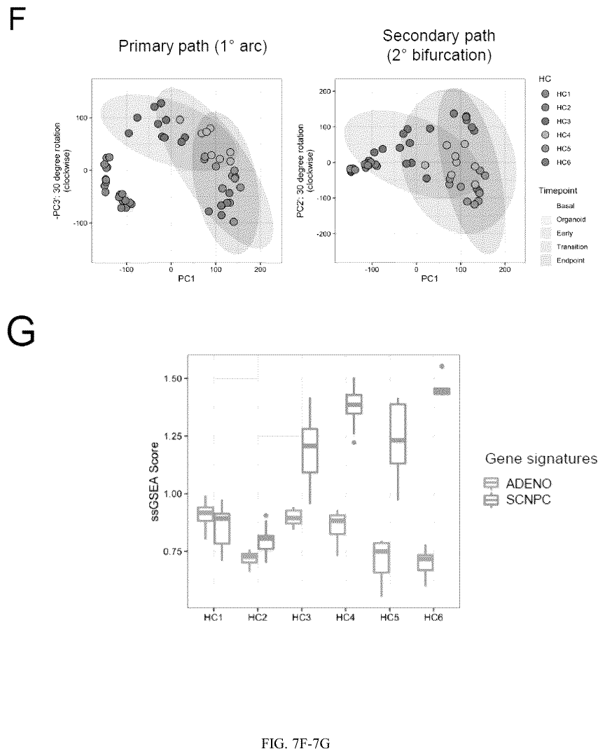

To determine the timing of SCN differentiation events during prostate cancer development, the inventors utilized the PARCB model system (6). Independent transformations were performed on basal cells extracted from benign regions of epithelial tissue from 10 prostate adenocarcinoma patients. Basal cells were transformed by the oncogenic lentiviral PARCB cocktail and subsequently cultured in an organoid system in vitro (6). Transformed organoid-expanded cells from each patient tissue sample were subcutaneously implanted into multiple immunocompromised mice to allow for time-course collection of tumors from the matched starting material (FIG. 1A). The tumors were collected at approximately two-week intervals until reaching 1 cm³ in size or occurrence of ulceration, whichever came first. The transformed tumor cells were triply fluorescent due to the lentiviral integration (6), which allowed for cancer cell purification by fluorescence-activated cell sorting (FACS) followed by multi-omics sequencing and analysis (FIG. 1A). Each patient series (P1-P10) contains five to six time point samples ranging from basal cells (TP1) to organoids (TP2) to tumors (TP3-TP5/TP6) (FIG. 1A). Upon histological examination of the tumor issues by pathologists, the inventors found that the time course tumors transitioned from squamous, to adenocarcinoma, then to mixed and eventually SCN phenotypes (FIG. 1A and FIG. 7A-C). Furthermore, clinically defined neuroendocrine markers, including SYP and NCAM1, emerge during the transition to late stages of the tumor progression (FIG. 1A). The basal cell marker p63 were only positive in early-stage tumors by immunohistochemistry (IHC) staining (FIG. 7D).

The inventors first performed a temporal analysis of gene expression using bulk RNA sequencing to understand the changes in the transcriptional landscape during SCNPC trans-differentiation. By projection of PARCB samples onto principal component analysis (PCA) of clinical lung and prostate cancer tumor samples (4,10,32-36), the inventors validated that PARCB time course samples follow the transcriptionally defined convergence trajectory from adenocarcinoma to SCN states (FIG. 1B and FIG. 7E). Additional SCNPC associated factors including ASCL1 and NEUROD1 were also elevated during the progression (FIG. 1C). Despite that the mRNA of androgen receptor (AR) was expressed in tumors at the early stage (FIG. 1C), the protein level was not detectable by immunostaining (FIG. 7D). Taken together, the histological and omics data indicate that PARCB time course tumors recapitulate both the phenotypic and transcriptional changes observed in the clinic and provide a model system for studying the temporal evolution of SCNPC development.

To determine the transformation trajectories among the time course series generated from the 10 independent patient samples (P1-P10), the inventors performed clustering and PCA analysis of the transcriptomic data. To account for potential asynchronous development among each patient series and each individual tumor, the inventors defined hierarchical clusters (HCs) of samples by their corresponding differential gene modules and found the resulting 6 clusters (HC1-6) to generally correspond with the time of collection (FIG. 1D and Table 1). This provides a clustering-based trans-differentiation reference frame and informs the subsequent multi-omics analyses. Unsupervised PCA analysis demonstrates that the individual transformation paths of each series follow a generally consistent “arc-like” trajectory with a discernable bifurcation in late-stage samples (FIG. 1E and FIG. 7E-F). The late tumors were hence further defined as “Class I” and “Class II” tumors with correspondent HC5 and HC6 gene modules, respectively. HC2 to HC6 had elevated SCNPC signature scores compared to adenocarcinoma signature score (FIG. 7G). This last finding supports the existence of two transcriptional programs or end points defining the terminal SCNPC tumor phenotypes.

Gene ontology enrichment analysis of the corresponding 6 differential gene modules identified biological processes enriched uniquely or shared among HCs, including Inflammatory response (HC1 and HC3, patient derived basal cells and early tumors, respectively), cell proliferation (HC2, in vitro organoids), epidermis development (HC3, early tumors), cell activation (HC4, transitional tumors), stem cell differentiation (HC5, Class I late tumors) and neuro-/chemical synapse (HC5 and HC6, both classes of late tumors) (FIG. 1E and Table 2). The transcriptome evolution supports the idea that trans-differentiation from adenocarcinoma to the SCN state is a systematically coordinated process, that involves a transitional stage followed by bifurcated pathways enriched in neuronal/neuroendocrine gene signatures.

### 2. Sequential Transcription Regulators Modulate Reprogramming and Neuroendocrine Programs Through a Highly Entropic and Accessible Chromatin State

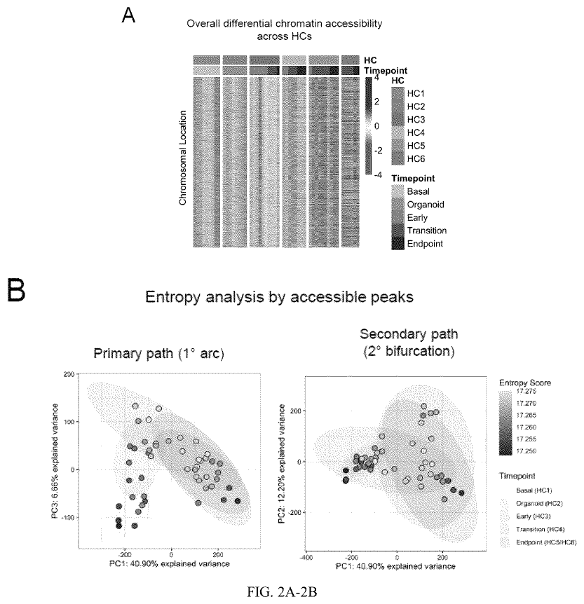

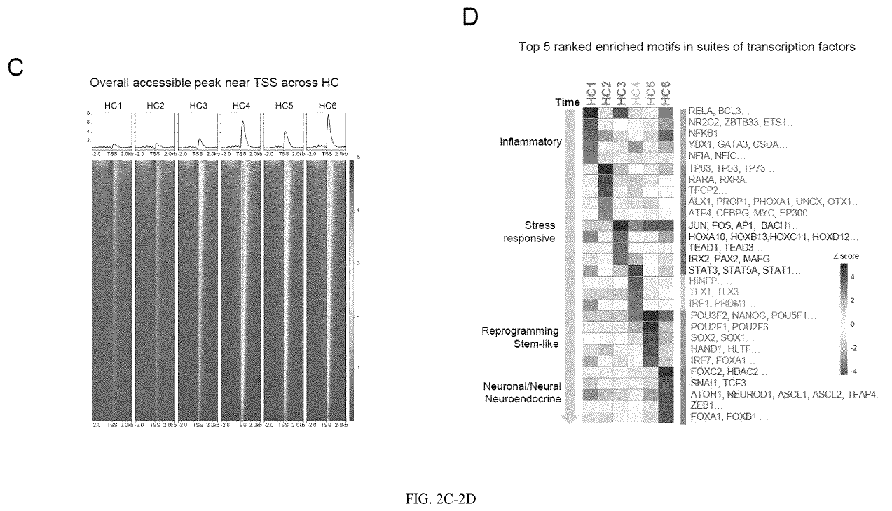

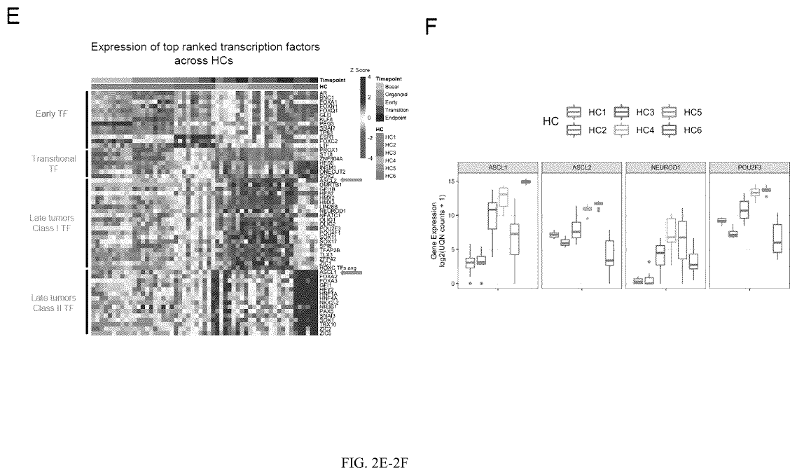

Temporal analyses on single transcription factor defined subtypes of small cell lung cancer (SCLC) models have delineated lineage plasticity in the development of lung neuroendocrine tumors (18). The inventors sought to define the transcriptional evolution in SCNPC through an extensive survey of over 1600 transcription factors (37) by chromatin accessibility analyses using ATAC sequencing (38). A significant increase in overall accessible chromatin peaks across chromosomes is observed starting at the tumors at transitional stage (HC4) to late stages (HC5 and HC6) (FIG. 2A). Unsupervised PCA analysis using ATAC-seq data showed an arc-like and bifurcated trajectory consistent with the pattern observed using the RNA-seq data (FIGS. 1D and 2B). The Shannon entropy has been used to estimate the plasticity potential of a biological sample to change cellular state (39,40). The inventors found that transitional samples (HC4) have the highest entropy (FIG. 2B), suggesting there exists a high potential and less well-defined transcriptional state during the trans-differentiation process.

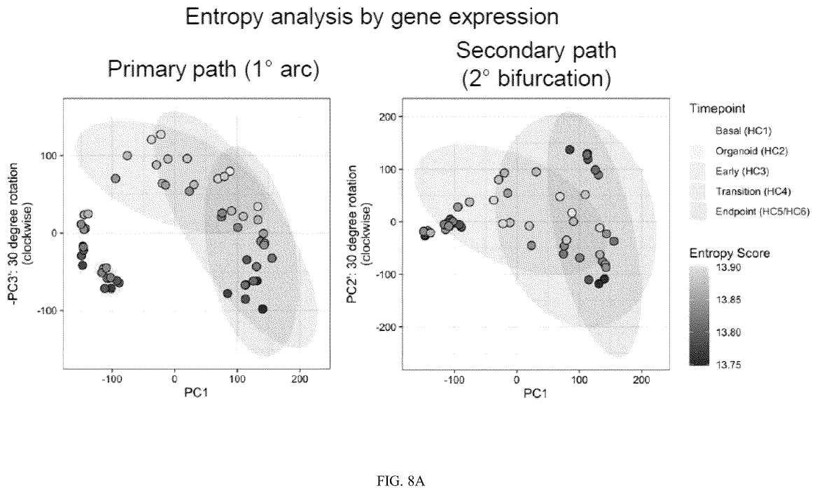

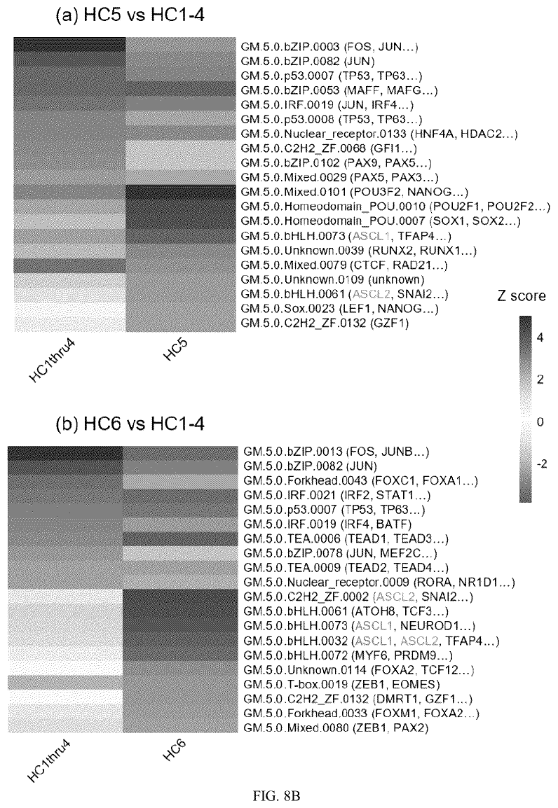

To identify transcription factors that recognize the chromatin accessible regions at each stage of the transformation trajectory, the inventors first looked at the overall accessibility near the transcription start sites (TSS) (FIG. 2C). Transitional samples (HC4) have a strong increase in the accessible peaks as estimated by Shannon entropy calculations (FIGS. 2B and 2C), consistent with the gene-expression-based entropy calculations (FIG. 8A). Next, motif enrichment analysis was performed on the accessible peaks from each HCs in a “one versus the rest” fashion. Since transcription factors from the same family share similar motifs and are deposited into a variety of databases, the inventors used a pipeline that applies an ensemble of existing computational tools and suites of motifs (de novo and known) (41) (FIG. 2D). Motif enrichment analysis implicated that 1) representative stress-responsive factors such as NFκB, JUN, ATF and STAT proteins were active from early to transition stage (HC1-4), 2) reprogramming factors such as POU/OCT and SOX families were active in Class I (HC5) tumors, and 3) neuronal/neural factors including ASCL and NEUROD family proteins were found at the later stage in Class II (HC6) tumors (FIG. 2D). Due to ASCL1, ASCL2 and other bHLH factors sharing the same E-box motif, and the stringent “one HC versus the rest” differential accessibility analysis, the motif suite containing ASCL1 and ASCL2 factors is highly enriched and ranked in HC6 compared to HC5 (FIG. 2D). Nonetheless, when HC6 is left out of the analysis, HC5 does demonstrate strong enrichment for the motif suite containing ASCL1 and ASCL2 factors, compared to HC1-4. (FIG. 8B). The enrichment of stem-like and neuroendocrine programs in HC4-5 and HC6, respectively, was further confirmed by signature scores derived from the literature (33,42) (FIG. 8C). This analysis provides a view of the overall transcriptional shift of the chromatin accessibility during trans-differentiation.

To determine whether expression of the transcription factors corresponds to their inferred activity from the motif enrichment analysis, the inventors summarized the top ranked transcription factors (based on PC1, PC2 and PC3 loadings) across the transformation stages (HC1-HC6) (FIG. 2E and FIG. 8D, Table 3) from the perspective of the PCA-based transformation trajectory (FIG. 1E and FIG. 7F). Overall, the inventors observed that 1) AR mRNA expression is lost during progression towards late-stage tumors, 2) FOXA1, a known transcription factor of SCNPC (14,15), is shown to emerge at the early-transition stage, and 3) well-known neuroendocrine transcription factors such as ASCL1, NEUROD1, ONECUT2, SOX2, INSM1 and FOXA2 were increased towards the late stage (FIG. 2E and FIG. 8D) (43-45). This analysis also revealed additional candidate stage-specific transcription factors that are largely understudied in SCNPC, such as LTF, ESR1, ZIC2 and TBX10 (FIG. 2E). ASCL1 and ASCL2 expression were elevated in the late tumor stages (FIG. 1C and FIG. 2E-F). Notably, their expression was enriched in separate tumor endpoints (HC5: ASCL2+ and HC6: ASCL1+), supporting their probable contribution to the bifurcated trajectories (FIG. 2E-F and FIG. 8D).

### 3. Transcription Factor-Defined Cell Populations Contribute to Lineage Divergence and Tumor Heterogeneity

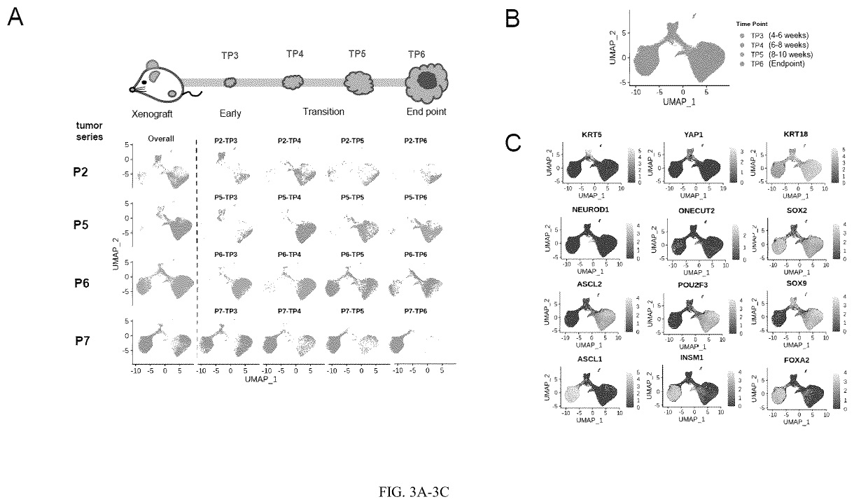

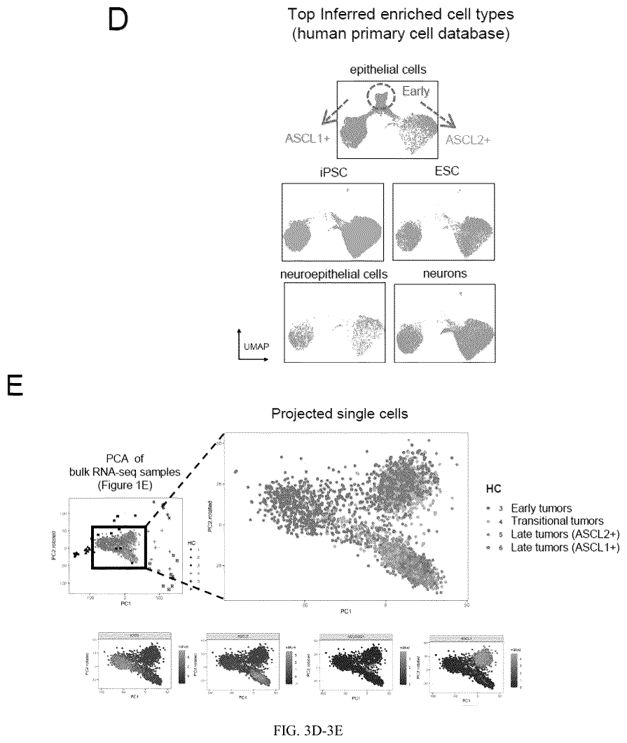

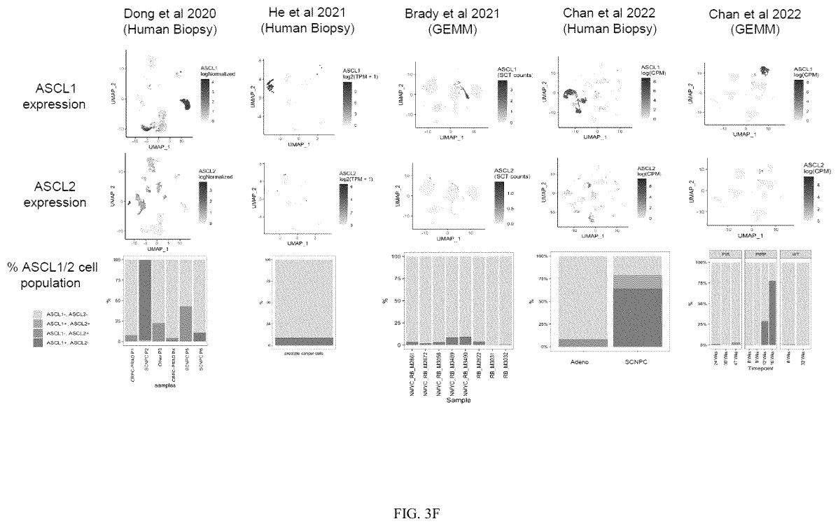

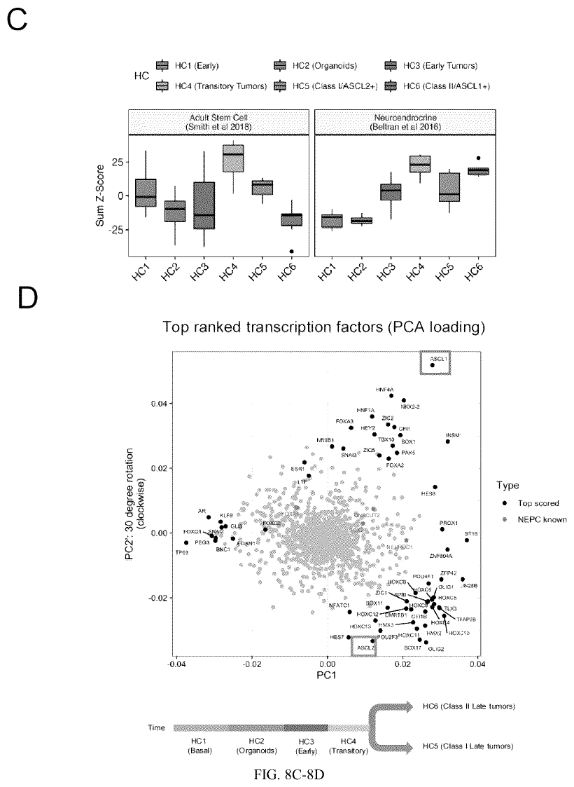

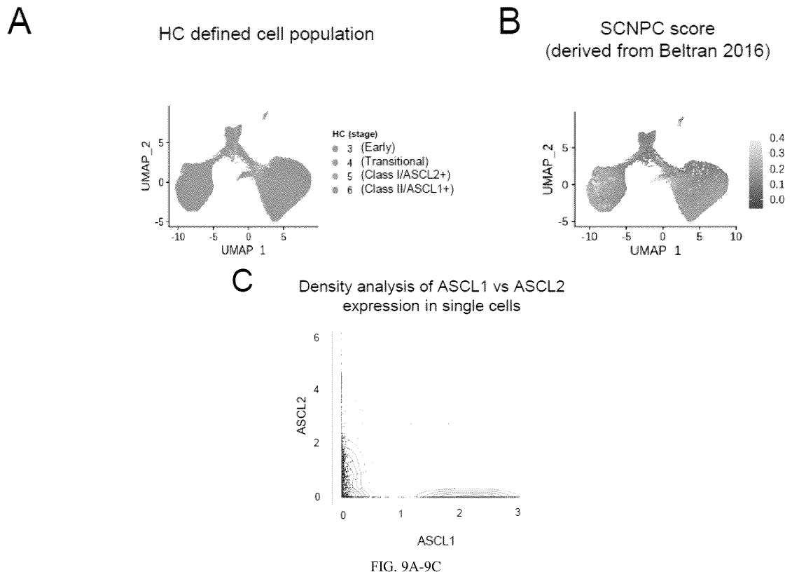

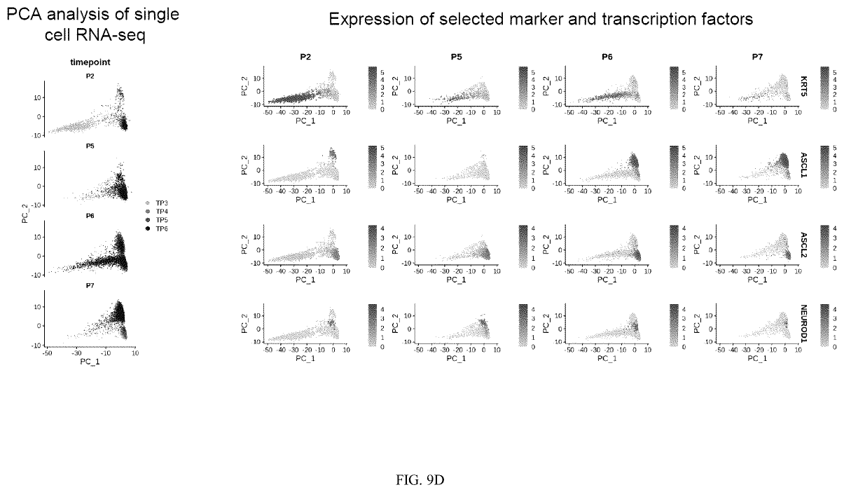

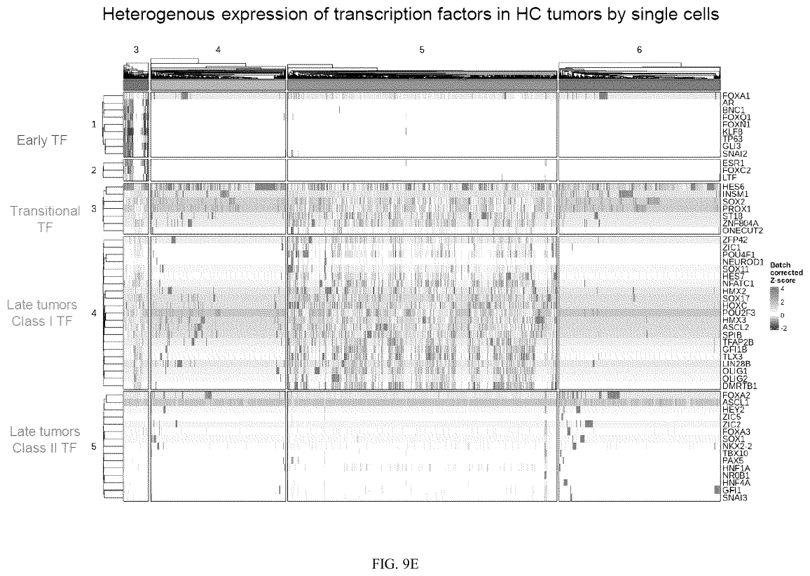

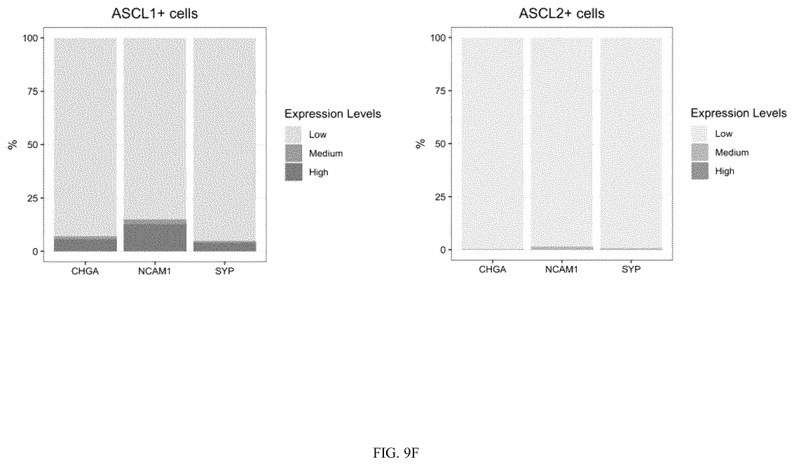

To determine the degree of heterogeneity within the time course tumors, the inventors performed single cell RNA sequencing on four time-defined serial tumor sets: P2, P5, P7 and P8 (TP3-TP6) (FIG. 3A). Dimension reduction analysis (UMAP) was used to visualize the overall distribution of cell populations at each time point of SCNPC development (FIG. 3A-B and FIG. 9A). Overall, a lineage differentiation from basal (KRT5+) to luminal (KRT18+) was observed from early to late tumors (FIG. 3B-C). YAP1, whose expression defines a non-neuroendocrine SCLC subtype (46) and whose high expression is frequently seen in CRPC-PRAD but not SCNPC (47), is enriched in the early tumor cell populations (FIG. 3B-C).

To understand the association of known SCN transcription factors in contributing to intra-tumoral heterogeneity, the inventors first assigned a SCNPC score (33) to each cell (FIG. 9B). Despite the high SCNPC scores across populations of single cells, the number of NEUROD1 and/or ONECUT2 positive cells is very low, while deeper single cell sequencing depth would be required to fully investigate this result (FIG. 3C and FIG. 9B). Other well-known neuroendocrine transcription factors such as ASCL1, INSM1 and FOXA2 are enriched in the same cell cluster with high SCNPC score (FIG. 3C and FIG. 9B). However, in another cell cluster, ASCL2, POU2F3 and SOX9 were co-expressed with a medium level of SCNPC score (FIG. 3C and FIG. 9B). The general mutual exclusivity of ASCL1 and ASCL2 in single cells further supports ASCL1 and ASCL2 contributing to the bifurcated endpoint trajectories observed in the bulk tumors (FIG. 3C and FIG. 9C-D).

Single cell datasets available as reference from longitudinal clinical samples in advanced prostate cancer are rare, thus a cell type inference analysis using reference pure cell types was applied to infer the identity of individual cells in PARCB tumors (48). Five out of a total of 36 reference cell types from the Human Primary Cell Database were highly enriched in the PARCB time course tumor samples (FIG. 3D). All tumor cells share a similar transcriptome as epithelial cells (FIG. 3D). Particularly, a majority of tumor cells (other than early stage cells) exhibit stem-like gene expression patterns reflective of embryonic stem cells and induced pluripotent stem cells, indicative of a de-differentiation shift during SCNPC development and trans-differentiation (FIG. 3D). Additionally, later-stage cells expressing either ASCL1 or ASCL2 had neuronal-like gene expression profiles, confirming the emergence of SCN differentiation (FIG. 3B-D).

Single cell analysis supports the overall gene expression and chromatin accessibility patterns observed in bulk tumors. Projection of single cells onto the PCA framework generated from bulk RNA-seq samples (FIG. 1E and FIG. 7F) demonstrated that tumors clustering distinctly by bulk RNA-seq indeed consist primarily of single cells in the corresponding different transcriptional states, with some degree of heterogeneity (FIG. 3E). Furthermore, transcription factors with high expression in tumors defined by bulk RNA-sequencing analysis (FIG. 2E) show heterogenous patterns among single cells (FIG. 9E). Tumors at transitional stage (HC4) and late stage (HC5) have the highest degree of gene fluctuation, further highlighting a potential role for increased intratumoral heterogeneity during the trans-differentiation process (FIG. 9E). Importantly, the inventors further validated the mutually exclusive expression pattern of ASCL1 and ASCL2 in multiple clinical and GEMM single cell RNA-seq datasets (31,49-51). This analysis confirmed that ASCL2 is generally enriched in non-NE cells/adenocarcinoma and ASCL1 is more abundant in high NE cells/SCNPC clinically (FIG. 3F), consistent with the PARCB temporal study (FIG. 9F). ASCL1 and ASCL2 double-positive cells are observed at a low frequency, primarily in SCNPC tumors, and may reflect a transitional state between adenocarcinoma and SCN phenotypes (FIG. 3F).

### 4. ASCL1 and ASCL2 Specify Independent Transcriptional Programs and Sub-Lineages in SCNPC

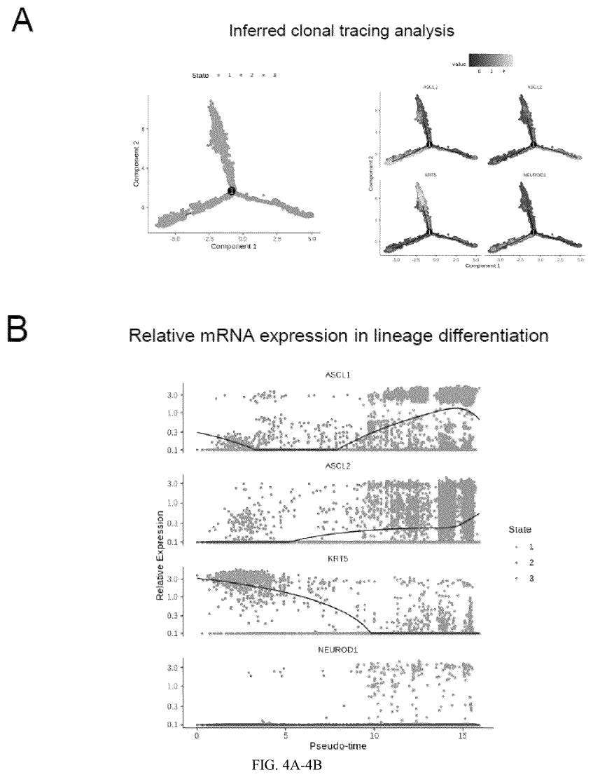

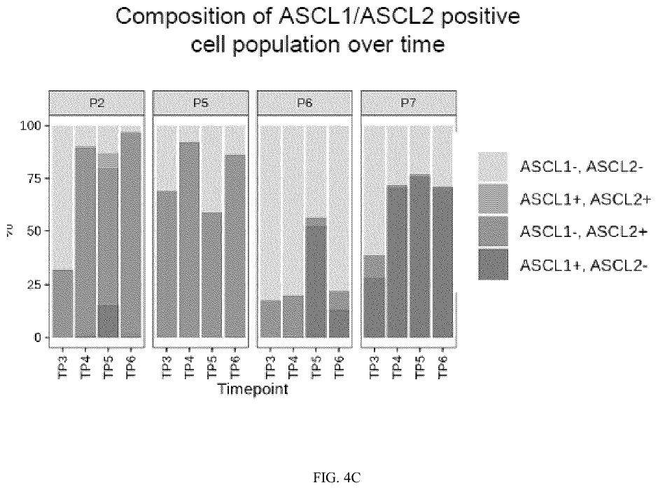

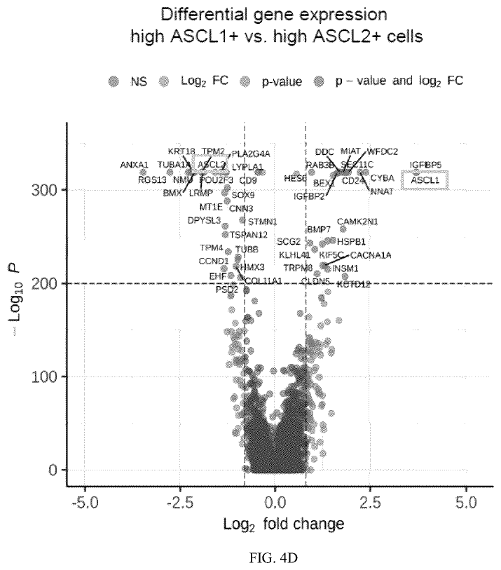

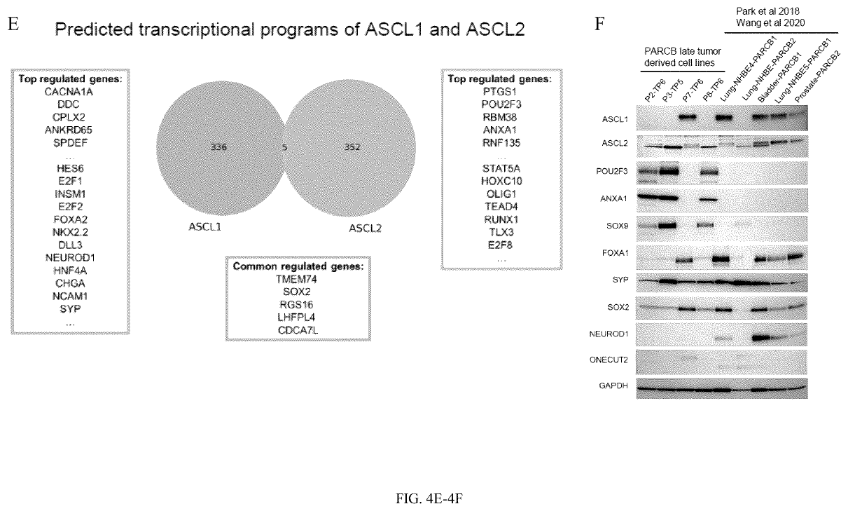

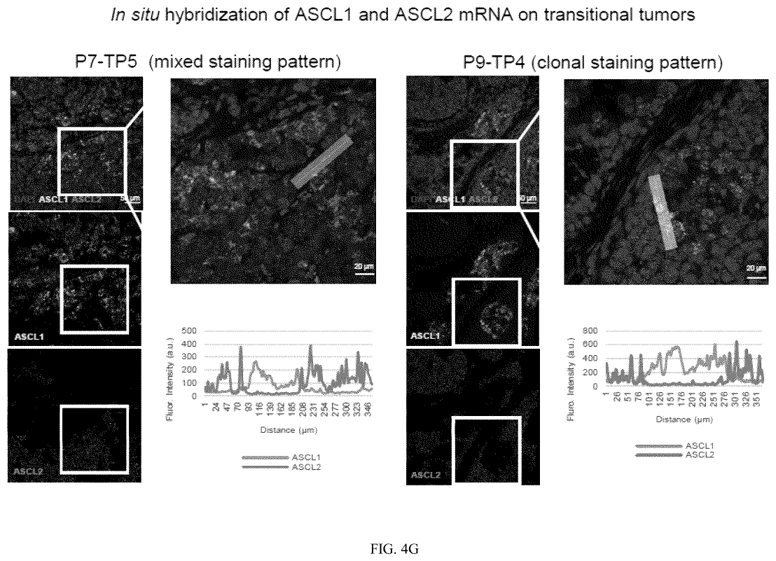

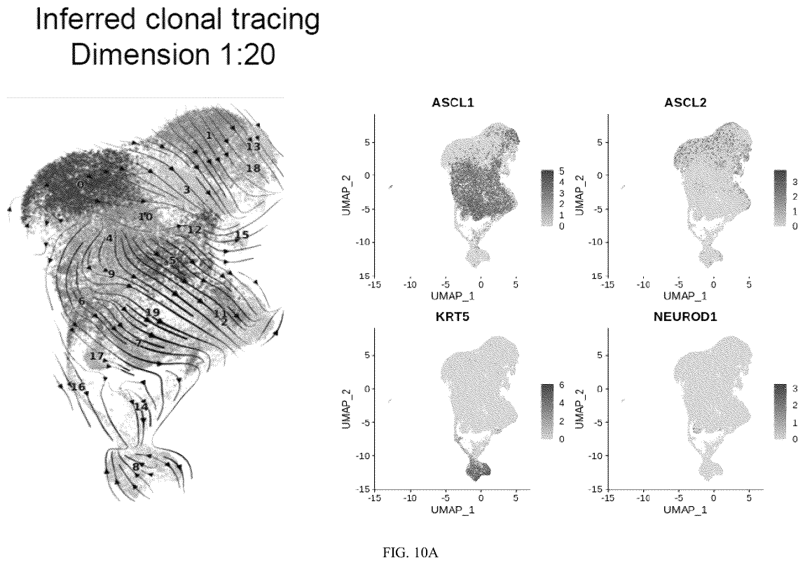

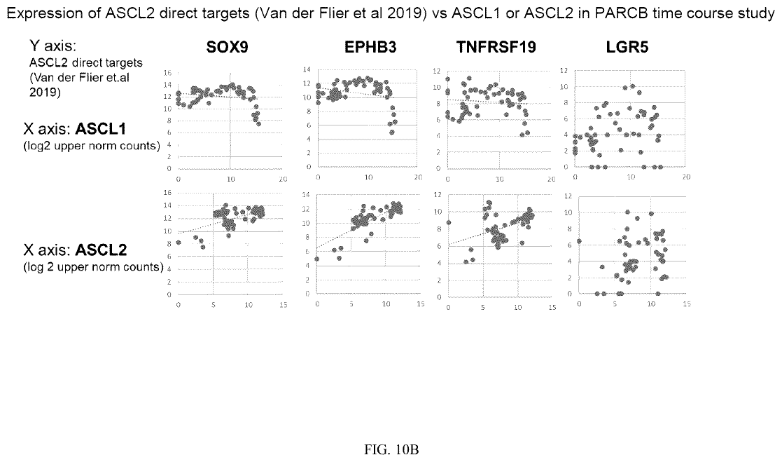

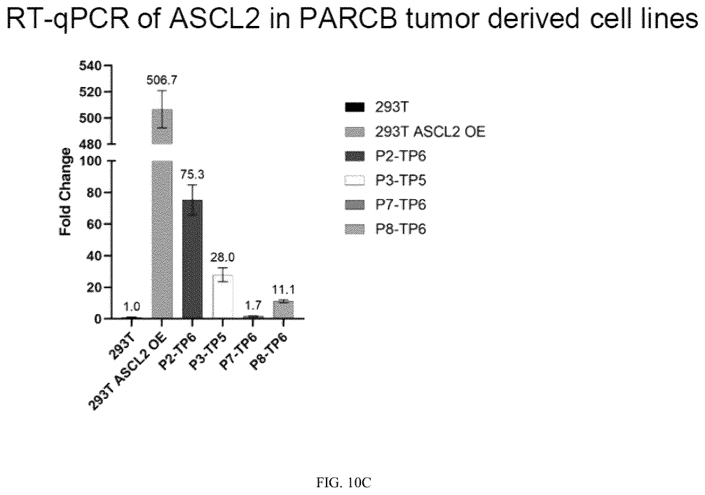

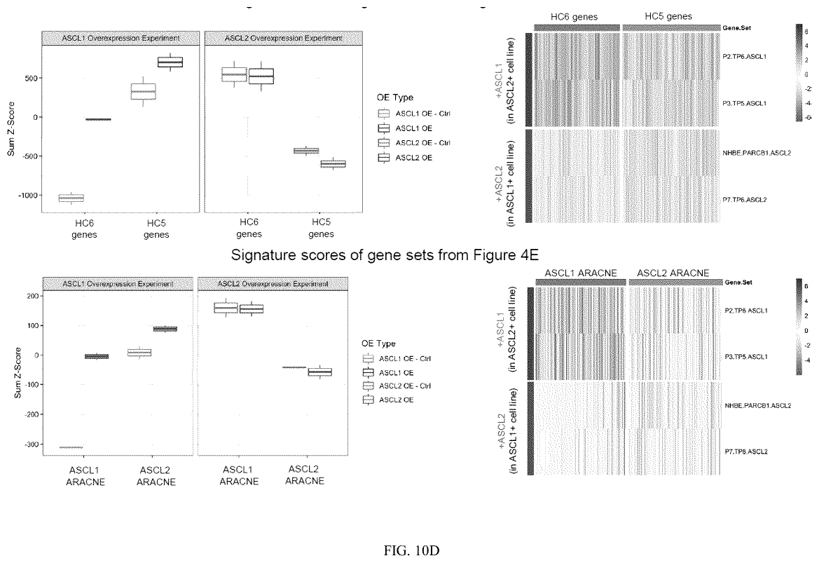

Given that ASCL1 and ASCL2 expression levels are mutually exclusive in single cells, the inventors asked whether ASCL1 and ASCL2 represent separate cellular sub-lineages by inferred clonal tracing analyses (52). With KRT5 (basal marker) set as the beginning of the tracing, the inferred tracing results in three primary lineage branches/states (FIG. 4A). As hypothesized, single cells expressing either ASCL1 or ASCL2 are enriched in different lineage branches (FIG. 4A-B). This result is further supported by a different analytic tool (RNA velocity) that measures the temporal ratio of un-spliced to spliced mRNAs to infer lineage trajectory (53) (FIG. 10A). The inferred clonal tracing results complemented the real-time-based analysis visualized as the total composition of ASCL1- or ASCL2-positive, double positive and negative populations over time (FIG. 4C), supporting that ASCL1 and ASCL2 are associated with independent sub-lineages. Double-positive cells are very infrequent in the PARCB temporal tumors. The double-positive population observed in the P2-TP5 tumor may capture the cells undergoing the transitional state (FIG. 4C), and the overall low double-positive frequency is consistent with the clinical results above (FIG. 3F).

To further characterize the transcriptional difference between cells expressing a high level of ASCL1 or ASCL2, the inventors analyzed their differential gene expression profiles (FIG. 4D and Table 4). Genes that are involved in synaptic and neuroendocrine regulation such as DDC, CACNA1A and INSM1 are enriched in ASCL1+ cells. ASCL2+ cells express genes with stem-like characteristics such as SOX9 and POU2F3 (FIG. 4D). SOX9 is directly regulated by ASCL2 in intestinal stem cells (29), suggesting a possible contribution to stem-like properties in SCNPC trans-differentiation. Upon further investigation, the inventors observed that genes implicated in the intestinal stem cell program such as EPBH3 and TNFRSF9 (29) are positively correlated with ASCL2, but not ASCL1 (FIG. 10B). In contrast, a well-known intestinal stem cell marker, LGR5 (54), has no correlation with either ASCL1 or ASCL2, consistent with it having a more tissue specific intestinal role (FIG. 10B).

To identify the transcriptional programs that are associated with either ASCL1 or ASCL2 in prostate cancer, the inventors constructed an inferred network (55) using multiple bulk RNA sequencing prostate cancer and model datasets including The Cancer Genome Atlas (TCGA), additional patient tumor (Beltran), and SCNPC model (Park) datasets (6,33). The analysis identified 336 and 352 genes regulated independently by ASCL1 or ASCL2 (FIG. 4E and Table 5). Strikingly, there are only 5 genes from the inference analysis that are regulated by both factors: TMEM74, RGS16, LHFPL4, CDCA7L and SOX2 (FIG. 4E). This result is consistent with the demonstrated role of SOX2 in regulating neuroendocrine differentiation in null TP53 and RB1 backgrounds (13), hence showing that SOX2 is involved in both ASCL1 and ASCL2 associated neuroendocrine sub-lineages. Genes that are regulated by ASCL1 are enriched in neuroendocrine differentiation markers and factors such as SYP, CHGA, NCAM1, and NEUROD1 (FIG. 4E). ASCL2 is associated with genes including PTGS1/COX1, POU2F3, ANXA1 which are generally immune and stress responsive, and stem-like (FIG. 4D-E). The inventors further confirmed that PARCB tumor-derived cell lines from different tissues of origin (prostate, bladder, and lung) all have only one or the other gene expression patterns associated with either ASCL1 or ASCL2 expression (FIG. 4F and FIG. 10C). The inventors next validated the predicted transcriptional programs of ASCL1 and ASCL2 by exogenously expressing either ASCL1 or ASCL2 in ASCL2+ or ASCL1+ cell lines, respectively. ASCL1 exogenous expression in ASCL2+ cells, increased the ASCL1 transcriptional program as indicated by increased signature score (FIG. 10D). However, ASCL2 exogenous expression in ASCL1+ cells, did not have notable effect, suggesting that the ASCL1 endpoint state has higher stability (FIG. 10D).

In situ hybridization of ASCL1 and ASCL2 mRNA in the transitional PARCB tumor samples further confirmed the mutually exclusive expression pattern (FIG. 4G). The staining patterns demonstrated both ASCL1 and ASCL2 mixed populations (left, FIG. 4G), as well as patch regions potentially resulting from local clonal expansion (right, FIG. 4G) of ASCL1+ or ASCL2+ cells. The combined results support that ASCL1 and ASCL2 define independent cellular sub-lineages and transcriptional programs with stem-like and neuroendocrine enrichment in SCNPC.

### 5. ASCL1 and ASCL2 as Pan-Cancer Classifiers

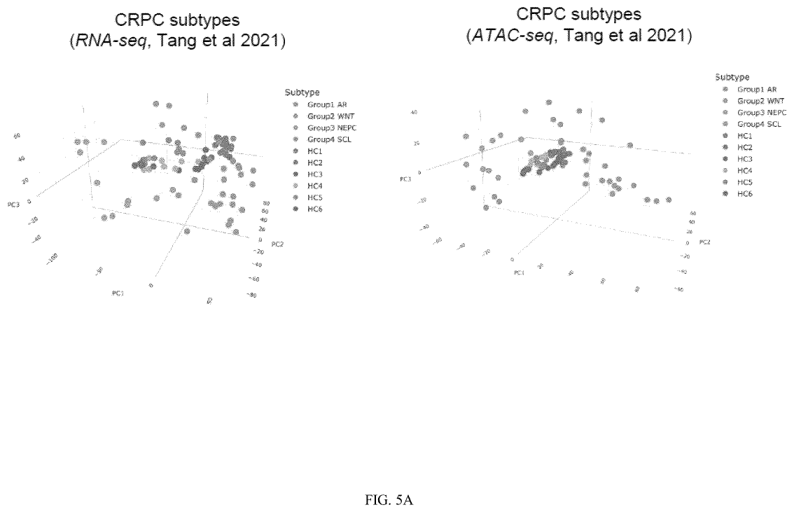

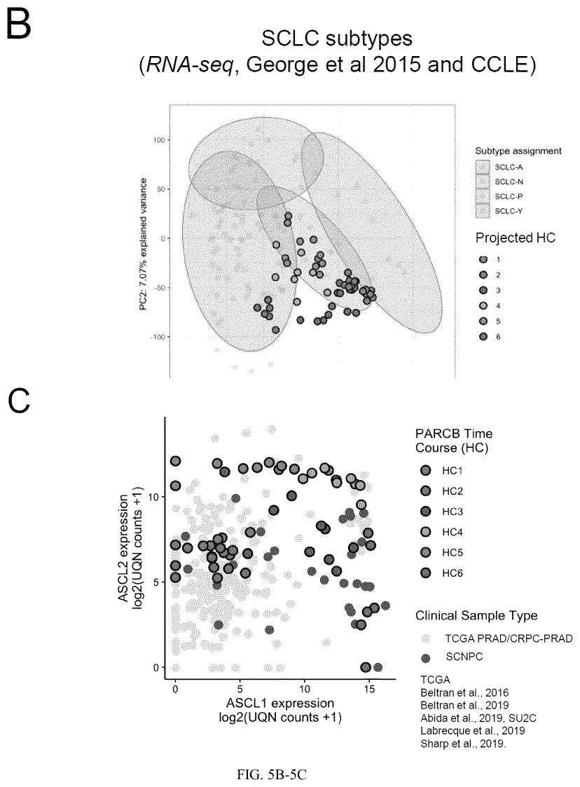

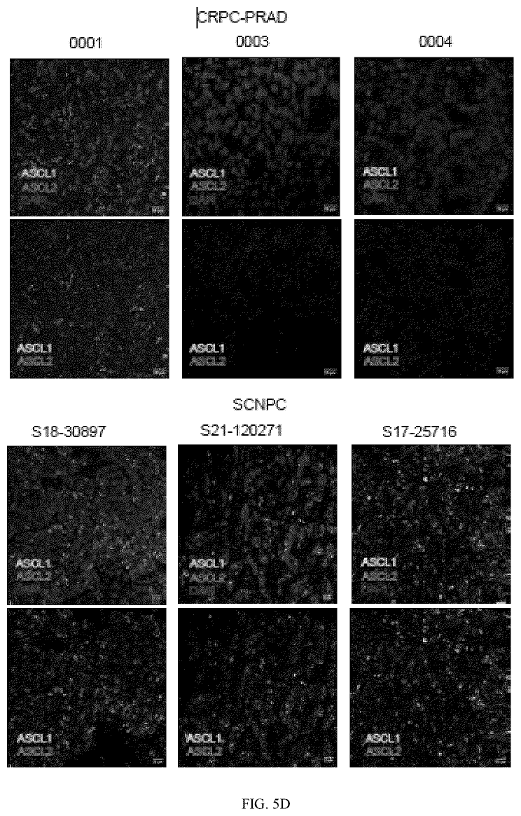

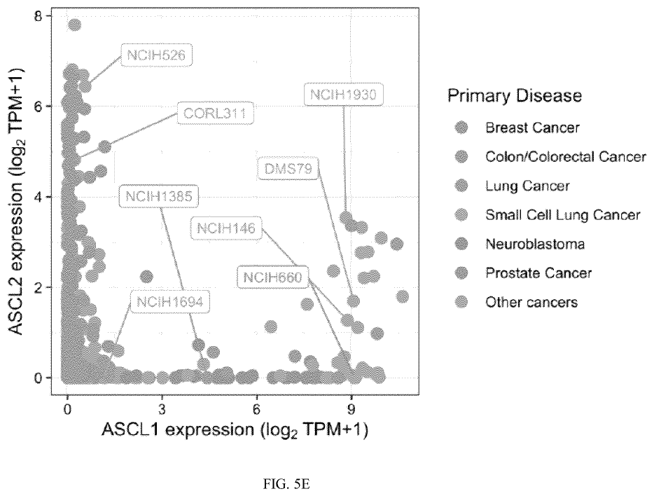

Clinical subtypes are fairly well-defined in SCLC (46,56), but molecular subtyping remains an evolving challenge in SCNPC 8. By performing projection analysis of the samples onto a gene expression or chromatin accessibility PCA framework defined by the Tang et al. dataset of patient metastatic CRPC phenotypes (57), the inventors found that PARCB temporal samples share similar transcriptome and epigenome signatures, including a shared stem-cell like (SCL) group and a shared NEPC group (FIG. 5A).

Given the high degrees of similarity in transcriptional profiles of SCLC and SCNPC (4), the inventors compared the HC classification of the PARCB time course samples to the SCLC clinical subtypes: ASCL1 (A), NEUROD1 (N), POU2F3 (P) and YAP1 (Y) (FIG. 5B) (32,46). The class I/ASCL2+ (HC5) tumor group shares transcriptome similarity with SCLC-P (FIG. 5B), which is consistent with the co-expression pattern of ASCL2 and POU2F3 observed in multiple analyses (FIGS. 3C and 4D). Likewise, and concordant, the Class II/ASCL1+ (HC6) tumor group is transcriptionally aligned to SCLC-A (FIG. 5B).

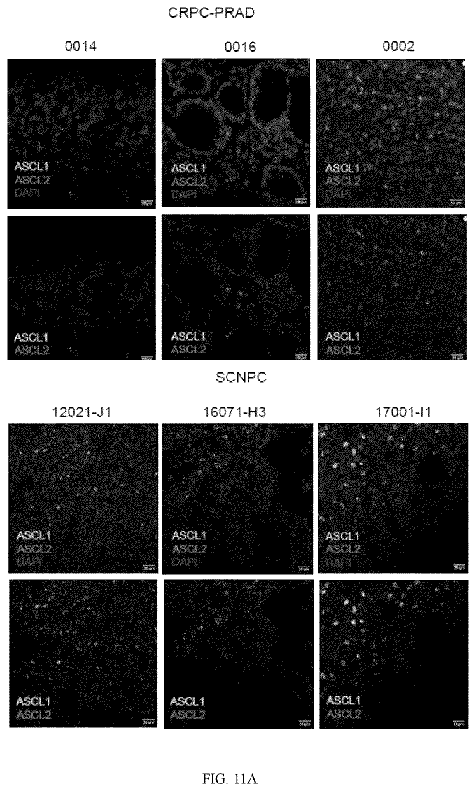

To investigate whether the ASCL1 and ASCL2 sub-classes from PARCB temporal study recapitulate patterns observed in clinical samples of prostate cancer, the inventors compared ASCL1 and ASCL2 expression in PARCB temporal samples versus numerous clinical profiling datasets (10,33-36). The results demonstrate that the expression levels of ASCL1 and ASCL2 are comparable between the PARCB model and clinical samples, and the transcriptional patterns of HC1 to HC6 generally corresponded with the transition from PRAD/CRPC-PRAD to SCNPC (FIG. 5C). The inventors further confirmed the general mutual exclusivity and low double positivity of ASCL1/2 expression using an RNA in situ hybridization assay on both CRPC-PRAD and SCNPC clinical samples and CRPC PDX models (FIG. 5D and FIG. 11A-B).

By comparing the expression levels of ASCL1 and ASCL2 across a broad panel of pan-cancer cell lines, the inventors found that almost all cancers, apart from lung cancers, can be divided into three categories (i) demonstrating expression of ASCL1 (neuroblastoma), (ii) of ASCL2 (colorectal and breast cancers), and (iii) double negative (other cancers) (FIG. 5E). Only SCLC and other lung cancer cell lines have mixed levels of ASCL1 and ASCL2. Combined with the inventors' results, this suggests a potential role for ASCL2 and POU2F3 in specifying intermediate, and/or heterogenous states in (small cell) lung cancer (FIG. 5E). Protein expression analysis in lung squamous carcinoma (NCI-H1385), SCLC-A (NCI-H1385, NCI-H146 and DMS79), SCLC-P (NCI-H526 and COR-L311) and SCNPC (NCI-H660) cell lines further highlighted a mutually exclusive pattern of ASCL1 and ASCL2 (FIG. 11C). SCLC-N(NCI-H1694) is double negative for ASCL1 and ASCL2 and positive for NEUROD1 as expected (FIG. 11C). Last but not the least, in patient tumor pan-cancer data, the exclusive expression of either ASCL1 or ASCL2 is again observed, highlighting that binary distinctions defined by ASCL1 and ASCL2 occur across multiple tissue types (FIG. 5F). In sum, an inverse and generally mutually exclusive relationship between ASCL1 and ASCL2 is observed in multiple and pan-cancer contexts, and mutual exclusivity is strongly observed at the single cell level. 6. Alternating ASCL1 and ASCL2 expression through reciprocal interaction and TFAP4 epigenetic regulation

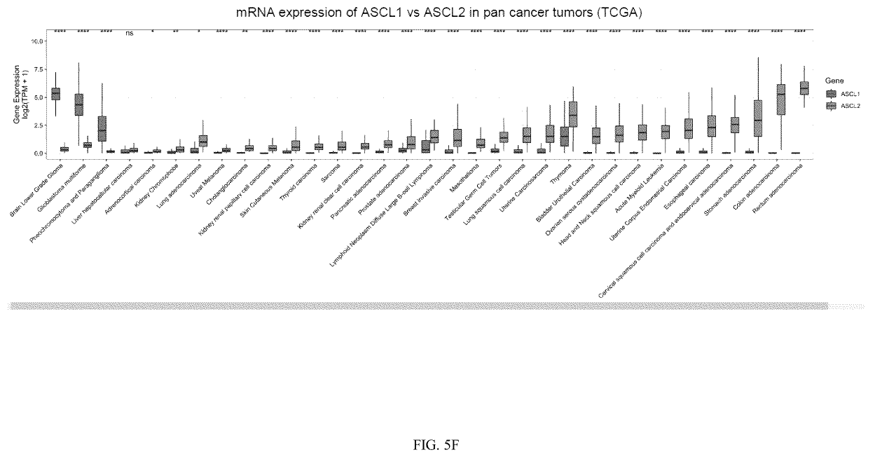

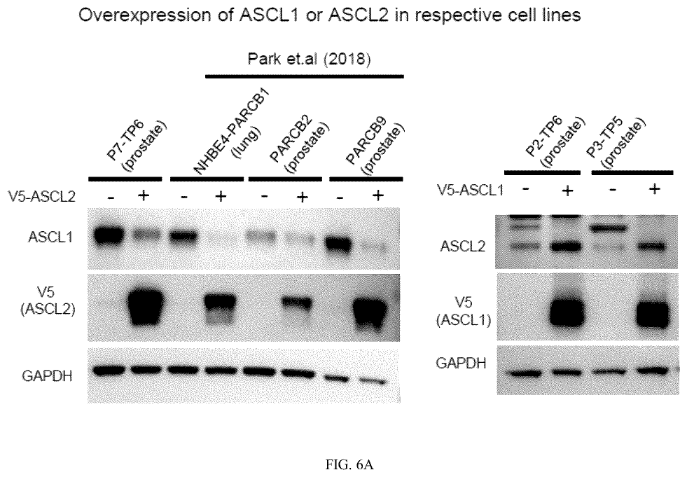

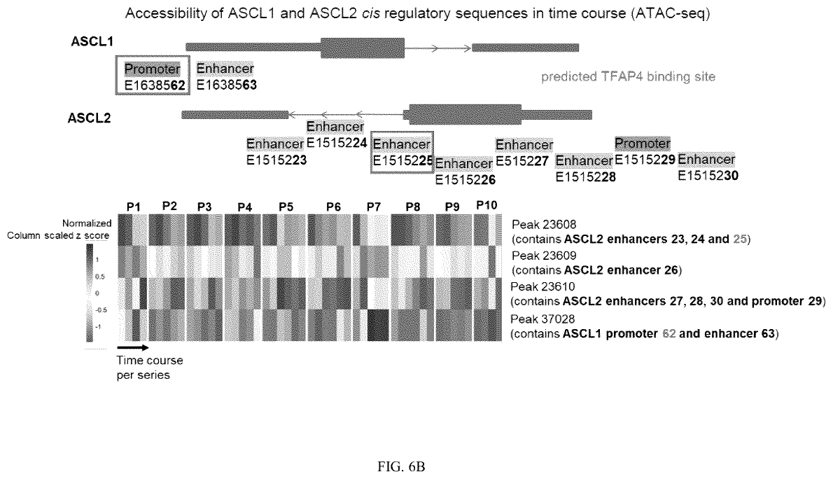

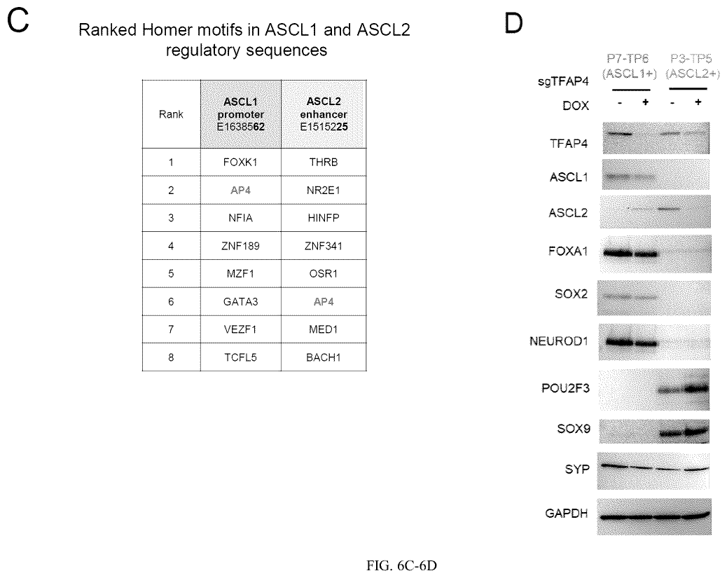

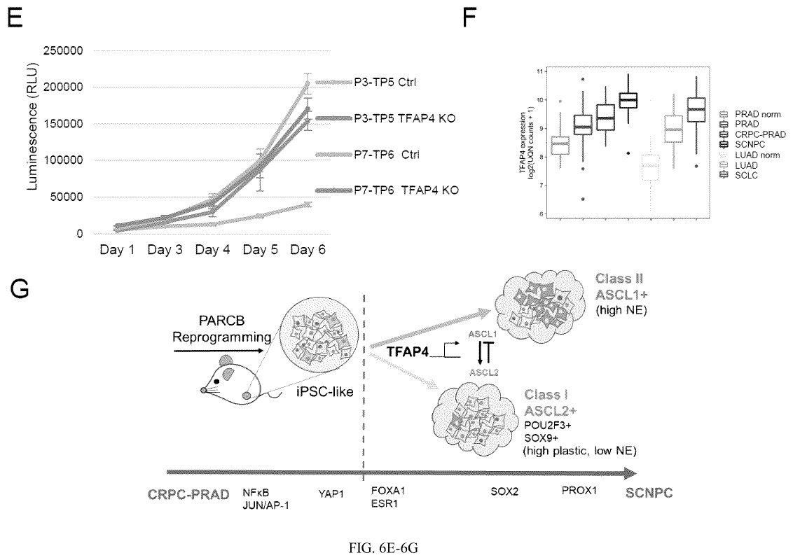

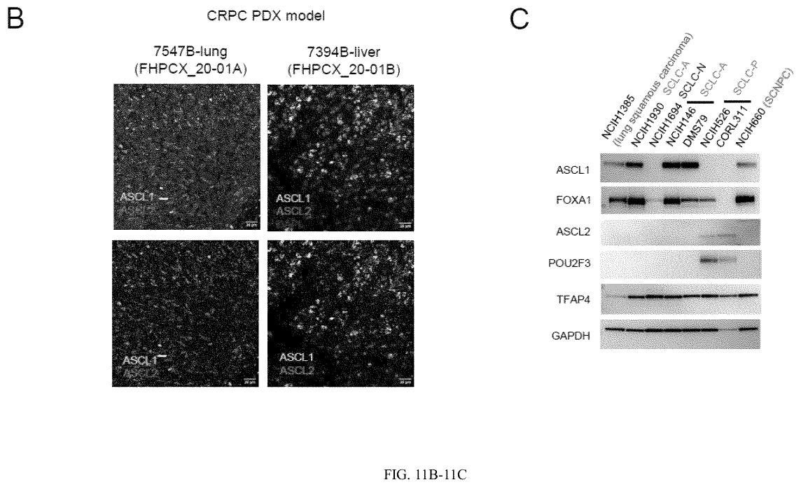

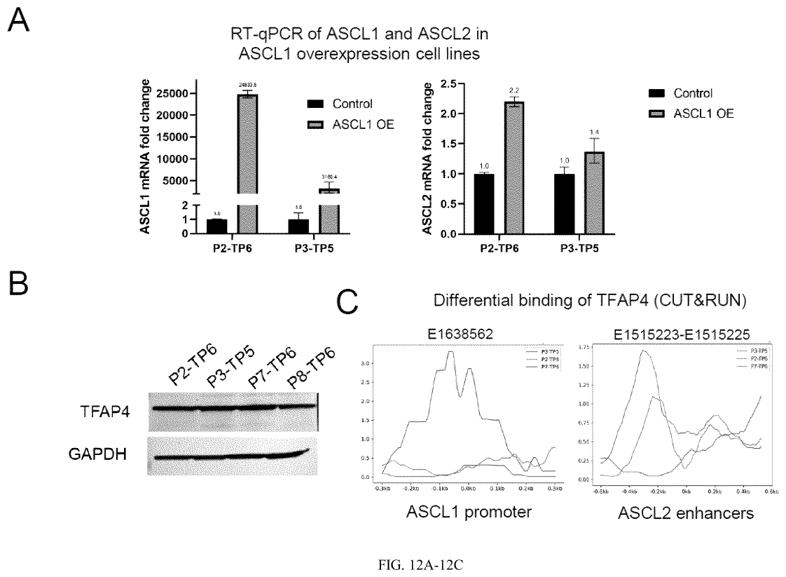

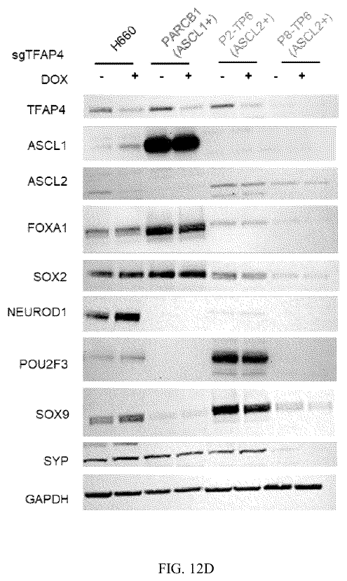

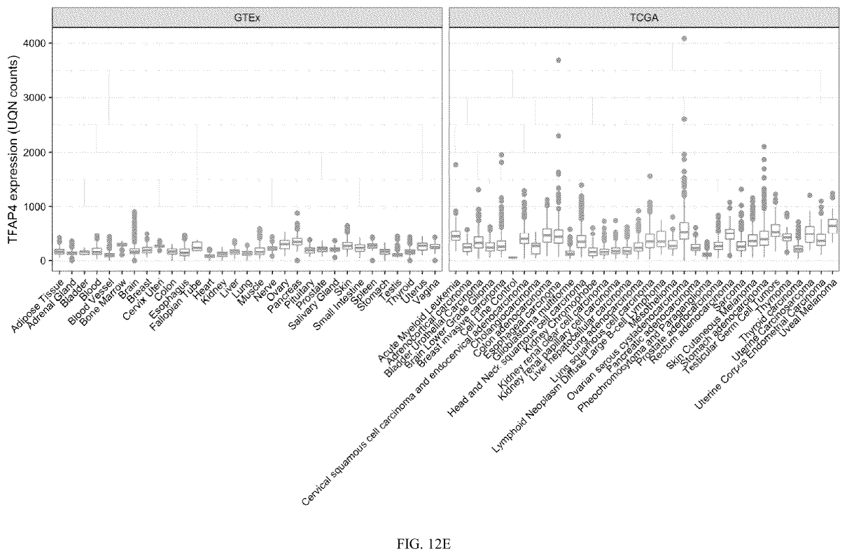

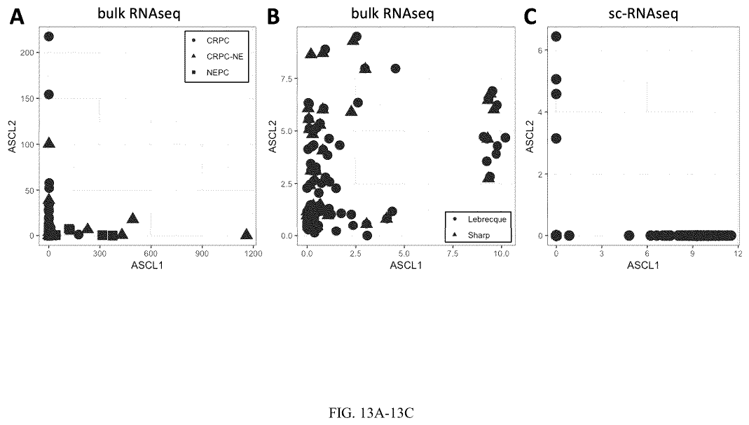

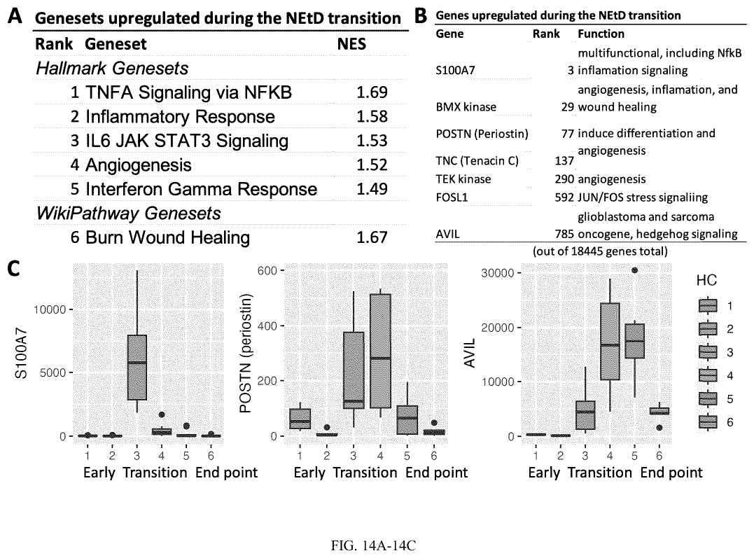

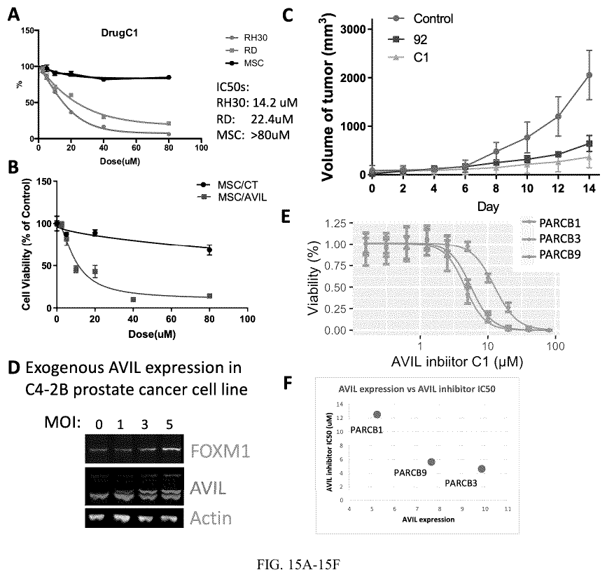

With the evidence that ASCL1 or ASCL2 expression levels are mutually exclusive in single cells during SCNPC trans-differentiation, the inventors explored two hypotheses: 1) These two factors mutually regulate each other's expression, or 2) they share a common upstream transcription factor that alternates their transcription through regulated differential binding to respective gene regulatory elements. To test the first hypothesis, the inventors expressed V5-tagged ASCL2 in multiple PARCB tumor derived cell lines (lung and prostate) and observed that ASCL1 protein expression was significantly suppressed in these cells (FIG. 6A). In contrast, expression of V5-tagged ASCL1 increased ASCL2 expressions both at protein and mRNA levels (FIG. 6A and FIG. 12A). Thus in the model cells, ASCL1 and ASCL2 mutually regulate each other at the protein level, but each in the opposite manner.

To test the second hypothesis of a common regulator, known promoter and enhancer regions of ASCL1 and ASCL2 were first annotated in the PARCB time course ATAC-seq data (FIG. 6B). An opposing pattern of open and closed chromatin formation is found on both the ASCL1 promoter and the ASCL2 enhancer regions (FIG. 6B). A rank list of transcription factors that have matching motifs in the regions was generated to determine potential shared regulators (58) (FIG. 6C). An extensive literature search of all the factors whose motifs were found in both ASCL1 and ASCL2 regulatory regions, revealed that TFAP4 was reported to form different transcription complex to either activate or repress target genes, including facilitating epithelial-to-mesenchymal transition in colorectal cancer and repressing neuronal programs in non-neuronal cells (59,60). The TFAP4 motif was shared in both the ASCL1 promoter (ranked 2^{nd}) and the ASCL2 enhancer region (ranked 6^{th}) in the top 8 list of shared transcription factor motifs (FIG. 6C), and is expressed across all the SCLC, SCNPC and PARCB tumor derived cell lines tested (FIGS. 11C and 12B). Interestingly, NCI-H1385, a lung squamous carcinoma (non-small cell) has lower TFAP4 expression compared to other small cell neuroendocrine cell lines (FIG. 11C).

The direct differential binding of TFAP4 to the ASCL1 promoter and the ASCL2 enhancer region was confirmed by the CUT&RUN technique (61), a chromatin immunoprecipitation experiment using TFAP4 antibody in both ASCL1+ and ASCL2+ PARCB tumor derived cell lines. Despite cell lines having various degrees of TFAP4 binding signals due to potential mixed cell clones within the cell lines, TFAP4 was found to have higher binding affinity near the ASCL1 promoter in ASCL1+ cell lines (P7-TP6) than ASCL2+ cell lines (P2-TP6 and T3-TP5) (FIG. 12C). In contrast, TFAP4 consistently bound to ASCL2 enhancer regions in ASCL2+ cell lines compared to ASCL1+ cell line (FIG. 12C). This result supports that TFAP4 potentially regulates transcription of ASCL1 and ASCL2 in a context-specific manner.

To determine whether TFAP4 directly regulates the expression of ASCL1 and ASCL2, the inventors introduced a doxycycline-inducible CRISPR sgRNA targeting TFAP4 in multiple ASCL1+ and ASCL2+ cell lines, including PARCB tumor-derived cell lines and the patient-derived cell line NCI-H660. Both ASCL1 and ASCL2 expression decreased, with various strength, after TFAP4 knockout was induced by the addition of doxycycline in the respective cell lines (FIG. 6F and FIG. 12D). However, other lineage associated proteins did not change (FIG. 6F and FIG. 12D). Cell growth assays showed a mild decrease in P7-TP6 (ASCL1+) cell growth, and in contrast a drastic increase in P3-TP5 (ASCL2+) growth upon the knockout of TFAP4 (FIG. 6E). To explore the clinical relevance of TFAP4 in cancer and SCNPC, the inventors surveyed the expression of TFAP4 across subtypes of cancers compared to normal tissue. There is a substantial increase in TFAP4 expression in small cell cancers compared to adenocarcinoma, and compared to normal tissue, in both prostate and lung cancer indications (FIG. 6F), as well as in pan cancer tumors (TCGA) vs. normal tissue (GTEx) (FIG. 12E).

Thus in the inventors' transcriptional regulatory circuit studies, the inventors found a reciprocal, non-symmetric regulatory relationship between ASCL1 and ASCL2; and that within this circuit, ASCL1 and ASCL2 have a shared positive regulatory factor, TFAP4. In the sum of these studies, the PARCB model provided a blueprint of SCNPC trans-differentiation as specified by temporal transcription factors (FIG. 6G). In particular, ASCL1 and ASCL2 define distinct bifurcated sub-lineage trans-differentiation trajectories in small cell cancers, and binary transcriptional profiles in a pan-cancer context.

### C. Discussion

SCNPC has a rare de novo presentation, however, trans-differentiation from prostate adenocarcinoma to SCN cancer is a frequent adverse consequence of cancer cells acquiring resistance to therapeutics repressing AR signaling (8,9). In a pan-cancer context, therapy-induced trans-differentiation from adenocarcinoma to SCN cancer is a growing clinical challenge in lung cancer with the expansion of effective targeted therapies, such as EGFR, ALK, BRAF, KRAS inhibitors (62). Genetically engineered mouse models of SCNPC and SCLC have been generated to provide insight into the tumorigenesis of SCN cancers (12,18,31,43,63,64), with some models demonstrating evidence of the adenocarcinoma to SCN cancer transition (13,31,65,66). Patient tumor-derived organoids and circulating tumor cells have also provided models for monitoring differentiation state transitions (50,67), including reversion to non-SCN states via specific signaling inhibition (50). The inventors' PARCB froward genetics in vivo temporal transformation model further adds to these resources by being human cell-based, recapitulating the adenocarcinoma to SCN phenotype trans-differentiation at both the histological and molecular signature levels, and providing the temporal resolution to reveal an arc-like plasticity trajectory and associated stem cell-like (reprogrammed) intermediate states. A limitation of the PARCB model is that inhibition of the AR axis is not an initiating component of the trans-differentiation process.

Such an arc-like trajectory is commonly observed in unbiased profiling of development and differentiation processes, including in cancer contexts (39,68-74). The pattern is reminiscent of temporal regulation in development, with the differentiation transition stage promoted by temporally regulated epigenetic and transcriptomic plasticity programs (75-77).

The transcription profiles of the transition stage from adenocarcinoma to SCNPC provide evidence for an initial de-differentiation or reprogramming step when cells enter the trans-differentiation process, with enrichment of stem cell and iPSC programs. Furthermore, the inventors find samples in the transitional state have a higher degree of entropy at both the epigenetic and gene expression level. Together these findings support the idea that de-differentiation, and epigenetic loosening and/or cellular heterogeneity are prerequisites for further lineage trans-differentiation during cancer evolution.

At the end-stages, the trans-differentiation trajectory demonstrates a bifurcation, resulting in two neuroendocrine states, one characterized by ASCL2 and POU2F3 expression (Class | tumors), the other by ASCL1 expression (class II tumors). Throughout the trans-differentiation trajectory, individual cells demonstrate mutually exclusive expression of either ASCL1 or ASCL2, with emergence of ASCL2 generally earlier and more prominent than ASCL1. Thus, the ASCL2 state and double positive state may reflect a semi-stable and transitional state. The molecular switch from ASCL2 to ASCL1 demonstrates the dynamic transcriptional control in SCNPC. An analogous temporal shift from FOXA1 to FOXA2 orchestrated transcriptional programs was observed in an independent SCNPC temporal GEMM model (43), and the FOXA1 to FOXA2 transition is reflected in the PARCB model (FIG. 8D).

A dynamic lineage plasticity among subtypes of SCLC has been reported (18). However, the triggers and mechanisms underlying cancer cells switching to different lineages remain elusive. In SCNPC, beyond the discovery of the reciprocal regulation between ASCL1 and ASCL2, the results identified TFAP4 as an additional candidate member of this transcriptional circuitry. In particular, TFAP4 can alternate the expression of ASCL1 and ASCL2 by differential binding to cis regulatory elements on both genes. TFAP4 has been shown to have both activating and repressing properties in gene regulation through interactions with distinct transcription factors (59,60). TFAP4 demonstrates substantial increased expression in small cell vs. non-small cell cancers and is elevated in cancers compared to normal tissue. Future mechanistic and functional studies on TFAP4 will help clarify its master regulator role in lineage trans-differentiation in SCNPC and SCLC.

In clinical therapy, different forms of tumor plasticity define the battle grounds for acquired resistance. In the primary prostate cancer setting, the vast majority of prostate cancers are adenocarcinomas while all other histologic types are rare. In the castration-resistant setting, especially with the clinical introduction of next-generation anti-AR therapies, many different variant histology has been observed, including rare cases of squamous carcinoma (80). In this combat, trans-differentiation to a small cell neuroendocrine state in response to otherwise effective molecular therapies is an emerging challenge across multiple cancer types. The temporal profiling of SCNPC development in the human cell based PARCB model revealed that trans-differentiation from an adenocarcinoma to neuroendocrine state is a temporally complicated, yet systematically coordinated process. The combination of bulk and single cell profiling approaches allowed for the identification of an arc-like trajectory and a transitory period characterized by epigenetic loosening, which are shared in general by other differentiation and development processes. Consistent with genetically engineered mouse SCNPC models, and with the multiple subtypes of SCLC, the inventors find a role for both ASCL1 and ASCL2/POU2F3 in trans-differentiation to SCNPC. The results from the model have provided insight into the regulatory crosstalk between different neuroendocrine master regulators and provide a resource for identifying candidate approaches for blocking this clinically challenging case of trans-differentiation.

### D. Methods

### 1. Date and Code Availability

Bulk RNA-seq data, bulk ATAC-seq data, single cell RNA-seq data and ChIP-seq (CUT&RUN) data have been deposited at dbGAP (phs003230.v1). In addition, the gene expression counts of Bulk RNA-seq and single cell RNA-seq data have been deposited at GEO (GSE240058,). Accession numbers are also listed in the key resources table. Both data depositories will be made publicly available as of the date of publication.

### 2. PARCB Transformation Temporal Model

Prostate tissues from donors were provided in a de-identified manner and therefore exempt from Institutional Review Board (IRB) approval. Processing of human tissue, isolation of basal cells, organoid transformation, and xenograft assay were described in detail previously (6). 20,000 cells FACS-sorted cells per organoid were plated in 18-20 μl of growth factor-reduced Matrigel (Cat #356234, Corning) with PARCB lentiviruses (MOI=50/lentivirus). Organoids were cultured in the prostate organoid media (82) for about 10-14 days. Transduced organoids were harvested by dissociation of Matrigel with 1 mg/ml Dispase (Cat #17105041, Thermo Fisher Scientific). The organoids were washed three times with 1×PBS to remove Dispase and re-suspended in 10 μl of growth factor reduced Matrigel and 10 μl Matrigel with High Concentration (Cat #354248, Corning). The organoid-Matrigel mixture was implanted subcutaneously in immunodeficient NOD.Cg-Prkdescid II2rgtm1Wjl/SzJ (NSG) mice (83). Tumors were extracted in every two-week window, with the last tumor collection of the time course series determined by either reaching around 1 cm in diameter in tumor size or ulceration, whichever came first. NSG mice had been transferred from the Jackson Laboratories and housed and bred under the care of the Division of Laboratory Animal Medicine at the University of California, Los Angeles (UCLA). All animal handling and subcutaneous injections were performed following the protocols approved by UCLA's Animal Research Committee.

### 3. Cell Lines

NCI-H1385 (Cat #CRL-5867), NCI-H1930 (Cat #CRL-5906), NCI-H1694 (Cat #CRL-5888), NCI-H146 (Cat #HTB-173), DMS79 (Cat #CRL-2049), NCI-H526 (Cat #CRL-5811), and NCI-H660 (Cat #CRL-5813) were purchased from American Type Culture Collection (ATCC). COR-L311 was obtained from Sigma Aldrich (Cat #96020721). All commercially available cell lines were cultured and maintained based on the instruction from the vendors. PARCB tumor derived cell lines were generated using the previous method (6). All the cell lines in the study are free of Mycoplasma using a MycoAlert™ PLUS Mycoplasma Detection Kit (Cat #LT07-703, Lonza).

### 4. Lentiviral Vectors and Lentiviruses

The myristoylated AKT1 vector (FU-myrAKT1-CGW), exogenous expression of cMYC and BCL2 (FU-cMYC-P2A-BCL2-CRW), dominant TP53 mutant (R175H) and shRNA targeting RB1 vector (FU-shRB1-TP53DN-CYW) have been described previously 6. Exogenous expression of V5 tagged ASCL1 (pLENTI6.3-V5-ASCL1) is obtained from DNASU (Cat #: HsCD00852286) (84). For making exogenous expression of ASCL2 containing vector (pLENTI6.3-V5-ASCL2), Gateway cloning (Cat #11791020, Thermo Fisher) was performed using pLenti6/V5-DEST Gateway Vector (Cat #V49610, Thermo Fisher) and the entry plasmid (pDONR221-ASCL2) was obtained from DNASU (Cat #HsCD00829357) (84). For making doxycycline-inducible sgTFAP4 (TLCv2-Cas9-BFP-sgTFAP4), TLCv2 (Cat #87360, Addgene) was first digested with BamHI-HF (Cat #R3136, New England Biolabs) and Nhel-HF (Cat #3131, New England Biolabs) at 37° C. for 1 hour and inserted with a synthesized fragment containing T2A-Hpal-BFP sequence (gBlock service provided by IDT) using Gibson Assembly (Cat #E5510, New England Biolabs). sgTFAP4 sequence was cloned into the previously described TLCv2-BFP vector using an established protocol (85). sgTFAP4-primers are listed in the key resources table. Lentiviruses were produced and purified by a previously established method (86).

### 5. Tissue Section, Histology, and Immunohistochemistry (IHC)

PARCB model tumor tissues were fixed in 10% buffered formaldehyde overnight at 4° C. and followed by 70% ethanol solution. Tissue microarray construction and hematoxylin and eosin (H&E) staining were performed by Translation Pathology Core Laboratory (TPCL) in UCLA using standard protocol. TPCL is a CAP/CLIA certified research facility in the UCLA Department of Pathology and Laboratory Medicine and a UCLA Jonsson Comprehensive Cancer Center Shared Facility. For immunohistochemistry staining, formalin-fixed, paraffin-embedded (FFPE) sections were deparaffinized and rehydrated with a washing sequence of xylene and different concentration of ethanol. Citrate buffer (pH6.0) was used to retrieve antigens. The sections were incubated in citrate buffer and heated in a pressure cooker. 3% of H2O2 in methanol was used to block endogenous peroxidase activity for 10 mins at room temperature. The sections were blocked then incubated with primary antibodies overnight at 4° C. Anti-mouse/rabbit secondary antibodies were used to detect proteins of interest and DAB EqV substrate was used to visualize the staining. All components were included in the ImmPRESS Kit (MP-7801-15 and MP-7802-15, Vector Laboratories) The slides were then dehydrated and mounted with Xylene-based drying medium (Cat #22-050-262, Fisher Scientific).

### 6. Western Blot

Cells were lysed on ice using UREA lysis buffer (8M UREA, 4% CHAPS, 2× protease inhibitor cocktail (Cat #11697498001, Millipore Sigma)). Genomic DNA was removed by ultracentrifuge (Beckman Optima MAX-XP, rotor TLA-120.1, 48,000 rpm for 90 min). Protein concentrations were measured using the Pierce BCA Protein Assay Kit (Cat #: 23227, Thermo Scientific). Samples were electrophoresed on polyacrylamide gels (Cat #NW04120BOX, Thermo Fisher), transferred to nitrocellulose membranes (Cat #88018, Thermo Fisher). Western blots were visualized using iBright CL1500 Imaging system (Cat #44114, Thermo Fisher).

### 7. RT-qPCR

Total RNA was isolated from cells using miRNeasy Mini Kit (Cat #217004, Qiagen). cDNA was synthesized from 2 μg of total RNA using the SuperScript IV First-Strand Synthesis System (Cat #18091050, Thermo Fisher). RT-qPCR was performed using SYBR Green PCR Master Mix (Cat #4309155, Thermo Fisher). Amplification was carried out using the StepOne Real-Time PCR System (Cat #4376357, Thermo Fisher) and analysis was performed using the StepOne Software v2.3. with the following primers were used at a concentration of 250 uM: Relative quantification was determined using the Delta-Delta Ct Method. Primer sequences are listed in the key resources table.

### 8. In Situ RNA Hybridization Assay and Image Analysis

The RNAscope Multiplex Fluorescent V2 kit was used to perform in situ hybridization on FFPE tissue microarray slides following the manufacturer's protocol (Cat #323270, ACDBio). The Institutional Review Board of the University of Washington approved this study (protocol no. 2341). All rapid autopsy tissues were collected from patients who signed written informed consent under the aegis of the Prostate Cancer Donor Program at the University of Washington. The establishment of the patient-derived xenografts was approved by the University of Washington Institutional Animal Care and Use Committee (protocol no. 3202-01). For multiplex hybridization, the Double Z probes targeting ASCL1 (Cat #459721-C2, ACDBio) and ASCL2 (Cat #323100, ACDBio) were hybridized to the samples for 2 hours at 40° C. ASCL1 signal was visualized using Opal dye 520 (Cat #FP1487001KT, Akoya Biosciences) and ASCL2 signal was visualized using Opal dye 570 (Cat #FP1488001KT, Akoya Biosciences). DAPI (Cat #D3571, Thermo Fisher) was used to visualize nuclei. Confocal fluorescence images were acquired using an inverted Zeiss LSM 880 confocal microscope. All images were processed using Fiji.

### 9. Cell Proliferation Assay

3000 cells per cell line in five replicates were seeded on 96-well plates on Day 0. Cell viability was measured on Day 1, 3, 4, 5 and 6. using Cell Titer-Glo Luminescent Cell Viability Assay (Cat #G7570, Promega). Luminescence was measured at an integration time of 0.5 second per well.

### 10. Bulk RNA Sequencing and Dataset Collection

Tumors were dissociated into single cells, followed by cell sorting of triple colors (RFP, GFP and YFP) by flow cytometry. Total RNA was extracted from the cell lysates using miRNeasy mini kit (Cat #217084, Qiagen). Libraries for RNA-Seq of PARCB time course samples were prepared with KAPA Stranded mRNA-Seq Kit (Cat #KK8420, Roche). The workflow consists of mRNA enrichment and fragmentation. Sequencing was performed on Illumina Hiseq 3000 or NovaSeq 6000 for PE 2×150 run. Data quality check was done on Illumina SAV. Demultiplexing was performed with Illumina Bcl2fastq v2.19.1.403 software. Raw sequencing reads were processed through the UCSC TOIL RNA Sequencing pipeline1 for quality control, adapter trimming, sequence alignment, and expression quantification. Briefly, sequence adapters were trimmed using CutAdapt v1.9, sequences were then aligned to human reference genome GRCh38 using STAR v2.4.2a and gene expression quantification was performed using RSEM v1.2.25 with transcript annotations from GENCODE v23 (87).

The FASTQ files of the Park dataset (6), Beltran dataset (33), George dataset (32) and Tang dataset (57) were all processed through the TOIL pipeline with the same parameters to get RSEM expected counts. The TOIL-RSEM expected counts of TCGA pan cancer samples were obtained directly from UCSC Xena browser (available online at xenabrowser.net/datapages) and gene expression (log 2 (TPM+1)) of pan-cancer cell lines from the Cancer Cell Line Encyclopedia (CCLE) were downloaded from DepMap Portal (DepMap Public 22Q1). The RSEM counts of all combined datasets were upper quartile normalized, log 2 (x+1) transformed (referred to as log 2 (UQN+1) counts) and filtered down to HUGO protein coding genes (http://www.genenames.org/) for the downstream analyses. SCLC subtypes (46) and CRPC subtypes (57) were previously defined 11. Differential gene expression analysis and hierarchical clustering PARCB Time Course Samples were Grouped into 6 Hierarchical Clusters (HC) by performing Ward's hierarchical clustering (k=6) on log 2 (UQN+1) counts using the hclust function from the base R package, Stats. Differential gene expression analysis was then performed on each HC in a “one vs. rest” fashion, i.e., between one cluster vs. the remaining five clusters, using DESeq2 with the following parameters: independentFiltering=F, cooksCutoff=FALSE, alpha=0.1 (88). For each HC vs. rest comparison, genes with a log 2FC >2 and p-adjusted value <0.05 were considered upregulated for that HC gene module. However, four genes (IL1RL1, KRT36, PIK3CG, NPY) were upregulated among multiple HC vs. rest comparisons. As a result, these genes were assigned to the HC gene module with the smaller p-adjusted value for that gene. Z-scores for upregulated genes in each cluster were then plotted in a heatmap using pheatmap function. PARCB time course samples were subsequently categorized by this HC definition in downstream analyses.

### 12. GO Enrichment Analysis

Enrichment analysis was performed using the “GO_Biological_Process_2021” database and the enrichr function from the R package, enrichR, using upregulated genes for each HC (89). Pathways were selected based on their adjusted p-value for each HC. The results were plotted using ggplot ( )

### 13. Bulk ATAC Sequencing and Dataset Collection

Tumors were dissociated into single cells, followed by cell sorting of triple colors (RFP, GFP and YFP) by flow cytometry. ATAC-seq samples were prepared following the previously published protocol (38). Bulk ATAC sequencing was performed in the Technology Center for Genomics & Bioinformatics Core in UCLA. Sequencing was performed on Illumina NovaSeq 6000 for PE 2×50 run. Data quality check was done on Illumina SAV. Demultiplexing was performed with Illumina Bcl2fastq v2.19.1.403 software. The raw FASTQ files were processed using the published ENCODE ATAC-Seq Pipeline. The reads were trimmed and aligned to hg38 using bowtie2. Picard was used to de-duplicate reads, which were then filtered for high-quality paired reads using SAMtools. All peak calling was performed using MACS2. The optimal irreproducible discovery rate (IDR) thresholded peak output was used for all downstream analyses, with a threshold P value of 0.05. Other ENCODE3 parameters were enforced with the flag-encode3. Reads that mapped to mitochondrial genes or blacklisted regions, as defined by the ENCODE pipeline, were removed. The peak files were merged using bedtools merge to create a consensus set of peaks across all samples, and the number of reads in each peak was determined using bedtools multicov (90). A variance stabilizing transformation was performed on peak counts using DESeq2 (88) and batch effects were removed using removeBatchEffect( ) from limma (91). All downstream ATAC-seq analysis was performed using this matrix (referred to as VST peak counts), unless otherwise specified.

Raw FASTQ files of Tang ATAC-seq dataset were downloaded from GSE193917 (57). The raw FASTQ files were processed using the same ENCODE pipeline described above with the same parameters.

### 14. Differential Chromatin Accessibility and Transcription Start Site (TSS) Analysis

Differential peak analysis was performed on each HC in a one vs. rest fashion, as described above in the bulk RNA-sequencing analysis. Peaks were called hyper- or hypo-accessible if the log 2 fold change was greater than 2 or less than 2, respectively, and had an adjusted p-value of less than 0.05. The z-scores of the union of all differentially accessible peaks were used to plot the heatmap using VST peak counts, with the rows ordered by chromosomal location.

For mapping peaks near TSS sites, the bigwig files containing ATAC-seq readings were first converted into wig files. Wig files from samples within the same HC were then merged by calculating the mean across peak regions using wiggleTools (92). The TSS analysis was performed using deepTools and computeMatrix in reference-point mode with parameters referencePoint=TSS, a=2000, b=2000 to compute the scores from merged bigwigs in regions 2 kbp flanking the region of interest. plotHeatmap was used with parameters zMin=0, zMax=5, binSize=10 was to plot the TSS figure from the score matrix (93).

### 15. PCA and Projection Analyses

Unsupervised PCA analysis of the PARCB time course samples using log 2 (UQN+1) counts was performed using the prcomp function from the stats package available on R (described above). PC2 and PC3 sample scores were then multiplied by a 30-degree clockwise rotation matrix. Ellipses were drawn around samples with 95% confidence based on real time labels using stat_ellipse( ) from ggplot2. The PCA projection of PARCB time course samples onto the framework using pan small cell cancer combined gene expression datasets have been discussed previously (4). In brief, the input matrix for this PCA was centered but not scaled. PARCB time course samples were then projected by multiplying the data matrix by the PCA loadings. For projection of PARCB time course samples onto the framework using gene expression data of CRPC subtypes (57) or SCLC subtypes (46), the same methodology was applied.

For projection of PARCB time course samples onto the framework using ATAC-seq data of CRPC subtypes (57), peak coverage of the Tang dataset was determined using the consensus set of peaks from the PARCB time course data with function bedtools multicov (90). Tang dataset peak read counts were then variance stabilized transformed using DESeq2 (88). PCA was performed on VST peak read counts of the Tang dataset using the prcomp function with the parameters center=T, scale=F. PARCB time course samples were then projected onto the framework by multiplying PARCB time course VST peak read counts by PCA loadings.

For projection of PARCB time course single cells onto the framework defined by the bulk RNA-seq data, the single cell data after integration by batch was down-sampled for 1000 cells within each patient series or cluster. The single cell and bulk RNA-seq data were centered separately prior to projection. The projection was carried out by multiplying the single cell data matrix by PCA loadings of PARCB bulk samples.

### 16. Transcription Factor Analysis

Top ranked transcription factors (TF) were selected using the gene loading scores derived from the unsupervised PCA analysis of gene expression described above. PC2 and PC3 loading scores were rotated 30 degrees clockwise by multiplying a 30-degree clockwise rotation matrix to the gene loading scores (resulting components called PC2′ and PC3′, respectively). The loading scores were then filtered to include only transcription factors (37). The center of the TF loading scores was determined by taking the average of PC1, PC2′, and PC3′. The Euclidean distance from the center was calculated for each TF, and the top 60 TFs furthest from center were selected. Hierarchical clustering (k=5) was performed on the log 2 (UQN+1) counts of the top 60 TFs. The z-scores for each TF were plotted using pheatmap. Average z-score of HOXC genes was calculated from HOXC 4-13 (except for HOXC7) in each PARCB time course sample.

### 17. Shannon Entropy Analysis

Shannon entropy for each PARCB time course sample was calculated on variance stabilized transformed (VST) ATAC-seq peak counts using the Entropy ( ) function from the R package DescTools. PARCB samples falling within the 95th percentile of calculated Shannon entropy scores were included in the following PCA analysis. PCA was performed on VST peak counts and was plotted using ggplot2 with samples colored by their Entropy scores and ellipses with 95% confidence were drawn around each time point group using stat_ellipse.

### 18. Prostate Cancer Gene Regulatory Network Analysis

The RNA-seq data of PARCB time course study, Park dataset (6), Beltran dataset (33), and TCGA PRAD/PRAD-norm dataset were included in this analysis. TCGA PRAD/PRAD-norm data was down sampled to match the sample size of other cohorts. Gene network was built on the combined datasets using ARACNe-AP (81).

### 19. Signature Scores (Adult Stem Cell, Adenocarcinoma and SCNPC)

SCNPC signature was derived using Beltran dataset (33), following the methods described previously 6. The adult stem cell (ASC) signature in the analysis is defined in literature 42. For prostate adenocarcinoma signature, differential gene expression analysis was performed on TCGA PRAD samples vs CRPC-PRAD and SCNPC samples from the Beltran dataset (10,33) using DESeq2. The adenocarcinoma signature was defined by all the upregulated genes (log 2FoldChange >2 and padj <0.05) from the differential gene expression analysis. Adenocarcinoma and SCNPC signature scores of the PARCB time course samples were calculated using gsva with method= “ssgsea”.

### 20. Motif Analysis

Hyper-accessible peaks in each HC from the differential peak analysis described previously were used for motif enrichment analysis using GimmeMotifs 41,90. Differential motif analysis was performed on hyper-accessible peaks for each HC against a hg38 whole-genome background using the maelstrom function with default parameters. The top 5 enriched motifs and their aggregated z-scores for each HC are shown in the heatmap (each individual HC vs all others). Additionally, the inventors performed differential peak analysis on HC5 vs HC1-HC4 and HC6 vs HC1-HC4 with the same parameters as described previously using DESeq2. Likewise, hyper-accessible peaks for HC5 and HC6 in these comparisons were defined by a threshold of log 2FoldChange >2 and padj <0.05. Differential motif analysis was performed on the set of hyper-accessible peaks from HC5 vs HC1-4 and HC6 vs HC1-4 using the maelstrom function as described above. Note that in the GimmeMotif enrichment analysis, transcription factors are culled to minimize redundancy, and this step is impacted by the exact input data and sample group comparison indicated. Thus, each motif suite may contain slightly different enriched transcription factors. However the transcription factor sets remain highly consistent between each case.

For identifying transcription factors that recognize ASCL1 and ASCL2 regulatory sequences, ASCL1 and ASCL2 promoter and enhancer regions were mapped using UCSC Genome Browser Gateway (available online at genome.ucsc.edu/cgi-bin/hgGateway). Motif analysis was then performed on each ASCL1 and ASCL2 promoter and enhancer region using the findMotifGenome function from HOMER with the parameters -size 200 and -mask (58). Resulting motifs were then ranked by their p-value. Additionally, ASCL1 and ASCL2 enhancer and promoter regions were mapped to accessible peaks from ATAC-seq data of the PARCB time course to identify chromatin changes of ASCL1 and ASCL2 cis-regulatory sequences. Peak regions from the PARCB consensus peak set overlapping with the ASCL1 and ASCL2 enhancer and promoter regions were then plotted in a heatmap using VST peak counts and scaled per sample.

### 21. Single-Cell RNA Sequencing

PARCB time course samples were sequenced in two batches: P2/P5 and P6/P7 series. Single cell gene expression libraries were created using Chromium Next GEM Single Cell 3′ (v3.1 Chemistry) (Cat #PN1000123, 10× Genomics), Chromium Next GEM Chip G Single Cell Kit (Cat #PN1000120, 10× Genomics), and Single Index Kit T Set A (Cat #PN1000213, 10× Genomics) according to the manufacturer's instructions. Briefly, cells were loaded to target 10,000 cells to form GEMs and barcode individual cells. GEMs were then cleaned cDNA and libraries were also created according to manufacturer's instructions. Library quality was assessed using 4200 TapeStation System (Cat #G2991BA, Agilent) and D1000 ScreenTape (Cat #5067-5582, Agilent) and Qubit 2.0 (Cat #Q32866, Invitrogen) for concentration and size distribution. Samples were sequenced using Novaseq 6000 sequencer (Catl #A00454, Illumina) using 100 cycles (28+8+91). The illumina base calling files were converted to FASTQ using the mkfastq function in Cell Ranger suite. The reads were then aligned to GRCh38 for UMI counting with cellranger count function.

### 22. UMAP Analysis

The downstream quality control, statistics and visualization of PARCB single cell RNAseq data were performed mainly using the Seurat (v3.2.3) R package (94). Briefly, the data from all four patient series was first filtered for cells with total number of unique features above 500 and below 10000 as well as mitochondria feature counts below 10%. The mitochondrial genes and ribosomal genes were then removed from the count matrix for the downstream analysis. To overcome batch effect, the inventors performed Seurat integration between batch 1 (Series P2 and P5) and 2 (Series P6 and P7). Briefly, for each batch, the two corresponding matrices were combined first, and log transformation and library size normalization were performed with NormalizeData function. Then the 2500 most variable genes were selected as anchor features to integrate for all coding genes. After integration, the top 30 principal components were used to perform UMAP analysis.

Processed single cell RNA-seq data of advanced prostate cancers were downloaded from the Single Cell Portal hosted by Broad Institute (49). For this dataset, UMAP analysis was performed on TPM values of prostate cancer cells only as defined in the paper using the umap function in base R. For UMAP visualization of this dataset, TPM values were log 2 transformed with a pseudo count of +1. Single cell RNA-seq data of N-myc GEMM tumors (31), and human biopsy and GEMM tumors (50) were downloaded from the Gene Expression Omnibus (GEO) database with the accession numbers GSE151426 and GSE21035, respectively, and processed with cellranger count. In the Brady et al paper, single-cell data were first filtered for cells with total number of unique features >200 and <10000 as well as mitochondrial feature counts <10%. The inventors then performed Seurat SCTransform integration on each sample. Briefly, for each sample, the matrices were first combined and normalized using SCTransform function. Then the top 3000 most variable genes were selected as anchor features to integrate all genes. After integration, the top 15 principal components were used to perform UMAP analysis. In the Chan et al paper, GEMM single-cell data were filtered with the following thresholds nFeature_RNA >200 & nFeature_RNA <8000 & percent.mt<5 and human biopsy tissues single-cell data were filtered with nFeature_RNA >200 & nFeature_RNA <10000 & percent.mt<5. Seurat integration of filtered cells for both datasets were then performed as described above. After integration, the top 50 principal components were used to perform UMAP analysis.

In the Dong et al analysis, the human biopsy scRNA-seq data was downloaded from GSE137829. The inventors used the filtration parameters of the manuscript, total number of unique features >500 and <7000, and mitochondrial feature counts <10%. The inventors filtered cells to only include epithelial (cancer) cells, as described by the CellType column in the annotation. Seurat NormalizeData was used with the LogNormalize method and a scale factor of 10000. The top 30 principal components were used to perform UMAP analysis.

### 23. Inferred Cell Type and Cellular Lineages Analysis

The cell type inferences of PARCB single cells were implemented using the singleR R package (48). For scoring each cell for each general cell type, the Human Primary Cell Atlas data from LTLA/celldex package that contains normalized expression values was used as the reference.

Single cell trajectory analysis of PARCB samples was performed using two different methods, expression-based method Monocle2 (52) and RNA Velocity based method scVelo (53). For Monocle2, the integrated Seurat object was used as the input for the program. DDRtree was used as the reduction method. Cells were ordered by the most variable 3000 genes in Seurat. For calculating pseudotime, the KRT5 population was selected as the root state. For RNA velocity, the spliced and unspliced counts were quantified by velocyto accounting for repeat masking. The spliced counts were then normalized using Seurat sctransform method followed by integration by batch. The integrated data was used for UMAP visualization. In scVelo, the data was filtered for genes with a minimum of 5 shared counts. The top 3000 highly variable genes were extracted based on the dispersion. Velocities were estimated by dynamical model and then projected onto the UMAP embedding.

### 24. Differential Gene Expression Analysis in Single Cells

FindMarkers function in Seurat R package (described above) was used to identify differential expressed genes between ASCL1+ and ASCL2+single cell populations. Patient series was regressed out by including it as the covariate. ASCL1+ cells and ASCL2+ cells are defined as cells with log normalized expression counts >0 for ASCL1 or ASCL2, respectively. Genes that are differentially expressed in ASCL1+ population were defined by the difference of gene expression in ASCL1+ cells minus the one in ASCL2 expression (log and library size normalized) above 3. Genes that are differentially expressed in ASCL2+ cells were defined by such a difference below −1.

### 25. CUT&RUN Sequencing

The CUT&RUN experiment was performed using previously established method (61) (Skene et al., 2018) and the manufacturer's protocol (Cat #86652, Cell Signaling). 100k live cells were used per reaction. 50 pg of Spike-In DNA (Cat #12931, Cell Signaling) was added per reaction for downstream normalization. DNA was purified using MinElute PCR Purification Kit (Cat #28004, Qiagen), followed by fragmentation by using sonicator (Cat #M202, Covaris). Dual size selection was applied using KAPA Pure beads (Cat #KR1245, Roche). DNA Libraries were prepared with the KAPA DNA HyperPrep kit (Cat #KK8504, Roche).

Sequencing was performed on Illumina HiSeq3000 for a SE 1×50 run. Data quality check was done on Illumina SAV. Demultiplexing was performed with Illumina Bcl2fastq v2.19.1.403 software. Raw FASTQ files were processed using the published ENCODE-TF CHIP Seq pipeline. Batch 1 samples (P3-TP5 and P7-TP6) were processed with the parameter “chip.paired_end”: false while Batch 2 sample (P2-TP6) were processed with the parameter “chip.paired_end”: true. For all samples, the reads were trimmed and aligned to hg38 (target) and S. cerevisiae strain S288C (spike-in) reference genomes using bowtie2. After alignment, Picard was used to remove PCR duplicates reads and SAMtools was used to further filter high-quality paired reads (i.e., remove reads that were unmapped, not primary alignment, reads failing platform, and/or multi-mapped). Peak calling was performed using MACS2. Peaks overlapping with blacklisted regions were removed. Lastly, spike-in normalization factors were calculated following established protocol (95).

### E. Tables

TABLE 1
Upregulated genes in each hierarchical clusters (HC) (Related to FIG. 1D)

| HC1 | HC2 | HC3 | HC4 | HC5 | HC6 |
|---|---|---|---|---|---|
| gene | gene | gene | gene | gene | gene |
| POLB | MST1 | HTR2B | PLXDC1 | FEZF1 | HMHA1 |
| ADAMTS9 | SMIM10L2A | KRT10 | COL3A1 | DNAH2 | ZNF517 |
| MEIS3 | SCD | CFAP52 | LEFTY2 | GPR22 | SPPL2B |
| FGR | TMEM91 | TMEM88 | PLN | TMOD1 | RAP1GAP2 |
| FAM107A | ZNF404 | WDR38 | ADAMTS16 | HPCA | TNFAIP8L1 |
| GABRR2 | ADAMTS13 | ESR1 | THY1 | ADGRB2 | PXYLP1 |
| NXPE2 | STRC | SPAG17 | MAP4K1 | GNG2 | ZDHHC11 |
| NAV3 | ZC3H6 | OCA2 | HOXB9 | HOXD8 | PRSS53 |
| MRPS6 | ADHFE1 | CARD6 | COL1A2 | SFTPA1 | LRRC27 |
| PTCHD1 | ITPR1 | CA3 | TSPAN32 | RIMS3 | CCL27 |
| ARNTL2 | AGBL2 | ACE2 | TBX2 | SFTPA2 | WDR25 |
| MAP4K4 | CRTAP | GLRA4 | SPOCK2 | ADCYAP1R1 | ROR1 |
| KISS1 | WDR31 | HECW2 | TMEM235 | CDH19 | ANO9 |
| AEBP1 | SMIM10L2B | CRISP3 | PDGFRB | FAM124A | DMPK |
| CACNB1 | LRRC29 | ANGPTL7 | BARHL1 | SLC8A2 | MSRB1 |
| CD93 | C5 | KRT4 | SYNGR4 | FAM69C | ERO1LB |
| GIMAP8 | GSDMB | SPRR3 | DMRT2 | CD79B | GPR35 |
| DRAP1 | CTBS | ATP12A | TTBK1 | AZIN2 | CSF1R |
| ADGRL4 | OCEL1 | HYDIN | SLC6A1 | B3GAT2 | MICAL1 |
| FGFR1 | CD164L2 | HERC6 | CD248 | ZNF804B | DDX11 |
| LPXN | ALDOC | KCNE1 | AVPR1A | MEGF10 | SERPINI1 |
| LOXL4 | CD72 | ROPN1L | HAND1 | ZNF385C | GRK1 |
| IL2RG | DDB2 | KRTDAP | LRRN3 | CAPN6 | LYG2 |
| CUX2 | MAP1LC3B2 | CAPN13 | CXCL12 | PTGER3 | IGSF22 |
| F2RL3 | FBXO24 | ITPKC | ATP1A2 | ARPP21 | BMP8B |
| ITGA10 | SPATA7 | SLAMF7 | MUC5B | APOB | SLC12A7 |
| SLC30A2 | SLC25A27 | MAP3K19 | WFDC1 | C1QTNF4 | SORBS1 |
| SOD2 | UCN | C5orf49 | ARHGAP36 | FAM49A | SLC16A11 |
| C7 | NTN5 | KLHL6 | COL9A1 | MFAP4 | COL4A4 |
| NTF3 | CNTNAP1 | SNTN | CETP | PIPOX | TTYH2 |
| TNIP1 | LMNTD2 | KMO | LILRA2 | ARHGAP22 | STARD10 |
| FERMT1 | DBP | AKAP14 | POSTN | PNMA6A | STK32C |
| ANO1 | TSPAN7 | C9orf24 | SLC14A2 | ELMO1 | HID1 |
| PECAM1 | LEPR | CA1 | CDK15 | POU3F2 | NTHL1 |
| TMEM204 | TNNT2 | S100A7 | PIK3CG | CCDC177 | TCN2 |
| RB1 | GOLGA8R | S100A8 | PYY | NCAN | CDHR5 |
| SMCO2 | FBXO15 | S100A12 | MYBPC2 | ALOX5 | MAP6D1 |
| TRAF1 | GUCY2D | CD36 | NTM | NAALAD2 | CACNA2D1 |
| BEGAIN | PRAM1 | CA2 | REM1 | DLG2 | FAM174B |
| ARHGEF15 | ZCWPW2 | TMEM190 | SYCE1 | FNDC5 | BIN1 |
| PTGDS | SLC22A17 | MOGAT2 | HRH3 | RNF212 | HIPK4 |
| CCDC71L | SMIM1 | TEKT1 | NPFF | ACADSB | FSCN2 |
| PLA2G4C | LPIN3 | SAMD9 | BMP5 | GATA4 | LCTL |
| NOTCH4 | PIP5KL1 | PLBD1 | C11orf21 | MNX1 | TMED6 |
| SEMA3B | GOLGA8N | SLC5A1 | | HOXA4 | MGAT4A |
| IL4I1 | ACBD4 | C9orf171 | | TMPRSS6 | PRSS27 |
| PRDM16 | C11orf65 | GABRG3 | | LRFN1 | FMO5 |
| GALNT15 | DMGDH | GSTA3 | | PRH1 | RAB30 |
| GIMAP6 | AIFM3 | DRC1 | | ASTN2 | POLN |
| TIE1 | HIST2H4A | PIH1D3 | | GPC2 | SUSD3 |
| GK | TNXB | C16orf89 | | MAMSTR | RNF224 |
| ANGPT2 | RNF125 | OLFM4 | | GPC5 | CYBA |
| PLVAP | RBM43 | SHISA2 | | TAC3 | CEP126 |
| MYO1B | ATP6AP1L | TNNC1 | | SH2D2A | ADAMTS17 |
| MET | PRRT1 | BIN2 | | HOXD4 | SMCO4 |
| PPAP2B | SIAE | CCDC170 | | CAMK4 | KLF15 |
| PDE9A | ICAM5 | FAM92B | | TCF15 | ADRA2C |
| EMP3 | HAPLN2 | IRX6 | | NKX6-1 | GIPC2 |
| TNFRSF1B | P2RX7 | CHRNA6 | | DOCK2 | KCTD12 |
| SLIT2 | PPM1J | BAALC | | POU4F3 | BCAN |
| CEACAM1 | MYO15B | CLEC7A | | PDE3A | USHBP1 |
| WIF1 | CCK | PIP | | HOXD3 | INPPL1 |
| RASSF8 | CFAP53 | UGT2B17 | | HPR | BSPRY |
| GRB7 | C1orf228 | KANK4 | | RTN1 | ALG1L |
| SHH | C1QTNF6 | CFAP99 | | INHBB | F10 |
| SLC7A2 | GSTM2 | ZMYND10 | | HOXA2 | SNAI3 |
| ZBTB16 | CDNF | ADH7 | | MAB21L1 | TGFB3 |
| BIRC3 | PPP1R32 | IL36A | | NTN3 | TIGD3 |
| NOG | FSBP | ENDOU | | SOBP | SLC25A34 |
| HLA-G | FUT5 | IFNK | | NCKAP1L | PNPLA7 |
| C17orf67 | DHDH | TMPRSS11E | | LRAT | STX1A |
| FAM65C | P4HA2 | CHL1 | | PALD1 | JPH1 |
| HS3ST2 | ITPKA | ABCA13 | | LHX2 | GALNT8 |
| REN | TRIM73 | SERPINA3 | | LIMD2 | CLDN18 |
| FAM19A3 | DNM1 | CARD17 | | KCNT1 | PLLP |
| KLF9 | RSPH14 | BCAS1 | | VASH2 | FAM149A |
| CXorf36 | DRD5 | BST2 | | RNF157 | COL4A3 |
| PXN | KIAA0319 | MUCL1 | | ARNT2 | SEC11C |
| BATF3 | KLHDC8B | CCL5 | | IMPG2 | LGI2 |
| TRIM47 | ZNF20 | CFAP126 | | TSPAN18 | TBX20 |
| TRIB2 | LOXL2 | FAM166B | | GNB3 | BEST4 |
| CRTAC1 | ORAI3 | C5orf46 | | CNIH3 | ATAD3C |
| TNFRSF21 | PGF | CXCL14 | | KCNS1 | PITPNM2 |
| FSTL3 | EDAR | PLEKHS1 | | OVOL3 | GPR157 |
| SRD5A2 | USH1G | MOXD1 | | GRK5 | GPR155 |
| GPR176 | CPS1 | SEPP1 | | SEMA3G | TTC39A |
| PTPRG | CNTNAP3 | DNASE1L3 | | FAM19A4 | AGMAT |
| RAI2 | CFAP69 | STEAP4 | | FIGN | TM6SF2 |
| FLT4 | MRC2 | HLA-DMA | | MYH7B | EPHB1 |
| EVA1C | TXNIP | FABP5 | | LANCL3 | PLAC8 |
| TGM1 | NPC2 | IVL | | LY6H | SYBU |
| FBXW10 | DCST2 | ASPG | | TLX2 | MANEAL |
| IL18RAP | AMT | SPINK5 | | DLX5 | GPR37L1 |
| ESAM | CACNA1F | SYT8 | | STAC3 | SEPT14 |
| ACHE | N4BP2L1 | TMEM71 | | ADAMTS18 | ANKRD65 |
| LYVE1 | DUSP13 | FAM81B | | POU4F1 | DAGLA |
| ADGRG2 | TMEM255A | SPRR4 | | NRG2 | MMP26 |
| CCDC116 | YPEL4 | PDZK1IP1 | | ELAVL3 | ESYT3 |
| PANX2 | CATSPERG | CALML5 | | ADGRD2 | MPP1 |
| SLC5A3 | CNTN1 | FAM3D | | C2orf82 | SCN8A |
| RELB | IL13 | ENPP3 | | PNMA3 | PGM5 |
| NPTX2 | DNHD1 | HMCN1 | | FAM171A2 | SNTB1 |
| CYP24A1 | VAV3 | PIGR | | FAM122C | PSD |
| NOTUM | XKR5 | AADAC | | AGRP | TBC1D30 |
| SIGLEC15 | P2RY1 | TNIP3 | | ATRNL1 | SMPD3 |
| CYP1B1 | NRP1 | CLDN4 | | ANGPT4 | ERICH5 |
| JPH3 | ATP8B3 | SLC10A6 | | PRTG | DENND6B |
| CLVS2 | FADS6 | LY6D | | ACADL | GPR160 |
| SLC14A1 | SH3D21 | ARL14 | | MT3 | AKR7L |
| GCNT3 | ZNF862 | CCL24 | | ILDR2 | KCNIP3 |
| FNDC3B | NQO1 | UGT2B7 | | SHD | GGT1 |
| PITPNC1 | CFAP70 | SLC6A2 | | ASCL2 | CAMK2B |
| MUC17 | LEMD1 | CFTR | | NTRK1 | RASA4 |
| PGC | PNCK | CDKN2B | | GRIN2D | GRIN2C |
| PPARD | GRIN3B | NOSTRIN | | STAT4 | TLDC2 |
| MUC3A | PYROXD2 | DNAH3 | | ISLR2 | SMLR1 |
| KLK2 | CLDN2 | DNAAF1 | | HES7 | COLEC11 |
| BCAR3 | LRRC4B | RNASE6 | | TSPAN12 | RAP1GAP |
| DDR2 | FAXDC2 | CST6 | | C14orf37 | RFX6 |
| ZNF853 | MYOM1 | SPRR1B | | CACNA2D2 | TMEM198 |
| FMOD | KLRF1 | SPSB1 | | KCNIP4 | C3orf14 |
| ZNF366 | PAGE2B | A2ML1 | | RBM46 | MAPK12 |
| KCNK1 | KLHDC1 | S100A9 | | KCNN3 | CORO7 |
| BACE2 | GAMT | C15orf26 | | MYOZ3 | SDSL |
| TM4SF18 | TGFBR3L | FRMD4B | | FOXE3 | TMEM156 |
| IRAK2 | NR4A1 | SLC28A1 | | TUBB4A | CEP112 |
| PLIN2 | SESN3 | PLAC4 | | PDGFRA | NPTXR |
| FLNA | GPRASP1 | GDNF | | DRAXIN | BMP8A |
| SH2D3A | FTCD | TMPRSS11A | | TUBA1A | NR3C2 |
| KIAA1549L | TCTE1 | FOLR1 | | CSF3R | LAT2 |
| NOS2 | CD302 | SLC13A2 | | COL25A1 | GALR2 |
| ACVRL1 | CDRT4 | SPRR2A | | CYP3A7 | IGSF11 |
| SYNDIG1 | RHCE | SLC28A3 | | NALCN | ASPHD1 |
| CD59 | NGEF | OAS2 | | GCK | CCDC154 |
| SCARF2 | BMP4 | DTHD1 | | SOX17 | ACE |
| VCAN | MYOZ1 | GNA14 | | NHLH1 | MOBP |
| ADRA2B | ACOX2 | HLA-DQA1 | | TFAP2E | SHANK3 |
| ESM1 | KLRD1 | PSAPL1 | | KCNMB1 | GAL3ST2 |
| TPSAB1 | ENDOD1 | GABRQ | | KCNJ4 | CCDC181 |
| TMIE | FCN3 | PTPRZ1 | | DOK5 | ABHD12B |
| IL15RA | ERICH2 | ST6GALNAC1 | | NRTN | DLGAP1 |
| ZBTB46 | ACSF2 | FAM46B | | CDH4 | RUNDC3A |
| KRT7 | GPR179 | FABP4 | | FOLR2 | SLC34A3 |
| GGT5 | CCDC183 | SAA2 | | SYNE3 | HNF4A |
| IL23A | NPB | FOXJ1 | | MEGF11 | PRR15L |
| DPCR1 | GJA3 | IFI44 | | COL19A1 | FBXL16 |
| TAL1 | ALOX15B | SLC15A2 | | PKNOX2 | SLC39A5 |
| ARHGAP31 | PLIN1 | HYAL4 | | FAM135B | SGSM1 |
| NXPE1 | DQX1 | C20orf85 | | SLC10A4 | NDNF |
| MYCT1 | ACSM1 | AKR1B15 | | LMCD1 | PRPH2 |
| PTGES | RGL3 | KRT84 | | ESX1 | LAMA1 |
| KLK3 | ZNF610 | KYNU | | NEUROD6 | BHLHA15 |
| NR1D1 | ANKRD29 | FAM83C | | PTPN7 | PPP1R36 |
| AOX1 | GLIPR1L2 | OASL | | CCM2L | SLC38A3 |
| GNG11 | ST6GALNAC2 | WNT5A | | IL12RB2 | JAM3 |
| SMR3B | SULT1A2 | VTCN1 | | C5orf58 | T |
| CILP2 | SYNDIG1L | HLA-DRA | | FAM184B | PTPRN2 |
| ALDH1A3 | MLXIPL | EPSTI1 | | ASMT | STAB1 |
| MT2A | DCST1 | C1QTNF2 | | WAS | CACNA1H |
| GALNT5 | EGR2 | CYP4Z1 | | NEUROD2 | HNF4G |
| VEGFA | CDK18 | B3GNT6 | | CSF2RB | FAM69B |
| KCNQ5 | PARK2 | LGALS9 | | TIGIT | GAB3 |
| MS4A7 | LGALS1 | ENKUR | | ZYG11A | RGS17 |
| HBB | WISP2 | ANKRD66 | | EPHA7 | FRY |
| DRAM1 | TRPV1 | PGLYRP4 | | NRXN2 | TMEM178B |
| CYP1A2 | HHAT | LAMB4 | | C11orf85 | RAB19 |
| ALPP | DNAJB13 | MX1 | | PTGES3L | SMIM6 |
| FOXD4 | HAVCR2 | TGFBI | | SAMD11 | ADCY5 |
| IL6ST | UPP2 | AMTN | | ZFHX4 | C1orf95 |
| LMO7 | GM2A | DRC7 | | XK | SPATA12 |
| ITGA3 | TMEM139 | ADGB | | VAV1 | MAPK8IP1 |
| MYH11 | MATN2 | TTC29 | | NXPH4 | KIF12 |
| LAYN | HPN | KRT6B | | CTNNA3 | FAM189A1 |
| ART4 | LTB4R | NPR3 | | EBF3 | KIAA2022 |
| CD34 | TNNI3K | CXCL2 | | PRSS21 | NPC1L1 |
| CLIP2 | ENO2 | SCGB3A1 | | PLP1 | DLL4 |
| NYAP1 | C4orf47 | CDC20B | | RBFOX1 | FSTL4 |
| TNFAIP2 | ARID3C | MMP9 | | PAK3 | GAS2 |
| CEACAM3 | UPK3A | STX11 | | CHSY3 | RNF148 |
| SYPL2 | SLC52A1 | CLEC3A | | CORO1A | BOLL |
| TXNDC2 | PLEKHH2 | THEM5 | | EMX2 | FBLN7 |
| CELF6 | SNCG | DAW1 | | ANO2 | GREB1L |
| IRF1 | ASPDH | LTF | | PRDM13 | DOK1 |
| RAPGEF3 | SYCE3 | C6orf118 | | SCN1A | LDHAL6A |
| GJC2 | TLR5 | TYRP1 | | PDZD7 | FAM105A |
| BCL6B | MT-ATP8 | TRIML2 | | OPRD1 | NAALADL1 |
| ALOX12 | SLC16A14 | LYPD2 | | ARHGAP4 | ADA |
| GPR4 | CTGF | C2orf72 | | RAB33A | KCNIP2 |
| OXTR | BCAM | C1orf194 | | NHLH2 | LONRF2 |
| STAT5A | GUCY1B3 | TSPAN8 | | ZIC3 | MUC5AC |
| NRXN3 | MERTK | CXorf49 | | PREX1 | IKZF1 |
| FAM129B | SLC35F3 | LRRC71 | | COL11A1 | COL22A1 |
| CEBPD | CGB7 | C9orf135 | | POU2F3 | OCSTAMP |
| NFKB2 | SRPX | DEFB4A | | PLCG2 | TMEM255B |
| CBLN3 | KLRK1 | MYBPC1 | | PRR23D1 | TFEB |
| HSD11B1 | CREG2 | PLA2G10 | | PRR23D2 | KCNK15 |
| EBI3 | ZC4H2 | PTHLH | | ADORA1 | MED12L |
| AGPAT9 | SLC16A4 | PTPRB | | TSPAN11 | HSH2D |
| GIMAP5 | SH3GL3 | SERPINB12 | | BZRAP1 | CHRM5 |
| MUC12 | MROH7 | ANPEP | | SLC24A4 | SLC29A4 |
| A4GALT | TCEA3 | GABRA3 | | ZFR2 | ANKS4B |
| APLNR | DNAH6 | RSAD2 | | KIF26A | SDK2 |
| PAOX | C11orf70 | CARD18 | | NPPA | RPRM |
| ELK3 | ZFP2 | IGFL1 | | KCNH1 | GPHA2 |
| SEMA3C | ASB9 | C10orf107 | | CHST4 | TRPM6 |
| CSF3 | FER1L5 | EGFL6 | | RIMS4 | HCN2 |
| ABTB2 | GOLGA6A | APOD | | ISM2 | STMN3 |
| SQRDL | PODNL1 | EDN2 | | CCDC141 | ZACN |
| AQP9 | FAM132A | NCCRP1 | | HMX2 | KRTAP10-4 |
| SFRP4 | MR1 | SLC3A1 | | TMEFF1 | VIPR2 |
| ARHGEF40 | CAPS2 | SLC6A14 | | PLA2G4A | CCDC78 |
| C1S | CNTN3 | TMEM45A | | RGAG1 | IHH |
| CHST2 | TNFAIP8L3 | CCL22 | | MGAT5B | DLX1 |
| RAB31 | NRN1L | CLCNKB | | PATE2 | EPHA10 |
| HP | TMEM59L | HOPX | | CLGN | IL12B |
| TINAGL1 | ACSS2 | FAM26E | | SPP1 | CORIN |
| SLC25A52 | ZNF90 | HLA-DPA1 | | PHOSPHO1 | ADPRHL1 |
| TNS1 | LGI4 | GPNMB | | FOXI3 | OLFM1 |
| ALOXE3 | PCP2 | MUC15 | | ATCAY | SPTBN5 |
| C1QTNF1 | SMTNL1 | KRT79 | | FAM159A | KCNAB2 |
| PGLYRP2 | RBP7 | UGT2B15 | | CASQ2 | EIF4E3 |
| SSTR5 | KCNJ2 | ALPK2 | | NKAIN2 | FAM196A |
| NPR1 | GPX3 | CFAP47 | | ADGRB3 | AKR7A3 |
| C16orf45 | ZNF528 | CRCT1 | | HOXC13 | BHMT2 |
| GSAP | CCDC8 | FUT6 | | ASIC2 | RAB3D |
| ROBO4 | CNGA1 | SERPINB11 | | GAS7 | DNMT3B |
| ADAM8 | PPP1R3C | BPIFB1 | | NES | KCNG3 |
| PDK4 | TMEM200B | HLA-DOA | | IL17RB | HNF1A |
| UBASH3B | ZNF160 | C6 | | CALHM1 | CLDN25 |
| LY6K | IFITM10 | NTRK2 | | CNDP1 | SLC25A47 |
| SOX18 | C21orf62 | C1orf158 | | SOX8 | SPON2 |
| GCM1 | ABCB5 | RRAD | | FAM196B | TMEM74 |
| AKAP12 | OXER1 | GPR1 | | NKX2-4 | EXOC3L1 |
| SH2D4B | ACSM3 | ITIH6 | | DPYSL3 | TMEM52 |
| TTLL8 | ZNF563 | DNAH12 | | HRASLS | CCDC13 |
| ITGA2 | ZNF492 | ACKR2 | | SLC17A7 | IGFALS |
| SQSTM1 | OR13J1 | SULF2 | | ADD2 | AMACR |
| COL6A2 | GOLGA8O | DKK2 | | PNPLA5 | THEMIS2 |
| ECM1 | ADSSL1 | SDR9C7 | | CBX2 | RASA4B |
| KCNN4 | MAP2K6 | PZP | | DLX6 | FAM155B |
| B4GALNT1 | HACD1 | CH25H | | CALHM3 | F2 |
| NFKB1 | NXNL2 | CROCC2 | | SLC6A15 | Mar. 3 |
| ABCC3 | REEP6 | SPRR2E | | OR13A1 | SOWAHA |
| KIAA0040 | CD52 | ARSF | | MATK | PSTPIP1 |
| GLIS1 | EPHX2 | GABRP | | AKNAD1 | PKIB |
| EFNB1 | HRCT1 | CMPK2 | | ENHO | TEX29 |
| MYH3 | KANK3 | IQGAP2 | | FAM57B | MARCO |
| TYMP | PAK7 | GAS2L2 | | SOX11 | FAXC |
| PI16 | FRRS1L | TMCC3 | | NOVA2 | GRIN1 |
| NR5A2 | RNF225 | MYO16 | | SCRT1 | A1CF |
| HLX | EGR3 | GLYATL2 | | TRIM49D2 | CKMT2 |
| STK32B | HIST1H2BD | SERPINA1 | | STMN2 | GNAO1 |
| SSH1 | COX6B2 | SLC5A8 | | SFTPB | CILP |
| ADCY8 | ZNF843 | REG4 | | ELFN1 | FOXA3 |
| SH3RF3 | GPX8 | KIAA2012 | | EPHA8 | GADD45G |
| SHANK1 | GOLGA6B | SLC22A2 | | GAL | SCAMP5 |
| XDH | AKAP3 | LGR5 | | UBTFL1 | SARDH |
| TPSB2 | ZNF703 | SELENBP1 | | ZNF280A | ZP2 |
| KCND3 | IL20RB | IFI44L | | ART5 | TRIM72 |
| ADAMTS10 | SCN4B | IFIT3 | | PPP1R1A | DNAJC6 |
| IL32 | H1FNT | OVOL1 | | ZNF114 | C9orf173 |
| CPM | RASSF10 | FAM216B | | PATE3 | BTNL9 |
| RASGRP3 | HAMP | ZBBX | | CYBB | ADGRF2 |
| MSRB3 | NOTCH3 | MORN5 | | DPYS | SLC7A4 |
| SMAD3 | KRT222 | S100A7A | | KCNQ4 | SLC26A9 |
| VSTM2L | PDZD3 | IL5RA | | CHN1 | LRRC36 |
| IL13RA2 | TBC1D10C | C7orf57 | | SPSB4 | FRMPD3 |
| CCL14 | SLC16A5 | TNF | | CSH2 | ADCY2 |
| EPHB2 | MCHR1 | SLC9A4 | | HMX3 | DTNA |
| SH3TC2 | CD300A | IL19 | | CCL25 | PRR18 |
| CRP | FAT2 | LRRC4 | | MYEOV | TMEM163 |
| APCDD1L | SOX21 | ROS1 | | DDI1 | LUZP2 |
| PLAT | TRIM34 | NOD2 | | RBM20 | C2CD4B |
| CARD11 | ZMAT1 | GJB6 | | PNLIP | PRODH |
| C1R | HAAO | C11orf16 | | CPVL | SLC43A1 |
| TRABD2B | KRT74 | ERP27 | | CHRDL1 | CPNE9 |
| MRGPRF | DUSP4 | ARSI | | POU3F3 | MAPK8IP2 |
| SGPP2 | MTRNR2L12 | ADAM12 | | FAM159B | LHFPL4 |
| FGF2 | SLC19A3 | MRAP2 | | PLD4 | RIPPLY3 |
| RSPO1 | C16orf96 | ROBO2 | | P2RY14 | GAL3ST1 |
| SHE | KLRC2 | SLC24A3 | | VEPH1 | CELSR3 |
| CSF1 | ALX4 | KRT1 | | RYR2 | MYO7B |
| WNT6 | TMC1 | CFAP221 | | TBX5 | PPARGC1A |
| RAB3IL1 | WNT10A | CARD16 | | GJC1 | NKX1-2 |
| PLCD3 | KCNJ1 | CYP2C18 | | CORO2B | DEGS2 |
| PODN | PNMA5 | RBP2 | | P2RY10 | TIMP4 |
| TFPI2 | USP17L2 | CDH26 | | ZIC1 | KLK11 |
| NLRP2P | MIOX | FGG | | FAM19A5 | RTN4RL2 |
| WBSCR17 | USP17L7 | CFI | | SLC17A6 | USP2 |
| ANXA3 | PPEF2 | MX2 | | ELOVL2 | DNASE1L2 |
| RBP4 | KCNA7 | UNC93A | | ZNF560 | CIDEC |
| OAF | CLIP4 | DIO2 | | GRASP | MCOLN3 |
| ABLIM3 | TRIM74 | CCL19 | | PLCXD3 | KRT81 |
| FGD5 | ABCB4 | LTK | | ZNF536 | SLC2A12 |
| DRD1 | C22orf23 | MYO18B | | LINGO1 | IGFBP5 |
| MMP21 | IQSEC2 | KRT6C | | LSAMP | C4orf48 |
| CD180 | BLVRB | TACR1 | | C11orf53 | TMTC1 |
| LAMC2 | FAM131B | LIF | | PROK2 | GBA3 |
| SH3BGRL3 | SLC34A1 | SCGB1A1 | | FAM198A | BSN |
| SHISA6 | ARL4D | CLDN14 | | PCK1 | AP3B2 |
| LTB | RNF112 | ELF5 | | TRPC5 | MAGEL2 |
| CDH5 | BAIAP3 | TMPRSS11D | | DNAH10 | PABPC4L |
| RASAL3 | HRNR | FN1 | | LCK | CECR6 |
| GIMAP4 | C2orf73 | CCDC185 | | TTYH1 | EPB41L3 |
| SELE | ZNF737 | CD38 | | GRAMD1B | SLC5A4 |
| PSG1 | PTGER4 | MRVI1 | | NCF4 | DRP2 |
| A2M | ZSCAN18 | SH2D1B | | NMU | VWA5B2 |
| GFPT2 | ALPK1 | KRT75 | | BTBD17 | DIO1 |
| CYP26B1 | NDRG1 | PTPRR | | TRIM49 | CRMP1 |
| TCF21 | ZNF816 | FOLH1 | | AVPR2 | P2RX6 |
| HSD17B2 | LPAR3 | SV2C | | LHX1 | RHBDL1 |
| GCLM | KLK5 | PLAUR | | HELT | FGF13 |
| CHIT1 | ODF3 | OLR1 | | TDRD9 | CHRD |
| SH2D5 | LPAR5 | PGLYRP3 | | WSCD2 | MOV10L1 |
| FOXF2 | CEMIP | IFI27 | | ADAMTS4 | ABCA3 |
| TGM2 | APOBEC3H | TMEM52B | | DYNC111 | CCRL2 |
| CCL2 | OSR1 | HEPHL1 | | P2RX3 | SLC35D3 |
| CXCL9 | PROP1 | KRT3 | | ADAMTS19 | SSTR3 |
| CD200 | C17orf78 | BPIFB2 | | NOXO1 | CDH7 |
| MFSD4 | ZSCAN10 | LYPD3 | | EBF1 | FAM167A |
| PAGE4 | ST8SIA6 | HCG22 | | NEUROG1 | RNF128 |
| ANP32D | CCDC146 | LGALS7B | | LCP2 | FAM163A |
| CCR7 | PLSCR4 | PRDM1 | | RXRG | UTS2R |
| ICAM2 | WEE2 | CLEC1A | | AVIL | LCE1E |
| TSGA10IP | TNFSF13B | COL5A2 | | LRMP | LINGO2 |
| PAPPA | CCDC7 | HLA-DRB1 | | HCK | ISG20 |
| STEAP1 | GSTM5 | BRINP1 | | EMILIN3 | FOXA2 |
| TGFB1I1 | EMB | FATE1 | | DAZ1 | SRMS |
| ITGAM | ADAD2 | SAMD9L | | C8orf48 | ADAMTS14 |
| RGCC | RAB7B | CLCA1 | | RSPO4 | KSR2 |
| GRID1 | KLK7 | ARSH | | CNR1 | MAP4K2 |
| KLKB1 | S1PR5 | NAT8B | | SYN3 | TRIM55 |
| IGFBP6 | TG | UPK2 | | CD33 | PHF21B |
| RHBDF2 | NLRP6 | FZD8 | | DMRTB1 | BIK |
| CLMP | EFEMP1 | ZG16 | | TRPM5 | KCNK13 |
| DUOXA2 | CDA | CDHR4 | | KRT72 | CAMK2N2 |
| PPARG | LRRC3C | GRIA3 | | ZIC4 | CXXC4 |
| CPED1 | STX19 | KCNF1 | | LY86 | MYO1A |
| GUCY2C | SEC16B | TMEM213 | | ACTC1 | RPH3AL |
| RUNX3 | KRT36 | KIAA1683 | | DPYSL5 | HEY2 |
| PRKD1 | BCL2L14 | SPRR2D | | RGS21 | PDE4C |
| SP6 | HIST3H3 | IDO1 | | IGLL1 | BMP7 |
| MSN | AHSG | KRT20 | | OLIG1 | NCALD |
| FGF7 | MAF | CYP27C1 | | CHRND | PHYHIPL |
| TMEM92 | KCNE3 | SLCO2B1 | | SLITRK1 | ANKRD33 |
| NPAS2 | ID4 | C17orf99 | | CD69 | KCTD16 |
| LDB2 | DYNLRB2 | SUSD4 | | TESPA1 | MB |
| ABCB1 | KCNG1 | CEACAM6 | | DCLK3 | SYP |
| ALDH1A2 | MAGEE1 | PRR16 | | TMEM108 | CCDC108 |
| OLAH | RBMS3 | DSC2 | | MPO | KIF5C |
| TBX21 | FAM124B | ATP6V0A4 | | SCIN | RIPPLY2 |
| ITGA6 | TCEAL7 | WISP3 | | FAM150A | SEMA6B |
| CDRT1 | KRT15 | CXorf49B | | GSG1 | RTBDN |
| ZNF320 | SLAMF8 | TMPRSS11B | | SYT6 | PHACTR3 |
| IGFBP3 | DSC1 | CXCL10 | | BMX | MYBPC3 |
| ADAM33 | CCND2 | TNFSF14 | | RASSF2 | DES |
| KLK10 | KLK8 | ZBP1 | | OGDHL | RIMKLA |
| JCHAIN | TCAF2 | RAET1L | | GFRA1 | MEP1B |
| ITPR3 | SLC1A6 | CHP2 | | NEUROG2 | NRCAM |
| PGA3 | TRIM7 | WFDC12 | | PTPRT | HTR1D |
| SAMD4A | P3H3 | C4BPA | | DIRAS2 | ZIC2 |
| SHC2 | SLITRK6 | GDPD2 | | EPHX4 | FAM178B |
| AXL | CYSLTR1 | NR4A3 | | SLC30A10 | TM4SF5 |
| NLRP12 | C1orf177 | SLC6A11 | | PDZRN4 | SOAT2 |
| PPP4R4 | ZNF442 | STOML3 | | PDE2A | KCNE4 |
| TWIST2 | COL20A1 | PRSS22 | | TRIM71 | ENTPD8 |
| CLEC14A | MAOB | EPHX3 | | RALYL | KCNN1 |
| S1PR1 | PABPC5 | C1orf186 | | TLR9 | TMEM150B |
| VEGFC | TRIM6 | GREM2 | | ABCC8 | FRMD3 |
| GALNT9 | KRTAP29-1 | HTR1F | | PAX7 | MYBPHL |
| HAPLN4 | CPA6 | LEFTY1 | | TMEM100 | RET |
| PGA4 | KLF8 | UPK1A | | TRIM58 | CAMK1D |
| SLC22A14 | FGF1 | DAPP1 | | ZPBP2 | RASSF4 |
| TRNP1 | KCNK7 | CP | | EBF2 | ENTPD2 |
| ETS1 | SCN2B | GPA33 | | MRO | RNF186 |
| EGR4 | IGFL4 | EMP1 | | GFRA2 | PROC |
| AQP3 | MALRD1 | KRT13 | | HOXD1 | TOX3 |
| SPRY4 | NTSR1 | GSDMC | | OR2W3 | C12orf42 |
| TDRD10 | TEDDM1 | PRR29 | | THEMIS | BTN1A1 |
| LTBP2 | DPP6 | KRTAP3-1 | | TFAP2B | TEX101 |
| FAM150B | LGALS9B | LBH | | HORMAD1 | RAMP1 |
| LRRC38 | IRGC | VIT | | PIK3R5 | ONECUT2 |
| SELP | EQTN | PF4V1 | | APBA2 | RGS16 |
| FAS | TM4SF20 | SLC27A6 | | KBTBD12 | KIAA1324 |
| GLIS2 | OLFML2A | MROH9 | | COL2A1 | INA |
| KL | LAX1 | CYP4X1 | | CACNA1S | TRIM9 |
| DPT | ADTRP | TRIM15 | | OLIG2 | CAMK2N1 |
| NID2 | CASP14 | SLC22A3 | | RGS13 | SDK1 |
| MSMB | RIMBP3 | CXCL17 | | TMEM229A | RIMS2 |
| RRH | TMEM95 | SAA4 | | GUCA1A | DBH |
| GHSR | NID1 | C5orf52 | | SLITRK4 | CNTNAP2 |
| ISM1 | KCNJ5 | C4orf22 | | PADI4 | ATP4A |
| NCF2 | BTBD11 | FABP12 | | SLITRK3 | ADCY1 |
| SULT2B1 | TMEM31 | EGF | | RD3 | IRAK3 |
| VSX1 | ALAS2 | TNNI2 | | SYCP1 | BEX1 |
| MYOCD | PPP2R2C | LOR | | ADH4 | GLB1L3 |
| SMAD6 | LRIT2 | AIM2 | | NCMAP | ANXA13 |
| TIMP1 | TBL1Y | NOX1 | | FAT3 | SRPK3 |
| OR8S1 | TRIL | CLLU1OS | | PSD2 | NEU4 |
| SPRED3 | LAMP5 | FGB | | GFI1B | SKOR1 |
| PTGS2 | RNF39 | FBP1 | | CERKL | DCDC2 |
| DOCK5 | WBSCR28 | PI3 | | TNFSF8 | ADGRG5 |
| TAGLN | MXRA5 | FBXO39 | | IGFBPL1 | STXBP5L |
| IFNGR1 | GDF5 | PALMD | | OR2T33 | IRF4 |
| SPRY2 | XG | DOCK10 | | ATP8A2 | SYN1 |
| IL34 | DUSP6 | VWA3A | | SCML4 | NEFM |
| DUOX2 | ARR3 | KRTAP2-3 | | PRAME | HGD |
| EDN3 | NTF4 | LGALS7 | | ADGRD1 | SYT2 |
| ZIM2 | IQCF1 | IFI6 | | CLEC17A | ADORA2A |
| TLL2 | PPP2R2B | PSCA | | GNGT2 | RAB3B |
| CDH13 | RBP3 | SLPI | | AGTR1 | PIRT |
| PROSER2 | ODF3L2 | THRSP | | C1orf141 | SLC26A5 |
| CACNG8 | PLTP | EPPIN | | ST6GALNAC5 | GJB1 |
| ITGB8 | SMCO1 | IFIT1 | | CNTN6 | AFF3 |
| MSMP | PIWIL2 | MAP3K8 | | SLCO6A1 | HCN4 |
| CLIC2 | SOWAHC | C1orf87 | | SPIB | NEURL1 |
| F11 | UBQLNL | AGR3 | | NMUR1 | CXCR4 |
| MAGEA8 | CASP5 | LRRC55 | | MMP16 | PPFIA2 |
| NPHS1 | ZNF135 | FMO3 | | GAB4 | CYP4F3 |
| CPA3 | TSKS | CPA4 | | HIST1H2AJ | CCDC83 |
| CMKLR1 | EFCAB1 | HLA-DQA2 | | IQCJ | ASGR2 |
| GCSAML | EXOC3L2 | S100P | | SMTNL2 | SNAP25 |
| SSUH2 | GPR32 | ATP6V1B1 | | KCNQ2 | ADAMTSL2 |
| RNASE7 | PLXDC2 | MMP12 | | SLC35F4 | DLX2 |
| CXCL16 | NUGGC | SERPINA6 | | CRX | APOH |
| ADGRF1 | ART1 | VWA3B | | FSD1 | MAP1A |
| LRRC32 | FCN2 | FMO2 | | MORC1 | CLDN5 |
| NGFR | SIGLEC1 | SAA1 | | ADAMTS20 | SOGA3 |
| AJUBA | PRTN3 | VSIG2 | | MFNG | TNFRSF13C |
| CYP27A1 | LEP | WNT9A | | BCAT1 | GFI1 |
| FCAMR | FAM153A | HSPB8 | | SLC17A3 | KIAA0408 |
| PALM | DDX4 | MUC13 | | HOXC8 | LDB3 |
| SOX10 | GSG1L | ERN2 | | LRRTM3 | TBXA2R |
| NTN4 | PLK5 | AMBP | | IGDCC3 | PRR5L |
| PIWIL1 | ITM2A | XAF1 | | CHRNA1 | ADAM2 |
| CPO | MIA2 | BBOX1 | | LIN28B | PAX5 |
| RHBDF1 | PI15 | SULT1E1 | | HOXC10 | ARHGAP9 |
| BCL3 | GOLGA8M | ARG1 | | SCRT2 | CACNA1A |
| FGF10 | ZNF471 | THBS2 | | ADRA1A | CHRM4 |
| SRGN | ZNF257 | DSG4 | | KIAA1211 | B3GNT8 |
| BATF | TNFRSF17 | CCDC37 | | GAD2 | CRHR2 |
| LRFN5 | NOS1 | ANKFN1 | | GNG13 | SLC2A7 |
| FLRT3 | C6orf10 | BCL2A1 | | HOXC5 | DLGAP3 |
| DIRAS1 | HNF1B | UBXN10 | | SIX3 | C1orf127 |
| POU5F1 | VCX3B | SOSTDC1 | | ZFP42 | SHISA8 |
| PGR | PCDH7 | CALML3 | | PCP4 | ACOXL |
| MAST4 | ABI3BP | DMBT1 | | MYT1 | KCNJ3 |
| PTRF | TPTE2 | AARD | | RFPL4B | C12orf56 |
| NBL1 | VSX2 | RDH12 | | TMEM132E | TEX15 |
| CYP1A1 | ZNF665 | SORCS2 | | ONECUT1 | UNC79 |
| COL8A2 | EFHC2 | ANOS1 | | KCNB2 | ESRRG |
| BACH2 | C2orf57 | ARHGDIB | | GAP43 | PTH2R |
| CSPG4 | CLCA2 | HMGCS2 | | KIAA1210 | MRC1 |
| MT1M | KCNU1 | CIDEA | | TLX3 | NPTX1 |
| CD40 | AR | ACTBL2 | | HOXC6 | IGFBP1 |
| TLE2 | GCNT4 | DSG1 | | OR14A16 | PTGIS |
| RAB43 | CATSPERD | CD74 | | AIPL1 | REEP1 |
| NACAD | SNX31 | THBD | | MASP1 | AQP7 |
| TNFRSF12A | LHFPL1 | CLDN17 | | GNAT3 | KLHL32 |
| PROCR | ADAMTSL5 | CXCL3 | | OLFM3 | KIAA1644 |
| CDH16 | FOXN1 | SBSN | | COL9A3 | TRPM8 |
| ST8SIA1 | SLC36A3 | CSNK1A1L | | LHX3 | SLC7A14 |
| CACNA1C | DSC3 | C11orf96 | | SLIT1 | TNFRSF11B |
| SERPING1 | PAMR1 | NPY | | HTR3E | CHRNB2 |
| AMN | GLIPR1L1 | TM4SF1 | | ZDHHC22 | KRT86 |
| ABCA9 | SP8 | SLC38A11 | | CPLX3 | RNF182 |
| LCN6 | CYP4A22 | CYP4F8 | | PIK3R6 | TBX10 |
| SIX6 | EPGN | KRTAP4-6 | | CTCFL | TCP10L2 |
| IGFBP7 | GOLGA6C | TMPRSS11F | | CNTN2 | TPO |
| PARM1 | PAX3 | SLC26A4 | | HOXC11 | SYTL2 |
| SPEM1 | SCNN1B | UGT2B11 | | AVP | MMP11 |
| NNMT | HBE1 | WNT5B | | ST6GALNAC3 | GNG4 |
| NFKBIA | CGB2 | ARMC3 | | GRAP2 | PADI2 |
| ARHGAP29 | CEBPE | C11orf88 | | ADCYAP1 | PLCL1 |
| ARHGAP42 | KLRC3 | LRP2 | | C7orf33 | GFRA3 |
| CTSS | ZNF677 | FAM83A | | HOXC12 | ST8SIA5 |
| SAMSN1 | C10orf67 | C1orf110 | | DGKI | PTPN5 |
| DMRTC1B | CYP4A11 | CYP8B1 | | GABRA4 | CAPN9 |
| FOXF1 | TEX35 | KRT78 | | EPHA5 | DMRTA1 |
| PTPRH | ZNF415 | PDCD1LG2 | | C7orf77 | SH3GL2 |
| MYL9 | CT62 | VNN2 | | GABRB1 | KCNH6 |
| RNF207 | BTNL2 | GRHL3 | | SHCBP1L | ZP1 |
| GJB4 | GIMAP1 | IL31RA | | SORCS3 | BEST3 |
| TRIP10 | ZNF835 | GPRC5A | | ALOX5AP | PRUNE2 |
| AFAP1L1 | ZBED2 | AWAT2 | | SUCNR1 | KLK12 |
| ADAMTSL4 | RHOXF1 | PPP1R42 | | RAX2 | GRM4 |
| ARHGAP25 | CGB5 | TDRD1 | | C1orf61 | SLC16A6 |
| ABCB11 | LINC00961 | TCN1 | | SYNPR | TREML2 |
| ATOH8 | IL37 | ETV3L | | GPR26 | RIIAD 1 |
| EHBP1L1 | GCM2 | MMP13 | | MYL1 | ADAMTSL1 |
| SERPINC1 | KRTAP16-1 | TMEM211 | | DBX1 | NCAM1 |
| IL1R2 | LRRC10 | RNASE1 | | SLC25A48 | CACNA1E |
| HTR1B | ACSM6 | CTXN3 | | HOXC9 | C4BPB |
| CLCF1 | KRTAP1-3 | TREM2 | | PRL | C18orf42 |
| ITGA1 | KBTBD13 | AKR1B10 | | ST18 | KCNA10 |
| PTPRU | CPN2 | FAM181A | | ST8SIA2 | WNT11 |
| SCN4A | WDR64 | AOC1 | | OR2T8 | SSTR1 |
| DLK2 | ZNF727 | KRT2 | | CPB1 | TMEM176A |
| WDFY4 | PRSS37 | TEK | | C8A | TMEM176B |
| PDE4B | MPEG1 | RAET1E | | OLIG3 | MT1A |
| FXYD5 | DNAJB8 | FMO1 | | RPH3A | RANBP3L |
| TMEM173 | SPATA19 | CFB | | UNC5D | NGB |
| FLNC | COX8C | GRIN2A | | PHOX2B | TERT |
| RGMA | ZNF98 | SOST | | GPR12 | ZMAT4 |
| HCAR1 | CYP2A7 | MAL | | TRIM49D1 | CRYBA2 |
| SLC5A5 | | DPP4 | | SGCZ | HABP2 |
| CXorf65 | | BLK | | CLPSL2 | ENPP2 |
| DIO3 | | IL36RN | | SPN | PPM1E |
| RARRES1 | | SPRR1A | | NEUROD1 | WDR72 |
| C3orf36 | | SERPINA9 | | HOXC4 | RAB39A |
| RBPMS2 | | SLC4A4 | | PIK3CG | ALDH1A1 |
| ITPRIPL2 | | RNF212B | | PADI6 | KISS1R |
| BMPER | | NTRK3 | | AMER2 | CSMD3 |
| KIF25 | | MUC2 | | MYOG | MYH6 |
| PLA2G4E | | LBP | | LGALS14 | DNAJC5B |
| FAM65B | | IL36G | | RGS8 | NROB2 |
| LSP1 | | UPK1B | | DYNAP | LAPTM5 |
| C3orf20 | | ITLN1 | | MYOD1 | RAB26 |
| SLC16A2 | | KPNA7 | | SLC17A8 | CA8 |
| EPS8L1 | | FAP | | SST | SULT1C2 |
| EPAS1 | | HEPH | | DAZ3 | SRRM4 |
| CAV2 | | C1orf189 | | TCL1B | SLC38A8 |
| ASB11 | | AQP5 | | UNCX | SGCA |
| LAMB3 | | MUC16 | | NAA11 | FAM3B |
| IGF2 | | DAPL1 | | TUSC5 | LRRC31 |
| WNT2 | | KRT6A | | PRLHR | FZD9 |
| ERMN | | GPRC6A | | SHOX2 | TCERG1L |
| SDR16C5 | | TRIM31 | | PROKR1 | DISP2 |
| PDLIM4 | | SERPINA12 | | SLC4A10 | BCO1 |
| C10orf128 | | CYP2C9 | | MYT1L | PEG10 |
| FSTL1 | | IL1RL1 | | TRIM64B | CA5A |
| LMO7DN | | FOXL1 | | | FIBCD1 |
| C22orf31 | | TRIM10 | | | LMO3 |
| GJA4 | | CCL26 | | | KIF19 |
| NEURL3 | | CXCL1 | | | ISLR |
| KRT23 | | GPR78 | | | UNC13A |
| TCEAL2 | | ACER1 | | | GPR142 |
| CHI3L1 | | PPBP | | | NPY |
| CYP3A5 | | RTP1 | | | FAR2 |
| AKR1C1 | | FST | | | KCNK3 |
| CST7 | | SLURP1 | | | DDC |
| CTSE | | BPIFC | | | GLYATL3 |
| MUC22 | | F3 | | | CLDN3 |
| GBP4 | | SPRR2F | | | METTL7B |
| HAS2 | | SHISA9 | | | ACR |
| NRG1 | | TENM2 | | | GABBR2 |
| ECSCR | | FGA | | | HHIP |
| FZD10 | | APOBEC3A | | | RFX4 |
| SFRP1 | | BMP2 | | | SPP2 |
| ITGAX | | PNLIPRP3 | | | PDIA2 |
| OSMR | | HCAR3 | | | CADPS |
| BTG4 | | MUC4 | | | FGF9 |
| C20orf195 | | LCE1C | | | VGF |
| ZDHHC15 | | PCDH8 | | | PCSK2 |
| BLID | | HOXB1 | | | LDLRAD4 |
| PDE10A | | CYP11A1 | | | LGSN |
| TSHZ3 | | IRX4 | | | DMRT3 |
| CD53 | | SLC34A2 | | | USH1C |
| MROH2A | | KRT36 | | | MOG |
| TGM5 | | ATP13A4 | | | PDLIM3 |
| ACSL5 | | ADH1A | | | CELF3 |
| ANXA2 | | CLDN8 | | | AMER3 |
| PCDH11Y | | TAS2R38 | | | OXT |
| SYNPO | | CA6 | | | C3orf80 |
| TNFSF15 | | HLA-DRB5 | | | RUNX1T1 |
| SLCO2A1 | | LCN2 | | | ASB5 |
| PSG9 | | FFAR4 | | | HES6 |
| FBXO2 | | CDSN | | | NRAP |
| SPOCD1 | | CRNN | | | PEX5L |
| APOL3 | | VNN3 | | | ANO3 |
| LAMA3 | | VCAM1 | | | WDR17 |
| SERPINB7 | | HCAR2 | | | DCDC2C |
| APOL1 | | SLC39A2 | | | KCNH4 |
| CYP2A6 | | PAEP | | | KCTD8 |
| BHLHE41 | | AKR1C4 | | | UNC5A |
| CYSLTR2 | | KCNK10 | | | CTXN2 |
| MMP1 | | CD244 | | | CNTN4 |
| RSPO3 | | ALOX15 | | | GALNT13 |
| ZSCAN5B | | LIX1 | | | PAH |
| RNASE13 | | DSG3 | | | UGT2B4 |
| SEMA3D | | CCL20 | | | UGT3A1 |
| PDCD1 | | IRG1 | | | LRRC26 |
| TPPP2 | | TM4SF4 | | | TMEM72 |
| GUCA2A | | SERPINE1 | | | ADARB2 |
| FAM25C | | ATOH1 | | | TESC |
| CHST15 | | IL20 | | | LIN28A |
| FOXI2 | | SERPINB4 | | | UCN3 |
| AJAP1 | | GPR50 | | | SYN2 |
| CCL7 | | SPIC | | | CABP7 |
| CXCL5 | | Dec1 | | | KRTAP10-2 |
| FRMD6 | | CEACAM7 | | | UNC80 |
| CYP2C8 | | SERPINB13 | | | KCNH2 |
| NUDT10 | | GSTA2 | | | OTOG |
| EFEMP2 | | SFTA2 | | | SKAP1 |
| XYLT1 | | KLK9 | | | FGF14 |
| EDNRB | | FAM25A | | | PKD1L3 |
| CYTL1 | | SPRR2B | | | HCRT |
| FGL2 | | RHCG | | | KAAG1 |
| PEG3 | | SERPINB10 | | | MAP3K15 |
| MMP10 | | NTS | | | FRMPD1 |
| TNFRSF6B | | WFDC5 | | | PTCHD3 |
| SGK1 | | CERS3 | | | NNAT |
| EHD2 | | COL12A1 | | | CCNI2 |
| CXCL6 | | TRIM40 | | | ASCL1 |
| EBF4 | | HHATL | | | RAX |
| ABI3 | | GSTA1 | | | ARMC4 |
| GAST | | IL7R | | | SYT14 |
| SLAMF9 | | KRTAP4-8 | | | P2RY6 |
| TREML1 | | C10orf99 | | | CDK5R2 |
| PTGIR | | DUSP27 | | | SMOC2 |
| ODF3B | | MYF6 | | | GPRIN3 |
| IGF1 | | IFNL1 | | | NBPF4 |
| XIRP1 | | | | | BEND4 |
| DKK4 | | | | | COL6A6 |
| GNA15 | | | | | SLC15A1 |
| TMEM265 | | | | | KRT31 |
| PPL | | | | | KCNK9 |
| FAM71D | | | | | LRFN2 |
| GIMAP7 | | | | | FRMPD2 |
| CDC42EP5 | | | | | TRPM2 |
| ADH1B | | | | | CALN1 |
| ORM1 | | | | | NAT8L |
| DKK1 | | | | | PHGR1 |
| ZFPM2 | | | | | RETNLB |
| CNN1 | | | | | PKD2L1 |
| FLT1 | | | | | CHGA |
| ADGRE1 | | | | | TFF3 |
| HRH1 | | | | | C1orf168 |
| S100A16 | | | | | SOX1 |
| GBP5 | | | | | JAKMIP2 |
| NEDD9 | | | | | AQP12A |
| NAP1L2 | | | | | CCL15 |
| CYP2B6 | | | | | OPRK1 |
| ICAM1 | | | | | LDLRAD1 |
| NAP1L3 | | | | | VIL1 |
| THRB | | | | | CFHR3 |
| CPAMD8 | | | | | PARVG |
| TNC | | | | | OR51G2 |
| GLYAT | | | | | LPL |
| FAM71A | | | | | RGS7 |
| TNS4 | | | | | FOLR3 |
| MAGEH1 | | | | | AMPH |
| FABP2 | | | | | RPRML |
| PSG8 | | | | | P2RX5 |
| RASL12 | | | | | NBPF6 |
| ITGA8 | | | | | SCGN |
| ITGB4 | | | | | POTEC |
| SERPINA10 | | | | | SCG3 |
| WNT7B | | | | | SPPL2C |
| SYDE1 | | | | | LCN15 |
| P2RY2 | | | | | CCDC33 |
| G0S2 | | | | | NKX2-2 |
| EMCN | | | | | KLHL14 |
| C3 | | | | | BSND |
| HRASLS2 | | | | | PLA1A |
| PDGFA | | | | | INHBE |
| MDFI | | | | | RIMBP2 |
| TIMP3 | | | | | ALX1 |
| ID1 | | | | | NOL4 |
| NTNG1 | | | | | CPLX2 |
| RCSD1 | | | | | NELL1 |
| SERPINE2 | | | | | SEC14L5 |
| FEZ1 | | | | | BHMT |
| LUM | | | | | CA9 |
| EGFR | | | | | KCNJ16 |
| TSLP | | | | | MRGPRE |
| MAOA | | | | | POTEI |
| CAV1 | | | | | Mar. 4 |
| FPR1 | | | | | ADGRV1 |
| ABCA4 | | | | | DNAI2 |
| PCDH17 | | | | | LRRC30 |
| ZP4 | | | | | APOBEC1 |
| TNFAIP6 | | | | | SNTG2 |
| C16orf74 | | | | | MGAT4C |
| VGLL1 | | | | | IYD |
| GYPC | | | | | UMOD |
| C17orf105 | | | | | SLC22A16 |
| MYOC | | | | | GPR6 |
| TRIM63 | | | | | ITIH2 |
| CCL21 | | | | | IKZF3 |
| WNT7A | | | | | PKHD1L1 |
| KLHDC7B | | | | | TNFRSF13B |
| LURAP1 | | | | | INSM1 |
| FABP3 | | | | | KLK4 |
| TFF2 | | | | | SLC22A31 |
| SPARCL1 | | | | | LCN8 |
| OR52W1 | | | | | NRSN1 |
| PSG5 | | | | | SULT4A1 |
| GJB5 | | | | | FGF12 |
| ICAM4 | | | | | NDST4 |
| PEBP4 | | | | | AVPR1B |
| CD84 | | | | | MLIP |
| OR2C1 | | | | | TGM3 |
| UBL4B | | | | | SCG2 |
| IL6 | | | | | TTR |
| FAM71E2 | | | | | UBD |
| PRODH2 | | | | | CPNE4 |
| WT1 | | | | | TMEM200A |
| NXPE4 | | | | | CHGB |
| TRPC4 | | | | | CAMKV |
| FPR2 | | | | | SLC35F1 |
| CRLF2 | | | | | FXYD4 |
| CLDN1 | | | | | GOLGA6L2 |
| NLRP7 | | | | | BRSK2 |
| SPINK6 | | | | | PLD5 |
| COL23A1 | | | | | RAB3C |
| VNN1 | | | | | DSCAM |
| SCNN1G | | | | | SYT4 |
| HBA2 | | | | | C2orf70 |
| IL1RN | | | | | HCN1 |
| IGFBP4 | | | | | XKR7 |
| GPR45 | | | | | SPAG6 |
| CD22 | | | | | RXFP3 |
| SERPINB2 | | | | | GLDC |
| SMR3A | | | | | SCGB2A1 |
| FGF19 | | | | | SYT1 |
| FAM43B | | | | | PON1 |
| CLU | | | | | TCP10 |
| COL17A1 | | | | | CALCA |
| TRPV5 | | | | | ZIC5 |
| PLA2G2A | | | | | CRB1 |
| KCNJ15 | | | | | CALY |
| IFNA1 | | | | | KLHL41 |
| MUSK | | | | | CD1E |
| LRRC4C | | | | | MEIOB |
| CXCL13 | | | | | LYPD8 |
| TNFSF18 | | | | | PLA2G12B |
| PSG3 | | | | | NROB1 |
| MMP3 | | | | | PENK |
| TMEM233 | | | | | DOK6 |
| ZNF728 | | | | | TRPA1 |
| C17orf47 | | | | | KCNJ12 |
| OSM | | | | | HBA1 |
| TPRG1 | | | | | MEOX2 |
| DCN | | | | | |
| KCNC2 | | | | | |
| ANXA8 | | | | | |
| ZSCAN1 | | | | | |
| ZNF676 | | | | | |
| MS4A14 | | | | | |
| CLDN16 | | | | | |
| IL1RL1 | | | | | |
| FOXC2 | | | | | |
| CASP12 | | | | | |
| COMP | | | | | |
| TLR8 | | | | | |
| PRRX2 | | | | | |
| NPFFR2 | | | | | |
| SORCS1 | | | | | |
| MYH2 | | | | | |
| SLC22A11 | | | | | |
| FHL5 | | | | | |
| CYP4F22 | | | | | |
| WFDC13 | | | | | |
| MMP28 | | | | | |
| MUC21 | | | | | |
| CSMD1 | | | | | |
| GATA5 | | | | | |
| ZNF208 | | | | | |
| WFDC10B | | | | | |
| PRR33 | | | | | |
| VWC2 | | | | | |
| PSG11 | | | | | |
| NFE4 | | | | | |
| CCL3 | | | | | |
| COL6A3 | | | | | |
| HAO2 | | | | | |
| FAM25G | | | | | |
| CXCR1 | | | | | |
| PCDH11X | | | | | |
| RAMP3 | | | | | |

TABLE 2
Enrichr GO Biological Processes 2021 results of upregulated genes per HC (Related to FIG. 1F)

| Term | Genes | HC |
|---|---|---|
| cytokine- | CXCL6; CSF3; IL1RN; CD40; PLVAP; CXCL9; ITGAM; TNFRSF6B; CSF1; | HC1 |
| mediated | IFNA1; CXCL13; FGF2; CXCL5; ICAM1; MT2A; IL18RAP; ITGAX; TIMP1; | |
| signaling | IL13RA2; IL15RA; SERPINB2; ANXA2; TNFRSF12A; IFNGR1; MMP1; IL1R2; | |
| pathway | MMP3; TRAF1; TNFRSF1B; OSMR; HLA-G; CEACAM1; IL23A; IRF1; TFF2; | |
| | LTB; IL6ST; SQSTM1; BIRC3; CCL14; CEBPD; MAOA; EBI3; FPR1; PTGS2; | |
| | IL2RG; IL1RL1; CCL7; IRAK2; CCL3; S1PR1; CCL2; DUOX2; STAT5A; | |
| | TNFSF18; IL32; TSLP; CCL21; NOS2; TNFSF15; IL34; OSM; MSN; SOD2; | |
| | NFKB1; BATF; VEGFA; NFKBIA; IL6; CLCF1; CRLF2 | |
| extracellular | COL17A1; ITGAM; ITGB4; LAMA3; ICAM2; TNC; PDGFA; ICAM4; LAMC2; | HC1 |
| structure | FGF2; NID2; ADAMTS10; MMP21; ICAM1; COMP; ADAMTSL4; MMP28; | |
| organization | ITGAX; ITGB8; ADAMTS9; LAMB3; ITGA3; MMP1; LUM; ITGA2; COL23A1; | |
| | ITGA1; MMP3; MMP10; DCN; VCAN; ITGA10; COL6A2; COL8A2; ITGA8; | |
| | PECAM1; COL6A3; ITGA6; DDR2 | |
| positive | STAT5A; ECM1; FLT4; VEGFC; WNT7A; IGF1; CLDN1; FGF2; EGFR; VEGFA; | HC1 |
| regulation of | TGM1; FGF7; SFRP1; CDH13; PRKD1; FGF10 | |
| epithelial cell | | |
| proliferation | | |
| nervous | ACHE; LDB2; FGF2; HAPLN4; NPAS2; GLIS2; SHH; EDNRB; FLRT3; NTF3; | HC1 |
| system | DRD1; EPHB2; WNT2; ZBTB16; SPINK6; NOG; NRG1; SOX10; VEGFA; | |
| development | VCAN; NAV3; AXL; FEZ1; FGF19; ALDH1A2; MET; NBL1 | |
| positive | BMP4; NR4A1; NRP1; EPGN; EGR3; OSR1; HPN; ZNF703; MST1; FGF1; PGF | HC2 |
| regulation of | | |
| epithelial cell | | |
| proliferation | | |
| cytokine- | IL20; IL5RA; CXCL1; IFIT1; CXCL3; TNF; CXCL2; IFIT3; OASL; CA1; | HC3 |
| mediated | IL36A; MAP3K8; LBP; CD36; PF4V1; HLA-DPA1; EDN2; RSAD2; IL19; F3; | |
| signaling | MMP9; GREM2; AIM2; IFI27; OAS2; IFNK; ALOX15; IFI6; NOD2; IL1RL1; | |
| pathway | IFNL1; PTPRZ1; CCL5; IL36RN; CCL 19; HLA-DQA2; HLA-DQA1; CCL24; | |
| | HLA-DRB5; CCL22; VCAM1; TNFSF14; CCL20; MX2; MX1; FN1; IL31RA; LIF; | |
| | IL36G; PPBP; BST2; CXCL10; GSTA2; LCN2; SAA1; HLA-DRA; XAF1; TRIM31; | |
| | IL7R; HLA-DRB1; CCL26 | |
| epidermal cell | CERS3; SPRR2F; SPRR3; CDSN; SPINK5; WNT5A; KRT10; OVOL1; ACER1; | HC3 |
| differentiation | SPRR2A; SPRR2B; SPRR1A; IVL; SPRR1B; S100A7 | |
| positive | CRNN; MMP12; CCL24; BMP2; NR4A3; EGF; WNT5A; HTR2B; NOD2; TEK; | HC3 |
| regulation of | F3; CCL26 | |
| epithelial cell | | |
| proliferation | | |
| extracellular | VIT; FGB; FGA; VCAM1; SERPINE1; SPINK5; FGG; FN1; MMP9; MMP12; | HC3 |
| structure | MMP13; ADAM12; COL5A2; TGFBI | |
| organization | | |
| extracellular | ADAMTS16; POSTN; COL3A1; COL1A2; SPOCK2; COL9A1 | HC4 |
| structure | | |
| organization | | |
| positive | LILRA2; TTBK1 | HC4 |
| regulation | | |
| of cell | | |
| activation | | |
| chemical | HRH3; NPFF; SLC6A1 | HC4 |
| synaptic | | |
| transmission | | |
| nervous | TRIM71; MYT1L; DLX5; AVIL; NCAN; DLX6; SHOX2; NRXN2; NEUROD1; | HC5 |
| system | NEUROD2; NHLH2; SCIN; DPYSL5; SLITRK1; ZIC3; NHLH1; ZIC1; ADORA1; | |
| development | SOX8; NRTN; ASIC2; MNX1; ARNT2; RBFOX1; EMX2; TRPC5; CNTN6; | |
| | ST8SIA2; LSAMP; GFRA1; ZIC4; POU4F1; GFRA2; POU3F2; POU3F3; | |
| | PHOX2B; ADGRB2; LHX1; KCNQ2; LY6H; NES; APBA2; NEUROG1 | |
| chemical | CHRNA1; GABRB1; GABRA4; HTR3E; STAC3; GAD2; NRXN2; SYN3; GR | HC5 |
| synaptic | IN2D; CHRND; DLG2; SST; KCNMB1; CAMK4; KCNQ2; PLP1; SLC17A6; | |
| transmission | SLC17A7; SLC17A8; ASIC2; APBA2 | |
| positive | TCF15; GATA4; TBX5 | HC5 |
| regulation | | |
| of stem cell | | |
| differentiation | | |
| neurotransmitter | NRXN2; GAD2; SYN3; CPLX3 | HC5 |
| secretion | | |
| extracellular | ADAMTS4; COL2A1; MMP16; ADAMTS19; ADAMTS18; NCAN; COL11A1; SPP1; | HC5 |
| structure | COL9A3; COL19A1; ADAMTS20 | |
| organization | | |
| chemical | SNAP25; CHRM4; CHRM5; CACNA1A; DBH; CACNA1E; GRM4; NPY; PENK; | HC6 |
| synaptic | KCNN1; DLGAP1; NPTX1; BSN; HCRT; SLC12A7; CHRNB2; GABBR2; | |
| transmission | UNC13A; DTNA; PTPRN2; SYT1; KCNIP2; HTR1D; OPRK1; GRIN2C; | |
| | SYN2; SYN1; GRIN1; RIMBP2; AMPH; ZACN; STX1A; KCNK3 | |
| neurotransmitter | RIMS2; SNAP25; GRM4; PTPRN2; UNC13A; SYT1; CPLX2; SYN2; SYN1; | HC6 |
| secretion | STX1A | |
| nervous system | NRSN1; DAGLA; STMN3; CXCR4; CRMP1; ASCL1; SNTG2; MOBP; ZIC2; | HC6 |
| development | NRCAM; NPTX1; EPHB1; SH3GL2; SEMA6B; SRRM4; ADGRV1; MOG; DSCAM; | |
| | FZD9; ZIC5; GFRA3; BCAN; NELL1; SDK1; P2RX5; GJB1; ADORA2A; | |
| | SCN8A; NEURL1; SERPINI1; CNTN4; FGF13; FGF12; SHANK3; SDK2 | |
| extracellular | LAMA1; COL22A1; NDNF; ADAMTSL1; BCAN; MMP11; SMOC2; ADAMTS14; TTR; | HC6 |
| structure | MMP26; COL4A4; ADAMTS17; ADAMTSL2; COL4A3; JAM3 | |
| organization | | |

TABLE 3
List of weighted transcription factors for
PCA loadings of PARCB time course data
(Related to FIG. 8C)

| Gene | PC1 | PC2rot30 | PC3rot30 |
|---|---|---|---|
| ADNP | −2.60E−04 | −2.26E−03 | −6.46E−04 |
| ADNP2 | 1.34E−03 | −3.69E−04 | −1.24E−03 |
| AEBP1 | −1.22E−02 | −8.81E−03 | −1.38E−02 |
| AEBP2 | −2.63E−03 | −1.17E−03 | −2.19E−03 |
| AHCTF1 | 3.32E−04 | −5.18E−03 | −2.08E−03 |
| AHDC1 | −5.07E−03 | 5.01E−04 | −1.95E−03 |
| AHR | −1.49E−02 | 3.00E−03 | −3.62E−03 |
| AHRR | −5.13E−03 | −1.79E−02 | −5.99E−03 |
| AIRE | −1.02E−03 | −1.52E−03 | −2.60E−03 |
| AKAP8 | 6.97E−04 | −2.17E−03 | −1.93E−03 |
| AKAP8L | −4.62E−05 | 2.05E−03 | −7.61E−04 |
| AKNA | −6.82E−04 | 2.05E−03 | −1.12E−03 |
| ALX1 | 5.98E−03 | 4.59E−03 | 7.23E−04 |
| ALX3 | −1.88E−03 | −2.76E−03 | −1.11E−03 |
| ALX4 | −1.07E−02 | −3.05E−03 | 3.11E−03 |
| ANHX | −1.12E−04 | −5.06E−05 | −6.58E−05 |
| ANKZF1 | −7.89E−04 | 1.45E−03 | 3.01E−03 |
| AR | −3.15E−02 | 4.84E−03 | −1.49E−02 |
| ARGFX | 3.85E−04 | −7.90E−04 | 8.65E−04 |
| ARHGAP35 | −1.73E−03 | −3.65E−04 | 1.05E−04 |
| ARID2 | 1.55E−03 | −1.90E−03 | −2.52E−03 |
| ARID3A | 9.04E−03 | 1.02E−03 | −2.51E−03 |
| ARID3B | −7.53E−04 | 2.57E−03 | −6.00E−03 |
| ARID3C | −5.57E−03 | −7.39E−04 | 6.71E−03 |
| ARID5A | −5.47E−03 | 1.70E−03 | −4.33E−03 |
| ARID5B | −1.54E−02 | 1.62E−03 | −1.33E−02 |
| ARNT | −1.34E−03 | 1.05E−03 | 1.30E−03 |
| ARNT2 | 1.22E−02 | −9.92E−03 | −9.93E−03 |
| ARNTL | −4.33E−03 | −1.33E−03 | −4.40E−03 |
| ARNTL2 | −8.07E−03 | −4.03E−03 | −3.96E−03 |
| ARX | −1.42E−02 | 1.33E−02 | 2.21E−03 |
| ASCL1 | 2.78E−02 | 5.18E−02 | −2.54E−02 |
| ASCL2 | 1.20E−02 | −3.33E−02 | −2.06E−02 |
| ASCL3 | 7.33E−04 | −1.92E−03 | −1.01E−02 |
| ASCL4 | −1.84E−03 | −5.74E−04 | −5.61E−03 |
| ASCL5 | 1.59E−03 | −6.58E−03 | −4.50E−04 |
| ASH1L | −2.96E−03 | −2.65E−03 | −1.63E−03 |
| ATF1 | −2.32E−03 | −5.41E−04 | −2.29E−03 |
| ATF2 | −1.58E−03 | −1.01E−05 | −1.27E−03 |
| ATF3 | −8.22E−03 | 1.23E−02 | −7.74E−03 |
| ATF4 | −3.11E−03 | 4.62E−04 | −8.64E−04 |
| ATF5 | 2.44E−03 | 1.44E−03 | −1.46E−03 |
| ATF6 | −1.97E−03 | −1.56E−03 | −2.55E−03 |
| ATF6B | −1.17E−03 | 1.50E−03 | −2.38E−03 |
| ATF7 | −2.97E−03 | −2.21E−03 | −1.14E−03 |
| ATMIN | −4.04E−04 | −4.83E−03 | −9.98E−04 |
| ATOH1 | 4.44E−03 | 4.27E−03 | −9.96E−03 |
| ATOH7 | 1.01E−02 | 8.48E−04 | −5.86E−03 |
| ATOH8 | −1.05E−02 | −1.30E−03 | −4.60E−03 |
| BACH1 | −7.76E−04 | 5.63E−04 | −5.22E−03 |
| BACH2 | −7.63E−03 | −1.79E−03 | −2.06E−03 |
| BARHL1 | 2.22E−03 | −7.28E−04 | −3.47E−03 |
| BARHL2 | 1.87E−03 | −3.01E−03 | 1.62E−03 |
| BARX1 | 2.17E−03 | −1.28E−03 | −2.65E−04 |
| BARX2 | −2.30E−02 | 2.86E−03 | −1.60E−02 |
| BATF | −1.47E−02 | 3.77E−03 | −1.59E−02 |
| BATF2 | −5.29E−03 | 7.00E−03 | 2.56E−04 |
| BATF3 | −5.97E−03 | −9.73E−03 | −1.09E−02 |
| BAZ2A | −2.25E−03 | −6.13E−04 | −9.43E−04 |
| BAZ2B | −2.12E−03 | 2.75E−03 | −3.43E−03 |
| BBX | −1.86E−03 | −3.64E−03 | −4.01E−03 |
| BCL11A | −1.70E−04 | −1.37E−03 | 2.15E−03 |
| BCL11B | −6.74E−03 | −1.80E−02 | −1.11E−02 |
| BCL6 | −1.28E−02 | −4.12E−03 | −6.10E−03 |
| BCL6B | −2.32E−03 | −1.26E−02 | −6.21E−03 |
| BHLHA15 | 7.94E−03 | 1.97E−02 | −8.31E−03 |
| BHLHA9 | −1.04E−03 | −4.25E−04 | 2.31E−04 |
| BHLHE22 | 6.32E−03 | 1.04E−02 | −7.38E−03 |
| BHLHE40 | −1.30E−02 | 5.75E−03 | −5.55E−03 |
| BHLHE41 | −2.32E−02 | 1.64E−03 | −3.77E−03 |
| BNC1 | −2.97E−02 | −2.39E−03 | −2.37E−02 |
| BNC2 | −3.89E−03 | 1.09E−03 | 5.20E−04 |
| BPTF | −5.65E−04 | −1.34E−03 | −7.94E−04 |
| BRF2 | 8.70E−04 | −3.57E−03 | 2.08E−04 |
| BSX | −8.28E−04 | −3.81E−04 | −1.28E−05 |
| C11orf95 | 5.63E−03 | −1.01E−02 | −7.89E−03 |
| CAMTA1 | −3.78E−03 | −2.11E−03 | −9.30E−04 |
| CAMTA2 | 1.14E−03 | −2.21E−03 | −7.82E−04 |
| CARF | −3.78E−03 | −1.33E−03 | 8.26E−04 |
| CASZ1 | 3.69E−03 | −2.17E−05 | −4.70E−03 |
| CBX2 | 1.65E−02 | −1.09E−02 | −1.74E−02 |
| CC2D1A | 4.10E−04 | 1.39E−03 | −2.34E−03 |
| CCDC17 | −4.38E−03 | 2.62E−03 | −5.01E−03 |
| CDC5L | 1.84E−03 | −9.75E−04 | −1.69E−03 |
| CDX1 | 4.46E−03 | 2.22E−04 | −1.19E−02 |
| CDX2 | 2.48E−03 | 2.89E−05 | −9.46E−03 |
| CDX4 | −1.48E−05 | −2.19E−05 | −2.96E−04 |
| CEBPA | −1.95E−02 | −1.01E−02 | −1.19E−02 |
| CEBPB | −1.00E−02 | −3.28E−03 | −9.66E−03 |
| CEBPD | −1.70E−02 | 5.40E−03 | −5.15E−03 |
| CEBPE | −3.59E−03 | 1.73E−04 | 8.45E−04 |
| CEBPG | −9.91E−04 | −1.69E−03 | −8.34E−04 |
| CEBPZ | −5.15E−05 | −2.68E−03 | −2.95E−03 |
| CENPA | 1.67E−02 | 9.29E−04 | −1.64E−03 |
| CENPB | 1.16E−03 | −1.54E−03 | −5.99E−04 |
| CENPBD1 | 9.17E−05 | −1.06E−03 | 1.53E−03 |
| CENPT | 1.14E−03 | −2.59E−03 | −2.87E−03 |
| CGGBP1 | −1.19E−03 | −2.98E−03 | −3.59E−04 |
| CHAMP1 | 2.20E−03 | −3.44E−03 | 4.81E−03 |
| CHCHD3 | 2.19E−03 | −1.43E−03 | 3.20E−03 |
| CIC | −2.22E−03 | −2.06E−03 | −2.41E−03 |
| CLOCK | −1.30E−03 | −1.81E−03 | −2.79E−03 |
| CPEB1 | 1.91E−03 | 1.09E−02 | −4.34E−03 |
| CREB1 | −1.23E−03 | −3.43E−03 | −3.48E−03 |
| CREB3 | −4.59E−03 | 8.30E−04 | −1.70E−03 |
| CREB3L1 | −2.28E−02 | 7.34E−03 | −1.22E−02 |
| CREB3L2 | −1.57E−03 | 6.43E−03 | −2.19E−03 |
| CREB3L3 | −9.45E−04 | 1.00E−03 | 1.05E−03 |
| CREB3L4 | 1.45E−03 | −3.10E−03 | −4.18E−03 |
| CREB5 | −9.47E−03 | −4.06E−03 | −1.18E−02 |
| CREBL2 | −2.27E−03 | −1.74E−04 | −1.81E−03 |
| CREBZF | 1.33E−03 | 2.55E−04 | 5.61E−04 |
| CREM | −2.40E−03 | 1.53E−03 | −4.06E−03 |
| CRX | 4.21E−03 | −8.24E−03 | −4.58E−03 |
| CSRNP1 | −8.03E−03 | 3.27E−03 | −7.29E−03 |
| CSRNP2 | −1.91E−03 | −1.47E−03 | −3.33E−03 |
| CSRNP3 | 1.46E−02 | −3.78E−03 | −6.55E−03 |
| CTCF | 1.71E−03 | −3.59E−03 | −1.49E−03 |
| CTCFL | 7.13E−03 | −9.21E−03 | −5.77E−04 |
| CUX1 | −6.48E−04 | −3.06E−03 | −3.32E−03 |
| CUX2 | −2.06E−02 | −9.69E−03 | −1.89E−03 |
| CXXC1 | 2.52E−03 | −5.03E−04 | −2.71E−03 |
| CXXC4 | 1.32E−02 | 2.44E−02 | −5.58E−03 |
| CXXC5 | 3.90E−03 | −1.48E−02 | −8.56E−03 |
| DACH1 | −6.87E−03 | 1.88E−03 | −2.02E−02 |
| DACH2 | 7.15E−03 | 2.65E−03 | −8.68E−03 |
| DBP | −3.15E−03 | −8.66E−03 | 4.55E−03 |
| DBX1 | 7.85E−03 | −4.66E−03 | 1.28E−03 |
| DBX2 | −6.17E−04 | 1.57E−04 | −2.58E−03 |
| DDIT3 | −4.90E−03 | 3.82E−04 | 3.24E−03 |
| DEAF1 | 2.02E−03 | 3.32E−03 | 1.75E−03 |
| DLX1 | 3.07E−03 | 1.29E−02 | 6.12E−03 |
| DLX2 | 2.31E−03 | 1.51E−02 | −1.84E−03 |
| DLX3 | −1.02E−02 | −1.06E−02 | −8.94E−03 |
| DLX4 | 3.71E−03 | −1.22E−02 | −4.03E−03 |
| DLX5 | 1.88E−02 | −1.26E−02 | −9.82E−03 |
| DLX6 | 1.70E−02 | 2.53E−03 | 2.51E−03 |
| DMBX1 | 6.57E−04 | 1.19E−02 | 1.97E−03 |
| DMRT1 | 3.06E−03 | 9.46E−03 | −7.87E−04 |
| DMRT2 | 6.70E−03 | 1.37E−02 | −9.71E−03 |
| DMRT3 | 5.16E−03 | 8.48E−03 | −2.49E−03 |
| DMRTA1 | −5.54E−03 | 2.64E−02 | −6.60E−04 |
| DMRTA2 | 3.53E−03 | −1.07E−03 | 8.48E−04 |
| DMRTB1 | 2.22E−02 | −2.36E−02 | −1.28E−02 |
| DMRTC2 | −2.59E−05 | 2.99E−04 | −3.65E−04 |
| DMTF1 | −2.66E−03 | −1.45E−03 | 3.45E−04 |
| DNMT1 | 4.35E−03 | −2.30E−03 | −3.80E−03 |
| DNTTIP1 | −8.05E−04 | −8.01E−04 | −3.16E−03 |
| DOT1L | 2.74E−03 | 1.31E−03 | −3.69E−03 |
| DPF1 | 1.12E−02 | −4.74E−03 | −9.90E−03 |
| DPF3 | 5.44E−03 | −7.64E−03 | −7.14E−04 |
| DPRX | −6.90E−04 | −2.72E−04 | 3.72E−04 |
| DR1 | 5.72E−04 | −2.73E−03 | −1.59E−03 |
| DRAP1 | −5.91E−03 | −3.35E−03 | −3.87E−03 |
| DRGX | 3.33E−03 | −4.71E−03 | −1.02E−04 |
| DUX4 | 4.86E−04 | 1.92E−03 | −5.57E−04 |
| DUXA | 1.24E−04 | 4.59E−05 | −1.57E−04 |
| DZIP1 | 1.52E−03 | 9.27E−03 | 4.09E−04 |
| E2F1 | 1.99E−02 | 1.56E−03 | −6.01E−03 |
| E2F2 | 1.86E−02 | 5.83E−03 | −7.74E−03 |
| E2F3 | 4.89E−03 | −4.73E−03 | −6.28E−03 |
| E2F4 | −9.39E−04 | −5.40E−04 | −2.64E−03 |
| E2F5 | 2.79E−03 | −2.56E−03 | −2.04E−03 |
| E2F6 | 7.37E−04 | −4.25E−03 | −1.69E−03 |
| E2F7 | 7.57E−03 | 3.58E−04 | −2.94E−03 |
| E2F8 | 1.98E−02 | 4.52E−05 | −7.65E−03 |
| E4F1 | 2.48E−03 | −2.47E−03 | −6.62E−04 |
| EBF1 | 1.08E−02 | −5.80E−03 | −2.08E−02 |
| EBF2 | 8.56E−03 | −1.08E−02 | −3.44E−03 |
| EBF3 | 7.14E−03 | −9.68E−03 | −9.74E−03 |
| EBF4 | −1.90E−02 | 3.78E−03 | −5.02E−03 |
| EEA1 | −2.50E−03 | 2.74E−04 | −2.65E−03 |
| EGR1 | −2.23E−02 | 9.25E−03 | −1.33E−03 |
| EGR2 | −2.13E−02 | 8.91E−03 | −6.68E−03 |
| EGR3 | −1.78E−02 | 6.22E−03 | −1.02E−02 |
| EGR4 | −1.06E−02 | 8.44E−03 | −4.66E−03 |
| EHF | −6.48E−03 | −1.63E−02 | −1.16E−02 |
| ELF1 | −1.37E−03 | 2.17E−03 | −4.24E−03 |
| ELF2 | 6.77E−04 | −1.97E−03 | −9.29E−04 |
| ELF3 | −2.29E−03 | 3.90E−03 | −4.70E−03 |
| ELF4 | −3.66E−03 | 7.01E−04 | −4.48E−03 |
| ELF5 | 6.36E−03 | −3.05E−03 | −2.04E−02 |
| ELK1 | −1.62E−03 | −2.18E−03 | −2.16E−03 |
| ELK3 | −1.49E−02 | −6.06E−03 | −1.20E−02 |
| ELK4 | −4.80E−03 | −4.93E−03 | −1.75E−03 |
| EMX1 | 3.82E−03 | −4.19E−04 | 5.46E−04 |
| EMX2 | 8.07E−03 | −8.90E−03 | −1.10E−02 |
| EN1 | 1.06E−03 | 7.71E−05 | −9.17E−03 |
| EN2 | 5.61E−03 | −1.46E−02 | −1.04E−02 |
| EOMES | 8.68E−03 | −9.07E−03 | −1.15E−02 |
| EPAS1 | −2.36E−02 | 5.70E−03 | −1.03E−02 |
| ERF | −1.17E−03 | −4.30E−03 | −8.20E−03 |
| ERG | −8.14E−04 | −1.75E−02 | −1.88E−02 |
| ESR1 | −6.11E−03 | 2.18E−02 | −2.66E−02 |
| ESR2 | 1.07E−03 | −4.05E−03 | −4.02E−03 |
| ESRRA | 1.81E−03 | −3.24E−03 | −3.30E−03 |
| ESRRB | −2.19E−03 | −9.58E−03 | −2.15E−04 |
| ESRRG | 1.29E−02 | 1.12E−02 | −3.31E−03 |
| ESX1 | 9.29E−03 | −1.30E−02 | −5.24E−03 |
| ETS1 | −9.66E−03 | −8.35E−03 | −1.55E−02 |
| ETS2 | −1.27E−02 | 1.08E−02 | −7.05E−03 |
| ETV1 | −4.19E−03 | −5.41E−03 | 1.46E−04 |
| ETV2 | 1.85E−03 | −3.56E−04 | −5.95E−03 |
| ETV3 | −3.74E−03 | −1.17E−03 | −4.74E−03 |
| ETV3L | 1.42E−03 | 4.65E−03 | −8.83E−03 |
| ETV4 | −8.95E−03 | −1.87E−02 | −8.30E−03 |
| ETV5 | −9.90E−03 | −1.71E−02 | −3.90E−03 |
| ETV6 | −3.78E−03 | 2.99E−03 | −4.42E−03 |
| ETV7 | 1.42E−03 | −6.21E−03 | −1.03E−02 |
| EVX1 | −9.31E−03 | −1.10E−03 | −8.31E−03 |
| EVX2 | −9.88E−03 | 4.04E−03 | 1.25E−02 |
| FAM170A | −1.95E−03 | 8.23E−05 | −2.77E−03 |
| FAM200B | 3.65E−03 | −1.10E−03 | −5.19E−04 |
| FBXL19 | 6.63E−05 | −4.18E−03 | −5.67E−03 |
| FERD3L | 9.33E−05 | 3.68E−04 | 2.66E−04 |
| FEV | 5.10E−03 | −3.47E−03 | −2.31E−03 |
| FEZF1 | 1.16E−02 | −1.67E−02 | 8.20E−04 |
| FEZF2 | 5.09E−03 | −4.38E−03 | −3.27E−04 |
| FIGLA | 3.92E−04 | −7.50E−04 | 4.33E−05 |
| FIZ1 | 3.65E−04 | −4.01E−03 | −1.94E−03 |
| FLI1 | 1.21E−02 | −5.30E−03 | −1.11E−02 |
| FLYWCH1 | −4.65E−03 | −6.31E−04 | 1.60E−03 |
| FOS | −1.87E−02 | 6.64E−03 | 1.96E−04 |
| FOSB | −1.67E−02 | 7.98E−03 | −1.20E−02 |
| FOSL1 | −2.23E−02 | 7.05E−03 | −2.06E−02 |
| FOSL2 | −9.36E−03 | 8.95E−03 | −3.04E−03 |
| FOXA1 | −8.18E−03 | 7.86E−03 | −2.93E−03 |
| FOXA2 | 1.62E−02 | 2.30E−02 | −3.48E−03 |
| FOXA3 | 6.25E−03 | 3.25E−02 | −1.13E−02 |
| FOXB1 | −2.69E−05 | 2.78E−04 | 3.50E−04 |
| FOXB2 | 1.92E−04 | −3.85E−04 | 5.40E−05 |
| FOXC1 | −8.15E−03 | −6.34E−03 | −1.40E−02 |
| FOXC2 | −1.65E−02 | 1.04E−03 | −2.98E−02 |
| FOXD1 | 2.02E−02 | 1.61E−03 | −9.95E−03 |
| FOXD2 | 3.65E−03 | −7.22E−03 | −3.03E−03 |
| FOXD3 | −9.56E−04 | −2.36E−03 | −5.33E−03 |
| FOXD4 | −3.19E−03 | −5.05E−03 | −1.86E−03 |
| FOXD4L1 | −6.51E−03 | −9.19E−03 | −4.93E−03 |
| FOXD4L3 | −5.26E−03 | −5.88E−04 | 1.71E−03 |
| FOXD4L4 | −2.19E−03 | −8.87E−03 | 5.52E−03 |
| FOXD4L5 | −3.10E−03 | −7.33E−03 | 2.75E−03 |
| FOXD4L6 | −2.70E−03 | −6.64E−03 | 1.00E−03 |
| FOXE1 | −1.44E−02 | −2.29E−02 | −7.22E−03 |
| FOXE3 | 8.64E−03 | −9.63E−03 | −8.28E−03 |
| FOXF1 | −1.36E−02 | 7.08E−04 | −1.75E−02 |
| FOXF2 | −1.02E−02 | −4.71E−03 | −6.65E−03 |
| FOXG1 | −1.73E−03 | −2.73E−03 | −7.46E−04 |
| FOXH1 | −6.44E−04 | 3.39E−03 | 2.64E−03 |
| FOXI1 | 6.56E−03 | −6.32E−03 | −2.78E−02 |
| FOX12 | −1.26E−02 | 8.71E−03 | 5.53E−03 |
| FOXI3 | 1.67E−02 | −5.99E−03 | −1.44E−02 |
| FOXJ1 | −6.68E−05 | 1.04E−02 | −1.24E−02 |
| FOXJ2 | −3.07E−03 | −4.35E−03 | −4.71E−04 |
| FOXJ3 | −7.65E−04 | 7.10E−04 | 1.23E−03 |
| FOXK1 | −4.50E−04 | −4.08E−03 | −3.07E−03 |
| FOXK2 | 8.36E−04 | −6.70E−04 | −8.01E−04 |
| FOXL1 | −1.73E−02 | 1.62E−04 | −2.34E−02 |
| FOXL2 | −7.47E−03 | −6.96E−03 | −1.95E−03 |
| FOXM1 | 1.85E−02 | 2.30E−03 | −5.23E−03 |
| FOXN1 | −2.50E−02 | −1.75E−03 | −2.08E−02 |
| FOXN2 | 8.15E−04 | −4.76E−03 | −2.98E−03 |
| FOXN3 | 1.36E−03 | 2.42E−03 | −3.66E−03 |
| FOXN4 | 1.88E−02 | 2.31E−03 | −1.24E−02 |
| FOXO1 | −1.55E−02 | 1.33E−02 | −4.98E−03 |
| FOXO3 | −3.79E−03 | −5.62E−03 | −4.37E−03 |
| FOXO4 | −5.19E−04 | −2.61E−03 | −5.95E−03 |
| FOXO6 | 1.34E−02 | 1.57E−03 | −6.89E−03 |
| FOXP1 | −3.73E−03 | 5.12E−03 | −4.35E−04 |
| FOXP2 | 1.33E−02 | 1.65E−03 | −8.93E−03 |
| FOXP3 | 1.28E−03 | −6.23E−03 | −5.72E−04 |
| FOXP4 | 1.30E−03 | −5.21E−04 | −4.04E−03 |
| FOXQ1 | −3.07E−02 | −8.88E−04 | −1.98E−02 |
| FOXR1 | 3.52E−04 | −1.20E−03 | 7.47E−06 |
| FOXS1 | 1.32E−03 | −2.40E−03 | −2.50E−03 |
| GABPA | 9.11E−04 | −3.37E−03 | −1.16E−03 |
| GATA1 | −1.04E−03 | 2.46E−04 | 2.76E−03 |
| GATA2 | −1.79E−02 | −1.52E−02 | −6.94E−03 |
| GATA3 | −2.00E−02 | 5.81E−03 | −7.74E−03 |
| GATA4 | 4.17E−03 | −7.16E−03 | −2.79E−03 |
| GATA5 | −2.62E−03 | −8.63E−04 | −4.25E−04 |
| GATA6 | −1.19E−02 | 2.06E−02 | −9.92E−03 |
| GATAD2A | 2.80E−03 | −3.12E−03 | −4.96E−03 |
| GATAD2B | −1.44E−03 | −3.91E−03 | −1.70E−03 |
| GBX1 | −7.35E−04 | −5.05E−03 | 1.73E−03 |
| GBX2 | 9.45E−04 | 1.59E−03 | −2.09E−03 |
| GCM1 | −5.48E−03 | −4.28E−03 | 2.00E−03 |
| GCM2 | −2.96E−03 | −1.88E−03 | 3.56E−03 |
| GFI1 | 1.77E−02 | 3.27E−02 | −1.02E−02 |
| GFI1B | 2.59E−02 | −2.43E−02 | −3.31E−02 |
| GLI1 | −3.14E−03 | 7.17E−04 | −2.05E−02 |
| GLI2 | 6.37E−04 | −1.28E−02 | −1.27E−02 |
| GLI3 | −2.70E−02 | 2.05E−03 | −2.01E−02 |
| GLI4 | −2.93E−03 | −1.32E−03 | 4.35E−03 |
| GLIS1 | −3.11E−03 | −4.35E−03 | −3.74E−03 |
| GLIS2 | −1.74E−02 | −5.79E−03 | −7.99E−03 |
| GLIS3 | −6.14E−03 | 1.63E−02 | −1.02E−02 |
| GLMP | 4.24E−03 | 3.87E−03 | −2.04E−03 |
| GLYR1 | −2.72E−04 | −6.81E−04 | −2.30E−03 |
| GMEB1 | 7.94E−04 | −2.10E−03 | −4.23E−03 |
| GMEB2 | −3.07E−03 | −2.02E−04 | −7.57E−04 |
| GPBP1 | −8.57E−04 | −1.94E−03 | −3.27E−03 |
| GPBP1L1 | −1.31E−03 | −7.68E−05 | −1.10E−03 |
| GRHL1 | −1.13E−02 | 3.76E−03 | −6.16E−03 |
| GRHL2 | −5.17E−03 | −3.31E−03 | −4.32E−03 |
| GRHL3 | −1.75E−02 | 5.89E−03 | −2.20E−02 |
| GSC | −1.48E−05 | −9.69E−04 | −3.93E−03 |
| GSC2 | −5.77E−04 | 1.97E−05 | −2.58E−04 |
| GSX1 | −1.17E−04 | −1.75E−04 | −2.96E−05 |
| GSX2 | 8.91E−04 | −2.08E−03 | −7.23E−04 |
| GTF2B | 1.86E−04 | 1.65E−03 | −3.22E−03 |
| GTF2I | 1.09E−04 | −1.49E−03 | −2.86E−03 |
| GTF2IRD1 | −3.30E−03 | −2.25E−03 | −4.37E−04 |
| GTF2IRD2 | −6.29E−03 | 2.14E−04 | −3.24E−03 |
| GTF2IRD2B | −5.47E−03 | −9.46E−04 | −1.60E−03 |
| GTF3A | 6.19E−03 | −4.13E−03 | −2.85E−03 |
| GZF1 | 8.92E−04 | −2.98E−03 | 2.08E−03 |
| HAND1 | 7.53E−03 | −3.02E−03 | −1.12E−02 |
| HAND2 | 1.57E−03 | −1.31E−03 | −9.54E−04 |
| HBP1 | −5.63E−03 | 9.09E−04 | −5.85E−03 |
| HDX | −5.22E−04 | 3.47E−03 | −6.64E−03 |
| HELT | 2.55E−03 | −4.26E−03 | −7.01E−04 |
| HES1 | −8.58E−03 | 6.63E−03 | −3.87E−04 |
| HES2 | −9.47E−03 | −3.01E−03 | −1.35E−02 |
| HES3 | −6.99E−04 | −1.04E−03 | 3.97E−04 |
| HES4 | 1.81E−03 | −8.42E−03 | −1.11E−02 |
| HES5 | −1.14E−03 | −2.15E−03 | −1.39E−02 |
| HES6 | 2.85E−02 | 1.41E−02 | 1.12E−02 |
| HES7 | 5.54E−03 | −3.22E−02 | −1.01E−02 |
| HESX1 | 3.96E−03 | −1.13E−03 | 4.23E−03 |
| HEY1 | 3.51E−03 | 9.37E−03 | 4.50E−03 |
| HEY2 | 1.24E−02 | 3.04E−02 | −6.43E−03 |
| HEYL | 8.13E−03 | 5.48E−04 | −1.38E−02 |
| HHEX | 3.66E−03 | 1.09E−02 | −2.69E−03 |
| HIC1 | −3.60E−03 | −2.73E−03 | −1.16E−02 |
| HIC2 | 4.84E−03 | −1.11E−03 | −2.09E−03 |
| HIF1A | −4.40E−03 | −1.32E−03 | −3.70E−03 |
| HIF3A | 2.50E−03 | 1.24E−03 | −2.95E−03 |
| HINFP | −5.59E−04 | −1.61E−03 | −3.12E−04 |
| HIVEP1 | −8.15E−03 | −8.18E−04 | −9.20E−03 |
| HIVEP2 | −7.29E−03 | −6.76E−03 | −1.09E−02 |
| HIVEP3 | −7.04E−03 | 5.59E−03 | −5.74E−03 |
| HKR1 | −2.06E−03 | −3.31E−03 | 6.68E−04 |
| HLF | 2.77E−03 | −6.30E−03 | −1.59E−04 |
| HLX | −5.64E−03 | −8.64E−03 | −6.63E−03 |
| HMBOX1 | −2.28E−03 | −3.27E−03 | −8.40E−04 |
| HMG20A | 6.03E−04 | −6.21E−03 | 1.47E−04 |
| HMG20B | 1.60E−03 | 1.44E−03 | −1.44E−03 |
| HMGA1 | 1.65E−03 | −3.93E−03 | −2.63E−03 |
| HMGA2 | −3.96E−03 | −2.45E−02 | −1.13E−02 |
| HMGN3 | 5.85E−03 | 2.33E−03 | −2.42E−04 |
| HMX1 | 1.19E−03 | −3.98E−03 | −1.86E−03 |
| HMX2 | 2.59E−02 | −2.86E−02 | −2.94E−02 |
| HMX3 | 2.27E−02 | −2.76E−02 | −3.03E−02 |
| HNF1A | 1.18E−02 | 3.59E−02 | −5.86E−03 |
| HNF1B | −2.29E−02 | −9.80E−03 | −5.99E−03 |
| HNF4A | 1.69E−02 | 4.24E−02 | −5.46E−03 |
| HNF4G | 8.55E−03 | 1.66E−02 | −7.26E−03 |
| HOMEZ | −6.90E−03 | 1.09E−03 | 1.43E−03 |
| HOXA1 | 3.86E−03 | −8.36E−03 | 1.49E−03 |
| HOXA10 | 4.99E−03 | 1.30E−03 | 5.45E−04 |
| HOXA11 | −1.40E−03 | 2.02E−03 | 1.53E−03 |
| HOXA13 | −7.97E−03 | −1.11E−02 | −6.36E−04 |
| HOXA2 | 7.40E−03 | −1.38E−02 | −4.02E−04 |
| HOXA3 | 5.14E−03 | −1.29E−02 | −2.04E−03 |
| HOXA4 | 1.05E−02 | −1.14E−02 | −3.43E−03 |
| HOXA5 | 1.22E−03 | 7.23E−04 | 5.66E−03 |
| HOXA6 | −1.57E−03 | 1.11E−02 | 5.83E−03 |
| HOXA7 | 1.05E−03 | 6.24E−03 | −8.55E−04 |
| HOXA9 | 2.49E−03 | −1.16E−03 | 4.62E−03 |
| HOXB1 | −1.62E−03 | 1.53E−03 | −5.04E−03 |
| HOXB13 | −6.12E−03 | 1.38E−03 | −4.15E−03 |
| HOXB2 | −8.71E−03 | 1.13E−02 | −7.43E−03 |
| HOXB3 | 2.68E−03 | 3.85E−03 | −3.22E−03 |
| HOXB4 | 6.78E−03 | −2.36E−03 | −3.97E−03 |
| HOXB5 | 2.10E−02 | −7.63E−03 | −1.26E−02 |
| HOXB6 | 1.99E−02 | −4.63E−03 | −1.10E−02 |
| HOXB7 | 1.78E−02 | −1.68E−03 | −1.09E−02 |
| HOXB8 | 1.37E−02 | 1.98E−02 | −8.50E−03 |
| HOXB9 | 9.69E−03 | 5.26E−03 | −4.42E−03 |
| HOXC10 | 3.09E−02 | −2.56E−02 | −2.91E−02 |
| HOXC11 | 2.37E−02 | −2.95E−02 | −1.98E−02 |
| HOXC12 | 2.08E−02 | −2.34E−02 | −1.43E−02 |
| HOXC13 | 1.27E−02 | −2.70E−02 | −1.97E−02 |
| HOXC4 | 2.78E−02 | −2.29E−02 | −3.11E−02 |
| HOXC5 | 2.82E−02 | −2.20E−02 | −2.95E−02 |
| HOXC6 | 2.77E−02 | −2.06E−02 | −2.76E−02 |
| HOXC8 | 2.33E−02 | −1.85E−02 | −2.28E−02 |
| HOXC9 | 2.64E−02 | −2.13E−02 | −2.87E−02 |
| HOXD1 | 1.82E−02 | −4.96E−03 | −1.57E−02 |
| HOXD10 | −5.26E−03 | −6.65E−03 | −2.68E−03 |
| HOXD11 | −5.84E−03 | −7.49E−03 | −4.54E−03 |
| HOXD12 | 5.77E−03 | −1.99E−03 | 3.08E−03 |
| HOXD13 | −3.99E−04 | −1.12E−02 | 3.06E−03 |
| HOXD3 | 1.46E−02 | −1.14E−02 | −8.56E−03 |
| HOXD4 | 1.61E−02 | −1.58E−02 | −9.29E−03 |
| HOXD8 | 6.67E−03 | −9.79E−03 | −6.72E−04 |
| HOXD9 | −5.32E−03 | −1.01E−02 | 6.50E−04 |
| HSF1 | −1.08E−03 | 7.32E−04 | −7.23E−04 |
| HSF2 | 5.71E−03 | −2.21E−03 | 1.99E−03 |
| HSF4 | −7.76E−03 | 7.64E−03 | 2.32E−03 |
| HSF5 | −3.65E−03 | −8.42E−04 | −4.84E−03 |
| HSFX1 | −3.10E−03 | −2.88E−03 | −8.79E−04 |
| HSFX2 | −2.62E−03 | 4.65E−03 | 3.82E−03 |
| HSFY1 | −3.83E−04 | −1.19E−03 | −1.42E−03 |
| HSFY2 | −7.17E−04 | −5.00E−03 | −1.59E−03 |
| IKZF1 | 8.51E−04 | 1.26E−02 | −4.16E−03 |
| IKZF2 | −1.79E−02 | 3.84E−03 | −6.31E−03 |
| IKZF3 | 1.97E−02 | 1.10E−02 | −8.97E−03 |
| IKZF4 | −8.45E−04 | −2.23E−03 | −1.27E−03 |
| IKZF5 | 2.00E−03 | −4.61E−03 | −1.78E−03 |
| INSM1 | 3.19E−02 | 2.82E−02 | −2.18E−02 |
| INSM2 | 1.25E−02 | 7.19E−03 | −7.29E−03 |
| IRF1 | −1.00E−02 | −1.93E−03 | −8.15E−03 |
| IRF2 | −4.20E−03 | −5.34E−03 | −4.50E−03 |
| IRF3 | −2.66E−03 | 1.37E−03 | −3.05E−04 |
| IRF4 | 7.64E−03 | 1.42E−02 | −1.69E−03 |
| IRF5 | −4.43E−03 | −5.38E−03 | −1.12E−03 |
| IRF6 | −1.49E−02 | 7.55E−03 | −7.22E−03 |
| IRF7 | −3.85E−03 | 1.22E−02 | −3.42E−03 |
| IRF8 | 5.51E−03 | −6.85E−03 | −1.58E−02 |
| IRF9 | −5.47E−03 | 4.47E−03 | −5.10E−03 |
| IRX1 | 5.46E−03 | −7.91E−03 | −1.34E−02 |
| IRX2 | −7.11E−03 | 9.88E−03 | −9.96E−03 |
| IRX3 | −1.81E−02 | 1.32E−02 | −8.41E−03 |
| IRX4 | −2.19E−02 | 3.16E−03 | −2.08E−02 |
| IRX5 | −1.52E−02 | 2.34E−02 | −5.61E−03 |
| IRX6 | −4.90E−04 | 5.86E−03 | −2.51E−02 |
| ISL1 | −4.12E−03 | 2.00E−02 | −1.47E−03 |
| ISL2 | 1.50E−03 | −2.93E−03 | 6.70E−04 |
| ISX | −1.29E−03 | 1.62E−03 | 4.86E−03 |
| JAZF1 | 1.04E−03 | −2.80E−03 | −4.50E−03 |
| JDP2 | −9.07E−03 | −2.77E−04 | −4.75E−03 |
| JRK | 2.78E−03 | −6.37E−03 | −8.26E−04 |
| JRKL | −3.32E−03 | −4.71E−03 | 3.76E−04 |
| JUN | −4.12E−03 | 4.18E−03 | −2.15E−03 |
| JUNB | −1.48E−02 | 3.15E−04 | −3.00E−03 |
| JUND | −1.13E−03 | −4.96E−04 | −4.74E−03 |
| KAT7 | −9.34E−04 | −2.48E−03 | 3.72E−04 |
| KCMF1 | −1.96E−03 | −1.29E−03 | −1.64E−03 |
| KCNIP3 | 1.26E−03 | 2.02E−02 | 6.08E−04 |
| KDM2A | −1.72E−03 | 3.10E−04 | −2.57E−03 |
| KDM2B | 4.94E−03 | −3.10E−03 | −3.12E−03 |
| KDM5B | −3.72E−03 | 3.66E−03 | −2.86E−03 |
| KIN | −1.58E−03 | −6.01E−04 | −1.08E−03 |
| KLF1 | 8.01E−03 | −1.32E−03 | −5.68E−03 |
| KLF10 | −6.97E−03 | 1.92E−03 | 6.45E−04 |
| KLF11 | −1.01E−03 | −4.38E−03 | −2.77E−03 |
| KLF12 | 3.64E−03 | −1.01E−02 | −3.87E−03 |
| KLF13 | 2.60E−03 | −3.13E−03 | −5.03E−03 |
| KLF14 | −5.58E−03 | −2.06E−03 | 9.15E−04 |
| KLF15 | 8.48E−03 | 1.02E−02 | −1.14E−04 |
| KLF16 | 2.36E−03 | −1.59E−03 | −4.83E−03 |
| KLF17 | −1.62E−03 | −1.75E−04 | 5.56E−04 |
| KLF2 | −4.45E−03 | 3.71E−03 | −8.02E−03 |
| KLF3 | −6.77E−03 | 2.45E−04 | −1.46E−03 |
| KLF4 | −5.85E−03 | 6.27E−03 | −1.03E−02 |
| KLF5 | −1.72E−02 | 6.04E−03 | −1.14E−02 |
| KLF6 | −5.08E−03 | 8.19E−03 | −8.42E−03 |
| KLF7 | −2.63E−03 | −4.76E−03 | −8.84E−03 |
| KLF8 | −2.84E−02 | 3.45E−03 | −1.40E−02 |
| KLF9 | −5.68E−03 | 4.30E−04 | 8.52E−05 |
| KMT2A | −2.67E−03 | −9.86E−04 | −6.44E−04 |
| KMT2B | −1.05E−03 | −1.44E−03 | −1.85E−03 |
| L3MBTL1 | −3.69E−03 | −9.65E−04 | 2.71E−03 |
| L3MBTL3 | 2.38E−03 | −5.16E−05 | −4.16E−03 |
| L3MBTL4 | −3.43E−03 | 7.73E−03 | −4.72E−03 |
| LBX1 | 4.32E−04 | −5.75E−04 | −3.72E−04 |
| LBX2 | −2.79E−04 | 4.13E−04 | −6.93E−04 |
| LCOR | 1.49E−03 | −1.10E−03 | −1.62E−03 |
| LCORL | 5.67E−03 | −1.44E−03 | −1.30E−04 |
| LEF1 | 2.77E−03 | 3.71E−03 | 1.70E−03 |
| LEUTX | 6.05E−04 | −5.71E−05 | −8.74E−04 |
| LHX1 | 6.48E−03 | −2.81E−03 | −5.39E−03 |
| LHX2 | 1.13E−02 | −1.01E−02 | −1.02E−02 |
| LHX3 | 1.56E−02 | 2.65E−03 | −6.06E−03 |
| LHX4 | −3.95E−04 | −3.71E−03 | −2.37E−03 |
| LHX5 | −3.62E−03 | 3.36E−03 | 1.27E−03 |
| LHX6 | 1.36E−02 | −4.47E−03 | −2.23E−03 |
| LHX9 | 7.83E−03 | −1.92E−03 | −1.59E−02 |
| LIN28A | 2.79E−03 | 1.08E−02 | 6.00E−03 |
| LIN28B | 3.60E−02 | −1.43E−02 | −2.60E−02 |
| LIN54 | 3.67E−03 | −1.43E−03 | −2.58E−03 |
| LMX1A | 1.75E−03 | 2.02E−03 | −3.48E−03 |
| LMX1B | 4.04E−03 | 3.89E−03 | −1.30E−02 |
| LTF | −4.96E−03 | 1.77E−02 | −4.15E−02 |
| LYL1 | −1.44E−03 | −8.68E−03 | −2.29E−03 |
| MAF | −1.30E−02 | 1.04E−02 | −1.10E−02 |
| MAFA | −3.11E−03 | 5.03E−03 | −7.05E−03 |
| MAFB | −1.50E−02 | 5.34E−03 | −1.01E−02 |
| MAFF | −5.73E−03 | −6.39E−03 | −1.57E−02 |
| MAFG | −2.17E−03 | 2.91E−05 | −5.17E−03 |
| MAFK | −5.68E−03 | 3.67E−03 | −8.03E−03 |
| MAX | −2.37E−03 | −1.69E−03 | −3.31E−03 |
| MAZ | 7.12E−03 | −7.83E−04 | −3.93E−03 |
| MBD1 | −6.81E−04 | 8.52E−04 | 2.71E−04 |
| MBD2 | −1.19E−03 | −3.97E−03 | −4.11E−03 |
| MBD3 | 1.62E−03 | −8.23E−04 | −1.01E−03 |
| MBD4 | 1.95E−03 | −2.60E−03 | −1.75E−03 |
| MBD6 | −2.02E−03 | 2.46E−04 | −3.31E−03 |
| MBNL2 | −6.38E−03 | −2.33E−03 | −5.01E−03 |
| MECOM | −1.70E−02 | 5.86E−03 | −1.16E−02 |
| MECP2 | −4.13E−04 | −1.74E−03 | −1.13E−03 |
| MEF2A | −2.65E−03 | −2.64E−03 | 2.18E−04 |
| MEF2B | 4.27E−03 | 6.80E−03 | 3.67E−03 |
| MEF2C | 1.22E−03 | −8.75E−03 | −1.01E−02 |
| MEF2D | −5.34E−04 | −3.08E−03 | −3.78E−03 |
| MEIS1 | −8.71E−03 | 1.56E−02 | −3.03E−03 |
| MEIS2 | −7.14E−03 | 5.11E−03 | 1.89E−03 |
| MEIS3 | −8.97E−03 | −6.33E−03 | −4.43E−03 |
| MEOX1 | −2.44E−03 | −3.15E−03 | −8.03E−03 |
| MEOX2 | 5.84E−03 | 1.02E−02 | −1.52E−03 |
| MESP1 | 1.58E−02 | 4.24E−03 | −6.87E−03 |
| MESP2 | 4.60E−03 | 2.64E−03 | −8.35E−03 |
| MGA | 1.96E−03 | −3.54E−03 | −2.02E−03 |
| MITF | 2.10E−04 | 1.15E−03 | −2.42E−03 |
| MIXL1 | 5.86E−03 | −6.04E−03 | −5.59E−03 |
| MKX | −1.90E−03 | 2.68E−03 | −4.56E−03 |
| MLX | 1.42E−04 | 3.32E−04 | −7.57E−04 |
| MLXIP | 3.30E−03 | 3.57E−03 | −3.55E−03 |
| MLXIPL | −9.20E−03 | 2.76E−03 | 1.14E−02 |
| MNT | −2.10E−03 | −1.02E−03 | −3.72E−03 |
| MNX1 | 1.33E−02 | −8.64E−03 | −2.07E−03 |
| MSANTD1 | −3.37E−03 | −2.04E−03 | −3.55E−03 |
| MSANTD3 | 3.00E−04 | −2.41E−03 | −4.97E−03 |
| MSANTD4 | 2.75E−03 | −2.98E−03 | 4.08E−04 |
| MSC | 1.04E−02 | −9.14E−03 | −2.44E−02 |
| MSGN1 | −3.11E−03 | 2.80E−03 | −6.02E−03 |
| MSX1 | −1.20E−03 | −1.31E−03 | −1.14E−02 |
| MSX2 | −1.48E−02 | 4.16E−03 | −6.46E−03 |
| MTERF1 | 1.01E−03 | −1.98E−03 | 3.41E−03 |
| MTERF2 | 4.42E−03 | 9.72E−03 | 3.39E−03 |
| MTERF3 | 7.50E−04 | −3.76E−03 | −1.73E−03 |
| MTERF4 | 8.56E−04 | 4.51E−04 | 1.85E−03 |
| MTF1 | −2.14E−03 | −1.09E−03 | −3.45E−03 |
| MTF2 | 2.43E−03 | −3.96E−03 | −2.16E−03 |
| MXD1 | −3.97E−03 | 6.11E−03 | −8.55E−03 |
| MXD3 | 3.72E−03 | −3.58E−03 | 5.29E−04 |
| MXD4 | 3.51E−03 | 6.11E−03 | −8.66E−04 |
| MX11 | 2.01E−03 | −4.16E−03 | −8.02E−04 |
| MYB | 1.39E−02 | −1.42E−03 | −8.17E−03 |
| MYBL1 | 1.03E−02 | −5.13E−03 | −3.17E−03 |
| MYBL2 | 2.43E−02 | 5.26E−03 | −7.82E−03 |
| MYC | −4.31E−04 | −1.39E−03 | −3.70E−03 |
| MYCL | 4.10E−04 | 4.74E−03 | −6.23E−04 |
| MYCN | 1.17E−02 | −1.53E−02 | −2.00E−02 |
| MYF5 | 3.29E−03 | −1.33E−03 | −8.05E−03 |
| MYF6 | 1.32E−03 | 6.44E−04 | −5.31E−03 |
| MYNN | −1.38E−03 | −2.69E−03 | −5.91E−04 |
| MYOD1 | 9.61E−03 | −1.70E−03 | −5.90E−03 |
| MYOG | 1.03E−02 | −4.10E−03 | −1.45E−02 |
| MYPOP | 2.56E−03 | 1.24E−03 | −1.60E−03 |
| MYRF | 4.46E−03 | −4.01E−03 | −6.04E−03 |
| MYRFL | −7.72E−03 | −1.88E−03 | −2.67E−03 |
| MYSM1 | 5.07E−05 | −3.05E−03 | −6.68E−04 |
| MYT1 | 2.21E−02 | 1.43E−03 | −1.14E−02 |
| MYT1L | 3.95E−03 | −2.40E−04 | −2.52E−03 |
| MZF1 | −9.41E−04 | −3.77E−04 | 1.17E−03 |
| NACC2 | −3.97E−03 | 1.27E−02 | −3.04E−03 |
| NAIF1 | −2.45E−04 | 3.13E−04 | 1.34E−03 |
| NANOG | −6.73E−04 | 1.65E−04 | −3.60E−03 |
| NANOGNB | 7.17E−05 | −3.48E−04 | 1.24E−04 |
| NCOA1 | 4.98E−04 | −4.39E−04 | −2.84E−03 |
| NCOA2 | −1.16E−03 | −2.11E−03 | −4.93E−03 |
| NCOA3 | −1.45E−03 | −8.51E−03 | −5.89E−03 |
| NEUROD1 | 2.09E−02 | −2.30E−03 | −2.35E−02 |
| NEUROD2 | 1.16E−02 | −2.15E−02 | −8.36E−03 |
| NEUROD4 | 1.91E−02 | −7.74E−03 | −1.60E−02 |
| NEUROD6 | 9.35E−03 | −1.13E−02 | −5.36E−04 |
| NEUROG1 | 1.38E−02 | −1.34E−02 | −8.24E−03 |
| NEUROG2 | 8.96E−03 | −1.14E−02 | −7.76E−03 |
| NEUROG3 | 2.08E−04 | 1.22E−03 | −5.50E−03 |
| NFAT5 | −8.56E−03 | −3.68E−03 | −6.12E−03 |
| NFATC1 | 5.88E−03 | −2.44E−02 | −2.83E−02 |
| NFATC2 | 2.47E−03 | 4.59E−03 | −5.62E−03 |
| NFATC3 | 5.01E−03 | −6.23E−03 | −3.71E−03 |
| NFATC4 | 1.59E−03 | 4.68E−03 | 5.67E−04 |
| NFE2 | 3.43E−03 | −2.88E−03 | −6.24E−03 |
| NFE2L1 | −5.11E−03 | −2.65E−03 | 1.26E−05 |
| NFE2L2 | −5.88E−03 | −2.20E−03 | −3.42E−03 |
| NFE2L3 | −2.91E−04 | −7.20E−03 | −3.96E−03 |
| NFE4 | −2.37E−03 | 1.92E−04 | 2.15E−03 |
| NFIA | −5.20E−03 | 5.90E−03 | −2.67E−03 |
| NFIB | −8.46E−03 | 1.06E−02 | −8.07E−04 |
| NFIC | −3.56E−03 | 6.02E−04 | −8.55E−04 |
| NFIL3 | −8.50E−03 | 5.18E−03 | −8.20E−03 |
| NFIX | −1.03E−02 | −6.21E−03 | −8.28E−03 |
| NFKB1 | −8.49E−03 | 1.91E−03 | −9.73E−03 |
| NFKB2 | −1.06E−02 | 4.88E−03 | −6.18E−03 |
| NFX1 | −1.31E−03 | 1.16E−03 | 1.86E−03 |
| NFXL1 | 1.01E−03 | −1.17E−03 | −5.78E−04 |
| NFYA | −1.78E−03 | −3.89E−03 | −8.17E−04 |
| NFYB | 7.04E−04 | −2.16E−04 | −9.12E−04 |
| NFYC | 1.01E−03 | −3.41E−03 | −1.42E−03 |
| NHLH1 | 6.05E−03 | −1.07E−02 | −1.64E−03 |
| NHLH2 | 1.45E−02 | −6.55E−03 | −1.20E−02 |
| NKRF | 3.64E−04 | −1.84E−03 | −1.51E−03 |
| NKX1-1 | −2.97E−04 | −1.43E−04 | −1.82E−04 |
| NKX1-2 | −1.74E−03 | 2.30E−02 | −9.75E−03 |
| NKX2-1 | 5.53E−04 | 4.27E−04 | −3.97E−03 |
| NKX2-2 | 2.02E−02 | 4.09E−02 | −1.33E−02 |
| NKX2-3 | 2.34E−04 | −3.23E−04 | −9.54E−04 |
| NKX2-4 | 8.26E−03 | −5.10E−03 | −1.28E−03 |
| NKX2-5 | 3.35E−04 | −4.83E−03 | −4.09E−03 |
| NKX2-6 | 5.42E−05 | −4.19E−05 | −3.63E−04 |
| NKX2-8 | −1.47E−02 | 6.66E−03 | 4.39E−03 |
| NKX3-1 | 1.24E−03 | 5.93E−03 | −5.85E−03 |
| NKX3-2 | −3.55E−03 | 4.14E−03 | 3.96E−03 |
| NKX6-1 | 7.71E−03 | −1.25E−02 | −5.58E−03 |
| NKX6-2 | −4.97E−04 | −3.77E−03 | −1.06E−04 |
| NKX6-3 | 1.53E−03 | −9.83E−04 | −2.54E−03 |
| NME2 | −4.85E−03 | 4.36E−04 | −8.97E−04 |
| NOBOX | 2.83E−04 | −2.01E−03 | 5.78E−04 |
| NOTO | −2.03E−03 | 1.21E−03 | 1.88E−04 |
| NPAS1 | −3.91E−03 | −4.60E−03 | 2.08E−03 |
| NPAS2 | −2.32E−02 | −5.62E−03 | −1.32E−02 |
| NPAS3 | 1.50E−03 | 4.74E−03 | 2.00E−03 |
| NPAS4 | 5.29E−03 | 7.04E−03 | −5.98E−03 |
| NROB1 | 1.18E−03 | 2.67E−02 | 5.65E−03 |
| NR1D1 | −1.05E−02 | −6.36E−03 | −1.26E−03 |
| NR1D2 | −3.94E−03 | −1.00E−03 | 9.88E−04 |
| NR1H2 | −7.21E−03 | −8.49E−04 | −1.80E−03 |
| NR1H3 | −5.90E−03 | −6.69E−04 | 3.03E−04 |
| NR1H4 | 2.41E−03 | 8.88E−04 | −6.14E−03 |
| NR1I2 | 6.20E−03 | 2.25E−03 | −6.69E−04 |
| NR1I3 | 1.23E−03 | −7.15E−03 | −3.42E−03 |
| NR2C1 | −8.27E−04 | −5.21E−03 | −2.36E−03 |
| NR2C2 | 2.26E−04 | −3.70E−03 | −1.62E−03 |
| NR2E1 | −4.53E−04 | 1.99E−03 | 1.31E−03 |
| NR2E3 | −4.99E−03 | 5.94E−03 | −6.54E−05 |
| NR2F1 | 1.75E−02 | 1.80E−02 | −6.13E−03 |
| NR2F2 | 3.66E−03 | −1.79E−03 | 7.51E−04 |
| NR2F6 | 3.54E−03 | 3.93E−03 | −2.17E−04 |
| NR3C1 | −8.99E−03 | 1.45E−02 | −1.88E−03 |
| NR3C2 | 4.39E−03 | 1.87E−02 | −1.05E−03 |
| NR4A1 | −8.84E−03 | 7.43E−04 | −3.88E−03 |
| NR4A2 | −8.12E−03 | −7.80E−04 | −8.00E−03 |
| NR4A3 | −9.30E−03 | 3.61E−03 | −1.74E−02 |
| NR5A1 | −2.75E−03 | −2.94E−03 | 9.67E−04 |
| NR5A2 | −3.85E−03 | −2.07E−03 | 9.97E−05 |
| NR6A1 | 6.14E−04 | −2.11E−03 | −2.10E−03 |
| NRF1 | 1.27E−03 | −3.46E−03 | −2.45E−03 |
| NRL | 6.86E−03 | −4.37E−03 | −5.70E−03 |
| OLIG1 | 2.81E−02 | −1.99E−02 | −2.27E−02 |
| OLIG2 | 2.61E−02 | −3.38E−02 | −2.39E−02 |
| OLIG3 | 6.69E−03 | −6.59E−03 | −5.24E−03 |
| ONECUT1 | 7.65E−03 | −2.54E−04 | 3.73E−03 |
| ONECUT2 | 1.21E−02 | 7.43E−03 | −3.34E−03 |
| ONECUT3 | −8.42E−04 | 4.32E−03 | −3.90E−03 |
| OSR1 | −1.40E−02 | 9.65E−03 | 4.28E−03 |
| OSR2 | 4.05E−03 | 1.79E−03 | −5.25E−03 |
| OTP | 2.13E−03 | 1.52E−03 | −1.15E−03 |
| OTX1 | −4.10E−03 | −2.34E−03 | −3.36E−04 |
| OTX2 | 1.89E−02 | −1.26E−02 | −4.57E−03 |
| OVOL1 | −1.06E−02 | 1.09E−03 | −1.68E−02 |
| OVOL2 | 5.84E−04 | 9.98E−04 | −3.39E−03 |
| OVOL3 | 1.05E−02 | −1.56E−02 | −1.02E−02 |
| PA2G4 | 2.33E−03 | −1.04E−03 | −3.03E−03 |
| PATZ1 | 5.00E−03 | −2.80E−03 | 6.05E−04 |
| PAX1 | 1.05E−02 | 2.25E−04 | −2.74E−02 |
| PAX2 | 1.23E−03 | −1.23E−02 | −4.69E−03 |
| PAX3 | −1.17E−03 | 3.29E−04 | 1.78E−03 |
| PAX4 | 1.09E−02 | −5.99E−03 | −1.34E−02 |
| PAX5 | 1.84E−02 | 2.47E−02 | −8.36E−03 |
| PAX6 | 1.27E−03 | −5.20E−03 | −3.12E−04 |
| PAX7 | 4.18E−03 | −9.99E−03 | −3.76E−03 |
| PAX8 | −9.17E−03 | −5.73E−03 | −1.46E−02 |
| PAX9 | −5.04E−03 | 9.36E−03 | −7.56E−04 |
| PBX1 | −1.13E−03 | −9.21E−03 | −8.69E−03 |
| PBX2 | −2.23E−03 | −4.17E−03 | −2.38E−03 |
| PBX3 | −1.68E−03 | 1.89E−02 | 2.16E−03 |
| PBX4 | 3.34E−03 | −3.16E−03 | −6.62E−06 |
| PCGF2 | −7.79E−04 | −5.69E−03 | −2.70E−03 |
| PCGF6 | 4.28E−03 | −8.90E−04 | 3.07E−04 |
| PDX1 | 1.01E−03 | 1.61E−03 | −8.96E−04 |
| PEG3 | −2.96E−02 | −1.58E−03 | 1.32E−02 |
| PGR | −1.18E−02 | −1.15E−03 | −9.79E−04 |
| PHF1 | −7.50E−03 | −7.74E−04 | −8.85E−04 |
| PHF20 | −3.80E−04 | −2.44E−03 | −5.75E−04 |
| PHF21A | −1.95E−03 | −1.50E−03 | 1.11E−03 |
| PHOX2A | 4.57E−03 | −5.40E−03 | −4.05E−03 |
| PHOX2B | 8.16E−03 | −7.38E−03 | −3.14E−03 |
| PIN1 | 3.46E−03 | −3.20E−04 | −1.65E−03 |
| PITX1 | −4.02E−03 | 2.92E−03 | −3.72E−04 |
| PITX2 | −8.22E−03 | 1.87E−03 | −3.79E−03 |
| PITX3 | 1.07E−02 | −2.71E−03 | −4.95E−03 |
| PKNOX1 | −2.58E−04 | −2.16E−03 | −1.85E−04 |
| PKNOX2 | 1.12E−02 | −2.23E−02 | −1.82E−02 |
| PLAG1 | 1.19E−03 | 1.93E−03 | 4.69E−03 |
| PLAGL1 | −7.57E−03 | 1.40E−02 | 1.86E−03 |
| PLAGL2 | 1.85E−03 | −4.58E−04 | −5.51E−03 |
| PLSCR1 | −3.18E−03 | 5.62E−03 | −9.01E−03 |
| POGK | −9.70E−04 | −3.86E−03 | −3.26E−04 |
| POU1F1 | −1.00E−03 | 5.45E−04 | −4.13E−03 |
| POU2AF1 | −1.67E−02 | 3.12E−03 | −1.39E−02 |
| POU2F1 | 3.34E−03 | −1.38E−03 | −3.36E−03 |
| POU2F2 | −5.21E−03 | −7.68E−05 | −4.49E−03 |
| POU2F3 | 1.40E−02 | −3.01E−02 | −2.51E−02 |
| POU3F1 | −6.27E−03 | −9.15E−04 | −7.05E−03 |
| POU3F2 | 1.37E−02 | −1.61E−02 | 7.86E−05 |
| POU3F3 | 3.44E−03 | −5.28E−03 | −1.29E−03 |
| POU3F4 | 4.00E−04 | 2.05E−04 | −6.32E−05 |
| POU4F1 | 2.68E−02 | −1.56E−02 | −1.59E−02 |
| POU4F2 | 1.37E−03 | 2.07E−03 | 1.76E−03 |
| POU4F3 | 3.82E−03 | −6.03E−03 | 1.32E−03 |
| POU5F1 | −1.39E−02 | 8.59E−04 | −5.94E−03 |
| POU5F1B | −3.32E−03 | −3.39E−03 | −1.56E−03 |
| POU5F2 | −3.13E−03 | −1.23E−03 | −4.17E−03 |
| POU6F1 | 1.14E−03 | −1.05E−02 | −3.86E−03 |
| POU6F2 | 6.98E−03 | 4.11E−04 | 2.96E−03 |
| PPARA | −1.07E−02 | 1.39E−04 | 3.32E−03 |
| PPARD | −1.02E−02 | −2.16E−03 | −3.57E−03 |
| PPARG | −1.61E−02 | 1.33E−02 | 9.96E−03 |
| PRDM1 | −1.54E−02 | 8.88E−03 | −2.24E−02 |
| PRDM10 | 1.20E−03 | −1.64E−03 | −2.65E−03 |
| PRDM12 | 5.21E−03 | −5.05E−03 | 4.65E−03 |
| PRDM13 | 1.83E−02 | −9.93E−03 | −7.72E−03 |
| PRDM14 | −6.92E−05 | 3.14E−04 | 1.21E−04 |
| PRDM15 | 5.92E−04 | 2.80E−03 | 3.47E−04 |
| PRDM16 | −1.45E−02 | −1.03E−03 | 1.95E−03 |
| PRDM2 | −5.43E−04 | 1.53E−03 | −2.87E−03 |
| PRDM4 | 4.44E−04 | 5.55E−04 | 4.35E−05 |
| PRDM5 | 3.36E−04 | 6.43E−03 | −2.07E−03 |
| PRDM6 | −4.20E−03 | −2.24E−03 | −1.88E−03 |
| PRDM8 | −6.96E−03 | −8.82E−03 | 1.14E−05 |
| PRDM9 | −7.69E−05 | 3.16E−04 | 6.46E−04 |
| PREB | 3.03E−03 | 8.72E−04 | −1.45E−03 |
| PRMT3 | 2.19E−03 | −1.25E−03 | −2.40E−03 |
| PROP1 | −2.76E−03 | −5.97E−04 | 1.97E−03 |
| PROX1 | 3.04E−02 | 1.13E−03 | −1.88E−02 |
| PROX2 | −2.35E−03 | −4.78E−03 | 2.89E−03 |
| PRR12 | −1.50E−03 | −1.71E−03 | 7.49E−04 |
| PRRX1 | 3.65E−03 | −1.25E−02 | −2.15E−02 |
| PRRX2 | −1.46E−02 | 1.02E−03 | −2.80E−02 |
| PTF1A | 6.05E−03 | 1.08E−02 | 4.63E−03 |
| PURA | −5.25E−04 | −5.72E−03 | 3.15E−04 |
| PURB | −2.55E−03 | −4.58E−03 | −3.82E−03 |
| PURG | 6.69E−03 | 4.13E−04 | −5.60E−03 |
| RAG1 | −2.28E−03 | 4.77E−03 | 9.89E−04 |
| RARA | −5.17E−03 | −5.33E−03 | −6.38E−03 |
| RARB | −1.29E−02 | 2.08E−03 | −5.71E−03 |
| RARG | −1.77E−02 | −8.31E−03 | −1.21E−02 |
| RAX | 7.60E−03 | 9.98E−03 | 2.80E−03 |
| RAX2 | 5.49E−03 | −7.23E−03 | −1.27E−03 |
| RBAK | −2.17E−04 | −3.59E−03 | 2.03E−03 |
| RBCK1 | −3.56E−03 | 2.51E−03 | 2.29E−03 |
| RBPJ | 1.87E−04 | −3.87E−03 | −3.48E−04 |
| RBPJL | −2.58E−03 | 2.41E−05 | −7.59E−04 |
| RBSN | −9.24E−04 | −4.85E−03 | 2.60E−04 |
| REL | −8.63E−03 | 1.88E−03 | −1.17E−02 |
| RELA | −5.13E−03 | −2.81E−04 | −2.66E−03 |
| RELB | −1.11E−02 | −1.33E−03 | −7.29E−03 |
| REPIN1 | 6.14E−03 | 1.68E−03 | −1.21E−03 |
| REST | −3.74E−03 | −8.83E−03 | −4.52E−03 |
| REXO4 | 5.12E−04 | −2.09E−03 | −1.74E−03 |
| RFX1 | −2.86E−04 | −1.84E−04 | −3.16E−03 |
| RFX2 | −8.67E−03 | 8.06E−03 | −2.45E−03 |
| RFX3 | 7.40E−03 | −1.42E−03 | −2.71E−03 |
| RFX4 | 7.46E−03 | 2.22E−03 | 1.23E−04 |
| RFX5 | 4.71E−03 | 1.27E−04 | −5.05E−03 |
| RFX6 | 8.31E−03 | 2.48E−02 | 5.08E−03 |
| RFX7 | −1.77E−03 | −2.64E−03 | 2.14E−04 |
| RFX8 | 3.04E−03 | 5.14E−03 | −4.84E−03 |
| RHOXF1 | −6.89E−04 | −5.49E−03 | −9.62E−05 |
| RHOXF2 | 4.14E−04 | −2.93E−04 | −3.89E−04 |
| RHOXF2B | −4.86E−04 | 3.76E−04 | −5.22E−04 |
| RLF | −3.30E−03 | −5.36E−03 | −3.77E−03 |
| RORA | −4.65E−03 | 1.07E−02 | −4.93E−03 |
| RORB | −7.08E−03 | −6.76E−03 | −9.22E−03 |
| RORC | −3.90E−03 | 2.44E−02 | 3.60E−03 |
| RREB1 | −3.02E−03 | −4.74E−04 | −2.76E−03 |
| RUNX1 | −3.36E−03 | −4.21E−03 | −7.75E−03 |
| RUNX2 | −1.12E−02 | −8.99E−03 | −2.95E−03 |
| RUNX3 | −6.74E−03 | −8.69E−03 | −1.82E−03 |
| RXRA | −1.07E−02 | −2.12E−03 | −3.53E−03 |
| RXRB | −2.96E−03 | −7.30E−04 | −5.09E−04 |
| RXRG | 1.66E−02 | −5.83E−03 | −3.13E−03 |
| SAFB | 6.91E−04 | −1.71E−03 | −2.45E−03 |
| SAFB2 | 2.01E−03 | 1.12E−03 | −1.53E−03 |
| SALL1 | 1.18E−03 | 6.48E−03 | −3.33E−03 |
| SALL2 | 1.12E−02 | −3.97E−03 | −1.88E−03 |
| SALL3 | −1.09E−02 | 7.61E−03 | −4.39E−03 |
| SALL4 | −5.56E−03 | −5.14E−03 | −1.72E−02 |
| SATB1 | −5.44E−04 | 1.49E−03 | 7.10E−04 |
| SATB2 | 2.91E−03 | 6.34E−03 | −5.46E−04 |
| SCMH1 | −9.84E−04 | −1.93E−03 | −1.91E−03 |
| SCML4 | 8.73E−03 | −8.39E−03 | −8.21E−03 |
| SCRT1 | 3.13E−03 | −2.87E−03 | −2.71E−05 |
| SCRT2 | 7.58E−03 | −8.85E−03 | −1.35E−03 |
| SCX | 3.38E−03 | 9.27E−03 | −4.14E−03 |
| SEBOX | −1.21E−03 | −1.02E−03 | 1.03E−03 |
| SETBP1 | −3.72E−03 | −1.25E−02 | −9.67E−03 |
| SETDB1 | −2.25E−04 | −2.03E−03 | −8.14E−04 |
| SETDB2 | −4.60E−04 | −1.12E−03 | 1.74E−03 |
| SGSM2 | −2.37E−03 | −1.69E−04 | −2.80E−03 |
| SHOX | 1.07E−04 | −4.96E−05 | −3.43E−04 |
| SHOX2 | 1.77E−02 | 1.08E−02 | −3.47E−03 |
| SIM1 | 1.04E−02 | 7.58E−03 | −7.05E−03 |
| SIM2 | −1.15E−02 | −1.26E−02 | −8.48E−03 |
| SIX1 | −3.68E−03 | 6.39E−03 | 3.53E−03 |
| SIX2 | 4.42E−03 | 8.53E−03 | −8.37E−04 |
| SIX3 | 1.80E−03 | −1.99E−03 | −6.55E−04 |
| SIX4 | −1.16E−04 | 2.71E−03 | −3.19E−03 |
| SIX5 | −9.04E−03 | −2.00E−04 | −3.48E−03 |
| SIX6 | −4.56E−03 | −4.63E−03 | 3.13E−03 |
| SKI | −1.15E−03 | −1.49E−03 | −1.59E−03 |
| SKIL | 2.22E−03 | −2.19E−03 | −1.30E−02 |
| SKOR1 | 7.11E−04 | 4.37E−03 | −1.57E−03 |
| SKOR2 | 3.66E−03 | −6.64E−03 | 6.90E−04 |
| SLC2A4RG | −4.77E−03 | 4.30E−03 | 3.36E−04 |
| SMAD1 | −1.75E−03 | −1.37E−03 | −1.12E−03 |
| SMAD3 | −1.66E−02 | 5.32E−03 | −3.43E−03 |
| SMAD4 | −1.24E−03 | −3.05E−03 | −4.38E−04 |
| SMAD5 | 2.49E−03 | 3.26E−03 | 2.00E−03 |
| SMAD9 | 1.20E−02 | 3.77E−03 | −1.91E−03 |
| SMYD3 | 8.87E−03 | 1.33E−03 | −3.06E−04 |
| SNAI1 | 1.19E−02 | 1.35E−03 | −6.56E−03 |
| SNAI2 | −2.81E−02 | 1.73E−03 | −1.63E−02 |
| SNAI3 | 4.20E−03 | 2.61E−02 | 7.23E−03 |
| SNAPC2 | −3.41E−03 | −8.11E−04 | 2.40E−05 |
| SNAPC4 | 1.83E−03 | −2.85E−03 | −2.33E−03 |
| SNAPC5 | 1.17E−03 | 4.22E−04 | −5.51E−04 |
| SOHLH1 | 3.73E−03 | 3.80E−04 | −1.57E−03 |
| SOHLH2 | 5.20E−03 | −9.42E−03 | −3.52E−03 |
| SON | −1.76E−03 | −2.00E−03 | −2.52E−03 |
| SOX1 | 1.93E−02 | 3.02E−02 | −4.49E−03 |
| SOX10 | −6.21E−03 | −4.03E−03 | 2.71E−03 |
| SOX11 | 1.59E−02 | −2.31E−02 | −1.58E−02 |
| SOX12 | 7.67E−03 | −2.63E−03 | 1.19E−03 |
| SOX13 | 2.85E−03 | −1.00E−02 | −1.33E−03 |
| SOX14 | 1.34E−03 | −1.31E−03 | −4.68E−04 |
| SOX15 | −1.68E−02 | 2.02E−03 | −7.27E−03 |
| SOX17 | 2.44E−02 | −3.30E−02 | −2.81E−02 |
| SOX18 | −8.49E−03 | −4.72E−03 | −3.48E−03 |
| SOX2 | 9.35E−03 | 2.86E−03 | −8.24E−04 |
| SOX21 | −4.65E−03 | 1.71E−02 | 2.35E−03 |
| SOX3 | 1.54E−04 | −5.11E−05 | −1.42E−04 |
| SOX30 | −5.97E−04 | 2.82E−03 | −4.01E−03 |
| SOX4 | 4.95E−03 | −1.99E−03 | −4.43E−03 |
| SOX5 | 2.29E−02 | 4.12E−03 | −9.97E−03 |
| SOX6 | 2.06E−03 | 7.63E−03 | −1.47E−02 |
| SOX7 | −2.53E−02 | −1.43E−03 | −1.30E−02 |
| SOX8 | 9.13E−03 | −1.29E−02 | −2.19E−02 |
| SOX9 | −8.91E−04 | −1.34E−02 | −1.28E−02 |
| SP1 | −3.74E−03 | −3.37E−03 | −7.94E−04 |
| SP100 | −8.01E−03 | 2.03E−03 | −5.12E−03 |
| SP110 | −5.02E−03 | 4.52E−03 | −5.10E−03 |
| SP140 | −3.68E−03 | −1.79E−03 | −2.74E−03 |
| SP140L | −1.26E−02 | 1.07E−02 | −4.57E−03 |
| SP2 | 1.06E−04 | −7.68E−04 | −1.98E−03 |
| SP3 | −1.06E−03 | −3.58E−03 | −2.31E−03 |
| SP4 | 4.14E−03 | −2.87E−03 | −3.43E−03 |
| SP5 | 5.38E−04 | −1.48E−03 | −1.02E−03 |
| SP6 | −1.13E−02 | 1.37E−02 | 1.92E−03 |
| SP7 | 2.92E−03 | −4.04E−03 | 1.70E−04 |
| SP8 | −2.46E−02 | −2.27E−05 | −1.23E−03 |
| SP9 | 5.83E−03 | −7.36E−03 | 4.66E−04 |
| SPDEF | −1.54E−02 | 1.11E−02 | −8.92E−03 |
| SPEN | −1.88E−03 | −2.07E−03 | −5.94E−03 |
| SPI1 | 8.57E−03 | −9.92E−03 | −1.06E−02 |
| SPIB | 2.66E−02 | −2.11E−02 | 3.28E−02 |
| SPIC | 2.24E−03 | −1.39E−03 | −1.44E−02 |
| SPZ1 | 1.36E−03 | 6.55E−05 | −1.87E−03 |
| SRCAP | −3.34E−04 | −1.48E−03 | −1.88E−03 |
| SREBF1 | −4.20E−03 | −2.46E−03 | 9.27E−04 |
| SREBF2 | −2.42E−03 | −2.41E−03 | 1.37E−03 |
| SRF | 1.49E−03 | −2.34E−03 | −4.69E−03 |
| SRY | 4.66E−03 | 5.08E−03 | −7.19E−03 |
| ST18 | 3.71E−02 | −2.27E−03 | −2.66E−02 |
| STAT1 | −1.69E−03 | 2.94E−03 | −5.00E−03 |
| STAT2 | −5.61E−03 | 1.34E−03 | −4.15E−03 |
| STAT3 | −6.56E−03 | −5.68E−04 | −4.28E−03 |
| STAT4 | 1.29E−03 | −2.32E−02 | −5.77E−03 |
| STAT5A | −1.31E−02 | 3.11E−03 | −9.48E−03 |
| STAT5B | −1.10E−03 | 2.42E−03 | 1.05E−03 |
| STAT6 | −7.74E−03 | −8.27E−03 | −5.56E−03 |
| T | 7.34E−03 | 1.54E−02 | 4.07E−04 |
| TAL1 | −5.98E−03 | −1.82E−03 | −4.01E−03 |
| TAL2 | 1.87E−04 | 3.39E−03 | 1.30E−05 |
| TBP | −5.41E−04 | −2.90E−03 | 1.88E−04 |
| TBPL1 | 5.16E−03 | 4.28E−03 | −3.85E−03 |
| TBPL2 | −2.89E−04 | −8.14E−04 | 9.35E−04 |
| TBR1 | −3.26E−03 | −4.59E−04 | −7.39E−06 |
| TBX1 | −1.63E−03 | −2.92E−04 | −3.08E−03 |
| TBX10 | 1.73E−02 | 2.69E−02 | −6.16E−03 |
| TBX15 | 3.06E−03 | −4.37E−03 | −6.37E−03 |
| TBX18 | −1.81E−03 | 3.49E−04 | 7.43E−04 |
| TBX19 | −5.99E−03 | −1.78E−03 | −2.24E−03 |
| TBX2 | −4.68E−03 | −2.96E−03 | −1.29E−02 |
| TBX20 | 1.01E−02 | 1.29E−02 | 7.46E−04 |
| TBX21 | −1.38E−03 | −5.71E−03 | 1.67E−03 |
| TBX3 | −5.76E−03 | −7.52E−03 | −4.34E−03 |
| TBX4 | −6.87E−03 | −4.50E−03 | −5.68E−03 |
| TBX5 | 7.25E−03 | −1.57E−02 | −1.70E−03 |
| TBX6 | −8.63E−03 | −2.26E−03 | −2.98E−03 |
| TCF12 | 5.73E−03 | 2.56E−03 | −3.17E−03 |
| TCF15 | 1.09E−02 | −1.91E−02 | −6.95E−03 |
| TCF20 | −2.01E−03 | −7.81E−04 | −1.90E−03 |
| TCF21 | −6.78E−03 | −2.92E−03 | −7.00E−04 |
| TCF23 | −2.32E−03 | −1.25E−03 | −4.70E−03 |
| TCF24 | 7.47E−03 | 6.13E−03 | −1.83E−03 |
| TCF3 | 3.18E−03 | −1.71E−03 | −4.28E−03 |
| TCF4 | 7.90E−03 | 2.29E−03 | −4.89E−03 |
| TCF7 | −6.30E−03 | −6.57E−03 | −5.41E−03 |
| TCF7L1 | −1.83E−02 | −1.90E−03 | −1.22E−02 |
| TCF7L2 | −9.18E−03 | 1.09E−02 | −4.70E−03 |
| TCFL5 | −1.57E−04 | −2.49E−03 | 1.14E−03 |
| TEAD1 | −3.18E−03 | 3.32E−03 | −1.70E−03 |
| TEAD2 | −2.20E−03 | −1.88E−02 | −2.19E−02 |
| TEAD3 | −7.53E−03 | −4.31E−03 | −5.28E−03 |
| TEAD4 | 4.83E−03 | −9.07E−03 | −6.11E−03 |
| TEF | −6.08E−04 | 6.52E−03 | 7.87E−03 |
| TERF1 | 1.89E−03 | −2.60E−03 | −1.74E−03 |
| TERF2 | 4.60E−04 | −2.17E−03 | −2.14E−04 |
| TET1 | 1.81E−02 | −6.60E−03 | −1.06E−02 |
| TET2 | 8.36E−04 | −2.84E−03 | −4.90E−03 |
| TET3 | 3.08E−03 | −1.43E−03 | −5.32E−03 |
| TFAP2A | −1.88E−02 | 1.19E−02 | −6.48E−03 |
| TFAP2B | 2.97E−02 | −2.32E−02 | −3.87E−02 |
| TFAP2C | −6.48E−03 | −1.34E−02 | −9.59E−03 |
| TFAP2D | 5.33E−04 | −8.62E−04 | −3.27E−04 |
| TFAP2E | 1.09E−02 | −1.61E−02 | −1.08E−02 |
| TFAP4 | 3.14E−03 | −4.16E−03 | −1.24E−03 |
| TFCP2 | −6.74E−04 | −2.97E−03 | −5.37E−05 |
| TFCP2L1 | 5.70E−04 | 9.95E−03 | −9.46E−03 |
| TFDP1 | 6.71E−03 | −5.61E−04 | −2.82E−03 |
| TFDP2 | 3.19E−03 | 2.04E−03 | 5.53E−05 |
| TFDP3 | 6.75E−04 | 5.09E−04 | −4.20E−04 |
| TFE3 | −5.92E−03 | −1.51E−03 | −2.77E−03 |
| TFEB | −3.35E−03 | 2.07E−02 | 9.65E−04 |
| TFEC | −1.36E−03 | 5.38E−04 | −2.12E−03 |
| TGIF1 | −9.27E−03 | 1.88E−03 | −6.50E−03 |
| TGIF2 | 2.73E−03 | −5.37E−03 | −7.89E−03 |
| THAP1 | −3.96E−04 | 2.28E−04 | −3.83E−04 |
| THAP10 | −1.90E−03 | −5.27E−03 | −2.68E−03 |
| THAP11 | 3.33E−03 | −4.49E−03 | −2.27E−03 |
| THAP2 | −4.93E−03 | −1.57E−03 | −5.08E−03 |
| THAP3 | −5.71E−05 | −1.13E−03 | 2.97E−03 |
| THAP4 | 2.12E−03 | 1.24E−04 | −1.43E−03 |
| THAP5 | 1.68E−03 | −2.69E−03 | 1.64E−03 |
| THAP6 | −2.45E−03 | 8.31E−04 | 2.55E−03 |
| THAP7 | 3.09E−03 | 3.37E−03 | 2.65E−03 |
| THAP8 | 1.30E−03 | 3.16E−03 | 2.41E−03 |
| THAP9 | 9.28E−04 | −3.92E−03 | −7.59E−04 |
| THRA | −1.03E−04 | −8.69E−03 | −3.51E−03 |
| THRB | −2.07E−02 | 1.74E−02 | −1.06E−02 |
| THYN1 | 5.06E−03 | 3.67E−04 | −6.41E−04 |
| TIGD1 | 2.32E−03 | −3.29E−03 | −2.98E−04 |
| TIGD2 | −1.20E−03 | 8.45E−04 | 3.27E−03 |
| TIGD3 | 9.26E−03 | 9.05E−03 | −6.40E−04 |
| TIGD4 | 2.82E−03 | −1.68E−03 | −1.39E−03 |
| TIGD5 | 1.27E−05 | −9.29E−04 | 1.36E−03 |
| TIGD6 | −4.45E−03 | −1.65E−04 | 2.57E−03 |
| TIGD7 | 3.18E−03 | −5.85E−03 | −1.10E−03 |
| TLX1 | 1.96E−03 | −8.85E−04 | −2.98E−03 |
| TLX2 | 8.23E−03 | −1.54E−02 | −2.59E−03 |
| TLX3 | 2.96E−02 | −2.30E−02 | −2.26E−02 |
| TMF1 | −2.40E−03 | 1.03E−03 | −4.58E−04 |
| TOPORS | −1.15E−03 | −2.09E−03 | −1.98E−03 |
| TP53 | 3.85E−03 | 7.01E−03 | −3.82E−03 |
| TP63 | −3.75E−02 | −3.01E−03 | −1.96E−02 |
| TP73 | −3.96E−03 | 1.21E−02 | 1.08E−03 |
| TPRX1 | −9.66E−04 | −1.95E−03 | 1.51E−03 |
| TRAFD1 | −3.57E−03 | 3.02E−05 | −3.80E−03 |
| TRERF1 | −1.67E−03 | 5.54E−04 | −1.18E−03 |
| TRPS1 | −8.51E−03 | 1.09E−02 | −6.44E−03 |
| TSC22D1 | −5.85E−03 | 9.73E−04 | −3.51E−03 |
| TSHZ1 | 4.20E−03 | −1.84E−03 | 4.69E−05 |
| TSHZ2 | −5.03E−03 | 1.65E−02 | −3.22E−03 |
| TSHZ3 | −1.64E−02 | −9.25E−03 | −3.71E−03 |
| TTF1 | 1.59E−03 | −4.98E−03 | −9.53E−04 |
| TWIST1 | −1.18E−02 | 2.33E−03 | −9.41E−03 |
| TWIST2 | −2.32E−02 | −2.00E−03 | −3.07E−03 |
| UBP1 | −7.95E−05 | −3.41E−03 | −2.80E−03 |
| UNCX | 4.53E−03 | −5.30E−03 | −2.42E−03 |
| USF1 | 9.44E−04 | −1.87E−03 | −3.75E−03 |
| USF2 | 6.43E−04 | −8.24E−04 | −1.40E−03 |
| VAX1 | 8.03E−05 | 1.16E−03 | −9.30E−04 |
| VAX2 | 3.67E−03 | −1.32E−02 | 1.34E−04 |
| VDR | −8.59E−03 | −1.17E−03 | −3.70E−03 |
| VENTX | 6.27E−04 | −2.52E−03 | −9.08E−04 |
| VEZF1 | −1.84E−03 | −2.18E−03 | −1.33E−03 |
| VSX1 | −4.82E−03 | −1.66E−03 | 9.75E−04 |
| VSX2 | −1.68E−03 | −2.49E−03 | 3.40E−03 |
| WIZ | 2.46E−04 | −2.93E−03 | −3.56E−03 |
| WT1 | −6.91E−03 | −1.50E−03 | −5.23E−03 |
| XBP1 | −3.39E−03 | 4.05E−03 | −3.04E−03 |
| XPA | 2.07E−03 | −3.44E−03 | −2.93E−04 |
| YBX1 | 1.34E−03 | −1.35E−03 | −2.46E−03 |
| YBX2 | 7.67E−03 | −1.03E−02 | −1.73E−03 |
| YBX3 | −7.40E−03 | −4.37E−04 | −1.70E−03 |
| YY1 | 1.41E−03 | −2.43E−03 | −2.75E−03 |
| YY2 | −4.96E−04 | −2.48E−03 | −6.45E−04 |
| ZBED1 | 1.61E−03 | 3.14E−03 | −1.17E−03 |
| ZBED2 | −2.85E−02 | −5.22E−03 | 3.67E−03 |
| ZBED3 | 1.93E−03 | −3.00E−04 | −1.89E−03 |
| ZBED4 | 2.72E−03 | −2.91E−03 | −2.24E−03 |
| ZBED5 | −2.31E−03 | −3.13E−03 | 1.93E−03 |
| ZBED6 | −4.16E−03 | −5.45E−03 | −3.12E−03 |
| ZBED9 | −2.35E−04 | −7.86E−03 | 2.38E−03 |
| ZBTB1 | −2.16E−03 | −2.11E−04 | −7.18E−04 |
| ZBTB10 | −3.57E−03 | −8.86E−05 | −3.29E−03 |
| ZBTB11 | −6.61E−05 | −1.94E−03 | −3.08E−03 |
| ZBTB12 | 2.93E−03 | −1.21E−02 | −4.54E−03 |
| ZBTB14 | 1.75E−03 | −8.67E−04 | 2.28E−03 |
| ZBTB16 | −1.19E−02 | −2.95E−03 | −3.63E−03 |
| ZBTB17 | −2.58E−05 | −4.42E−04 | −3.11E−03 |
| ZBTB18 | 8.97E−03 | 4.74E−03 | −1.51E−03 |
| ZBTB2 | −4.12E−04 | −8.43E−04 | −3.99E−03 |
| ZBTB20 | 6.76E−03 | −9.71E−03 | −1.11E−02 |
| ZBTB21 | −3.12E−03 | −2.79E−03 | −4.46E−03 |
| ZBTB22 | −6.92E−03 | −4.63E−04 | 1.80E−03 |
| ZBTB24 | 3.06E−03 | −1.71E−03 | 3.46E−04 |
| ZBTB25 | −2.32E−03 | 6.96E−04 | 4.38E−03 |
| ZBTB26 | 3.37E−03 | −5.28E−03 | 1.27E−04 |
| ZBTB3 | 8.22E−04 | −3.40E−03 | 2.43E−03 |
| ZBTB32 | −1.76E−03 | 1.03E−03 | −1.68E−03 |
| ZBTB33 | 2.00E−03 | −2.95E−03 | −1.25E−03 |
| ZBTB34 | −2.85E−04 | −3.26E−03 | −1.46E−03 |
| ZBTB37 | −1.03E−04 | −4.75E−03 | 2.31E−03 |
| ZBTB38 | −3.50E−03 | −5.61E−04 | −2.14E−03 |
| ZBTB39 | 2.37E−03 | −5.95E−03 | −5.75E−04 |
| ZBTB4 | −9.78E−03 | 7.55E−04 | −2.16E−04 |
| ZBTB40 | 1.99E−03 | −1.10E−03 | −2.17E−03 |
| ZBTB41 | 1.63E−04 | −1.78E−03 | 2.62E−03 |
| ZBTB42 | −4.53E−03 | 2.92E−03 | 3.41E−04 |
| ZBTB43 | −1.92E−03 | 2.16E−03 | −5.48E−03 |
| ZBTB44 | 1.48E−03 | −4.60E−03 | −1.15E−03 |
| ZBTB45 | 7.56E−04 | −1.35E−03 | 5.68E−04 |
| ZBTB46 | −1.29E−02 | −5.90E−04 | −2.73E−03 |
| ZBTB47 | −8.25E−04 | 2.17E−03 | −4.91E−04 |
| ZBTB48 | 4.73E−04 | −1.22E−03 | 9.19E−04 |
| ZBTB49 | −2.43E−03 | −2.33E−03 | −1.02E−03 |
| ZBTB5 | −2.38E−03 | −2.84E−03 | −2.18E−03 |
| ZBTB6 | 1.83E−03 | −4.35E−03 | 9.80E−04 |
| ZBTB7A | −3.26E−03 | 1.45E−03 | −3.36E−03 |
| ZBTB7B | −3.15E−03 | −4.46E−04 | 3.35E−04 |
| ZBTB7C | −2.22E−02 | 7.36E−03 | −1.14E−02 |
| ZBTB8A | −1.64E−03 | −1.75E−03 | −1.97E−03 |
| ZBTB8B | 8.99E−03 | −1.60E−02 | −7.46E−03 |
| ZBTB9 | −2.06E−03 | −4.40E−03 | −7.52E−04 |
| ZC3H8 | 4.32E−05 | −1.69E−03 | 6.23E−04 |
| ZEB1 | −4.08E−04 | −1.39E−03 | −5.03E−04 |
| ZEB2 | 1.24E−02 | −3.77E−04 | −1.85E−02 |
| ZFAT | −7.38E−05 | 2.30E−03 | −7.97E−04 |
| ZFHX2 | 1.43E−03 | −9.77E−03 | −4.93E−03 |
| ZFHX3 | 5.29E−03 | −8.03E−03 | −5.90E−03 |
| ZFHX4 | 2.07E−02 | −1.20E−02 | −1.37E−02 |
| ZFP1 | 1.22E−03 | −5.59E−03 | 8.18E−04 |
| ZFP14 | 7.77E−04 | −7.83E−03 | 2.08E−03 |
| ZFP2 | −9.04E−03 | −8.14E−03 | 7.91E−04 |
| ZFP28 | −1.26E−02 | −1.51E−02 | 1.98E−03 |
| ZFP3 | −2.68E−03 | −5.63E−03 | 3.41E−03 |
| ZFP30 | 6.09E−04 | 1.52E−03 | 2.55E−03 |
| ZFP37 | −3.59E−03 | −1.40E−02 | 3.16E−03 |
| ZFP41 | −1.73E−03 | −1.79E−03 | −1.71E−04 |
| ZFP42 | 3.02E−02 | −1.43E−02 | −3.05E−02 |
| ZFP57 | 1.02E−02 | 1.41E−02 | −1.01E−02 |
| ZFP62 | 1.22E−03 | −4.25E−03 | 5.08E−04 |
| ZFP64 | −3.09E−04 | −2.31E−03 | −2.57E−04 |
| ZFP69 | 3.98E−03 | −2.07E−03 | 2.05E−03 |
| ZFP69B | 5.49E−03 | −2.88E−03 | −3.98E−03 |
| ZFP82 | 7.07E−04 | −1.09E−02 | 2.63E−03 |
| ZFP90 | −8.61E−04 | −1.63E−03 | 2.52E−03 |
| ZFP91 | 8.55E−05 | −6.83E−04 | −3.03E−03 |
| ZFP92 | 4.87E−03 | −1.37E−02 | −6.10E−03 |
| ZFPM1 | −2.12E−03 | −7.79E−04 | 1.02E−04 |
| ZFPM2 | −8.38E−03 | −1.13E−02 | −3.66E−03 |
| ZFX | −8.17E−04 | −2.10E−03 | −2.08E−03 |
| ZFY | −1.33E−03 | 8.31E−04 | −1.64E−03 |
| ZGLP1 | −1.27E−03 | −8.48E−04 | −3.80E−03 |
| ZGPAT | −1.44E−03 | −1.96E−04 | −1.74E−03 |
| ZHX1 | −1.49E−03 | 1.89E−03 | −6.83E−04 |
| ZHX2 | −3.64E−03 | −3.40E−04 | −3.60E−03 |
| ZHX3 | 3.49E−03 | 2.59E−03 | −1.21E−03 |
| ZIC1 | 2.10E−02 | −2.11E−02 | −1.53E−02 |
| ZIC2 | 1.60E−02 | 3.35E−02 | −6.55E−03 |
| ZIC3 | 4.83E−03 | −7.62E−03 | −4.05E−03 |
| ZIC4 | 1.25E−02 | −1.56E−02 | −6.02E−03 |
| ZIC5 | 1.37E−02 | 2.39E−02 | −7.59E−03 |
| ZIK1 | 4.26E−03 | −1.13E−02 | 1.11E−03 |
| ZIM2 | −9.63E−03 | −3.25E−03 | 4.74E−03 |
| ZIM3 | −1.80E−04 | −9.66E−05 | −1.18E−04 |
| ZKSCAN1 | −4.58E−03 | 1.72E−03 | 4.33E−03 |
| ZKSCAN2 | 1.47E−03 | −3.92E−03 | 2.17E−03 |
| ZKSCAN3 | −1.58E−03 | −3.15E−03 | 2.77E−03 |
| ZKSCAN4 | 2.50E−04 | −3.27E−03 | 3.65E−03 |
| ZKSCAN5 | 1.26E−04 | −5.37E−03 | −1.14E−04 |
| ZKSCAN7 | −3.69E−03 | −1.36E−03 | 2.94E−03 |
| ZKSCAN8 | −5.63E−04 | −5.80E−03 | 1.04E−03 |
| ZMAT1 | −9.61E−03 | −1.75E−02 | −2.39E−03 |
| ZMAT4 | 1.36E−02 | 1.40E−02 | −3.64E−03 |
| ZNF10 | 2.53E−03 | −4.15E−03 | −1.93E−03 |
| ZNF100 | 1.92E−03 | −4.90E−03 | 6.18E−04 |
| ZNF101 | 2.28E−03 | −4.58E−03 | −4.16E−04 |
| ZNF107 | 3.24E−03 | −2.61E−03 | 2.25E−03 |
| ZNF112 | −5.51E−04 | −4.23E−03 | 2.04E−03 |
| ZNF114 | 1.12E−02 | −2.25E−02 | −1.39E−02 |
| ZNF117 | −6.88E−03 | −4.57E−03 | −3.70E−03 |
| ZNF12 | −1.57E−03 | −2.98E−03 | −3.10E−04 |
| ZNF121 | 4.17E−04 | −2.78E−03 | −2.79E−03 |
| ZNF124 | 4.65E−03 | −2.28E−03 | −2.95E−03 |
| ZNF131 | 8.65E−04 | −2.21E−03 | −3.30E−03 |
| ZNF132 | −3.93E−03 | −4.03E−03 | 2.79E−03 |
| ZNF133 | −7.77E−04 | −1.41E−03 | 4.42E−04 |
| ZNF134 | −3.03E−04 | −4.30E−03 | 1.30E−04 |
| ZNF135 | −1.80E−02 | −1.11E−02 | 3.47E−03 |
| ZNF136 | −5.86E−03 | −3.59E−03 | −3.04E−03 |
| ZNF138 | 1.26E−03 | −1.98E−03 | 1.85E−03 |
| ZNF14 | −3.48E−03 | −8.16E−03 | 3.29E−03 |
| ZNF140 | −2.14E−03 | −3.02E−03 | −1.65E−03 |
| ZNF141 | 1.60E−03 | −5.05E−03 | 2.19E−03 |
| ZNF142 | 1.81E−03 | −1.61E−03 | −3.31E−03 |
| ZNF143 | −1.92E−03 | −1.43E−03 | −1.99E−03 |
| ZNF146 | 4.67E−04 | −4.16E−03 | −1.18E−03 |
| ZNF148 | −2.06E−03 | −2.01E−03 | 1.00E−04 |
| ZNF154 | −8.74E−04 | −7.24E−03 | −5.34E−03 |
| ZNF155 | −3.59E−04 | −1.88E−03 | 6.73E−04 |
| ZNF157 | 7.46E−03 | −8.62E−04 | −4.72E−04 |
| ZNF16 | −4.06E−04 | −3.53E−03 | 5.78E−04 |
| ZNF160 | −1.23E−02 | 6.01E−03 | 4.91E−04 |
| ZNF165 | −1.36E−03 | 1.54E−03 | −7.14E−03 |
| ZNF169 | 2.19E−03 | −4.95E−03 | 1.05E−03 |
| ZNF17 | 1.08E−03 | −3.26E−03 | −1.09E−03 |
| ZNF174 | 1.07E−03 | −2.06E−03 | 2.21E−03 |
| ZNF175 | −8.30E−03 | −8.23E−03 | −3.31E−03 |
| ZNF177 | −1.32E−02 | −3.27E−03 | −2.44E−03 |
| ZNF18 | −5.35E−04 | −2.55E−03 | 1.83E−03 |
| ZNF180 | 2.13E−03 | −4.74E−03 | 1.40E−03 |
| ZNF181 | −3.13E−03 | −7.62E−03 | 2.84E−03 |
| ZNF182 | 1.07E−03 | −3.23E−03 | 1.89E−03 |
| ZNF184 | 2.62E−03 | −3.45E−03 | 1.54E−04 |
| ZNF189 | −1.61E−03 | −2.70E−03 | 5.51E−04 |
| ZNF19 | −3.29E−03 | −1.22E−03 | 2.75E−03 |
| ZNF195 | 2.11E−03 | −2.00E−03 | −1.09E−03 |
| ZNF197 | −1.27E−03 | −3.66E−03 | 3.53E−03 |
| ZNF2 | 8.45E−05 | −3.70E−03 | 1.76E−03 |
| ZNF20 | −7.62E−03 | −1.85E−03 | 3.48E−03 |
| ZNF200 | 3.74E−04 | −3.68E−03 | 2.45E−04 |
| ZNF202 | −8.76E−05 | −3.17E−03 | 2.63E−03 |
| ZNF205 | 1.03E−03 | −2.12E−03 | 1.85E−04 |
| ZNF207 | 9.54E−04 | −1.35E−03 | −3.13E−03 |
| ZNF208 | −1.89E−02 | −5.31E−03 | 9.96E−03 |
| ZNF211 | −5.93E−04 | −1.34E−03 | 6.68E−04 |
| ZNF212 | 4.44E−04 | −9.96E−04 | −2.09E−03 |
| ZNF213 | −1.85E−03 | −1.39E−03 | 7.06E−04 |
| ZNF214 | −2.32E−03 | 6.24E−04 | 3.89E−03 |
| ZNF215 | 7.51E−03 | 2.94E−03 | 1.27E−03 |
| ZNF217 | −8.84E−03 | 2.29E−03 | −5.74E−03 |
| ZNF219 | −4.64E−03 | −4.77E−03 | −6.38E−03 |
| ZNF22 | 1.98E−03 | −2.51E−03 | −5.52E−05 |
| ZNF221 | 5.37E−04 | −5.78E−03 | 2.94E−03 |
| ZNF222 | −2.19E−03 | −3.56E−03 | −1.08E−03 |
| ZNF223 | −2.26E−04 | −3.50E−03 | 2.23E−03 |
| ZNF224 | −3.33E−04 | −2.65E−03 | 1.47E−03 |
| ZNF225 | 1.57E−03 | −3.85E−03 | 1.83E−03 |
| ZNF226 | −1.45E−03 | −2.38E−03 | 1.99E−03 |
| ZNF227 | 1.00E−03 | −2.36E−03 | 5.78E−04 |
| ZNF229 | −4.11E−03 | −3.44E−03 | −1.71E−04 |
| ZNF23 | −9.18E−04 | −4.77E−03 | 2.68E−03 |
| ZNF230 | 1.22E−03 | −3.88E−03 | 4.59E−04 |
| ZNF232 | 1.86E−03 | −6.46E−03 | −2.45E−03 |
| ZNF233 | 8.11E−03 | −1.16E−03 | −3.75E−04 |
| ZNF234 | −4.30E−05 | −2.92E−03 | −2.18E−04 |
| ZNF235 | 4.15E−03 | −3.38E−03 | −1.98E−04 |
| ZNF236 | −8.92E−04 | 3.21E−04 | 2.03E−04 |
| ZNF239 | 5.11E−03 | −5.57E−03 | −4.99E−03 |
| ZNF24 | 8.96E−04 | −1.43E−03 | −7.69E−04 |
| ZNF248 | 1.33E−03 | −3.60E−03 | −2.69E−04 |
| ZNF25 | −3.74E−03 | −7.11E−03 | 2.32E−03 |
| ZNF250 | −2.07E−04 | −2.00E−03 | 1.52E−03 |
| ZNF251 | −6.47E−04 | −5.78E−03 | 1.23E−03 |
| ZNF253 | −3.80E−03 | −1.42E−03 | 4.81E−03 |
| ZNF254 | 3.80E−03 | −5.72E−04 | 1.08E−03 |
| ZNF256 | −1.41E−03 | −6.38E−03 | 9.87E−04 |
| ZNF257 | −1.71E−02 | −1.71E−04 | 3.49E−03 |
| ZNF26 | −6.51E−04 | −3.75E−03 | −1.64E−03 |
| ZNF260 | 1.76E−03 | −4.92E−03 | −1.52E−03 |
| ZNF263 | −8.82E−04 | −1.46E−03 | −2.04E−03 |
| ZNF264 | −3.85E−03 | −2.67E−03 | −5.17E−04 |
| ZNF266 | −1.86E−03 | −5.21E−03 | −3.48E−03 |
| ZNF267 | −1.88E−03 | −3.07E−03 | −5.52E−03 |
| ZNF268 | −1.41E−03 | −5.37E−03 | −6.80E−04 |
| ZNF273 | 2.57E−03 | −9.90E−04 | 6.67E−04 |
| ZNF274 | −4.10E−03 | −3.27E−03 | 2.42E−03 |
| ZNF275 | 2.03E−03 | −9.72E−04 | 1.87E−03 |
| ZNF276 | 4.52E−04 | −1.44E−03 | −2.63E−03 |
| ZNF277 | 1.09E−03 | −1.03E−03 | 8.61E−04 |
| ZNF28 | −4.17E−03 | −9.45E−04 | 7.21E−04 |
| ZNF280A | 1.37E−02 | −1.69E−02 | −4.46E−03 |
| ZNF280B | 9.90E−03 | −5.96E−03 | −5.35E−05 |
| ZNF280C | 6.13E−04 | −6.31E−03 | 1.45E−03 |
| ZNF280D | −5.37E−04 | −3.07E−03 | −2.94E−04 |
| ZNF281 | −1.51E−03 | −3.55E−03 | −3.91E−03 |
| ZNF282 | 3.74E−04 | −1.53E−03 | −1.94E−04 |
| ZNF283 | −1.16E−03 | −4.93E−03 | 7.67E−04 |
| ZNF284 | −5.49E−04 | −2.87E−03 | 7.32E−04 |
| ZNF285 | −1.85E−03 | −5.08E−03 | 3.79E−03 |
| ZNF286A | 2.49E−03 | −5.38E−03 | −2.75E−03 |
| ZNF286B | 2.77E−03 | −7.24E−03 | −5.94E−03 |
| ZNF287 | 1.36E−03 | −2.35E−03 | 2.59E−03 |
| ZNF292 | −1.41E−03 | −1.93E−03 | 2.41E−03 |
| ZNF296 | 2.24E−03 | 3.50E−04 | 6.43E−04 |
| ZNF3 | 1.93E−03 | −9.08E−04 | −1.97E−04 |
| ZNF30 | 2.96E−04 | −4.08E−03 | 6.57E−03 |
| ZNF300 | 3.18E−03 | −6.29E−03 | −2.68E−03 |
| ZNF302 | −1.56E−03 | −6.68E−03 | 3.47E−03 |
| ZNF304 | 1.73E−03 | −1.19E−03 | −2.89E−03 |
| ZNF311 | −1.08E−02 | −1.24E−02 | 8.07E−04 |
| ZNF316 | −7.45E−04 | −1.58E−03 | −1.11E−03 |
| ZNF317 | −1.32E−03 | −1.43E−03 | −1.57E−03 |
| ZNF318 | 2.61E−03 | −3.24E−03 | −1.59E−03 |
| ZNF319 | −3.18E−03 | −2.28E−03 | −7.52E−05 |
| ZNF32 | −1.47E−03 | −3.71E−03 | −2.52E−03 |
| ZNF320 | −1.37E−02 | 5.10E−03 | −5.41E−04 |
| ZNF322 | 7.94E−04 | 2.48E−03 | 2.46E−03 |
| ZNF324 | −2.78E−04 | 4.86E−05 | −1.68E−03 |
| ZNF324B | 1.02E−03 | −1.05E−03 | −2.54E−04 |
| ZNF326 | 2.18E−03 | −1.07E−03 | −4.02E−03 |
| ZNF329 | −5.54E−03 | −5.51E−03 | 1.48E−03 |
| ZNF331 | −3.69E−03 | −6.75E−03 | −1.38E−03 |
| ZNF333 | −3.16E−03 | −6.59E−03 | 2.03E−03 |
| ZNF334 | −4.69E−03 | −1.55E−03 | 8.36E−03 |
| ZNF335 | −9.04E−05 | 8.19E−04 | −3.19E−03 |
| ZNF337 | −1.78E−03 | 2.23E−03 | 5.29E−03 |
| ZNF33A | −2.88E−04 | −3.81E−03 | 3.45E−04 |
| ZNF33B | 1.33E−03 | −5.20E−03 | −8.25E−04 |
| ZNF34 | −1.63E−03 | −1.33E−03 | −3.28E−03 |
| ZNF341 | −1.25E−03 | −4.95E−04 | 1.48E−03 |
| ZNF343 | −6.98E−04 | −2.80E−03 | 4.36E−04 |
| ZNF345 | −2.77E−03 | −1.12E−02 | −5.40E−03 |
| ZNF346 | 2.46E−03 | −3.02E−03 | −1.39E−03 |
| ZNF347 | −2.82E−02 | −8.59E−04 | −3.49E−03 |
| ZNF35 | 1.20E−03 | −2.80E−03 | −3.60E−03 |
| ZNF350 | −2.56E−03 | −5.28E−03 | −3.75E−04 |
| ZNF354A | −1.27E−03 | −8.34E−03 | −3.63E−03 |
| ZNF354B | −1.02E−03 | −6.07E−03 | −2.77E−04 |
| ZNF354C | −9.35E−04 | −2.11E−02 | −3.10E−03 |
| ZNF358 | 3.86E−04 | −4.60E−03 | −2.99E−03 |
| ZNF362 | −2.03E−03 | −4.67E−03 | −3.16E−03 |
| ZNF365 | −1.15E−02 | 4.61E−03 | −9.69E−03 |
| ZNF366 | −7.10E−03 | −3.53E−03 | −1.65E−03 |
| ZNF367 | 1.47E−02 | 2.28E−03 | −3.88E−03 |
| ZNF37A | 7.99E−04 | −5.76E−04 | 6.08E−04 |
| ZNF382 | 4.12E−03 | −3.18E−03 | 3.34E−03 |
| ZNF383 | −4.05E−03 | −8.28E−03 | 1.56E−04 |
| ZNF384 | −1.63E−03 | −2.11E−03 | −1.50E−03 |
| ZNF385A | −8.06E−03 | −7.67E−03 | −7.22E−03 |
| ZNF385B | 1.10E−02 | −1.56E−02 | −1.13E−02 |
| ZNF385C | 3.44E−03 | −1.67E−02 | −7.38E−03 |
| ZNF385D | 5.00E−03 | −1.37E−02 | −2.16E−02 |
| ZNF391 | −1.72E−03 | −1.10E−02 | −8.66E−04 |
| ZNF394 | −1.89E−03 | −1.06E−03 | −3.35E−03 |
| ZNF395 | −1.99E−03 | −1.54E−04 | 2.70E−03 |
| ZNF396 | 1.64E−03 | 1.54E−03 | 3.54E−03 |
| ZNF397 | −1.71E−03 | −1.55E−03 | 3.08E−03 |
| ZNF398 | 1.40E−03 | −5.83E−03 | −2.96E−03 |
| ZNF404 | −4.40E−03 | −5.06E−03 | 7.21E−03 |
| ZNF407 | 2.22E−03 | −3.89E−03 | −4.05E−03 |
| ZNF408 | −1.19E−03 | −8.79E−05 | −1.27E−04 |
| ZNF41 | −1.32E−03 | −1.57E−05 | −1.31E−03 |
| ZNF410 | −3.24E−04 | −1.69E−03 | −2.90E−03 |
| ZNF414 | 9.84E−04 | −1.49E−04 | 5.81E−04 |
| ZNF415 | −2.93E−02 | −3.45E−04 | 2.28E−03 |
| ZNF416 | −1.08E−03 | −4.60E−03 | −7.25E−04 |
| ZNF417 | 1.11E−03 | −2.58E−03 | −1.79E−03 |
| ZNF418 | 3.35E−03 | −5.81E−03 | −8.38E−05 |
| ZNF419 | −6.63E−04 | −4.53E−03 | 2.06E−03 |
| ZNF420 | −2.42E−03 | −9.33E−03 | −5.47E−04 |
| ZNF423 | −3.14E−03 | −9.65E−03 | −8.37E−03 |
| ZNF425 | −6.50E−03 | −4.90E−03 | −1.36E−03 |
| ZNF426 | −1.75E−03 | −4.44E−03 | −1.18E−03 |
| ZNF428 | 5.40E−03 | −4.15E−03 | −6.22E−03 |
| ZNF429 | −1.36E−02 | 1.29E−02 | 5.82E−03 |
| ZNF43 | −3.74E−04 | −8.62E−03 | 6.64E−04 |
| ZNF430 | −2.38E−03 | −3.18E−03 | −2.29E−03 |
| ZNF431 | 1.96E−04 | −4.54E−03 | −2.93E−03 |
| ZNF432 | 7.03E−04 | −4.82E−03 | 3.86E−04 |
| ZNF433 | −9.99E−03 | −3.04E−03 | −3.94E−03 |
| ZNF436 | −3.70E−03 | −4.45E−03 | 1.94E−04 |
| ZNF438 | −6.87E−04 | −2.36E−03 | 6.61E−04 |
| ZNF439 | −1.18E−02 | −2.67E−03 | −5.64E−03 |
| ZNF44 | −4.53E−03 | 8.35E−04 | −9.41E−04 |
| ZNF440 | −8.11E−03 | 5.82E−03 | −4.98E−03 |
| ZNF441 | −5.05E−03 | 2.68E−03 | 8.12E−03 |
| ZNF442 | −1.42E−02 | −5.73E−03 | 2.51E−03 |
| ZNF443 | −4.18E−04 | −2.82E−03 | −2.56E−03 |
| ZNF444 | 2.34E−03 | −1.09E−03 | −1.49E−03 |
| ZNF445 | 1.31E−03 | −1.92E−03 | 2.31E−03 |
| ZNF446 | −2.51E−03 | −7.78E−05 | 8.55E−04 |
| ZNF449 | −4.69E−04 | −2.05E−03 | 5.58E−03 |
| ZNF45 | −6.79E−05 | −3.85E−03 | −3.59E−05 |
| ZNF451 | −3.67E−04 | −5.34E−04 | 4.41E−04 |
| ZNF454 | −1.41E−02 | −1.25E−02 | −2.08E−03 |
| ZNF460 | −2.06E−03 | −1.80E−03 | −4.99E−03 |
| ZNF461 | −1.88E−03 | −6.02E−03 | 6.22E−04 |
| ZNF462 | −1.29E−02 | −8.02E−03 | −9.08E−03 |
| ZNF467 | 2.49E−03 | 6.31E−03 | −1.59E−03 |
| ZNF468 | −4.34E−03 | 6.03E−04 | 3.41E−04 |
| ZNF469 | −1.71E−03 | −1.88E−02 | −2.32E−02 |
| ZNF470 | −4.73E−03 | −1.08E−02 | 4.15E−04 |
| ZNF471 | −2.09E−02 | −6.54E−03 | −2.06E−03 |
| ZNF473 | −8.26E−04 | −4.11E−03 | −7.99E−05 |
| ZNF474 | −4.11E−04 | −3.51E−03 | −1.31E−02 |
| ZNF48 | 3.75E−03 | −2.78E−03 | −1.89E−03 |
| ZNF480 | 2.30E−03 | −5.26E−03 | 1.23E−04 |
| ZNF483 | 4.79E−03 | −2.57E−03 | 3.09E−03 |
| ZNF484 | 2.12E−04 | −5.44E−03 | 1.07E−03 |
| ZNF485 | −6.54E−04 | −5.04E−03 | 1.60E−04 |
| ZNF486 | −1.51E−02 | 3.10E−04 | −6.61E−04 |
| ZNF488 | −1.86E−03 | 5.34E−03 | 8.88E−03 |
| ZNF490 | −1.45E−03 | −2.42E−03 | 1.89E−03 |
| ZNF491 | 2.16E−03 | 1.32E−02 | 4.29E−03 |
| ZNF492 | −1.57E−02 | −2.29E−03 | −5.08E−04 |
| ZNF493 | −3.06E−03 | −1.89E−03 | 1.21E−03 |
| ZNF496 | 1.01E−03 | 1.89E−04 | 1.10E−03 |
| ZNF497 | −3.07E−03 | −3.38E−03 | 3.27E−03 |
| ZNF500 | 4.29E−05 | −3.92E−03 | 3.11E−05 |
| ZNF501 | −3.82E−03 | −5.84E−03 | 4.86E−03 |
| ZNF502 | −3.40E−03 | −3.62E−03 | 4.68E−03 |
| ZNF503 | −5.47E−03 | −8.40E−03 | −4.12E−03 |
| ZNF506 | −4.39E−03 | −1.51E−03 | 2.59E−03 |
| ZNF507 | 5.08E−04 | −3.31E−03 | 5.80E−04 |
| ZNF510 | 7.58E−04 | −4.45E−03 | 2.33E−03 |
| ZNF511 | 5.07E−03 | 2.61E−03 | 1.41E−03 |
| ZNF512 | 3.93E−03 | −2.97E−03 | −1.75E−03 |
| ZNF512B | 4.58E−05 | 2.47E−03 | −7.82E−04 |
| ZNF513 | −5.08E−03 | 1.59E−03 | −2.99E−03 |
| ZNF514 | −1.62E−03 | −1.98E−03 | 2.83E−03 |
| ZNF516 | −5.76E−03 | 1.40E−02 | 1.09E−03 |
| ZNF517 | 3.81E−03 | 1.57E−02 | 5.62E−03 |
| ZNF518A | 3.08E−04 | −3.66E−03 | 2.28E−03 |
| ZNF518B | −7.33E−04 | −3.26E−03 | −2.25E−03 |
| ZNF519 | 6.88E−03 | −8.78E−03 | 1.24E−03 |
| ZNF521 | 1.43E−03 | −1.08E−02 | −1.51E−02 |
| ZNF524 | −3.04E−03 | 3.01E−03 | 7.23E−04 |
| ZNF525 | −3.60E−03 | −4.95E−03 | 2.27E−03 |
| ZNF526 | −1.48E−03 | −2.95E−03 | 3.23E−04 |
| ZNF527 | −9.08E−04 | −6.59E−03 | 4.45E−04 |
| ZNF528 | −1.08E−02 | −7.87E−03 | −1.92E−03 |
| ZNF529 | 4.42E−04 | −5.14E−03 | −2.29E−03 |
| ZNF530 | 4.50E−03 | −3.84E−03 | 1.37E−03 |
| ZNF532 | 4.82E−04 | −3.19E−03 | −6.55E−03 |
| ZNF534 | −1.88E−03 | −1.18E−03 | 9.25E−04 |
| ZNF536 | 4.74E−03 | −2.00E−02 | −6.11E−03 |
| ZNF540 | 5.67E−04 | 6.49E−04 | 2.64E−03 |
| ZNF541 | 7.74E−03 | 5.37E−03 | −1.19E−03 |
| ZNF543 | 1.83E−03 | −8.74E−04 | −1.08E−03 |
| ZNF544 | 2.59E−03 | −2.10E−04 | −9.74E−04 |
| ZNF546 | −1.44E−03 | −3.90E−03 | 6.41E−03 |
| ZNF547 | −7.79E−04 | −1.85E−03 | −9.42E−04 |
| ZNF548 | 7.89E−04 | −2.48E−03 | −3.85E−05 |
| ZNF549 | −3.65E−04 | −7.71E−03 | 4.09E−04 |
| ZNF550 | 1.99E−04 | −6.19E−03 | 1.16E−04 |
| ZNF551 | 4.48E−04 | −3.66E−03 | −1.17E−03 |
| ZNF552 | −1.20E−03 | −5.37E−03 | −3.26E−03 |
| ZNF554 | −5.02E−03 | −7.16E−03 | 2.57E−03 |
| ZNF555 | −3.32E−03 | −5.70E−03 | 1.15E−03 |
| ZNF556 | −1.34E−05 | −1.95E−03 | 2.75E−03 |
| ZNF557 | −1.92E−03 | −2.24E−03 | 1.58E−03 |
| ZNF558 | −1.29E−02 | 2.74E−03 | −1.32E−03 |
| ZNF559 | −1.19E−02 | −1.15E−02 | −2.27E−03 |
| ZNF560 | 2.88E−03 | −1.67E−02 | 6.11E−03 |
| ZNF561 | −3.46E−03 | −2.92E−03 | −4.53E−04 |
| ZNF562 | −2.72E−04 | −1.40E−03 | −3.65E−03 |
| ZNF563 | −1.03E−02 | −4.43E−03 | −6.04E−04 |
| ZNF564 | −1.98E−03 | −1.94E−03 | 3.63E−03 |
| ZNF565 | −3.74E−03 | −1.41E−03 | −1.00E−03 |
| ZNF566 | 2.41E−03 | −5.58E−03 | −3.98E−04 |
| ZNF567 | −1.21E−03 | −4.80E−03 | −3.25E−04 |
| ZNF568 | −7.18E−03 | −1.50E−02 | −2.81E−03 |
| ZNF569 | 2.34E−03 | −1.56E−02 | −2.70E−03 |
| ZNF57 | −1.71E−03 | −2.42E−03 | −1.89E−03 |
| ZNF570 | −2.03E−03 | −1.32E−02 | −2.36E−03 |
| ZNF571 | −3.29E−03 | −3.55E−03 | 3.27E−03 |
| ZNF572 | −2.17E−03 | −4.37E−03 | 5.57E−03 |
| ZNF573 | −2.51E−03 | −3.52E−03 | −1.43E−03 |
| ZNF574 | −9.64E−04 | −3.34E−03 | −2.95E−03 |
| ZNF575 | −1.92E−03 | −1.95E−03 | 6.92E−04 |
| ZNF576 | 1.48E−03 | 3.68E−04 | −1.20E−03 |
| ZNF577 | −9.39E−04 | −5.61E−03 | −1.63E−03 |
| ZNF578 | −1.06E−02 | −6.14E−03 | 7.13E−03 |
| ZNF579 | 3.86E−04 | −4.09E−03 | −4.19E−04 |
| ZNF580 | 5.16E−04 | −5.80E−04 | 8.07E−04 |
| ZNF581 | 7.41E−04 | −1.01E−03 | −2.69E−04 |
| ZNF582 | −2.93E−04 | −7.32E−03 | 2.04E−03 |
| ZNF583 | 3.93E−03 | −6.31E−03 | −2.01E−03 |
| ZNF584 | −4.85E−04 | −4.89E−03 | −1.66E−03 |
| ZNF585A | −7.06E−03 | −8.53E−03 | 6.26E−04 |
| ZNF585B | −7.64E−03 | −8.84E−03 | −1.26E−03 |
| ZNF586 | 2.19E−03 | −1.39E−03 | −3.04E−03 |
| ZNF587 | 3.57E−06 | −1.60 | −1.64 |
| ZNF587B | 2.10E−03 | −4.84E−03 | −3.90E−03 |
| ZNF589 | 9.45E−03 | 2.98E−03 | 1.50E−03 |
| ZNF592 | 2.03E−03 | −2.62E−04 | −1.70E−03 |
| ZNF594 | −3.11E−04 | −6.40E−03 | −1.49E−03 |
| ZNF595 | −2.84E−03 | −5.36E−03 | 3.56E−03 |
| ZNF596 | −3.38E−03 | −5.10E−03 | 8.79E−04 |
| ZNF597 | −3.67E−03 | −1.18E−03 | −9.88E−04 |
| ZNF598 | 8.97E−04 | −6.22E−05 | −1.26E−03 |
| ZNF599 | −2.70E−03 | −7.06E−03 | −4.90E−04 |
| ZNF600 | −7.57E−03 | 3.60E−04 | −1.73E−03 |
| ZNF605 | −9.77E−04 | −5.46E−03 | −1.07E−03 |
| ZNF606 | −1.50E−03 | −6.19E−03 | 2.45E−03 |
| ZNF607 | −1.88E−03 | −4.64E−03 | 1.59E−03 |
| ZNF608 | −2.15E−03 | −8.16E−03 | −3.37E−03 |
| ZNF609 | −2.39E−03 | 5.80E−03 | −9.32E−04 |
| ZNF610 | −9.82E−03 | −4.82E−03 | 4.55E−03 |
| ZNF611 | −2.77E−03 | −1.71E−03 | 1.04E−03 |
| ZNF613 | 1.90E−04 | −6.48E−03 | 1.06E−04 |
| ZNF614 | −1.19E−03 | −8.82E−03 | −1.81E−04 |
| ZNF615 | 3.44E−04 | −5.84E−03 | 1.81E−03 |
| ZNF616 | −2.02E−03 | −4.71E−03 | 2.32E−03 |
| ZNF618 | 3.85E−03 | −1.77E−02 | −1.40E−02 |
| ZNF619 | −3.70E−03 | −5.67E−04 | 3.56E−03 |
| ZNF620 | 8.12E−03 | 3.65E−03 | −3.45E−04 |
| ZNF621 | −9.99E−04 | −1.46E−03 | 2.96E−04 |
| ZNF623 | −2.87E−03 | −3.41E−03 | 2.34E−03 |
| ZNF624 | 7.74E−05 | −4.76E−03 | 5.08E−03 |
| ZNF625 | −3.53E−03 | −1.09E−02 | −1.32E−04 |
| ZNF626 | −1.61E−02 | 7.99E−03 | 2.70E−04 |
| ZNF627 | −1.31E−04 | −5.75E−03 | 6.82E−04 |
| ZNF628 | −1.41E−03 | −1.50E−03 | −2.99E−03 |
| ZNF629 | −2.41E−04 | −3.93E−03 | −2.42E−04 |
| ZNF630 | −5.01E−03 | −4.67E−04 | −1.27E−03 |
| ZNF639 | 3.26E−03 | −4.52E−03 | −9.53E−04 |
| ZNF641 | −3.62E−03 | −1.13E−03 | −2.40E−03 |
| ZNF644 | 1.11E−03 | −2.56E−03 | −4.67E−03 |
| ZNF645 | −2.68E−04 | −1.76E−04 | −1.85E−04 |
| ZNF646 | −8.57E−04 | 4.24E−05 | 1.02E−03 |
| ZNF648 | 6.42E−03 | −8.53E−03 | −1.61E−02 |
| ZNF649 | −6.36E−04 | −6.66E−03 | −2.96E−03 |
| ZNF652 | 2.38E−03 | −1.91E−03 | −5.39E−04 |
| ZNF653 | −1.83E−04 | −4.39E−04 | −1.05E−03 |
| ZNF654 | −2.00E−03 | −2.08E−03 | −1.10E−03 |
| ZNF655 | −3.59E−03 | −3.40E−03 | −1.37E−03 |
| ZNF658 | −5.45E−03 | −4.93E−03 | 6.90E−03 |
| ZNF660 | −1.77E−03 | −2.24E−03 | 2.85E−03 |
| ZNF662 | −6.64E−03 | −6.39E−03 | −1.23E−03 |
| ZNF664 | 4.94E−03 | −1.62E−03 | −2.53E−03 |
| ZNF665 | −2.38E−02 | −3.72E−03 | 6.14E−03 |
| ZNF667 | −2.44E−02 | −4.82E−03 | 7.32E−04 |
| ZNF668 | 3.79E−03 | −7.07E−04 | −1.84E−03 |
| ZNF669 | 9.41E−04 | −3.14E−03 | −8.44E−05 |
| ZNF670 | 7.24E−03 | −4.41E−03 | −3.12E−03 |
| ZNF671 | 3.57E−03 | −3.75E−03 | 1.53E−03 |
| ZNF672 | 2.23E−03 | −8.20E−04 | −4.91E−04 |
| ZNF674 | −3.38E−03 | −1.02E−04 | −1.87E−03 |
| ZNF675 | 8.14E−04 | −2.23E−03 | 1.83E−03 |
| ZNF676 | −1.09E−02 | −3.44E−03 | 5.95E−03 |
| ZNF677 | −2.72E−02 | −3.39E−03 | 1.10E−04 |
| ZNF678 | 6.20E−04 | −3.88E−03 | 1.29E−03 |
| ZNF680 | 9.53E−04 | 1.38E−03 | 5.26E−03 |
| ZNF681 | −8.05E−03 | −6.39E−03 | −6.74E−03 |
| ZNF682 | −1.06E−02 | −1.98E−03 | −1.92E−03 |
| ZNF683 | −1.03E−03 | 2.12E−04 | −1.89E−03 |
| ZNF684 | 6.17E−03 | −5.59E−03 | −8.54E−04 |
| ZNF687 | −8.41E−04 | −7.70E−04 | 1.91E−03 |
| ZNF688 | −4.05E−04 | 2.79E−04 | 5.92E−04 |
| ZNF689 | 2.56E−03 | −3.84E−03 | −4.70E−04 |
| ZNF69 | −3.86E−03 | −6.93E−04 | −1.12E−02 |
| ZNF691 | −1.51E−03 | −4.02E−03 | 2.27E−03 |
| ZNF692 | −5.46E−04 | −7.57E−04 | −5.75E−04 |
| ZNF695 | 1.63E−02 | −5.76E−04 | −2.32E−03 |
| ZNF696 | 8.15E−04 | −4.19E−03 | 1.65E−03 |
| ZNF697 | −3.83E−04 | −2.21E−03 | −3.44E−03 |
| ZNF699 | −1.80E−02 | −8.73E−03 | −4.01E−03 |
| ZNF7 | 1.20E−04 | 3.38E−04 | 1.68E−03 |
| ZNF70 | −2.06E−03 | −2.35E−03 | 2.41E−03 |
| ZNF700 | −1.61E−03 | 2.24E−03 | −1.76E−03 |
| ZNF701 | −3.81E−03 | −2.15E−03 | 3.46E−03 |
| ZNF703 | −1.23E−02 | 2.66E−03 | −5.93E−03 |
| ZNF704 | −7.19E−03 | 5.17E−03 | −6.04E−03 |
| ZNF705A | −2.04E−03 | −2.42E−03 | −9.82E−04 |
| ZNF705D | 1.02E−03 | 1.79E−03 | −2.02E−03 |
| ZNF705E | −1.29E−03 | −8.31E−03 | 2.02E−03 |
| ZNF705G | 1.12E−04 | 2.38E−03 | −2.61E−03 |
| ZNF706 | −6.85E−04 | 1.06E−03 | −8.77E−04 |
| ZNF707 | −1.13E−03 | −3.28E−03 | −1.54E−03 |
| ZNF708 | 1.37E−03 | −2.42E−03 | 3.22E−04 |
| ZNF709 | −1.78E−03 | −1.13E−02 | −1.70E−03 |
| ZNF71 | −7.34E−05 | −6.72E−03 | −4.08E−04 |
| ZNF710 | 2.98E−03 | −2.21E−03 | −4.66E−03 |
| ZNF711 | 2.96E−03 | −4.29E−03 | −4.70E−03 |
| ZNF713 | 3.19E−03 | −2.12E−03 | −2.52E−03 |
| ZNF714 | 6.69E−03 | −3.84E−03 | −1.08E−03 |
| ZNF717 | −5.47E−03 | −6.78E−04 | 3.25E−03 |
| ZNF718 | 9.65E−04 | 1.44E−03 | 4.28E−03 |
| ZNF721 | −1.03E−04 | −2.66E−03 | 1.59E−03 |
| ZNF726 | 9.54E−03 | −8.29E−04 | −5.61E−03 |
| ZNF727 | −2.14E−02 | −3.56E−03 | 1.15E−02 |
| ZNF728 | −5.25E−03 | −1.78E−03 | 2.22E−03 |
| ZNF729 | 6.93E−05 | 1.48E−04 | −2.39E−04 |
| ZNF730 | −3.82E−04 | −4.92E−03 | −4.80E−03 |
| ZNF732 | 4.15E−03 | −5.07E−03 | 5.78E−04 |
| ZNF736 | −7.72E−03 | 1.34E−02 | 2.00E−03 |
| ZNF737 | −1.17E−02 | 1.23E−02 | 4.79E−03 |
| ZNF74 | 4.67E−03 | −5.37E−03 | −1.31E−03 |
| ZNF740 | −1.40E−03 | −4.40E−03 | −6.46E−04 |
| ZNF746 | 1.25E−03 | −2.98E−03 | −6.74E−04 |
| ZNF747 | −5.27E−04 | −2.97E−03 | 1.83E−03 |
| ZNF749 | −1.64E−04 | −2.81E−03 | −2.92E−03 |
| ZNF750 | −1.23E−02 | 1.83E−02 | −9.60E−03 |
| ZNF75A | −1.35E−03 | −2.91E−03 | 3.31E−03 |
| ZNF75D | −1.89E−03 | 2.84E−03 | −7.89E−05 |
| ZNF76 | 1.93E−03 | 2.08E−04 | −2.48E−03 |
| ZNF761 | −1.25E−03 | −2.70E−03 | −2.07E−03 |
| ZNF763 | −1.09E−02 | −3.15E−03 | −3.35E−03 |
| ZNF764 | 2.59E−03 | −1.97E−03 | −1.45E−03 |
| ZNF765 | −9.36E−04 | −2.14E−03 | 1.33E−03 |
| ZNF766 | −6.29E−04 | −4.98E−03 | 3.38E−03 |
| ZNF768 | 5.91E−04 | −3.63E−03 | 8.47E−04 |
| ZNF77 | −1.34E−03 | −4.25E−03 | 2.54E−04 |
| ZNF770 | 5.57E−04 | −3.25E−03 | −1.60E−03 |
| ZNF771 | 1.51E−03 | −1.74E−03 | −1.82E−04 |
| ZNF772 | 3.60E−03 | −7.30E−03 | 1.20E−03 |
| ZNF773 | −1.94E−03 | −5.76E−03 | 2.93E−03 |
| ZNF774 | −3.28E−03 | 4.08E−04 | 4.01E−03 |
| ZNF775 | 6.14E−03 | −2.73E−03 | 1.70E−03 |
| ZNF776 | −2.28E−05 | −2.67E−03 | 8.40E−05 |
| ZNF777 | 9.65E−04 | −3.17E−04 | 5.61E−04 |
| ZNF778 | 4.17E−03 | 2.02E−03 | −1.08E−03 |
| ZNF780A | −1.01E−03 | −3.12E−03 | 1.87E−03 |
| ZNF780B | −6.31E−04 | −4.09E−03 | 1.68E−03 |
| ZNF781 | 8.49E−04 | −6.11E−03 | −1.55E−03 |
| ZNF782 | 1.07E−03 | −2.30E−03 | −3.41E−04 |
| ZNF783 | 1.19E−03 | 1.58E−03 | 1.09E−03 |
| ZNF784 | −2.82E−03 | −1.24E−03 | 3.11E−03 |
| ZNF785 | −5.55E−04 | −3.15E−03 | −1.80E−03 |
| ZNF786 | −1.12E−03 | −1.64E−03 | −1.75E−03 |
| ZNF787 | 1.53E−03 | −1.71E−03 | −1.76E−03 |
| ZNF788 | −1.59E−02 | 1.41E−03 | −4.03E−04 |
| ZNF789 | 5.60E−03 | −7.86E−04 | −1.21E−03 |
| ZNF79 | −2.48E−03 | −2.50E−03 | 1.35E−03 |
| ZNF790 | −5.52E−03 | −1.36E−02 | −5.05E−03 |
| ZNF791 | −2.53E−03 | −2.29E−03 | 8.15E−04 |
| ZNF792 | 4.63E−04 | −3.49E−03 | 1.38E−03 |
| ZNF793 | −1.21E−02 | −1.64E−02 | −6.15E−03 |
| ZNF799 | −6.12E−03 | 1.03E−03 | 1.07E−03 |
| ZNF8 | 3.88E−03 | −3.02E−03 | −2.46E−03 |
| ZNF80 | 6.70E−04 | −1.16E−03 | 1.20E−03 |
| ZNF800 | −1.60E−03 | −3.12E−04 | −2.12E−03 |
| ZNF804A | 3.18E−02 | −5.11E−03 | −2.19E−02 |
| ZNF804B | 9.37E−03 | −1.46E−02 | −2.10E−03 |
| ZNF805 | −2.43E−03 | −3.20E−03 | −3.60E−03 |
| ZNF808 | −3.52E−03 | −7.13E−04 | 1.27E−03 |
| ZNF81 | −5.98E−04 | −2.76E−03 | −2.96E−03 |
| ZNF813 | −4.24E−03 | −8.83E−03 | 5.34E−03 |
| ZNF814 | 2.79E−03 | −3.66E−03 | −3.79E−03 |
| ZNF816 | −1.31E−02 | 5.23E−03 | 3.85E−03 |
| ZNF821 | 2.41E−03 | 1.60E−04 | 1.02E−03 |
| ZNF823 | −3.84E−03 | 7.62E−04 | −1.33E−03 |
| ZNF827 | 2.83E−03 | −2.74E−03 | −5.12E−03 |
| ZNF829 | −6.76E−03 | −1.62E−02 | −2.17E−03 |
| ZNF83 | −8.85E−03 | 1.09E−03 | 1.63E−03 |
| ZNF830 | −1.07E−03 | −4.92E−04 | −2.53E−03 |
| ZNF831 | −3.91E−04 | −7.34E−04 | −1.90E−04 |
| ZNF835 | −1.48E−02 | −7.17E−03 | 1.14E−02 |
| ZNF836 | −5.85E−03 | −2.76E−03 | −2.12E−03 |
| ZNF837 | −2.05E−03 | 1.10E−03 | 1.79E−03 |
| ZNF84 | 2.13E−03 | −4.33E−03 | 1.10E−03 |
| ZNF841 | −3.42E−03 | −2.80E−03 | −6.16E−04 |
| ZNF843 | −1.11E−02 | −6.73E−03 | 5.30E−03 |
| ZNF844 | −1.84E−02 | −2.96E−03 | −3.67E−03 |
| ZNF845 | −2.17E−03 | −4.58E−03 | 2.94E−03 |
| ZNF846 | −7.49E−03 | −5.81E−04 | 2−.13E−03 |
| ZNF85 | −2.00E−03 | 2.44E−03 | 7.13E−04 |
| ZNF850 | 3.49E−03 | −1.38E−02 | −3.92E−03 |
| ZNF852 | 1.90E−03 | 2.78E−03 | −7.16E−04 |
| ZNF853 | −1.31E−02 | −1.45E−02 | −7.35E−04 |
| ZNF860 | −8.65E−03 | −3.89E−04 | −1.10E−02 |
| ZNF865 | −5.34E−04 | −5.49E−03 | −2.24E−04 |
| ZNF878 | −9.30E−03 | −5.11E−03 | 8.07E−04 |
| ZNF879 | −4.09E−03 | −1.67E−02 | −2.01E−03 |
| ZNF880 | −6.00E−03 | −5.48E−03 | 2.33E−03 |
| ZNF883 | 6.11E−03 | −3.12E−03 | −8.11E−04 |
| ZNF888 | −1.20E−02 | 3.96E−03 | 6.04E−04 |
| ZNF891 | 6.54E−04 | −5.70E−03 | 7.52E−04 |
| ZNF90 | −1.74E−02 | 2.77E−03 | 7.93E−03 |
| ZNF91 | −6.34E−03 | 1.11E−03 | 1.82E−03 |
| ZNF92 | 4.64E−03 | −5.96E−03 | 8.55E−04 |
| ZNF93 | −6.50E−03 | 2.41E−03 | 5.50E−03 |
| ZNF98 | −7.88E−03 | −1.90E−03 | 7.86E−03 |
| ZNF99 | −6.23E−04 | −2.16E−04 | 2.50E−04 |
| ZSCAN1 | −7.00E−03 | −2.40E−03 | 3.26E−03 |
| ZSCAN10 | −5.32E−03 | 5.11E−04 | 2.87E−03 |
| ZSCAN12 | 2.08E−03 | −5.57E−03 | 7.29E−04 |
| ZSCAN16 | 1.35E−03 | −1.04E−03 | 7.36E−04 |
| ZSCAN18 | −1.83E−02 | −1.23E−02 | 5.00E−04 |
| ZSCAN2 | 2.32E−03 | 1.31E−03 | 1.88E−04 |
| ZSCAN20 | −1.91E−03 | −1.87E−03 | 2.32E−03 |
| ZSCAN21 | −1.27E−03 | −4.24E−03 | 2.26E−03 |
| ZSCAN22 | −8.82E−04 | −6.75E−04 | −1.50E−03 |
| ZSCAN23 | −1.44E−03 | −7.66E−03 | 1.53E−03 |
| ZSCAN25 | −2.40E−03 | −3.66E−03 | 6.60E−04 |
| ZSCAN26 | −1.71E−03 | −3.98E−03 | 1.43E−03 |
| ZSCAN29 | 1.45E−03 | −3.15E−04 | −6.87E−04 |
| ZSCAN30 | −3.44E−03 | −2.34E−03 | 2.63E−03 |
| ZSCAN31 | 6.33E−04 | 2.19E−03 | −3.52E−03 |
| ZSCAN32 | −1.12E−05 | −3.20E−03 | 2.49E−03 |
| ZSCAN4 | −7.50E−03 | 2.01E−03 | −6.44E−03 |
| ZSCAN5A | −1.75E−03 | −5.89E−04 | −8.59E−04 |
| ZSCAN5B | −3.22E−03 | −2.73E−03 | 3.15E−03 |
| ZSCAN9 | 3.25E−04 | −3.16E−03 | −7.54E−04 |
| ZUFSP | −3.43E−04 | −1.54E−03 | −1.59E−04 |
| ZXDA | −4.41E−03 | −6.01E−03 | −1.78E−03 |
| ZXDB | −5.23E−03 | −6.54E−04 | −4.02E−03 |
| ZXDC | 8.92E−04 | 1.07E−04 | 1.17E−03 |
| ZZZ3 | 1.06E−03 | 4.24E−04 | −6.28E−04 |

TABLE 4
Differential genes in ASCL1 vs ASCL2 cell populations (Related to FIG. 4D)
ASCL1 vs ASCL2 DE genes, filtered P_val_adj < 0.05, abs(avg_logFC) > 1

| gene | p_val | avg_logFC | pct. 1 | pct. 2 | p_val_adj |
|---|---|---|---|---|---|
| IGFBP5 | 0 | 3.710384174 | 90.40% | 2.60% | 0 |
| ASCL1 | 0 | 3.695882333 | 100.00% | 0.20% | 0 |
| CYBA | 0 | 2.378026821 | 80.40% | 44.10% | 0 |
| NNAT | 0 | 2.227505209 | 42.00% | 0.80% | 0 |
| WFDC2 | 0 | 1.922919417 | 46.00% | 19.00% | 0 |
| SEC11C | 0 | 1.848215846 | 87.60% | 81.60% | 0 |
| CD24 | 0 | 1.847736752 | 65.80% | 34.80% | 0 |
| MIAT | 0 | 1.780336425 | 34.50% | 3.00% | 0 |
| DDC | 0 | 1.701829494 | 34.20% | 0.40% | 0 |
| RAB3B | 0 | 1.652949978 | 35.50% | 11.90% | 0 |
| CD9 | 0 | −1.285864678 | 9.20% | 88.20% | 0 |
| LYPLA1 | 0 | −1.309246088 | 24.60% | 92.60% | 0 |
| PLA2G4A | 0 | −1.424783928 | 0.40% | 81.40% | 0 |
| POU2F3 | 0 | −1.579754417 | 0.40% | 83.20% | 0 |
| ASCL2 | 0 | −1.879254891 | 0.00% | 100.00% | 0 |
| LRMP | 0 | −1.906793179 | 1.20% | 85.60% | 0 |
| TPM2 | 0 | −1.938792847 | 0.70% | 85.40% | 0 |
| BMX | 0 | −2.122883854 | 0.50% | 88.50% | 0 |
| KRT18 | 0 | −2.161883559 | 21.10% | 95.40% | 0 |
| NMU | 0 | −2.221072331 | 1.60% | 89.50% | 0 |
| TUBA1A | 0 | −2.292534819 | 10.00% | 94.90% | 0 |
| RGS13 | 0 | −2.768697008 | 1.30% | 94.80% | 0 |
| ANXA1 | 0 | −3.472530545 | 3.30% | 98.20% | 0 |
| IGFBP2 | 0.00E+00 | 1.621917979 | 29.80% | 7.90% | 0.00E+00 |
| BEX1 | 0.00E+00 | 1.522011382 | 31.80% | 10.80% | 0.00E+00 |
| SOX9 | 2.02E−307 | −1.261275597 | 0.60% | 77.10% | 7.36E−303 |
| MT1E | 4.10E−302 | −1.333307812 | 3.40% | 86.20% | 1.50E−297 |
| CNN3 | 2.21E−293 | −1.264815668 | 6.30% | 86.60% | 8.07E−289 |
| DPYSL3 | 1.20E−266 | −1.318553362 | 12.90% | 89.60% | 4.40E−262 |
| CAMK2N1 | 1.86E−263 | 1.782786204 | 35.80% | 0.80% | 6.77E−259 |
| TSPAN12 | 1.23E−257 | −1.315203536 | 2.30% | 81.50% | 4.49E−253 |
| HSPB1 | 2.18E−251 | 1.514356617 | 55.80% | 53.80% | 7.95E−247 |
| BMP7 | 6.18E−251 | 1.376455929 | 26.20% | 3.90% | 2.25E−246 |
| KIF5C | 1.91E−247 | 1.235985563 | 23.20% | 7.50% | 6.96E−243 |
| KLHL41 | 1.18E−241 | 1.038806052 | 15.70% | 0.40% | 4.30E−237 |
| TPM4 | 3.77E−239 | −1.237662148 | 1.40% | 79.60% | 1.38E−234 |
| CACNA1A | 1.42E−224 | 1.298890478 | 21.30% | 1.40% | 5.18E−220 |
| TRPM8 | 2.26E−224 | 1.248011565 | 22.10% | 3.00% | 8.25E−220 |
| COL11A1 | 1.51E−223 | −1.034430052 | 0.30% | 58.20% | 5.51E−219 |
| CCND1 | 3.55E−221 | −1.350150725 | 0.20% | 78.00% | 1.29E−216 |
| INSM1 | 1.39E−220 | 1.378539837 | 26.60% | 10.20% | 5.08E−216 |
| CLDN5 | 1.13E−215 | 1.097344546 | 15.60% | 0.80% | 4.12E−211 |
| EHF | 9.12E−214 | −1.168519793 | 0.20% | 75.00% | 3.33E−209 |
| KCTD12 | 1.E−212 | 1.831645178 | 40.50% | 14.70% | 3.97E−208 |
| SPIB | 1.33E−203 | −1.109629693 | 0.10% | 71.40% | 4.87E−199 |
| NR2F1 | 2.19E−196 | 1.389329103 | 22.00% | 6.70% | 7.98E−192 |
| C11orf53 | 8.20E−193 | −1.169040151 | 0.20% | 76.10% | 2.99E−188 |
| MARCKSL1 | 9.41E−192 | −1.167776982 | 31.10% | 91.60% | 3.43E−187 |
| GNG4 | 3.43E−190 | 1.210776174 | 29.60% | 26.70% | 1.25E−185 |
| RAB11FIP1 | 3.85E−189 | 1.218079623 | 41.60% | 62.70% | 1.40E−184 |
| NTHL1 | 2.33E−183 | 1.287310299 | 42.20% | 64.70% | 8.50E−179 |
| KRT8 | 9.08E−177 | −1.138716727 | 35.10% | 92.50% | 3.31E−172 |
| TFF3 | 1.93E−165 | 1.678931344 | 61.00% | 51.50% | 7.03E−161 |
| LINC02802 | 5.52E−164 | 1.373287095 | 31.90% | 26.20% | 2.01E−159 |
| PIR | 1.41E−162 | −1.051178543 | 0.60% | 74.30% | 5.15E−158 |
| CMIP | 1.06E−159 | 1.192847196 | 41.00% | 61.30% | 3.86E−155 |
| CSRP2 | 2.01E−153 | −1.158923515 | 1.60% | 76.70% | 7.32E−149 |
| PLPP2 | 3.67E−153 | −1.269522908 | 3.20% | 81.90% | 1.34E−148 |
| AVIL | 9.34E−150 | −1.00073335 | 9.20% | 83.80% | 3.41E−145 |
| SOX17 | 5.68E−148 | −1.098615519 | 0.20% | 50.70% | 2.07E−143 |
| LMCD1 | 4.03E−145 | −1.07640318 | 0.90% | 74.70% | 1.47E−140 |
| FXYD3 | 2.39E−143 | 1.385612574 | 27.50% | 11.10% | 8.71E−139 |
| CYB5R3 | 5.98E−142 | 1.052546774 | 29.50% | 55.20% | 2.18E−137 |
| FOXA2 | 8.50E−141 | 1.136438372 | 19.70% | 3.80% | 3.10E−136 |
| LIMCH1 | 5.45E−140 | −1.057025022 | 0.50% | 70.40% | 1.99E−135 |
| TMEM176A | 7.07E−140 | 1.329902293 | 22.30% | 2.20% | 2.58E−135 |
| LINC00545 | 5.86E−137 | −1.125963153 | 0.20% | 42.70% | 2.14E−132 |
| ATP5F1E | 8.01E−137 | 1.155441721 | 86.00% | 92.60% | 2.92E−132 |
| CAMK2B | 3.12E−134 | 1.486696476 | 25.60% | 4.40% | 1.14E−129 |
| NUPR1 | 4.76E−132 | 1.123497234 | 16.70% | 1.60% | 1.74E−127 |
| PPP1R1B | 2.41E−131 | −1.1408288 | 1.10% | 76.60% | 8.79E−127 |
| BIK | 1.92E−130 | 1.470564805 | 59.00% | 68.30% | 6.99E−126 |
| SCNN1A | 2.13E−128 | 1.18579208 | 29.80% | 39.60% | 7.78E−124 |
| POLN | 1.99E−127 | 1.002623591 | 15.00% | 3.20% | 7.25E−123 |
| CXXC5 | 1.86E−123 | −1.045761894 | 1.00% | 74.50% | 6.78E−119 |
| NES | 2.12E−122 | −1.002185664 | 0.20% | 71.40% | 7.75E−118 |
| TMEM54 | 3.19E−120 | −1.033661353 | 0.60% | 72.40% | 1.16E−115 |
| IMP4 | 3.63E−118 | −1.2179401 | 12.70% | 86.30% | 1.32E−113 |
| STARD10 | 3.90E−115 | 1.063619027 | 23.90% | 38.60% | 1.42E−110 |
| NARS2 | 1.34E−111 | 1.022976013 | 26.80% | 46.60% | 4.89E−107 |
| ATP5MD | 1.34E−104 | 1.15533562 | 81.10% | 91.00% | 4.88E−100 |
| GAL | 9.68E−102 | −1.031671728 | 10.80% | 82.90% | 3.53E−97 |
| TMEM176B | 8.80E−95 | 1.048999422 | 15.30% | 1.60% | 3.21E−90 |
| TOMM7 | 1.05E−91 | 1.127029578 | 63.00% | 80.80% | 3.82E−87 |
| KRT19 | 1.23E−88 | 1.1321172 | 31.40% | 19.90% | 4.49E−84 |
| ADIRF | 9.18E−87 | 1.162641987 | 16.90% | 1.10% | 3.35E−82 |
| S100A6 | 7.68E−85 | 1.243062539 | 79.00% | 86.30% | 2.80E−80 |
| NDUFA4L2 | 2.13E−83 | −1.070258115 | 1.60% | 72.10% | 7.77E−79 |
| GLRX | 1.18E−81 | −1.100436784 | 1.80% | 74.10% | 4.30E−77 |
| SH3TC1 | 1.96E−75 | 1.235310822 | 17.80% | 0.80% | 7.17E−71 |
| PEG10 | 6.63E−74 | 1.216960146 | 26.30% | 17.20% | 2.42E−69 |
| CENPV | 9.12E−72 | −1.021343245 | 11.70% | 83.80% | 3.33E−67 |
| CLDN18 | 6.65E−66 | 1.474617227 | 122.0% | 0.10% | 2.43E−61 |
| TMCO3 | 2.59E−54 | 1.014810003 | 25.40% | 46.40% | 9.45E−50 |
| SEC61G | 2.07E−53 | 1.253814095 | 70.40% | 85.30% | 7.56E−49 |
| PRAC1 | 1.27E−48 | 1.274580691 | 44.70% | 44.00% | 4.65E−44 |
| UBE2S | 2.59E−45 | −1.062038047 | 26.10% | 489.0% | 9.47E−41 |
| PRDX5 | 1.85E−44 | −1.02864244 | 11.20% | 84.20% | 6.76E−40 |
| LRRC75A | 5.32E−35 | 1.172208086 | 50.20% | 61.60% | 1.94E−30 |
| C4orf48 | 2.33E−30 | 1.036187487 | 529.0% | 47.30% | 8.50E−26 |
| SNHG25 | 9.19E−23 | 1.251797236 | 43.10% | 44.10% | 3.35E−18 |
| SNHG5 | 7.56E−17 | 1.023140057 | 69.50% | 83.60% | 2.76E−12 |

TABLE 5
List of predicted gene network of ASCL1 and ASCL2 (Related to FIG. 4E)

| Regulator | Target GENE | MI | pvalue |
|---|---|---|---|
| ASCL1 | CACNA1A | 6.67E−01 | <1E−09 |
| ASCL1 | DDC | 6.49E−01 | <1E−09 |
| ASCL1 | CPLX2 | 6.38E−01 | <1E−09 |
| ASCL1 | ANKRD65 | 6.06E−01 | <1E−09 |
| ASCL1 | SPDEF | 6.05E−01 | 1.82E−06 |
| ASCL1 | SCG2 | 6.05E−01 | <1E−09 |
| ASCL1 | TXNDC11 | 6.04E−01 | <1E−09 |
| ASCL1 | VWA5B2 | 6.02E−01 | <1E−09 |
| ASCL1 | FAM174B | 5.99E−01 | 5.93E−08 |
| ASCL1 | BAIAP3 | 5.97E−01 | 5.06E−05 |
| ASCL1 | GJB1 | 5.97E−01 | 0.0298735 |
| ASCL1 | NR0B2 | 5.95E−01 | <1E−09 |
| ASCL1 | SLCO3A1 | 5.95E−01 | <1E−09 |
| ASCL1 | CELF3 | 5.93E−08 | <1E−09 |
| ASCL1 | UHRF1 | 5.93E−08 | 0.0012893 |
| ASCL1 | HES6 | 5.92E−01 | <1E−09 |
| ASCL1 | SCG3 | 5.91E−01 | <1E−09 |
| ASCL1 | KIF19 | 5.87E−01 | <1E−09 |
| ASCL1 | E2F2 | 5.86E−01 | 5.06E−05 |
| ASCL1 | CRMP1 | 5.84E−01 | <1E−09 |
| ASCL1 | TRIM9 | 5.84E−01 | <1E−09 |
| ASCL1 | SOX2 | 5.84E−01 | 0.0298735 |
| ASCL1 | NELL1 | 5.83E−01 | <1E−09 |
| ASCL1 | SLC36A4 | 5.83E−01 | <1E−09 |
| ASCL1 | CHRNB2 | 5.81E−01 | <1E−09 |
| ASCL1 | GFI1 | 5.79E−01 | <1E−09 |
| ASCL1 | NEURL1 | 5.78E−01 | <1E−09 |
| ASCL1 | TMEM176A | 5.76E−01 | 5.06E−05 |
| ASCL1 | ADA | 5.72E−01 | <1E−09 |
| ASCL1 | CDK5R2 | 5.70E−01 | <1E−09 |
| ASCL1 | RNASEH2A | 5.70E−01 | 5.93E−08 |
| ASCL1 | INSM1 | 5.69E−01 | <1E−09 |
| ASCL1 | ANK2 | 5.68E−01 | <1E−09 |
| ASCL1 | HEPACAM2 | 5.67E−01 | <1E−09 |
| ASCL1 | TAOK3 | 5.67E−01 | 0.0298735 |
| ASCL1 | DNMT3B | 5.66E−01 | <1E−09 |
| ASCL1 | SRRM4 | 5.65E−01 | <1E−09 |
| ASCL1 | GPRIN1 | 5.65E−01 | <1E−09 |
| ASCL1 | HCN4 | 5.64E−01 | <1E−09 |
| ASCL1 | PLS1 | 5.63E−01 | 5.93E−08 |
| ASCL1 | CALCOCO1 | 5.63E−01 | 0.0298735 |
| ASCL1 | FAM120AOS | 5.63E−01 | 0.0012893 |
| ASCL1 | SEZ6 | 5.62E−01 | <1E−09 |
| ASCL1 | R3HDM1 | 5.62E−01 | 0.0012893 |
| ASCL1 | URB2 | 5.61E−01 | 3.30E−09 |
| ASCL1 | IGF1 | 5.58E−01 | 1.82E−06 |
| ASCL1 | GNAO1 | 5.57E−01 | <1E−09 |
| ASCL1 | DLGAP3 | 5.57E−01 | <1E−09 |
| ASCL1 | ELAVL4 | 5.56E−01 | <1E−09 |
| ASCL1 | E2F1 | 5.55E−01 | 0.0298735 |
| ASCL1 | FOXA2 | 5.54E−01 | <1E−09 |
| ASCL1 | TMEM178B | 5.53E−01 | <1E−09 |
| ASCL1 | BTBD3 | 5.53E−01 | <1E−09 |
| ASCL1 | TMEM74 | 5.53E−01 | <1E−09 |
| ASCL1 | VIL1 | 5.52E−01 | 5.06E−05 |
| ASCL1 | ZNF311 | 5.52E−01 | <1E−09 |
| ASCL1 | CABP7 | 5.52E−01 | <1E−09 |
| ASCL1 | C1S | 5.51E−01 | 5.06E−05 |
| ASCL1 | JAKMIP2 | 5.51E−01 | <1E−09 |
| ASCL1 | DBH | 5.51E−01 | <1E−09 |
| ASCL1 | PIGH | 5.50E−01 | 0.0012893 |
| ASCL1 | SYN1 | 5.48E−01 | <1E−09 |
| ASCL1 | DLL3 | 5.47E−01 | 1.82E−06 |
| ASCL1 | GLS2 | 5.47E−01 | 0.0298735 |
| ASCL1 | INHBE | 5.47E−01 | 0.0298735 |
| ASCL1 | SNAP25 | 5.46E−01 | <1E−09 |
| ASCL1 | SYP | 5.45E−01 | 1.82E−06 |
| ASCL1 | USH1C | 5.44E−01 | <1E−09 |
| ASCL1 | RNF183 | 5.43E−01 | <1E−09 |
| ASCL1 | PYGB | 5.43E−01 | 0.0298735 |
| ASCL1 | SEZ6L2 | 5.42E−01 | 1.82E−06 |
| ASCL1 | RAB3C | 5.39E−01 | 3.30E−09 |
| ASCL1 | NRSN1 | 5.39E−01 | 0.0012893 |
| ASCL1 | ANXA7 | 5.39E−01 | 1.82E−06 |
| ASCL1 | ADGRG5 | 5.37E−01 | <1E−09 |
| ASCL1 | KIAA0408 | 5.36E−01 | <1E−09 |
| ASCL1 | STX1A | 5.35E−01 | <1E−09 |
| ASCL1 | SESTD1 | 5.35E−01 | 5.06E−05 |
| ASCL1 | FAXC | 5.34E−01 | 0.0012893 |
| ASCL1 | RUNDC3A | 5.33E−01 | <1E−09 |
| ASCL1 | SYN2 | 5.33E−01 | <1E−09 |
| ASCL1 | DDX11 | 5.33E−01 | 5.93E−08 |
| ASCL1 | ANO9 | 5.33E−01 | <1E−09 |
| ASCL1 | FBLN7 | 5.33E−01 | <1E−09 |
| ASCL1 | KLHL32 | 5.32E−01 | <1E−09 |
| ASCL1 | CHGB | 5.32E−01 | <1E−09 |
| ASCL1 | TAGLN3 | 5.32E−01 | <1E−09 |
| ASCL1 | SOGA3 | 5.32E−01 | <1E−09 |
| ASCL1 | CACNA1E | 5.32E−01 | <1E−09 |
| ASCL1 | PEX5L | 5.31E−01 | <1E−09 |
| ASCL1 | KSR2 | 5.31E−01 | <1E−09 |
| ASCL1 | CHGA | 5.29E−01 | <1E−09 |
| ASCL1 | FAM166C | 5.28E−01 | 5.93E−08 |
| ASCL1 | LRWD1 | 5.27E−01 | <1E−09 |
| ASCL1 | KLHL41 | 5.27E−01 | <1E−09 |
| ASCL1 | KCNH2 | 5.27E−01 | <1E−09 |
| ASCL1 | TCERG1L | 5.27E−01 | <1E−09 |
| ASCL1 | ADIRF | 5.27E−01 | 0.0298735 |
| ASCL1 | TNFSF10 | 5.25E−01 | 0.0298735 |
| ASCL1 | ZNF620 | 5.25E−01 | 0.0012893 |
| ASCL1 | FBLN1 | 5.24E−01 | 0.0298735 |
| ASCL1 | SCN8A | 5.24E−01 | <1E−09 |
| ASCL1 | FAM102A | 5.24E−01 | 0.0298735 |
| ASCL1 | DNAJC6 | 5.23E−01 | <1E−09 |
| ASCL1 | CDCA7L | 5.22E−01 | 0.0298735 |
| ASCL1 | CALCA | 5.22E−01 | <1E−09 |
| ASCL1 | INA | 5.21E−01 | 3.30E−09 |
| ASCL1 | UNC5A | 5.21E−01 | <1E−09 |
| ASCL1 | RIMKLA | 5.21E−01 | <1E−09 |
| ASCL1 | ZBTB42 | 5.20E−01 | 0.0298735 |
| ASCL1 | TTYH2 | 5.20E−01 | <1E−09 |
| ASCL1 | NCAM1 | 5.20E−01 | <1E−09 |
| ASCL1 | BSND | 5.19E−01 | 0.0298735 |
| ASCL1 | CTDSPL | 5.19E−01 | <1E−09 |
| ASCL1 | DRP2 | 5.19E−01 | 1.82E−06 |
| ASCL1 | DUSP26 | 5.18E−01 | <1E−09 |
| ASCL1 | TRPM8 | 5.17E−01 | <1E−09 |
| ASCL1 | XKR7 | 5.16E−01 | <1E−09 |
| ASCL1 | RIMS2 | 5.16E−01 | 0.0298735 |
| ASCL1 | NNAT | 5.16E−01 | <1E−09 |
| ASCL1 | MEIOB | 5.15E−01 | 3.30E−09 |
| ASCL1 | PGBD5 | 5.15E−01 | <1E−09 |
| ASCL1 | C1orf127 | 5.14E−01 | <1E−09 |
| ASCL1 | ENTPD8 | 5.14E−01 | <1E−09 |
| ASCL1 | SPTBN5 | 5.12E−01 | <1E−09 |
| ASCL1 | AP3B2 | 5.12E−01 | <1E−09 |
| ASCL1 | UNC79 | 5.11E−01 | 5.93E−08 |
| ASCL1 | CDKN2A | 5.11E−01 | <1E−09 |
| ASCL1 | GRM4 | 5.11E−01 | <1E−09 |
| ASCL1 | CALCB | 5.10E−01 | <1E−09 |
| ASCL1 | KCNH7 | 5.10E−01 | <1E−09 |
| ASCL1 | TMEM163 | 5.10E−01 | <1E−09 |
| ASCL1 | FRMD3 | 5.10E−01 | 5.93E−08 |
| ASCL1 | PELI2 | 5.10E−01 | 0.0012893 |
| ASCL1 | BEX4 | 5.09E−01 | 0.0298735 |
| ASCL1 | BEX1 | 5.08E−01 | <1E−09 |
| ASCL1 | PHYHIPL | 5.08E−01 | <1E−09 |
| ASCL1 | SORBS1 | 5.08E−01 | <1E−09 |
| ASCL1 | BMP8B | 5.08E−01 | <1E−09 |
| ASCL1 | BSN | 5.08E−01 | <1E−09 |
| ASCL1 | CWH43 | 5.07E−01 | 0.0298735 |
| ASCL1 | CA8 | 5.07E−01 | <1E−09 |
| ASCL1 | PRKAR2A | 5.07E−01 | 5.93E−08 |
| ASCL1 | NRXN1 | 5.07E−01 | 3.30E−09 |
| ASCL1 | MAPK13 | 5.07E−01 | <1E−09 |
| ASCL1 | UNC13A | 5.06E−01 | <1E−09 |
| ASCL1 | PCSK1 | 5.06E−01 | <1E−09 |
| ASCL1 | PLAC8 | 5.06E−01 | <1E−09 |
| ASCL1 | INPPL1 | 5.05E−01 | <1E−09 |
| ASCL1 | RIPPLY2 | 5.04E−01 | <1E−09 |
| ASCL1 | CCDC160 | 5.04E−01 | 0.0298735 |
| ASCL1 | COL21A1 | 5.04E−01 | 0.0298735 |
| ASCL1 | IGFBP5 | 5.04E−01 | <1E−09 |
| ASCL1 | ACAP3 | 5.03E−01 | 5.93E−08 |
| ASCL1 | AKR7A3 | 5.02E−01 | <1E−09 |
| ASCL1 | KCNAB2 | 5.02E−01 | 5.06E−05 |
| ASCL1 | BMP8A | 5.01E−01 | 5.06E−05 |
| ASCL1 | RNF182 | 5.01E−01 | 5.06E−05 |
| ASCL1 | ATP2B2 | 5.01E−01 | 5.06E−05 |
| ASCL1 | PITPNM2 | 5.01E−01 | <1E−09 |
| ASCL1 | KCNH6 | 5.01E−01 | <1E−09 |
| ASCL1 | CCDC28B | 5.01E−01 | 1.82E−06 |
| ASCL1 | TMOD2 | 5.01E−01 | <1E−09 |
| ASCL1 | BEX5 | 4.97E−01 | 5.06E−05 |
| ASCL1 | COL4A4 | 4.96E−01 | 5.06E−05 |
| ASCL1 | RGS7 | 4.95E−01 | <1E−09 |
| ASCL1 | CAMK2N2 | 4.95E−01 | 0.0012893 |
| ASCL1 | MFSD9 | 4.94E−01 | 0.0298735 |
| ASCL1 | ANXA13 | 4.94E−01 | <1E−09 |
| ASCL1 | PSD | 4.94E−01 | <1E−09 |
| ASCL1 | NKX2-2 | 4.93E−01 | <1E−09 |
| ASCL1 | NOS1AP | 4.92E−01 | 0.0298735 |
| ASCL1 | PPP1R36 | 4.92E−01 | <1E−09 |
| ASCL1 | RGS16 | 4.92E−01 | 5.06E−05 |
| ASCL1 | PDLIM3 | 4.91E−01 | 1.82E−06 |
| ASCL1 | SFRP1 | 4.90E−01 | 5.06E−05 |
| ASCL1 | TSKU | 4.90E−01 | 1.82E−06 |
| ASCL1 | LPL | 4.90E−01 | 3.30E−09 |
| ASCL1 | SCGN | 4.88E−01 | 0.0012893 |
| ASCL1 | PAX5 | 4.87E−01 | 5.06E−05 |
| ASCL1 | PRR18 | 4.87E−01 | <1E−09 |
| ASCL1 | FGF9 | 4.86E−01 | <1E−09 |
| ASCL1 | ATP4A | 4.85E−01 | <1E−09 |
| ASCL1 | IL33 | 4.85E−01 | 3.30E−09 |
| ASCL1 | GALNT13 | 4.85E−01 | 5.06E−05 |
| ASCL1 | MST1R | 4.84E−01 | 1.82E−06 |
| ASCL1 | FRY | 4.84E−01 | <1E−09 |
| ASCL1 | SLC26A9 | 4.83E−01 | <1E−09 |
| ASCL1 | SYT4 | 4.82E−01 | 0.0298735 |
| ASCL1 | CAMK2B | 4.82E−01 | <1E−09 |
| ASCL1 | EAPP | 4.82E−01 | 1.82E−06 |
| ASCL1 | AKR7L | 4.81E−01 | <1E−09 |
| ASCL1 | FSTL5 | 4.80E−01 | 0.0012893 |
| ASCL1 | SLC25A34 | 4.79E−01 | 0.0298735 |
| ASCL1 | MPPED2 | 4.78E−01 | 0.0298735 |
| ASCL1 | OCIAD2 | 4.78E−01 | 5.06E−05 |
| ASCL1 | BEST4 | 4.77E−01 | 5.06E−05 |
| ASCL1 | IFT46 | 4.76E−01 | 0.0298735 |
| ASCL1 | FEZ2 | 4.76E−01 | 5.93E−08 |
| ASCL1 | SRPK3 | 4.75E−01 | 5.06E−055 |
| ASCL1 | ACTL6B | 4.75E−01 | <1E−09 |
| ASCL1 | OPRD1 | 4.73E−01 | <1E−09 |
| ASCL1 | SLC35D3 | 4.72E−01 | <1E−09 |
| ASCL1 | CABYR | 4.72E−01 | 0.0012893 |
| ASCL1 | NPC1L1 | 4.72E−01 | <1E−09 |
| ASCL1 | LEFTY1 | 4.71E−01 | 0.0298735 |
| ASCL1 | GPR6 | 4.71E−01 | <1E−09 |
| ASCL1 | SVOP | 4.71E−01 | 0.0298735 |
| ASCL1 | PKD2L1 | 4.71E−01 | 0.0012893 |
| ASCL1 | MAP1A | 4.70E−01 | 1.82E−06 |
| ASCL1 | CAMKK1 | 4.70E−01 | 1.82E−06 |
| ASCL1 | COL22A1 | 4.69E−01 | <1E−09 |
| ASCL1 | LINGO2 | 4.69E−01 | <1E−09 |
| ASCL1 | IGSF10 | 4.68E−01 | 1.82E−06 |
| ASCL1 | SGSM1 | 4.68E−01 | <1E−09 |
| ASCL1 | BMP7 | 4.68E−01 | 0.0012893 |
| ASCL1 | RCAN3 | 4.66E−01 | <1E−09 |
| ASCL1 | KCTD16 | 4.66E−01 | <1E−09 |
| ASCL1 | CHRM4 | 4.66E−01 | <1E−09 |
| ASCL1 | LHFPL4 | 4.65E−01 | 5.06E−05 |
| ASCL1 | CYP2W1 | 4.64E−01 | <1E−09 |
| ASCL1 | MCOLN3 | 4.63E−01 | <1E−09 |
| ASCL1 | PXYLP1 | 4.62E−01 | 5.93E−08 |
| ASCL1 | SH3BGRL2 | 4.59E−01 | 0.0298735 |
| ASCL1 | PHACTR3 | 4.59E−01 | <1E−09 |
| ASCL1 | GRIN2C | 4.59E−01 | 5.93E−08 |
| ASCL1 | RPRM | 4.59E−01 | 0.0012893 |
| ASCL1 | LPAR3 | 4.58E−01 | 0.0298735 |
| ASCL1 | NDST4 | 4.58E−01 | <1E−09 |
| ASCL1 | GRP | 4.58E−01 | 3.30E−09 |
| ASCL1 | SLC7A14 | 4.58E−01 | <1E−09 |
| ASCL1 | ASPHD1 | 4.58E−01 | 5.06E−05 |
| ASCL1 | HECTD3 | 4.56E−01 | <1E−09 |
| ASCL1 | ATAD3C | 4.56E−01 | <1E−09 |
| ASCL1 | FGF14 | 4.56E−01 | 5.06E−05 |
| ASCL1 | CHP2 | 4.56E−01 | 0.0298735 |
| ASCL1 | C2CD4B | 4.54E−01 | <1E−09 |
| ASCL1 | FAM163B | 4.54E−01 | <1E−09 |
| ASCL1 | FAM178B | 4.52E−01 | <1E−09 |
| ASCL1 | ST8SIA3 | 4.51E−01 | 0.0012893 |
| ASCL1 | PKD1L3 | 4.51E−01 | <1E−09 |
| ASCL1 | HABP2 | 4.51E−01 | <1E−09 |
| ASCL1 | DZIP1L | 4.50E−01 | 0.0012893 |
| ASCL1 | SOWAHA | 4.50E−01 | <1E−09 |
| ASCL1 | CBFA2T2 | 4.50E−01 | 5.06E−05 |
| ASCL1 | TNFAIP8L1 | 4.50E−01 | 5.06E−05 |
| ASCL1 | DCX | 4.50E−01 | 3.30E−09 |
| ASCL1 | MCF2L | 4.49E−01 | 0.0298735 |
| ASCL1 | SLC38A3 | 4.49E−01 | <1E−09 |
| ASCL1 | DUSP8 | 4.49E−01 | 0.0012893 |
| ASCL1 | ELAVL3 | 4.48E−01 | 5.06E−05 |
| ASCL1 | PLEKHO2 | 4.48E−01 | 0.0298735 |
| ASCL1 | WNT11 | 4.46E−01 | 0.0298735 |
| ASCL1 | CHRM5 | 4.45E−01 | 0.0298735 |
| ASCL1 | HOXD9 | 4.45E−01 | 1.82E−06 |
| ASCL1 | MYBPHL | 4.44E−01 | <1E−09 |
| ASCL1 | TRIM72 | 4.42E−01 | <1E−09 |
| ASCL1 | MED25 | 4.41E−01 | <1E−09 |
| ASCL1 | JPH1 | 4.41E−01 | 5.06E−05 |
| ASCL1 | SCNN1A | 4.41E−01 | <1E−09 |
| ASCL1 | C12orf56 | 4.40E−01 | <1E−09 |
| ASCL1 | RIPPLY3 | 4.38E−01 | <1E−09 |
| ASCL1 | ERC2 | 4.38E−01 | 1.82E−06 |
| ASCL1 | MARVELD3 | 4.37E−01 | 1.82E−06 |
| ASCL1 | HEY1 | 4.34E−01 | <1E−09 |
| ASCL1 | CRHR2 | 4.34E−01 | <1E−09 |
| ASCL1 | TSEN54 | 4.33E−01 | <1E−09 |
| ASCL1 | BSPRY | 4.33E−01 | <1E−09 |
| ASCL1 | ITIH2 | 4.33E−01 | <1E−09 |
| ASCL1 | KCNF1 | 4.31E−01 | <1E−09 |
| ASCL1 | CCDC88C | 4.31E−01 | <1E−09 |
| ASCL1 | NEUROD1 | 4.28E−01 | <1E−09 |
| ASCL1 | ANKRD6 | 4.27E−01 | <1E−09 |
| ASCL1 | FBXL16 | 4.27E−01 | 0.0298735 |
| ASCL1 | C2CD4A | 4.26E−01 | <1E−09 |
| ASCL1 | ARHGEF7 | 4.24E−01 | <1E−09 |
| ASCL1 | NRCAM | 4.21E−01 | 3.30E−09 |
| ASCL1 | NGB | 4.20E−01 | <1E−09 |
| ASCL1 | TIGD3 | 4.19E−01 | 5.93E−08 |
| ASCL1 | TPH1 | 4.18E−01 | <1E−09 |
| ASCL1 | SOX1 | 4.11E−01 | <1E−09 |
| ASCL1 | PRODH | 4.10E−01 | <1E−09 |
| ASCL1 | HRH3 | 4.10E−01 | <1E−09 |
| ASCL1 | SLC6A5 | 4.10E−01 | <1E−09 |
| ASCL1 | EPB41L5 | 4.10E−01 | 0.0012893 |
| ASCL1 | PGAM2 | 4.09E−01 | <1E−09 |
| ASCL1 | HKDC1 | 4.09E−01 | 0.0012893 |
| ASCL1 | BAG2 | 4.08E−01 | 0.0012893 |
| ASCL1 | SPHKAP | 4.07E−01 | 3.30E−09 |
| ASCL1 | BTBD17 | 4.06E−01 | 3.30E−09 |
| ASCL1 | FSTL4 | 4.06E−01 | <1E−09 |
| ASCL1 | PCSK2 | 4.06E−01 | 0.0298735 |
| ASCL1 | SLC36A1 | 4.03E−01 | 0.0012893 |
| ASCL1 | MEP1B | 4.02E−01 | 5.93E−08 |
| ASCL1 | INSM2 | 3.99E−01 | 5.06E−05 |
| ASCL1 | MTURN | 3.97E−01 | 3.30E−09 |
| ASCL1 | CAMK1D | 3.95E−01 | 0.0298735 |
| ASCL1 | LCN15 | 3.94E−01 | <1E−09 |
| ASCL1 | AP3D1 | 3.94E−01 | 5.93E−08 |
| ASCL1 | INSYN2A | 3.92E−01 | 5.06E−05 |
| ASCL1 | KAAG1 | 3.92E−01 | 0.0298735 |
| ASCL1 | LYG2 | 3.90E−01 | 0.0298735 |
| ASCL1 | NKAIN2 | 3.89E−01 | 0.0012893 |
| ASCL1 | HNF4A | 3.89E−01 | <1E−09 |
| ASCL1 | P2RX6 | 3.88E−01 | 5.93E−08 |
| ASCL1 | RXFP3 | 3.87E−01 | 3.30E−09 |
| ASCL1 | RNF43 | 3.87E−01 | 0.0012893 |
| ASCL1 | SLC25A47 | 3.87E−01 | <1E−09 |
| ASCL1 | CER1 | 3.85E−01 | <1E−09 |
| ASCL1 | PPARGC1A | 3.81E−01 | 0.0012893 |
| ASCL1 | DNAI2 | 3.79E−01 | <1E−09 |
| ASCL1 | DOC2A | 3.77E−01 | 3.30E−09 |
| ASCL1 | PRSS2 | 3.72E−01 | <1E−09 |
| ASCL1 | TEX101 | 3.70E−01 | <1E−09 |
| ASCL1 | GET4 | 3.70E−01 | 5.06E−05 |
| ASCL1 | CALM1 | 3.69E−01 | 3.30E−09 |
| ASCL1 | NR0B1 | 3.67E−01 | <1E−09 |
| ASCL1 | TRIM55 | 3.67E−01 | 0.0298735 |
| ASCL1 | DCDC2 | 3.63E−01 | 5.93E−08 |
| ASCL1 | NBPF6 | 3.62E−01 | <1E−09 |
| ASCL1 | UCN3 | 3.61E−01 | <1E−09 |
| ASCL1 | SLC38A8 | 3.58E−01 | <1E−09 |
| ASCL1 | SF3A2 | 3.57E−01 | 0.0298735 |
| ASCL1 | TMPRSS7 | 3.56E−01 | 1.82E−06 |
| ASCL1 | NPPA | 3.52E−01 | 0.0298735 |
| ASCL1 | NMNAT2 | 3.51E−01 | 0.0298735 |
| ASCL1 | NBPF4 | 3.50E−01 | <1E−09 |
| ASCL1 | BMP3 | 3.50E−01 | 0.0298735 |
| ASCL1 | CPNE9 | 3.40E−01 | 0.0298735 |
| ASCL1 | HSD17B13 | 3.38E−01 | 1.82E−06 |
| ASCL1 | IL17C | 3.34E−01 | 0.0012893 |
| ASCL1 | HCRT | 3.27E−01 | <1E−09 |
| ASCL1 | FOXJ1 | 3.17E−01 | 3.30E−09 |
| ASCL1 | PRSS1 | 3.13E−01 | <1E−09 |
| ASCL1 | LSS | 3.11E−01 | 1.82E−06 |
| ASCL1 | IGFBP1 | 2.94E−01 | 5.06E−05 |
| ASCL1 | RETNLB | 2.91E−01 | <1E−09 |
| ASCL1 | NPPB | 2.57E−01 | 0.0012893 |
| ASCL1 | KCNK16 | 2.53E−01 | <1E−09 |
| ASCL1 | KRTAP10-4 | 2.20E−01 | 0.0298735 |
| ASCL1 | ADAD1 | 1.66E−01 | 0.0012893 |
| ASCL2 | PTGS1 | 6.77E−01 | <1E−09 |
| ASCL2 | POU2F3 | 6.61E−01 | <1E−09 |
| ASCL2 | RBM38 | 6.56E−01 | <1E−09 |
| ASCL2 | ANXA1 | 6.54E−01 | <1E−09 |
| ASCL2 | RNF135 | 6.53E−01 | 0.0012893 |
| ASCL2 | C11orf53 | 6.53E−01 | <1E−09 |
| ASCL2 | NCAPG | 6.53E−01 | <1E−09 |
| ASCL2 | ARHGAP11A | 6.51E−01 | 1.82E−06 |
| ASCL2 | CD40 | 6.49E−01 | <1E−09 |
| ASCL2 | DTL | 6.48E−01 | <1E−09 |
| ASCL2 | PIK3R5 | 6.47E−01 | <1E−09 |
| ASCL2 | HCK | 6.46E−01 | 3.30E−09 |
| ASCL2 | UBE2S | 6.44E−01 | 0.0012893 |
| ASCL2 | CTSB | 6.39E−01 | <1E−09 |
| ASCL2 | DIAPH3 | 6.37E−01 | <1E−09 |
| ASCL2 | ARHGAP22 | 6.34E−01 | <1E−09 |
| ASCL2 | RAD51AP1 | 6.34E−01 | <1E−09 |
| ASCL2 | AZGP1 | 6.34E−01 | 0.0298735 |
| ASCL2 | TOP2A | 6.33E−01 | 0.0298735 |
| ASCL2 | RCOR2 | 6.32E−01 | 5.06E−05 |
| ASCL2 | CSK | 6.32E−01 | 0.0298735 |
| ASCL2 | RGS13 | 6.31E−01 | 0.0012893 |
| ASCL2 | DEPDC7 | 6.31E−01 | 0.0012893 |
| ASCL2 | LMNB1 | 6.30E−01 | <1E−09 |
| ASCL2 | SLC14A1 | 6.30E−01 | <1E−09 |
| ASCL2 | RFC5 | 6.30E−01 | 5.93E−08 |
| ASCL2 | STAT5A | 6.30E−01 | 5.93E−08 |
| ASCL2 | SKA3 | 6.30E−01 | <1E−09 |
| ASCL2 | CXCL16 | 6.28E−01 | <1E−09 |
| ASCL2 | CENPO | 6.26E−01 | 0.0298735 |
| ASCL2 | SPIB | 6.26E−01 | <1E−09 |
| ASCL2 | NTN4 | 6.26E−01 | <1E−09 |
| ASCL2 | STMN1 | 6.26E−01 | 5.93E−08 |
| ASCL2 | PLA2G4A | 6.26E−01 | <1E−09 |
| ASCL2 | CCNB1 | 6.26E−01 | 1.82E−06 |
| ASCL2 | LDHB | 6.26E−01 | <1E−09 |
| ASCL2 | APBA2 | 6.25E−01 | <1E−09 |
| ASCL2 | JAK3 | 6.25E−01 | 0.0012893 |
| ASCL2 | IL2RG | 6.22E−01 | 0.0298735 |
| ASCL2 | CCDC88A | 6.21E−01 | 3.30E−09 |
| ASCL2 | KIF18A | 6.21E−01 | 0.0012893 |
| ASCL2 | MAD2L1 | 6.20E−01 | <1E−09 |
| ASCL2 | SOX2 | 6.19E−01 | <1E−09 |
| ASCL2 | LAYN | 6.18E−01 | 5.93E−08 |
| ASCL2 | ERCC6L | 6.17E−01 | 5.06E−05 |
| ASCL2 | FAT3 | 6.17E−01 | <1E−09 |
| ASCL2 | ABCB9 | 6.16E−01 | 0.0298735 |
| ASCL2 | HOXC10 | 6.13E−01 | 0.0012893 |
| ASCL2 | TLR9 | 6.12E−01 | <1E−09 |
| ASCL2 | TET1 | 6.12E−01 | 1.82E−06 |
| ASCL2 | TUBB2B | 6.12E−01 | 3.30E−09 |
| ASCL2 | GBP2 | 6.10E−01 | <1E−09 |
| ASCL2 | HAAO | 6.10E−01 | 1.82E−06 |
| ASCL2 | ISM2 | 6.10E−01 | <1E−09 |
| ASCL2 | GAMT | 6.09E−01 | 5.06E−05 |
| ASCL2 | HMGB3 | 6.08E−01 | <1E−09 |
| ASCL2 | TYMP | 6.07E−01 | <1E−09 |
| ASCL2 | CSTA | 6.07E−01 | <1E−09 |
| ASCL2 | TTK | 6.07E−01 | 3.30E−09 |
| ASCL2 | MYEOV | 6.07E−01 | 3.30E−09 |
| ASCL2 | C16orf74 | 6.07E−01 | 0.0298735 |
| ASCL2 | C3 | 6.06E−01 | 5.06E−05 |
| ASCL2 | HLA-DMB | 6.06E−01 | 5.93E−08 |
| ASCL2 | IL17RB | 6.06E−01 | 0.0298735 |
| ASCL2 | S100A11 | 6.06E−01 | 0.0012893 |
| ASCL2 | BLM | 6.06E−01 | 5.06E−05 |
| ASCL2 | NRG2 | 6.05E−01 | <1E−09 |
| ASCL2 | CDCA2 | 6.04E−01 | <1E−09 |
| ASCL2 | C4orf19 | 6.03E−01 | <1E−09 |
| ASCL2 | SLC8B1 | 6.03E−01 | 0.0012893 |
| ASCL2 | TRPM5 | 6.03E−01 | 3.30E−09 |
| ASCL2 | SLC40A1 | 6.03E−01 | <1E−09 |
| ASCL2 | TMEM74 | 6.02E−01 | 5.93E−08 |
| ASCL2 | CDC45 | 6.02E−01 | 0.0012893 |
| ASCL2 | CD7 | 6.01E−01 | 5.06E−05 |
| ASCL2 | CKAP2 | 6.01E−01 | 0.0298735 |
| ASCL2 | KCNJ15 | 5.99E−01 | 0.0298735 |
| ASCL2 | ADAMTSL5 | 5.99E−01 | 0.001289 |
| ASCL2 | TIMP1 | 5.99E−01 | 5.06E−05 |
| ASCL2 | SV2A | 5.98E−01 | 3.30E−09 |
| ASCL2 | CYP4B1 | 5.97E−01 | 0.0012893 |
| ASCL2 | SLC2A9 | 5.97E−01 | 1.82E−06 |
| ASCL2 | HOXA4 | 5.97E−01 | 5.06E−05 |
| ASCL2 | PBK | 5.97E−01 | 0.0012893 |
| ASCL2 | MSI2 | 5.96E−01 | <1E−09 |
| ASCL2 | GALNT14 | 5.95E−01 | <1E−09 |
| ASCL2 | VAV1 | 5.95E−01 | 5.93E−08 |
| ASCL2 | TAPBP | 5.95E−01 | <1E−09 |
| ASCL2 | MAPK11 | 5.94E−01 | 0.0298735 |
| ASCL2 | SPN | 5.93E−08 | 3.30E−09 |
| ASCL2 | ADAM8 | 5.93E−08 | 3.30E−09 |
| ASCL2 | OLIG1 | 5.92E−01 | 5.93E−08 |
| ASCL2 | CD82 | 5.92E−01 | 5.93E−08 |
| ASCL2 | MEGF6 | 5.92E−01 | 0.0012893 |
| ASCL2 | KCNIP4 | 5.91E−01 | <1E−09 |
| ASCL2 | IL4R | 5.90E−01 | 1.82E−06 |
| ASCL2 | PDE2A | 5.90E−01 | 0.0012893 |
| ASCL2 | OTUD1 | 5.90E−01 | 5.06E−05 |
| ASCL2 | KPNB1 | 5.89E−01 | 0.0298735 |
| ASCL2 | DOCK5 | 5.89E−01 | 5.06E−05 |
| ASCL2 | ARHGEF19 | 5.89E−01 | <1E−09 |
| ASCL2 | C9orf40 | 5.89E−01 | 0.0298735 |
| ASCL2 | APOL1 | 5.88E−01 | <1E−09 |
| ASCL2 | SPINDOC | 5.87E−01 | <1E−09 |
| ASCL2 | NR3C1 | 5.87E−01 | 0.0012893 |
| ASCL2 | CARD16 | 5.87E−01 | 1.82E−06 |
| ASCL2 | CD8A | 5.87E−01 | 5.93E−08 |
| ASCL2 | PIR | 5.87E−01 | 0.0298735 |
| ASCL2 | B3GALT4 | 5.86E−01 | 1.82E−06 |
| ASCL2 | GABARAPL1 | 5.86E−01 | 1.82E−06 |
| ASCL2 | E2F8 | 5.86E−01 | 5.06E−05 |
| ASCL2 | MTHFD2 | 5.86E−01 | 1.82E−06 |
| ASCL2 | TFAP2E | 5.85E−01 | <1E−09 |
| ASCL2 | PBX3 | 5.85E−01 | <1E−09 |
| ASCL2 | SQOR | 5.84E−01 | 5.93E−08 |
| ASCL2 | NCF1 | 5.84E−01 | 0.0298735 |
| ASCL2 | TLX3 | 5.83E−01 | 0.0298735 |
| ASCL2 | WWC3 | 5.83E−01 | <1E−09 |
| ASCL2 | IFNGR1 | 5.83E−01 | <1E−09 |
| ASCL2 | GBP1 | 5.82E−01 | 0.0012893 |
| ASCL2 | METRNL | 5.82E−01 | 0.0012893 |
| ASCL2 | TEAD4 | 5.82E−01 | <1E−09 |
| ASCL2 | TPRG1 | 5.81E−01 | <1E−09 |
| ASCL2 | MOB3C | 5.81E−01 | 0.0012893 |
| ASCL2 | SKAP2 | 5.81E−01 | 0.0298735 |
| ASCL2 | CASP1 | 5.80E−01 | <1E−09 |
| ASCL2 | FOXO1 | 5.80E−01 | 5.06E−05 |
| ASCL2 | RGMB | 5.80E−01 | 5.06E−05 |
| ASCL2 | WWC2 | 5.79E−01 | 5.93E−08 |
| ASCL2 | NFKB2 | 5.78E−01 | 5.93E−08 |
| ASCL2 | CCNJ | 5.78E−01 | <1E−09 |
| ASCL2 | TSN | 5.78E−01 | 0.0012893 |
| ASCL2 | ACSL5 | 5.78E−01 | <1E−09 |
| ASCL2 | BCAS4 | 5.78E−01 | 0.0012893 |
| ASCL2 | MGAT5 | 5.78E−01 | 0.0012893 |
| ASCL2 | SH2D7 | 5.77E−01 | 0.0298735 |
| ASCL2 | DGKZ | 5.77E−01 | 0.0298735 |
| ASCL2 | WASF1 | 5.76E−01 | 5.06E−05 |
| ASCL2 | LPAR6 | 5.76E−01 | 3.30E−09 |
| ASCL2 | OIP5 | 5.76E−01 | 0.0298735 |
| ASCL2 | RFC3 | 5.76E−01 | 0.0012893 |
| ASCL2 | KIAA0895 | 5.76E−01 | 0.0298735 |
| ASCL2 | PRKCQ | 5.76E−01 | 5.93E−08 |
| ASCL2 | EPHX4 | 5.76E−01 | 0.0298735 |
| ASCL2 | RGS16 | 5.76E−01 | 0.0012893 |
| ASCL2 | SQSTM1 | 5.75E−01 | 1.82E−06 |
| ASCL2 | AGO1 | 5.75E−01 | 0.0012893 |
| ASCL2 | TMEM265 | 5.74E−01 | <1E−09 |
| ASCL2 | MRPL15 | 5.74E−01 | 5.06E−05 |
| ASCL2 | TMC8 | 5.74E−01 | <1E−09 |
| ASCL2 | IPO5 | 5.74E−01 | 0.001289 |
| ASCL2 | INSYN2B | 5.74E−01 | 5.93E−08 |
| ASCL2 | HLA-E | 5.73E−01 | 0.001289 |
| ASCL2 | APOL3 | 5.73E−01 | 1.82E−06 |
| ASCL2 | MPZL2 | 5.73E−01 | 5.06E−05 |
| ASCL2 | RPP25 | 5.72E−01 | <1E−09 |
| ASCL2 | RASAL1 | 5.71E−01 | <1E−09 |
| ASCL2 | PLCD1 | 5.71E−01 | 0.0298735 |
| ASCL2 | KCNN3 | 5.70E−01 | <1E−09 |
| ASCL2 | HPDL | 5.70E−01 | 3.30E−09 |
| ASCL2 | DESI2 | 5.70E−01 | 0.0298735 |
| ASCL2 | IL13RA1 | 5.70E−01 | <1E−09 |
| ASCL2 | CENPV | 5.70E−01 | <1E−09 |
| ASCL2 | ITPRIPL1 | 5.68E−01 | <1E−09 |
| ASCL2 | CDH4 | 5.68E−01 | <1E−09 |
| ASCL2 | ODF3B | 5.68E−01 | 5.06E−05 |
| ASCL2 | PTPRCAP | 5.67E−01 | 0.0012893 |
| ASCL2 | FAM216A | 5.67E−01 | 0.0012893 |
| ASCL2 | INPP5D | 5.66E−01 | 5.93E−08 |
| ASCL2 | DTX2 | 5.66E−01 | <1E−09 |
| ASCL2 | ABCC3 | 5.65E−01 | <1E−09 |
| ASCL2 | PTAFR | 5.65E−01 | <1E−09 |
| ASCL2 | PINLYP | 5.65E−01 | <1E−09 |
| ASCL2 | PLIN4 | 5.65E−01 | 5.06E−05 |
| ASCL2 | TNFRSF1A | 5.64E−01 | <1E−09 |
| ASCL2 | RSPO4 | 5.63E−01 | <1E−09 |
| ASCL2 | SCPEP1 | 5.62E−01 | <1E−09 |
| ASCL2 | OSR1 | 5.62E−01 | 0.0012893 |
| ASCL2 | ARNT2 | 5.61E−01 | 0.0012893 |
| ASCL2 | MTMR2 | 5.61E−01 | <1E−09 |
| ASCL2 | PSMD14 | 5.61E−01 | 1.82E−06 |
| ASCL2 | ACSF2 | 5.60E−01 | <1E−09 |
| ASCL2 | DGKI | 5.59E−01 | 5.93E−08 |
| ASCL2 | KIAA0040 | 5.58E−01 | 1.82E−06 |
| ASCL2 | HTR3E | 5.58E−01 | 0.0012893 |
| ASCL2 | PSPC1 | 5.58E−01 | 0.0012893 |
| ASCL2 | FXYD5 | 5.58E−01 | 3.30E−09 |
| ASCL2 | NCMAP | 5.58E−01 | 5.93E−08 |
| ASCL2 | SERTAD4 | 5.57E−01 | <1E−09 |
| ASCL2 | TRPV4 | 5.57E−01 | <1E−09 |
| ASCL2 | NTF4 | 5.57E−01 | <1E−09 |
| ASCL2 | STAC3 | 5.57E−01 | 0.0298735 |
| ASCL2 | TRAF3IP3 | 5.56E−01 | <1E−09 |
| ASCL2 | CNDP2 | 5.56E−01 | 0.001289 |
| ASCL2 | ITGB7 | 5.56E−01 | <1E−09 |
| ASCL2 | UBE2L6 | 5.56E−01 | 0.0298735 |
| ASCL2 | PIK3C2B | 5.55E−01 | 0.0012893 |
| ASCL2 | ACP5 | 5.55E−01 | <1E−09 |
| ASCL2 | SLC25A13 | 5.55E−01 | <1E−09 |
| ASCL2 | GADD45B | 5.54E−01 | 0.0012893 |
| ASCL2 | TRADD | 5.53E−01 | <1E−09 |
| ASCL2 | USP13 | 5.53E−01 | 0.0298735 |
| ASCL2 | SERPINA1 | 5.53E−01 | 0.0298735 |
| ASCL2 | TNIP1 | 5.53E−01 | 0.0012893 |
| ASCL2 | PRR7 | 5.53E−01 | 1.82E−06 |
| ASCL2 | CYP4F12 | 5.52E−01 | 5.06E−05 |
| ASCL2 | DPF1 | 5.52E−01 | 0.0298735 |
| ASCL2 | TLR5 | 5.52E−01 | <1E−09 |
| ASCL2 | INO80C | 5.51E−01 | <1E−09 |
| ASCL2 | MEX3B | 5.51E−01 | 1.82E−06 |
| ASCL2 | BATF | 5.50E−01 | 5.06E−05 |
| ASCL2 | SSH3 | 5.50E−01 | 1.82E−06 |
| ASCL2 | TSPAN12 | 5.50E−01 | 1.82E−06 |
| ASCL2 | CACYBP | 5.50E−01 | 0.0298735 |
| ASCL2 | PSORS1C1 | 5.50E−01 | 5.93E−08 |
| ASCL2 | MATK | 5.50E−01 | 0.0012893 |
| ASCL2 | CASP6 | 5.49E−01 | 1.82E−06 |
| ASCL2 | TBC1D2 | 5.49E−01 | 1.82E−06 |
| ASCL2 | IRS2 | 5.49E−01 | <1E−09 |
| ASCL2 | GRN | 5.48E−01 | 5.06E−05 |
| ASCL2 | KBTBD6 | 5.48E−01 | <1E−09 |
| ASCL2 | RTN4RL1 | 5.48E−01 | <1E−09 |
| ASCL2 | MUC20 | 5.47E−01 | 5.06E−05 |
| ASCL2 | RUNX1 | 5.47E−01 | 1.82E−06 |
| ASCL2 | MAP3K5 | 5.47E−01 | <1E−09 |
| ASCL2 | USP3 | 5.47E−01 | <1E−09 |
| ASCL2 | CCDC112 | 5.45E−01 | 1.82E−06 |
| ASCL2 | ALOX12B | 5.44E−01 | <1E−09 |
| ASCL2 | SAPCD2 | 5.44E−01 | 0.0012893 |
| ASCL2 | DLK2 | 5.44E−01 | <1E−09 |
| ASCL2 | CD68 | 5.44E−01 | <1E−09 |
| ASCL2 | CDS1 | 5.44E−01 | <1E−09 |
| ASCL2 | POGLUT2 | 5.44E−01 | 5.93E−08 |
| ASCL2 | TTYH1 | 5.43E−01 | 5.06E−05 |
| ASCL2 | GTF3A | 5.43E−01 | 5.06E−05 |
| ASCL2 | PIK3CD | 5.43E−01 | <1E−09 |
| ASCL2 | DENND2D | 5.43E−01 | 5.93E−08 |
| ASCL2 | THAP11 | 5.43E−01 | 5.93E−08 |
| ASCL2 | PLBD1 | 5.42E−01 | 0.001289 |
| ASCL2 | NEURL3 | 5.42E−01 | <1E−09 |
| ASCL2 | PLB1 | 5.42E−01 | <1E−09 |
| ASCL2 | BNIP5 | 5.42E−01 | <1E−09 |
| ASCL2 | VOPP1 | 5.41E−01 | <1E−09 |
| ASCL2 | SUV39H2 | 5.41E−01 | 3.30E−09 |
| ASCL2 | RTP4 | 5.41E−01 | <1E−09 |
| ASCL2 | APOBEC3G | 5.38E−01 | <1E−09 |
| ASCL2 | ZNF74 | 5.37E−01 | <1E−09 |
| ASCL2 | DBN1 | 5.37E−01 | <1E−09 |
| ASCL2 | RBM20 | 5.37E−01 | 0.0012893 |
| ASCL2 | GBP3 | 5.36E−01 | <1E−09 |
| ASCL2 | ITGB4 | 5.36E−01 | 1.82E−06 |
| ASCL2 | TLE4 | 5.35E−01 | 5.06E−05 |
| ASCL2 | SH3BGRL3 | 5.34E−01 | 0.0298735 |
| ASCL2 | ALOX5 | 5.34E−01 | 3.30E−09 |
| ASCL2 | YDJC | 5.33E−01 | 1.82E−06 |
| ASCL2 | XPOT | 5.31E−01 | 0.0298735 |
| ASCL2 | CLIC3 | 5.31E−01 | <1E−09 |
| ASCL2 | CNDP1 | 5.31E−01 | 3.30E−09 |
| ASCL2 | MZT1 | 5.29E−01 | 0.0012893 |
| ASCL2 | CMTM7 | 5.28E−01 | <1E−09 |
| ASCL2 | RTN4R | 5.27E−01 | 5.93E−08 |
| ASCL2 | SLC4A2 | 5.27E−01 | 5.93E−08 |
| ASCL2 | RNF24 | 5.26E−01 | 0.0298735 |
| ASCL2 | LRAT | 5.26E−01 | <1E−09 |
| ASCL2 | CARD10 | 5.25E−01 | 1.82E−06 |
| ASCL2 | STOX2 | 5.25E−01 | 5.06E−05 |
| ASCL2 | TMEM45B | 5.24E−01 | 0.0012893 |
| ASCL2 | ZFHX4 | 5.24E−01 | 5.06E−05 |
| ASCL2 | FLRT3 | 5.24E−01 | <1E−09 |
| ASCL2 | GPX3 | 5.22E−01 | 0.0298735 |
| ASCL2 | SPTSSA | 5.22E−01 | 5.06E−05 |
| ASCL2 | UCHL5 | 5.22E−01 | 0.0298735 |
| ASCL2 | CNTN6 | 5.21E−01 | <1E−09 |
| ASCL2 | SKIDA1 | 5.20E−01 | 5.93E−08 |
| ASCL2 | PHYHIP | 5.20E−01 | 5.06E−05 |
| ASCL2 | WNT3A | 5.20E−01 | <1E−09 |
| ASCL2 | GUCA1A | 5.18E−01 | 0.0012893 |
| ASCL2 | ZDHHC14 | 5.17E−01 | 5.06E−05 |
| ASCL2 | MVP | 5.17E−01 | 0.0298735 |
| ASCL2 | EVA1C | 5.16E−01 | 0.0298735 |
| ASCL2 | MAGOHB | 5.15E−01 | 0.0298735 |
| ASCL2 | HMGN1 | 5.15E−01 | 0.0012893 |
| ASCL2 | TGIF2 | 5.15E−01 | <1E−09 |
| ASCL2 | ZC3H12B | 5.14E−01 | 0.0012893 |
| ASCL2 | SECTM1 | 5.14E−01 | <1E−09 |
| ASCL2 | MALSU1 | 5.14E−01 | 0.0298735 |
| ASCL2 | CLDN16 | 5.14E−01 | 0.0298735 |
| ASCL2 | DDO | 5.12E−01 | 1.82E−06 |
| ASCL2 | MPP6 | 5.11E−01 | <1E−09 |
| ASCL2 | PSMB10 | 5.11E−01 | 0.0298735 |
| ASCL2 | SMIM13 | 5.09E−01 | 0.0298735 |
| ASCL2 | CAPG | 5.09E−01 | <1E−09 |
| ASCL2 | MORC1 | 5.08E−01 | 0.0012893 |
| ASCL2 | LGALS3 | 5.08E−01 | <1E−09 |
| ASCL2 | SLC52A1 | 5.07E−01 | <1E−09 |
| ASCL2 | PCGF6 | 5.07E−01 | 1.82E−06 |
| ASCL2 | GPR155 | 5.07E−01 | 3.30E−09 |
| ASCL2 | NINJ1 | 5.05E−01 | <1E−09 |
| ASCL2 | KCNS1 | 5.04E−01 | <1E−09 |
| ASCL2 | MYO3B | 5.04E−01 | 3.30E−09 |
| ASCL2 | SLC6A15 | 5.03E−01 | <1E−09 |
| ASCL2 | CHRNA5 | 5.03E−01 | <1E−09 |
| ASCL2 | NCF4 | 5.03E−01 | 0.0012893 |
| ASCL2 | INKA1 | 5.03E−01 | <1E−09 |
| ASCL2 | CCNI2 | 5.02E−01 | 5.06E−05 |
| ASCL2 | IRF2 | 5.02E−01 | 1.82E−06 |
| ASCL2 | NUP93 | 4.99E−01 | 0.0298735 |
| ASCL2 | NXPH4 | 4.99E−01 | 5.93E−08 |
| ASCL2 | IL1RL2 | 4.98E−01 | 0.0298735 |
| ASCL2 | MYZAP | 4.98E−01 | <1E−09 |
| ASCL2 | DUOX2 | 4.95E−01 | 0.0012893 |
| ASCL2 | CDCA7L | 4.95E−01 | 5.06E−05 |
| ASCL2 | PDZRN3 | 4.94E−01 | 0.0298735 |
| ASCL2 | CHAT | 4.94E−01 | 1.82E−06 |
| ASCL2 | SLPI | 4.93E−01 | 0.0298735 |
| ASCL2 | SMTNL2 | 4.93E−01 | 5.93E−08 |
| ASCL2 | BATF2 | 4.91E−01 | 5.93E−08 |
| ASCL2 | KCNN4 | 4.89E−01 | <1E−09 |
| ASCL2 | KLK11 | 4.86E−01 | 5.06E−05 |
| ASCL2 | QPCT | 4.84E−01 | 0.0298735 |
| ASCL2 | GATA3 | 4.82E−01 | 5.06E−05 |
| ASCL2 | VILL | 4.80E−01 | 1.82E−06 |
| ASCL2 | OXCT1 | 4.78E−01 | 0.0298735 |
| ASCL2 | UPK3B | 4.77E−01 | 1.82E−06 |
| ASCL2 | SPTSSB | 4.76E−01 | <1E−09 |
| ASCL2 | KLHDC7A | 4.73E−01 | <1E−09 |
| ASCL2 | PRR15 | 4.72E−01 | <1E−09 |
| ASCL2 | MAT1A | 4.71E−01 | <1E−09 |
| ASCL2 | OBSCN | 4.71E−01 | 0.0012893 |
| ASCL2 | CITED4 | 4.67E−01 | 5.93E−08 |
| ASCL2 | FAM171A2 | 4.67E−01 | 0.0298735 |
| ASCL2 | C3orf14 | 4.66E−01 | 0.0298735 |
| ASCL2 | LDAH | 4.62E−01 | 5.93E−08 |
| ASCL2 | ETV7 | 4.62E−01 | <1E−09 |
| ASCL2 | YBX2 | 4.57E−01 | 0.0298735 |
| ASCL2 | CEACAM1 | 4.56E−01 | 5.93E−08 |
| ASCL2 | NSFL1C | 4.56E−01 | 0.0012893 |
| ASCL2 | PYCARD | 4.54E−01 | <1E−09 |
| ASCL2 | TBX6 | 4.53E−01 | 0.0012893 |
| ASCL2 | TMPRSS3 | 4.52E−01 | <1E−09 |
| ASCL2 | PARP3 | 4.51E−01 | 0.0012893 |
| ASCL2 | COL25A1 | 4.51E−01 | 0.0298735 |
| ASCL2 | FAM124A | 4.50E−01 | 0.0298735 |
| ASCL2 | VWA2 | 4.41E−01 | 5.93E−08 |
| ASCL2 | TMEM63A | 4.41E−01 | <1E−09 |
| ASCL2 | SCIN | 4.40E−01 | 0.0298735 |
| ASCL2 | CDH22 | 4.36E−01 | 0.0298735 |
| ASCL2 | ETV6 | 4.26E−01 | 5.06E−05 |
| ASCL2 | CCDC9B | 4.25E−01 | 0.0298735 |
| ASCL2 | SLC22A15 | 4.21E−01 | 0.0012893 |
| ASCL2 | LHFPL4 | 4.21E−01 | 5.93E−08 |
| ASCL2 | C6orf15 | 4.17E−01 | 3.30E−09 |
| ASCL2 | AMT | 3.91E−01 | 5.93E−08 |
| ASCL2 | HSBP1L1 | 3.89E−01 | 3.30E−09 |
| ASCL2 | TMEM30A | 3.81E−01 | 0.0298735 |
| ASCL2 | TAS1R1 | 3.79E−01 | 0.0012893 |
| ASCL2 | TRIM49D1 | 3.04E−01 | <1E−09 |

TABLE 6
Genes implicated in the small cell neuroendocrine transdifferentiation transition

| Gene Name | Gene | Gene Name | Gene |
|---|---|---|---|
| FABP4 | fatty acid binding protein 4 | EPPIN | epididymal peptidase inhibitor |
| OLFM4 | olfactomedin 4 | SOX8 | SRY-box transcription factor 8 |
| S100A7 | S100 calcium binding protein A7 | PDZRN4 | PDZ domain containing ring finger 4 |
| KRT1 | keratin 1 | ZNF804A | zinc finger protein 804A |
| BPIFB1 | BPI fold containing family B | ADAMTS4 | ADAM metallopeptidase with |
| | member 1 | | thrombospondin type 1 motif 4 |
| TMPRSS11D | transmembrane serine protease | TEAD2 | TEA domain transcription factor 2 |
| | 11D | | |
| PIGR | polymeric immunoglobulin receptor | ALOX5AP | arachidonate 5-lipoxygenase |
| | | | activating protein |
| MMP13 | matrix metallopeptidase 13 | ANGPTL7 | angiopoietin like 7 |
| APOBEC3A | apolipoprotein B mRNA editing | ANXA8L1 | annexin A8 like 1 |
| | enzyme catalytic subunit 3A | | |
| SPRR2A | small proline rich protein 2A | SCGN | secretagogin, EF-hand calcium |
| | | | binding protein |
| SPRR2E | small proline rich protein 2E | INSM1 | INSM transcriptional repressor 1 |
| FMO3 | flavin containing dimethylaniline | OLR1 | oxidized low density lipoprotein |
| | monoxygenase 3 | | receptor 1 |
| HLA-DRA | major histocompatibility complex, | SLC6A15 | solute carrier family 6 member 15 |
| | class II, DR alpha | | |
| S100A7A | S100 calcium binding protein A7A | PROM1 | prominin 1 |
| SPRR2D | small proline rich protein 2D | CDH5 | cadherin 5 |
| S100A8 | S100 calcium binding protein A8 | COL5A1 | collagen type V alpha 1 chain |
| LTF | lactotransferrin | SPRR2F | small proline rich protein 2F |
| GABRP | gamma-aminobutyric acid type A | TENM2 | teneurin transmembrane protein 2 |
| | receptor subunit pi | | |
| DSG1 | desmoglein 1 | SERPINA11 | serpin family A member 11 |
| SH2D7 | SH2 domain containing 7 | CXCL5 | C-X-C motif chemokine ligand 5 |
| MMP3 | matrix metallopeptidase 3 | DACT2 | dishevelled binding antagonist of |
| | | | beta catenin 2 |
| SERPINB2 | serpin family B member 2 | VEPH1 | ventricular zone expressed PH |
| | | | domain containing 1 |
| ADH7 | alcohol dehydrogenase 7 (class | NTS | neurotensin |
| | IV), mu or sigma polypeptide | | |
| TMPRSS11A | transmembrane serine protease | HMOX1 | heme oxygenase 1 |
| | 11A | | |
| CXCL14 | C-X-C motif chemokine ligand 14 | KLK6 | kallikrein related peptidase 6 |
| KRT75 | keratin 75 | RARRES2 | retinoic acid receptor responder 2 |
| IL36G | interleukin 36 gamma | SLC1A3 | solute carrier family 1 member 3 |
| TFAP2B | transcription factor AP-2 beta | STRA6 | signaling receptor and transporter of |
| | | | retinol STRA6 |
| BMX | BMX non-receptor tyrosine kinase | SLCO2B1 | solute carrier organic anion |
| | | | transporter family member 2B1 |
| HCAR3 | hydroxycarboxylic acid receptor 3 | ZNF385D | zinc finger protein 385D |
| KRT6C | keratin 6C | CLCF1 | cardiotrophin like cytokine factor 1 |
| CRISP3 | cysteine rich secretory protein 3 | FYB | #N/A |
| MYBPC1 | myosin binding protein C1 | PRRX1 | paired related homeobox 1 |
| SPRR3 | small proline rich protein 3 | KCNQ4 | potassium voltage-gated channel |
| | | | subfamily Q member 4 |
| RGS13 | regulator of G protein signaling 13 | HLA-DPB1 | major histocompatibility complex, |
| | | | class II, DP beta 1 |
| HMCN1 | hemicentin 1 | DDC | dopa decarboxylase |
| TRPM5 | transient receptor potential cation | MEDAG | mesenteric estrogen dependent |
| | channel subfamily M member 5 | | adipogenesis |
| CA3 | carbonic anhydrase 3 | IL1RAPL2 | interleukin 1 receptor accessory |
| | | | protein like 2 |
| DGKI | diacylglycerol kinase iota | MXRA5 | matrix remodeling associated 5 |
| KRT79 | keratin 79 | THRSP | thyroid hormone responsive |
| FAM3D | FAM3 metabolism regulating | IFITM1 | interferon induced transmembrane |
| | signaling molecule D | | protein 1 |
| CCL2 | C-C motif chemokine ligand 2 | DTHD1 | death domain containing 1 |
| IFI27 | interferon alpha inducible protein | CPLX2 | complexin 2 |
| | 27 | | |
| IL1RL1 | interleukin 1 receptor like 1 | ANXA8 | annexin A8 |
| SCGB3A1 | secretoglobin family 3A member 1 | ISM2 | isthmin 2 |
| CLEC7A | C-type lectin domain containing 7A | TLL1 | tolloid like 1 |
| IVL | involucrin | ITPKC | inositol-trisphosphate 3-kinase C |
| FMO1 | flavin containing dimethylaniline | C6orf15 | chromosome 6 open reading frame |
| | monoxygenase 1 | | 15 |
| HTR3E | 5-hydroxytryptamine receptor 3E | OMG | oligodendrocyte myelin glycoprotein |
| MMP9 | matrix metallopeptidase 9 | PTPRQ | protein tyrosine phosphatase |
| | | | receptor type Q |
| KRT6B | keratin 6B | SLC18A3 | solute carrier family 18 member A3 |
| IGFL1 | IGF like family member 1 | SULT1E1 | sulfotransferase family 1E member |
| | | | 1 |
| FMO2 | flavin containing dimethylaniline | MPPED2 | metallophosphoesterase domain |
| | monoxygenase 2 | | containing 2 |
| AMER2 | APC membrane recruitment protein | AOAH | acyloxyacyl hydrolase |
| | 2 | | |
| DPYSL5 | dihydropyrimidinase like 5 | COL3A1 | collagen type III alpha 1 chain |
| VCAM1 | vascular cell adhesion molecule 1 | CYP4B1 | cytochrome P450 family 4 subfamily |
| | | | B member 1 |
| CEACAM6 | CEA cell adhesion molecule 6 | KRT5 | keratin 5 |
| KYNU | kynureninase | SFRP2 | secreted frizzled related protein 2 |
| SPINK5 | serine peptidase inhibitor Kazal | DAZ3 | deleted in azoospermia 3 |
| | type 5 | | |
| TNIP3 | TNFAIP3 interacting protein 3 | EDN1 | endothelin 1 |
| ROS1 | ROS proto-oncogene 1, receptor | KIAA1211 | #N/A |
| | tyrosine kinase | | |
| KRT13 | keratin 13 | SCN3A | sodium voltage-gated channel alpha |
| | | | subunit 3 |
| SPRR1B | small proline rich protein 1B | EEF1A2 | eukaryotic translation elongation |
| | | | factor 1 alpha 2 |
| PLEKHS1 | pleckstrin homology domain | CHGA | chromogranin A |
| | containing S1 | | |
| CP | ceruloplasmin | UGT2B17 | UDP glucuronosyltransferase family |
| | | | 2 member B17 |
| PSD2 | pleckstrin and Sec7 domain | CWH43 | cell wall biogenesis 43 C-terminal |
| | containing 2 | | homolog |
| LYZ | lysozyme | KANK4 | KN motif and ankyrin repeat |
| | | | domains 4 |
| PTPRB | protein tyrosine phosphatase | CLCNKB | chloride voltage-gated channel Kb |
| | receptor type B | | |
| MMP10 | matrix metallopeptidase 10 | APBA2 | amyloid beta precursor protein |
| | | | binding family A member 2 |
| IFI44L | interferon induced protein 44 like | BCAS1 | brain enriched myelin associated |
| | | | protein 1 |
| CA1 | carbonic anhydrase 1 | FGFBP1 | fibroblast growth factor binding |
| | | | protein 1 |
| KRT84 | keratin 84 | BMP3 | bone morphogenetic protein 3 |
| PI3 | peptidase inhibitor 3 | FOXN1 | forkhead box N1 |
| MUC5B | mucin 5B, oligomeric mucus/gel- | TSLP | thymic stromal lymphopoietin |
| | forming | | |
| LGR5 | leucine rich repeat containing G | IRX4 | iroquois homeobox 4 |
| | protein-coupled receptor 5 | | |
| LY6D | lymphocyte antigen 6 family | FLRT2 | fibronectin leucine rich |
| | member D | | transmembrane protein 2 |
| CYP27C1 | cytochrome P450 family 27 | ACTC1 | actin alpha cardiac muscle 1 |
| | subfamily C member 1 | | |
| POSTN | periostin | TMEM132D | transmembrane protein 132D |
| COL12A1 | collagen type XII alpha 1 chain | EBF1 | EBF transcription factor 1 |
| GFI1B | growth factor independent 1B | TTYH1 | tweety family member 1 |
| | transcriptional repressor | | |
| MMP12 | matrix metallopeptidase 12 | ZBP1 | Z-DNA binding protein 1 |
| ART3 | ADP-ribosyltransferase 3 (inactive) | KRT10 | keratin 10 |
| FST | follistatin | RAET1L | retinoic acid early transcript 1L |
| CHL1 | cell adhesion molecule L1 like | PTX3 | pentraxin 3 |
| KRTDAP | keratinocyte differentiation | ADORA1 | adenosine A1 receptor |
| | associated protein | | |
| CD36 | CD36 molecule | RHBDL3 | rhomboid like 3 |
| GNG13 | G protein subunit gamma 13 | CSF2RB | colony stimulating factor 2 receptor |
| | | | subunit beta |
| ATP6V0D2 | ATPase H+ transporting V0 subunit | SI | sucrase-isomaltase |
| | d2 | | |
| KLHDC7B | kelch domain containing 7B | DAPP1 | dual adaptor of phosphotyrosine |
| | | | and 3-phosphoinositides 1 |
| CXCL1 | C-X-C motif chemokine ligand 1 | MSN | moesin |
| SPIB | Spi-B transcription factor | ASCL2 | achaete-scute family bHLH |
| | | | transcription factor 2 |
| S100A9 | S100 calcium binding protein A9 | FOSL1 | FOS like 1, AP-1 transcription factor |
| | | | subunit |
| SLC12A3 | solute carrier family 12 member 3 | MAP3K19 | mitogen-activated protein kinase |
| | | | kinase kinase 19 |
| A2ML1 | alpha-2-macroglobulin like 1 | SLC25A48 | solute carrier family 25 member 48 |
| IL7R | interleukin 7 receptor | FN1 | fibronectin 1 |
| MUC13 | mucin 13, cell surface associated | ST6GALNAC3 | ST6 N-acetylgalactosaminide alpha- |
| | | | 2,6-sialyltransferase 3 |
| FBN3 | fibrillin 3 | CLEC3A | C-type lectin domain family 3 |
| | | | member A |
| SLC10A6 | solute carrier family 10 member 6 | PREX1 | phosphatidylinositol-3,4,5- |
| | | | trisphosphate dependent Rac |
| | | | exchange factor 1 |
| TRIM31 | tripartite motif containing 31 | GLI1 | GLI family zinc finger 1 |
| CHRDL1 | chordin like 1 | PGLYRP3 | peptidoglycan recognition protein 3 |
| NTRK2 | neurotrophic receptor tyrosine | TMPRSS11E | transmembrane serine protease |
| | kinase 2 | | 11E |
| CDC20B | cell division cycle 20B | ATP2A3 | ATPase sarcoplasmic/endoplasmic |
| | | | reticulum Ca2+ transporting 3 |
| HCK | HCK proto-oncogene, Src family | SPRR4 | small proline rich protein 4 |
| | tyrosine kinase | | |
| PIK3R5 | phosphoinositide-3-kinase | ELF5 | E74 like ETS transcription factor 5 |
| | regulatory subunit 5 | | |
| C11orf53 | #N/A | S1PR3 | sphingosine-1-phosphate receptor 3 |
| FAP | fibroblast activation protein alpha | TGM5 | transglutaminase 5 |
| CSF2 | colony stimulating factor 2 | MS4A8 | membrane spanning 4-domains A8 |
| SLC5A8 | solute carrier family 5 member 8 | NRG1 | neuregulin 1 |
| CPB1 | carboxypeptidase B1 | COL25A1 | collagen type XXV alpha 1 chain |
| CXCL2 | C-X-C motif chemokine ligand 2 | MSRB3 | methionine sulfoxide reductase B3 |
| LEFTY1 | left-right determination factor 1 | CD70 | CD70 molecule |
| INHBA | inhibin subunit beta A | ADAM19 | ADAM metallopeptidase domain 19 |
| PDE2A | phosphodiesterase 2A | PALMD | palmdelphin |
| TMEM213 | transmembrane protein 213 | ARHGDIB | Rho GDP dissociation inhibitor beta |
| CPA4 | carboxypeptidase A4 | TMEM108 | transmembrane protein 108 |
| C20orf85 | chromosome 20 open reading | NRXN1 | neurexin 1 |
| | frame 85 | | |
| LGALS7B | galectin 7B | DACH1 | dachshund family transcription |
| | | | factor 1 |
| SLC34A2 | solute carrier family 34 member 2 | C1QTNF5 | C1q and TNF related 5 |
| CALML5 | calmodulin like 5 | MUC15 | mucin 15, cell surface associated |
| MUCL1 | mucin like 1 | RGAG1 | #N/A |
| MGP | matrix Gla protein | KRTAP3-1 | keratin associated protein 3-1 |
| HOXC4 | homeobox C4 | SPSB4 | splA/ryanodine receptor domain and |
| | | | SOCS box containing 4 |
| ACKR3 | atypical chemokine receptor 3 | ANPEP | alanyl aminopeptidase, membrane |
| CCL20 | C-C motif chemokine ligand 20 | CAV1 | caveolin 1 |
| FAT3 | FAT atypical cadherin 3 | THBS1 | thrombospondin 1 |
| IDO1 | indoleamine 2,3-dioxygenase 1 | GLI3 | GLI family zinc finger 3 |
| MUC21 | mucin 21, cell surface associated | CXorf49 | chromosome X open reading frame |
| | | | 49 |
| MUC2 | mucin 2, oligomeric mucus/gel- | IL13RA2 | interleukin 13 receptor subunit alpha |
| | forming | | 2 |
| SLC6A14 | solute carrier family 6 member 14 | PMEPA1 | prostate transmembrane protein, |
| | | | androgen induced 1 |
| CFTR | CF transmembrane conductance | SLC38A5 | solute carrier family 38 member 5 |
| | regulator | | |
| SLC6A2 | solute carrier family 6 member 2 | HEPH | hephaestin |
| ABCA13 | ATP binding cassette subfamily A | TACR1 | tachykinin receptor 1 |
| | member 13 | | |
| DIO2 | iodothyronine deiodinase 2 | MYCN | MYCN proto-oncogene, bHLH |
| | | | transcription factor |
| B4GALNT2 | beta-1,4-N-acetyl- | TRPV6 | transient receptor potential cation |
| | galactosaminyltransferase 2 | | channel subfamily V member 6 |
| ZFP42 | ZFP42 zinc finger protein | CLEC17A | C-type lectin domain containing 17A |
| DSG4 | desmoglein 4 | CNN1 | calponin 1 |
| TNC | tenascin C | CFAP47 | cilia and flagella associated protein |
| | | | 47 |
| IL6 | interleukin 6 | C1orf110 | #N/A |
| SLC13A2 | solute carrier family 13 member 2 | EPSTI1 | epithelial stromal interaction 1 |
| HPGDS | hematopoietic prostaglandin D | ARSF | arylsulfatase F |
| | synthase | | |
| HMX3 | H6 family homeobox 3 | ADGRF4 | adhesion G protein-coupled |
| | | | receptor F4 |
| SERPINA3 | serpin family A member 3 | RPE65 | retinoid isomerohydrolase RPE65 |
| LRMP | #N/A | NPR3 | natriuretic peptide receptor 3 |
| GLYATL2 | glycine-N-acyltransferase like 2 | EPHA7 | EPH receptor A7 |
| IL1RN | interleukin 1 receptor antagonist | CD38 | CD38 molecule |
| STAC2 | SH3 and cysteine rich domain 2 | FOXQ1 | forkhead box Q1 |
| SLAMF7 | SLAM family member 7 | KPNA7 | karyopherin subunit alpha 7 |
| COL15A1 | collagen type XV alpha 1 chain | HOXC11 | homeobox C11 |
| C5orf46 | chromosome 5 open reading frame | C15orf26 | #N/A |
| | 46 | | |
| CSF3 | colony stimulating factor 3 | ATP13A4 | ATPase 13A4 |
| SPN | sialophorin | BZRAP1 | #N/A |
| NCCRP1 | NCCRP1, F-box associated | RCAN2 | regulator of calcineurin 2 |
| | domain containing | | |
| GJB6 | gap junction protein beta 6 | HSPB8 | heat shock protein family B (small) |
| | | | member 8 |
| CXCL8 | C-X-C motif chemokine ligand 8 | GDNF | glial cell derived neurotrophic factor |
| GUCA1A | guanylate cyclase activator 1A | OGDHL | oxoglutarate dehydrogenase L |
| COL11A1 | collagen type XI alpha 1 chain | TMEM229A | transmembrane protein 229A |
| FOXC2 | forkhead box C2 | SYN3 | synapsin III |
| SYNPO2 | synaptopodin 2 | SCG2 | secretogranin II |
| C4BPA | complement component 4 binding | KIF26A | kinesin family member 26A |
| | protein alpha | | |
| KIAA1755 | KIAA1755 | PADI3 | peptidyl arginine deiminase 3 |
| DCN | decorin | MX2 | MX dynamin like GTPase 2 |
| ADGRF5 | adhesion G protein-coupled | HOXC13 | homeobox C13 |
| | receptor F5 | | |
| EFHD1 | EF-hand domain family member D1 | TP63 | tumor protein p63 |
| NCMAP | non-compact myelin associated | LGALS9 | galectin 9 |
| | protein | | |
| HOXC5 | homeobox C5 | CNPY1 | canopy FGF signaling regulator 1 |
| SHISA2 | shisa family member 2 | SLC9A4 | solute carrier family 9 member A4 |
| CHAT | choline O-acetyltransferase | RNASE7 | ribonuclease A family member 7 |
| CYP4Z1 | cytochrome P450 family 4 | TCTEX1D1 | #N/A |
| | subfamily Z member 1 | | |
| TMEM45A | transmembrane protein 45A | BMPR1B | bone morphogenetic protein |
| | | | receptor type 1B |
| IL1A | interleukin 1 alpha | ERN2 | endoplasmic reticulum to nucleus |
| | | | signaling 2 |
| SHISA9 | shisa family member 9 | OCA2 | OCA2 melanosomal |
| | | | transmembrane protein |
| HMX2 | H6 family homeobox 2 | CYP1A1 | cytochrome P450 family 1 subfamily |
| | | | A member 1 |
| TRIML2 | tripartite motif family like 2 | NNMT | nicotinamide N-methyltransferase |
| AMTN | amelotin | CCM2L | CCM2 like scaffold protein |
| C3 | complement C3 | ALDH1A3 | aldehyde dehydrogenase 1 family |
| | | | member A3 |
| SERPINB13 | serpin family B member 13 | KIF26B | kinesin family member 26B |
| ENPP3 | ectonucleotide pyrophosphatase/ | SERPINB5 | serpin family B member 5 |
| | phosphodiesterase 3 | | |
| HEPHL1 | hephaestin like 1 | C2orf72 | chromosome 2 open reading frame |
| | | | 72 |
| VNN1 | vanin 1 | DOK5 | docking protein 5 |
| SH2D6 | SH2 domain containing 6 | SLC5A1 | solute carrier family 5 member 1 |
| KRT14 | keratin 14 | USH1C | USH1 protein network component |
| | | | harmonin |
| GRAP2 | GRB2 related adaptor protein 2 | KCNJ15 | potassium inwardly rectifying |
| | | | channel subfamily J member 15 |
| LGALS7 | galectin 7 | NELL2 | neural EGFL like 2 |
| SERPINB4 | serpin family B member 4 | B3GNT6 | UDP-GlcNAc:betaGal beta-1,3-N- |
| | | | acetylglucosaminyltransferase 6 |
| HOXC10 | homeobox C10 | LYPD2 | LY6/PLAUR domain containing 2 |
| CLIC5 | chloride intracellular channel 5 | TRPA1 | transient receptor potential cation |
| | | | channel subfamily A member 1 |
| SLC26A4 | solute carrier family 26 member 4 | DTX1 | deltex E3 ubiquitin ligase 1 |
| TCN1 | transcobalamin 1 | GRAMD1B | GRAM domain containing 1B |
| VNN3 | #N/A | HTR1F | 5-hydroxytryptamine receptor 1F |
| RHOJ | ras homolog family member J | CAPNS2 | calpain small subunit 2 |
| MMP7 | matrix metallopeptidase 7 | CRYM | crystallin mu |
| HOXC9 | homeobox C9 | VIL1 | villin 1 |
| GPR12 | G protein-coupled receptor 12 | S100A12 | S100 calcium binding protein A12 |
| PLP1 | proteolipid protein 1 | ERP27 | endoplasmic reticulum protein 27 |
| CNTN6 | contactin 6 | CFH | complement factor H |
| MGAM2 | maltase-glucoamylase 2 (putative) | EYA1 | EYA transcriptional coactivator and |
| | | | phosphatase 1 |
| FAM150A | #N/A | TRIM49 | tripartite motif containing 49 |
| EMP1 | epithelial membrane protein 1 | CALCA | calcitonin related polypeptide alpha |
| PIP | prolactin induced protein | HYAL4 | hyaluronidase 4 |
| LRRC55 | leucine rich repeat containing 55 | S100P | S100 calcium binding protein P |
| HCAR2 | hydroxycarboxylic acid receptor 2 | KRT78 | keratin 78 |
| SBSN | suprabasin | C11orf16 | chromosome 11 open reading frame |
| | | | 16 |
| ATP12A | ATPase H+/K+ transporting non- | SERPINE2 | serpin family E member 2 |
| | gastric alpha2 subunit | | |
| NFATC1 | nuclear factor of activated T cells 1 | WFDC5 | WAP four-disulfide core domain 5 |
| FAM83C | family with sequence similarity 83 | HRASLS5 | #N/A |
| | member C | | |
| TNF | tumor necrosis factor | NIPAL4 | NIPA like domain containing 4 |
| PLA2G4A | phospholipase A2 group IVA | S100A2 | S100 calcium binding protein A2 |
| KRT4 | keratin 4 | CD55 | CD55 molecule (Cromer blood |
| | | | group) |
| ARL14 | ADP ribosylation factor like | MRO | maestro |
| | GTPase 14 | | |
| BMP5 | bone morphogenetic protein 5 | CH25H | cholesterol 25-hydroxylase |
| ATP6V1B1 | ATPase H+ transporting V1 subunit | APOL3 | apolipoprotein L3 |
| | B1 | | |
| SOX17 | SRY-box transcription factor 17 | DSC2 | desmocollin 2 |
| IL20 | interleukin 20 | TMEM71 | transmembrane protein 71 |
| PRRX2 | paired related homeobox 2 | COL9A1 | collagen type IX alpha 1 chain |
| ATP13A5 | ATPase 13A5 | LCK | LCK proto-oncogene, Src family |
| | | | tyrosine kinase |
| SAA1 | serum amyloid A1 | ADRB2 | adrenoceptor beta 2 |
| TRIM58 | tripartite motif containing 58 | ROBO2 | roundabout guidance receptor 2 |
| PTGS2 | prostaglandin-endoperoxide | UPK1A | uroplakin 1A |
| | synthase 2 | | |
| PCDH8 | protocadherin 8 | GPA33 | glycoprotein A33 |
| DIO3 | iodothyronine deiodinase 3 | SLC24A3 | solute carrier family 24 member 3 |
| FOXI1 | forkhead box I1 | AGR3 | anterior gradient 3, protein |
| | | | disulphide isomerase family |
| | | | member |
| HLA-DPA1 | major histocompatibility complex, | WNT5A | Wnt family member 5A |
| | class II, DP alpha 1 | | |
| MMP2 | matrix metallopeptidase 2 | CNGB1 | cyclic nucleotide gated channel |
| | | | subunit beta 1 |
| PLAUR | plasminogen activator, urokinase | UNC5A | unc-5 netrin receptor A |
| | receptor | | |
| HOXC6 | homeobox C6 | SLPI | secretory leukocyte peptidase |
| | | | inhibitor |
| PIK3CG | phosphatidylinositol-4,5- | DKK1 | dickkopf WNT signaling pathway |
| | bisphosphate 3-kinase catalytic | | inhibitor 1 |
| | subunit gamma | | |
| MSLN | mesothelin | TGM2 | transglutaminase 2 |
| DAZ1 | deleted in azoospermia 1 | PIM1 | Pim-1 proto-oncogene, |
| | | | serine/threonine kinase |
| SERPINE1 | serpin family E member 1 | TNFSF18 | TNF superfamily member 18 |
| B3GALT5 | beta-1,3-galactosyltransferase 5 | KCNH5 | potassium voltage-gated channel |
| | | | subfamily H member 5 |
| MASP1 | MBL associated serine protease 1 | SPARC | secreted protein acidic and cysteine |
| | | | rich |
| RARRES1 | retinoic acid receptor responder 1 | VAV1 | vav guanine nucleotide exchange |
| | | | factor 1 |
| COL4A1 | collagen type IV alpha 1 chain | SUCNR1 | succinate receptor 1 |
| PAX1 | paired box 1 | ARHGAP4 | Rho GTPase activating protein 4 |
| KRT6A | keratin 6A | GLP1R | glucagon like peptide 1 receptor |
| STEAP4 | STEAP4 metalloreductase | MYOZ3 | myozenin 3 |
| CXCL3 | C-X-C motif chemokine ligand 3 | GLIPR1 | GLI pathogenesis related 1 |
| MORC1 | MORC family CW-type zinc finger 1 | ERG | ETS transcription factor ERG |
| DSG3 | desmoglein 3 | SEMA7A | semaphorin 7A (John Milton Hagen |
| | | | blood group) |
| SPAG17 | sperm associated antigen 17 | UCN2 | urocortin 2 |
| BAALC | BAALC binder of MAP3K1 and | PROX1 | prospero homeobox 1 |
| | KLF4 | | |
| RNASE1 | ribonuclease A family member 1, | TMCC3 | transmembrane and coiled-coil |
| | pancreatic | | domain family 3 |
| RGS21 | regulator of G protein signaling 21 | GJB3 | gap junction protein beta 3 |
| HLA-DRB1 | major histocompatibility complex, | APCDD1 | APC down-regulated 1 |
| | class II, DR beta 1 | | |
| CLDN14 | claudin 14 | AZGP1 | alpha-2-glycoprotein 1, zinc-binding |
| ARSI | arylsulfatase family member I | FAM101A | #N/A |
| CST6 | cystatin E/M | TMC5 | transmembrane channel like 5 |
| CEACAM5 | CEA cell adhesion molecule 5 | SERPINA6 | serpin family A member 6 |
| GPRC5A | G protein-coupled receptor class C | CDH13 | cadherin 13 |
| | group 5 member A | | |
| LRP2 | LDL receptor related protein 2 | PRLR | prolactin receptor |
| DMBT1 | deleted in malignant brain tumors 1 | FGF19 | fibroblast growth factor 19 |
| LYPD3 | LY6/PLAUR domain containing 3 | C1QTNF2 | C1q and TNF related 2 |
| VIP | vasoactive intestinal peptide | KCNMB1 | potassium calcium-activated |
| | | | channel subfamily M regulatory beta |
| | | | subunit 1 |
| GPNMB | glycoprotein nmb | ARMC3 | armadillo repeat containing 3 |
| MMP1 | matrix metallopeptidase 1 | SLC3A1 | solute carrier family 3 member 1 |
| ITGA5 | integrin subunit alpha 5 | COL9A2 | collagen type IX alpha 2 chain |
| VNN2 | vanin 2 | ELFN2 | extracellular leucine rich repeat and |
| | | | fibronectin type III domain |
| | | | containing 2 |
| THBD | thrombomodulin | COL27A1 | collagen type XXVII alpha 1 chain |
| IQGAP2 | IQ motif containing GTPase | MUC5AC | mucin 5AC, oligomeric mucus/gel- |
| | activating protein 2 | | forming |
| ST18 | ST18 C2H2C-type zinc finger | PGLYRP4 | peptidoglycan recognition protein 4 |
| | transcription factor | | |
| CNDP1 | carnosine dipeptidase 1 | CXCL12 | C-X-C motif chemokine ligand 12 |
| BCL2A1 | BCL2 related protein A1 | DNAI1 | dynein axonemal intermediate chain |
| | | | 1 |
| ESR1 | estrogen receptor 1 | L1TD1 | LINE1 type transposase domain |
| | | | containing 1 |
| KMO | kynurenine 3-monooxygenase | CAPSL | calcyphosine like |
| CXCL6 | C-X-C motif chemokine ligand 6 | CLPSL2 | colipase like 2 |
| CCL19 | C-C motif chemokine ligand 19 | MAP3K8 | mitogen-activated protein kinase |
| | | | kinase kinase 8 |
| PLCB2 | phospholipase C beta 2 | GPR132 | G protein-coupled receptor 132 |
| ATP6V0A4 | ATPase H+ transporting V0 subunit | COLGALT2 | collagen beta(1- |
| | a4 | | O)galactosyltransferase 2 |
| IGF2BP1 | insulin like growth factor 2 mRNA | ZEB2 | zinc finger E-box binding homeobox |
| | binding protein 1 | | 2 |
| CAPN13 | calpain 13 | KCNH6 | potassium voltage-gated channel |
| | | | subfamily H member 6 |
| NES | nestin | CHST4 | carbohydrate sulfotransferase 4 |
| TNFAIP3 | TNF alpha induced protein 3 | HLA-DMA | major histocompatibility complex, |
| | | | class II, DM alpha |
| C11orf96 | chromosome 11 open reading | ATP6V1G3 | ATPase H+ transporting V1 subunit |
| | frame 96 | | G3 |
| SPARCL1 | SPARC like 1 | WNT5B | Wnt family member 5B |
| CCL5 | C-C motif chemokine ligand 5 | B3GNT7 | UDP-GlcNAc:betaGal beta-1,3-N- |
| | | | acetylglucosaminyltransferase 7 |
| HAS2 | hyaluronan synthase 2 | C15orf48 | chromosome 15 open reading frame |
| | | | 48 |
| COL2A1 | collagen type II alpha 1 chain | KRT23 | keratin 23 |
| PAK3 | p21 (RAC1) activated kinase 3 | CSPG4 | chondroitin sulfate proteoglycan 4 |
| ADH1C | alcohol dehydrogenase 1C (class | CALHM3 | calcium homeostasis modulator 3 |
| | I), gamma polypeptide | | |
| CEACAM7 | CEA cell adhesion molecule 7 | FAM20A | FAM20A golgi associated secretory |
| | | | pathway pseudokinase |
| ALOX15 | arachidonate 15-lipoxygenase | EPHB3 | EPH receptor B3 |
| STX11 | syntaxin 11 | HS6ST3 | heparan sulfate 6-O- |
| | | | sulfotransferase 3 |
| BMP2 | bone morphogenetic protein 2 | GBP1 | guanylate binding protein 1 |
| AQP5 | aquaporin 5 | EPHX3 | epoxide hydrolase 3 |
| FAM216B | family with sequence similarity 216 | AVIL | advillin |
| | member B | | |
| MMP16 | matrix metallopeptidase 16 | ITGB6 | integrin subunit beta 6 |
| THY1 | Thy-1 cell surface antigen | PPP1R17 | protein phosphatase 1 regulatory |
| | | | subunit 17 |
| FAM83A | family with sequence similarity 83 | S100A3 | S100 calcium binding protein A3 |
| | member A | | |
| LIN28B | lin-28 homolog B | NCKAP5 | NCK associated protein 5 |
| TEK | TEK receptor tyrosine kinase | ITIH5 | inter-alpha-trypsin inhibitor heavy |
| | | | chain 5 |
| CERS3 | ceramide synthase 3 | HSD17B13 | hydroxysteroid 17-beta |
| | | | dehydrogenase 13 |
| CXCL17 | C-X-C motif chemokine ligand 17 | LY6K | lymphocyte antigen 6 family |
| | | | member K |
| GNAT3 | G protein subunit alpha transducin | CSTA | cystatin A |
| | 3 | | |
| PTN | pleiotrophin | GUCY1A2 | guanylate cyclase 1 soluble subunit |
| | | | alpha 2 |
| COL1A2 | collagen type I alpha 2 chain | HLA-DQA2 | major histocompatibility complex, |
| | | | class II, DQ alpha 2 |
| MFAP2 | microfibril associated protein 2 | PKNOX2 | PBX/knotted 1 homeobox 2 |
| SERPINB3 | serpin family B member 3 | ROPN1L | rhophilin associated tail protein 1 |
| | | | like |
| MATK | megakaryocyte-associated tyrosine | INSRR | insulin receptor related receptor |
| | kinase | | |
| DEFB4A | defensin beta 4A | SLIT1 | slit guidance ligand 1 |
| UGT2B15 | UDP glucuronosyltransferase | CFAP221 | cilia and flagella associated protein |
| | family 2 member B15 | | 221 |
| RHCG | Rh family C glycoprotein | HTR3C | 5-hydroxytryptamine receptor 3C |
| MYEOV | myeloma overexpressed | ALPL | alkaline phosphatase, |
| | | | biomineralization associated |
| SAA2 | serum amyloid A2 | STMN2 | stathmin 2 |
| SFTPA2 | surfactant protein A2 | GRASP | #N/A |
| UBD | ubiquitin D | EDIL3 | EGF like repeats and discoidin |
| | | | domains 3 |
| LIF | LIF interleukin 6 family cytokine | SLC30A10 | solute carrier family 30 member 10 |
| APOD | apolipoprotein D | ACKR2 | atypical chemokine receptor 2 |
| SERPINB11 | serpin family B member 11 | VGLL1 | vestigial like family member 1 |
| ASCL1 | achaete-scute family bHLH | NCKAP1L | NCK associated protein 1 like |
| | transcription factor 1 | | |
| CYP2C18 | cytochrome P450 family 2 | GABRE | gamma-aminobutyric acid type A |
| | subfamily C member 18 | | receptor subunit epsilon |
| GRIA3 | glutamate ionotropic receptor | C9orf135 | #N/A |
| | AMPA type subunit 3 | | |
| ALPK2 | alpha kinase 2 | HPGD | 15-hydroxyprostaglandin |
| | | | dehydrogenase |
| TSPAN8 | tetraspanin 8 | CARD17 | #N/A |
| GBP6 | guanylate binding protein family | EDN2 | endothelin 2 |
| | member 6 | | |
| IL24 | interleukin 24 | RYR1 | ryanodine receptor 1 |
| CRCT1 | cysteine rich C-terminal 1 | MRAS | muscle RAS oncogene homolog |
| REG4 | regenerating family member 4 | HDC | histidine decarboxylase |
| MAL | mal, T cell differentiation protein | MRAP2 | melanocortin 2 receptor accessory |
| | | | protein 2 |
| IL36RN | interleukin 36 receptor antagonist | GRIN2B | glutamate ionotropic receptor NMDA |
| | | | type subunit 2B |
| LCN2 | lipocalin 2 | CYP3A7 | cytochrome P450 family 3 subfamily |
| | | | A member 7 |
| CCDC129 | #N/A | SOCS3 | suppressor of cytokine signaling 3 |
| CLCA2 | chloride channel accessory 2 | MT1M | metallothionein 1M |
| IRX6 | iroquois homeobox 6 | ADGRA2 | adhesion G protein-coupled |
| | | | receptor A2 |
| TMPRSS11F | transmembrane serine protease | SERPINA5 | serpin family A member 5 |
| | 11F | | |
| ST6GALNAC5 | ST6 N-acetylgalactosaminide | DNAH9 | dynein axonemal heavy chain 9 |
| | alpha-2,6-sialyltransferase 5 | | |
| PCP4 | Purkinje cell protein 4 | ADAMTS5 | ADAM metallopeptidase with |
| | | | thrombospondin type 1 motif 5 |
| IL19 | interleukin 19 | TNNI2 | troponin I2, fast skeletal type |
| POU2F3 | POU class 2 homeobox 3 | PIK3AP1 | phosphoinositide-3-kinase adaptor |
| | | | protein 1 |
| CXCL10 | C-X-C motif chemokine ligand 10 | DAW1 | dynein assembly factor with WD |
| | | | repeats 1 |
| GRIN2A | glutamate ionotropic receptor | PLXDC2 | plexin domain containing 2 |
| | NMDA type subunit 2A | | |
| DAZ2 | deleted in azoospermia 2 | WNT11 | Wnt family member 11 |
| KIAA1210 | KIAA1210 | TLR1 | toll like receptor 1 |
| ICAM1 | intercellular adhesion molecule 1 | CARD18 | caspase recruitment domain family |
| | | | member 18 |
| PTPRT | protein tyrosine phosphatase | C1orf186 | #N/A |
| | receptor type T | | |
| GJB2 | gap junction protein beta 2 | CARD16 | caspase recruitment domain family |
| | | | member 16 |
| COL5A3 | collagen type V alpha 3 chain | ALPK3 | alpha kinase 3 |
| P3H2 | prolyl 3-hydroxylase 2 | 44896 | #N/A |
| CA2 | carbonic anhydrase 2 | TNNT3 | troponin T3, fast skeletal type |
| CORO2B | coronin 2B | MOXD1 | monooxygenase DBH like 1 |
| C1orf61 | #N/A | CCL7 | C-C motif chemokine ligand 7 |
| CXorf49B | chromosome X open reading frame | CFAP46 | cilia and flagella associated protein |
| | 49B | | 46 |
| VIT | vitrin | PLA2G7 | phospholipase A2 group VII |
| SLC27A6 | solute carrier family 27 member 6 | AVPR2 | arginine vasopressin receptor 2 |
| LBH | LBH regulator of WNT signaling | LAMB4 | laminin subunit beta 4 |
| | pathway | | |
| NTRK3 | neurotrophic receptor tyrosine | FUT6 | fucosyltransferase 6 |
| | kinase 3 | | |
| SOSTDC1 | sclerostin domain containing 1 | RRAD | RRAD, Ras related glycolysis |
| | | | inhibitor and calcium channel |
| | | | regulator |
| MSI1 | musashi RNA binding protein 1 | SPHKAP | SPHK1 interactor, AKAP domain |
| | | | containing |
| MUC4 | mucin 4, cell surface associated | PADI6 | peptidyl arginine deiminase 6 |
| DPYS | dihydropyrimidinase | FAM81B | family with sequence similarity 81 |
| | | | member B |
| DOCK10 | dedicator of cytokinesis 10 | IL33 | interleukin 33 |
| NWD2 | NACHT and WD repeat domain | CCDC170 | coiled-coil domain containing 170 |
| | containing 2 | | |
| C6orf222 | #N/A | TRIM22 | tripartite motif containing 22 |
| RASSF2 | Ras association domain family | IL18 | interleukin 18 |
| | member 2 | | |
| IL1R2 | interleukin 1 receptor type 2 | SPAG6 | sperm associated antigen 6 |
| DLC1 | DLC1 Rho GTPase activating | LTB | lymphotoxin beta |
| | protein | | |
| GAS7 | growth arrest specific 7 | DYSF | dysferlin |
| HCG22 | HLA complex group 22 | ACADL | acyl-CoA dehydrogenase long chain |
| | (gene/pseudogene) | | |
| COL5A2 | collagen type V alpha 2 chain | CRIP1 | cysteine rich protein 1 |
| CTNND2 | catenin delta 2 | ADGRL3 | adhesion G protein-coupled |
| | | | receptor L3 |
| TMPRSS5 | transmembrane serine protease 5 | WDR38 | WD repeat domain 38 |
| MSC | musculin | ACTL8 | actin like 8 |
| LRRC4 | leucine rich repeat containing 4 | MYO3B | myosin IIIB |
| ANO2 | anoctamin 2 | ADGB | androglobin |
| HEPACAM2 | HEPACAM family member 2 | COL8A1 | collagen type VIII alpha 1 chain |
| OLFM2 | olfactomedin 2 | GPR87 | G protein-coupled receptor 87 |
| GABRQ | gamma-aminobutyric acid type A | MFNG | MFNG O-fucosylpeptide 3-beta-N- |
| | receptor subunit theta | | acetylglucosaminyltransferase |
| ANOS1 | anosmin 1 | FOXF1 | forkhead box F1 |
| UPK1B | uroplakin 1B | FAM135B | family with sequence similarity 135 |
| | | | member B |
| RSPO4 | R-spondin 4 | S100A4 | S100 calcium binding protein A4 |
| SDR9C7 | short chain dehydrogenase/ | BSND | barttin CLCNK type accessory |
| | reductase family 9C member 7 | | subunit beta |
| KRT16 | keratin 16 | CPAMD8 | C3 and PZP like alpha-2- |
| | | | macroglobulin domain containing 8 |
| ACE2 | angiotensin converting enzyme 2 | MX1 | MX dynamin like GTPase 1 |
| CYP4X1 | cytochrome P450 family 4 | RHBG | Rh family B glycoprotein |
| | subfamily X member 1 | | |
| CDH11 | cadherin 11 | CDC42EP1 | CDC42 effector protein 1 |
| TNFSF8 | TNF superfamily member 8 | LMO2 | LIM domain only 2 |
| NRK | Nik related kinase | NR4A3 | nuclear receptor subfamily 4 group |
| | | | A member 3 |
| CRNN | cornulin | HAS3 | hyaluronan synthase 3 |
| COL14A1 | collagen type XIV alpha 1 chain | KCNN3 | potassium calcium-activated |
| | | | channel subfamily N member 3 |
| CDH4 | cadherin 4 | TTBK1 | tau tubulin kinase 1 |
| PSCA | prostate stem cell antigen | IL1R1 | interleukin 1 receptor type 1 |
| AOC1 | amine oxidase copper containing 1 | CBX2 | chromobox 2 |
| CFB | complement factor B | IFI16 | interferon gamma inducible protein |
| | | | 16 |
| COL9A3 | collagen type IX alpha 3 chain | LY6H | lymphocyte antigen 6 family |
| | | | member H |
| SCGB1A1 | secretoglobin family 1A member 1 | MYL9 | myosin light chain 9 |
| OLIG2 | oligodendrocyte transcription factor | HTR7 | 5-hydroxytryptamine receptor 7 |
| | 2 | | |
| EMILIN3 | elastin microfibril interfacer 3 | SLC17A8 | solute carrier family 17 member 8 |
| CAPN8 | calpain 8 | IFITM3 | interferon induced transmembrane |
| | | | protein 3 |
| CARD6 | caspase recruitment domain family | SNTN | sentan, cilia apical structure protein |
| | member 6 | | |
| FBLN5 | fibulin 5 | SRPX2 | sushi repeat containing protein X- |
| | | | linked 2 |
| TNFSF14 | TNF superfamily member 14 | FGF18 | fibroblast growth factor 18 |
| BNC1 | basonuclin 1 | PZP | PZP alpha-2-macroglobulin like |
| SYT8 | synaptotagmin 8 | COL17A1 | collagen type XVII alpha 1 chain |
| SLC6A11 | solute carrier family 6 member 11 | RIN3 | Ras and Rab interactor 3 |
| CLDN1 | claudin 1 | MOGAT2 | monoacylglycerol O-acyltransferase |
| | | | 2 |
| ENDOU | endonuclease, poly(U) specific | PDGFRA | platelet derived growth factor |
| | | | receptor alpha |
| PDZK1IP1 | PDZK1 interacting protein 1 | CYP2S1 | cytochrome P450 family 2 subfamily |
| | | | S member 1 |
| ADGRG7 | adhesion G protein-coupled | SLC52A3 | solute carrier family 52 member 3 |
| | receptor G7 | | |
| KCNJ12 | potassium inwardly rectifying | GAL | galanin and GMAP prepropeptide |
| | channel subfamily J member 12 | | |
| IFI44 | interferon induced protein 44 | CASP1 | caspase 1 |
| CAPN6 | calpain 6 | PLAC4 | placenta enriched 4 |
| HLA-DMB | major histocompatibility complex, | SLC15A2 | solute carrier family 15 member 2 |
| | class II, DM beta | | |
| CXCL11 | C-X-C motif chemokine ligand 11 | PDE1C | phosphodiesterase 1C |
| GPRC6A | G protein-coupled receptor class C | PDCD1LG2 | programmed cell death 1 ligand 2 |
| | group 6 member A | | |
| LYPD1 | LY6/PLAUR domain containing 1 | TSPAN12 | tetraspanin 12 |
| MYBPC2 | myosin binding protein C2 | NDRG2 | NDRG family member 2 |
| HLA-DRB5 | major histocompatibility complex, | SALL4 | spalt like transcription factor 4 |
| | class II, DR beta 5 | | |
| IGSF21 | immunoglobin superfamily member | UNC5C | unc-5 netrin receptor C |
| | 21 | | |
| ATP8A2 | ATPase phospholipid transporting | GABRA3 | gamma-aminobutyric acid type A |
| | 8A2 | | receptor subunit alpha3 |
| COL8A2 | collagen type VIII alpha 2 chain | DAPK1 | death associated protein kinase 1 |
| NEUROD1 | neuronal differentiation 1 | ANO1 | anoctamin 1 |
| KCNB2 | potassium voltage-gated channel | DKK2 | dickkopf WNT signaling pathway |
| | subfamily B member 2 | | inhibitor 2 |
| VSIG2 | V-set and immunoglobulin domain | RASAL1 | RAS protein activator like 1 |
| | containing 2 | | |
| FOXL1 | forkhead box L1 | GBP4 | guanylate binding protein 4 |
| CASP14 | caspase 14 | PLBD1 | phospholipase B domain containing |
| | | | 1 |
| AKR1B15 | aldo-keto reductase family 1 | PRR29 | proline rich 29 |
| | member B15 | | |
| F3 | coagulation factor III, tissue factor | SHROOM4 | shroom family member 4 |
| NMU | neuromedin U | CACNA1A | calcium voltage-gated channel |
| | | | subunit alpha1 A |
| TM4SF1 | transmembrane 4 L six family | LMO3 | LIM domain only 3 |
| | member 1 | | |
| TEKT1 | tektin 1 | GABRB2 | gamma-aminobutyric acid type A |
| | | | receptor subunit beta2 |
| FSD1 | fibronectin type III and SPRY | KCNQ3 | potassium voltage-gated channel |
| | domain containing 1 | | subfamily Q member 3 |
| CLDN8 | claudin 8 | IGDCC3 | immunoglobulin superfamily DCC |
| | | | subclass member 3 |
| OAS2 | 2′-5′-oligoadenylate synthetase 2 | BCAT1 | branched chain amino acid |
| | | | transaminase 1 |
| CYYR1 | cysteine and tyrosine rich 1 | UNC5D | unc-5 netrin receptor D |
| ANKRD33B | ankyrin repeat domain 33B | RNF152 | ring finger protein 152 |
| ACTG2 | actin gamma 2, smooth muscle | HMGCS2 | 3-hydroxy-3-methylglutaryl-CoA |
| | | | synthase 2 |
| DNAH10 | dynein axonemal heavy chain 10 | IGFBP7 | insulin like growth factor binding |
| | | | protein 7 |
| FOLR1 | folate receptor alpha | COL19A1 | collagen type XIX alpha 1 chain |
| ZNF469 | zinc finger protein 469 | KLHDC7A | kelch domain containing 7A |
| SELE | selectin E | AKAP12 | A-kinase anchoring protein 12 |
| PLA2G2A | phospholipase A2 group IIA | CDH6 | cadherin 6 |
| MRVI1 | #N/A | LIPH | lipase H |
| TNFAIP6 | TNF alpha induced protein 6 | EPGN | epithelial mitogen |
| PLAU | plasminogen activator, urokinase | TYRP1 | tyrosinase related protein 1 |
| ERBB4 | erb-b2 receptor tyrosine kinase 4 | SERTAD4 | SERTA domain containing 4 |
| CFAP126 | cilia and flagella associated protein | LSP1 | lymphocyte specific protein 1 |
| | 126 | | |
| CDH17 | cadherin 17 | WNT9A | Wnt family member 9A |
| PTHLH | parathyroid hormone like hormone | KCNS1 | potassium voltage-gated channel |
| | | | modifier subfamily S member 1 |
| DAZ4 | deleted in azoospermia 4 | C4orf19 | chromosome 4 open reading frame |
| | | | 19 |
| GJA1 | gap junction protein alpha 1 | TYMP | thymidine phosphorylase |
| PIK3C2G | phosphatidylinositol-4-phosphate 3- | ADGRF1 | adhesion G protein-coupled |
| | kinase catalytic subunit type 2 | | receptor F1 |
| | gamma | | |
| XAF1 | XIAP associated factor 1 | OVOL1 | ovo like transcriptional repressor 1 |
| RELN | reelin | TGFBI | transforming growth factor beta |
| | | | induced |
| EREG | epiregulin | TLR9 | toll like receptor 9 |
| GSDMC | gasdermin C | C8orf4 | #N/A |
| RSAD2 | radical S-adenosyl methionine | FBXO39 | F-box protein 39 |
| | domain containing 2 | | |
| GPR88 | G protein-coupled receptor 88 | SAMD9L | sterile alpha motif domain |
| | | | containing 9 like |
| GJC1 | gap junction protein gamma 1 | CYP7B1 | cytochrome P450 family 7 subfamily |
| | | | B member 1 |
| AKR1B10 | aldo-keto reductase family 1 | SYNPO | synaptopodin |
| | member B10 | | |
| CLCA4 | chloride channel accessory 4 | TRIM49C | tripartite motif containing 49C |
| SCIN | scinderin | ERICH3 | glutamate rich 3 |
| TGFB2 | transforming growth factor beta 2 | ADAM12 | ADAM metallopeptidase domain 12 |
| NCF4 | neutrophil cytosolic factor 4 | LRRC31 | leucine rich repeat containing 31 |
| TMPRSS2 | transmembrane serine protease 2 | OR2W3 | olfactory receptor family 2 subfamily |
| | | | W member 3 |
| HOXC8 | homeobox C8 | KIF19 | kinesin family member 19 |
| DNASE1L3 | deoxyribonuclease 1 like 3 | DCLK3 | doublecortin like kinase 3 |
| TMEM100 | transmembrane protein 100 | DIRAS2 | DIRAS family GTPase 2 |
| OLIG1 | oligodendrocyte transcription factor | RFTN1 | raftlin, lipid raft linker 1 |
| | 1 | | |
| SULF2 | sulfatase 2 | SERPING1 | serpin family G member 1 |
| BGN | biglycan | TMEM40 | transmembrane protein 40 |
| IFI6 | interferon alpha inducible protein 6 | TRIM49D1 | tripartite motif containing 49D1 |
| CYP24A1 | cytochrome P450 family 24 | FAM25A | family with sequence similarity 25 |
| | subfamily A member 1 | | member A |
| SPRR1A | small proline rich protein 1A | GEM | GTP binding protein overexpressed |
| | | | in skeletal muscle |
| CHP2 | calcineurin like EF-hand protein 2 | NEURL3 | neuralized E3 ubiquitin protein |
| | | | ligase 3 |
| SMOC2 | SPARC related modular calcium | FZD8 | frizzled class receptor 8 |
| | binding 2 | | |
| TLX3 | T cell leukemia homeobox 3 | NPFFR1 | neuropeptide FF receptor 1 |
| ATP10A | ATPase phospholipid transporting | PLA2G4D | phospholipase A2 group IVD |
| | 10A (putative) | | |
| P2RY8 | P2Y receptor family member 8 | KCNIP4 | potassium voltage-gated channel |
| | | | interacting protein 4 |
| FAM46B | #N/A | EPHA2 | EPH receptor A2 |
| PENK | proenkephalin | ADAMTS18 | ADAM metallopeptidase with |
| | | | thrombospondin type 1 motif 18 |
| AGTR1 | angiotensin II receptor type 1 | DKK3 | dickkopf WNT signaling pathway |
| | | | inhibitor 3 |
| KRT2 | keratin 2 | RSPO3 | R-spondin 3 |
| NOXO1 | NADPH oxidase organizer 1 | ANTXR1 | ANTXR cell adhesion molecule 1 |
| BST2 | bone marrow stromal cell antigen 2 | EPHX4 | epoxide hydrolase 4 |
| PRDM1 | PR/SET domain 1 | IL12RB2 | interleukin 12 receptor subunit beta |
| | | | 2 |
| HECW2 | HECT, C2 and WW domain | PALM3 | paralemmin 3 |
| | containing E3 ubiquitin protein | | |
| | ligase 2 | | |
| C10orf99 | #N/A | TSPAN18 | tetraspanin 18 |
| SERPINA1 | serpin family A member 1 | METRNL | meteorin like, glial cell differentiation |
| | | | regulator |
| SCUBE2 | signal peptide, CUB domain and | C16orf54 | chromosome 16 open reading frame |
| | EGF like domain containing 2 | | 54 |
| HLA-DQA1 | major histocompatibility complex, | TUBB2B | tubulin beta 2B class IIb |
| | class II, DQ alpha 1 | | |
| CLEC1A | C-type lectin domain family 1 | CMPK2 | cytidine/uridine monophosphate |
| | member A | | kinase 2 |
| GSTA1 | glutathione S-transferase alpha 1 | MGAT5B | alpha-1,6-mannosylglycoprotein 6- |
| | | | beta-N-acetylglucosaminyltransferase B |
| FAM92B | #N/A | BBOX1 | gamma-butyrobetaine hydroxylase 1 |
| MUC16 | mucin 16, cell surface associated | CACNA1E | calcium voltage-gated channel |
| | | | subunit alpha1 E |
| HLA-DOA | major histocompatibility complex, | PTGS1 | prostaglandin-endoperoxide |
| | class II, DO alpha | | synthase 1 |
| C16orf89 | chromosome 16 open reading | HSPB7 | heat shock protein family B (small) |
| | frame 89 | | member 7 |
| SLC22A3 | solute carrier family 22 member 3 | TAGLN | transgelin |
| CD74 | CD74 molecule | IL11 | interleukin 11 |
| KDR | kinase insert domain receptor | CHRM1 | cholinergic receptor muscarinic 1 |
| EPS8L3 | EPS8 like 3 | TDRP | testis development related protein |
| CROCC2 | ciliary rootlet coiled-coil, rootletin | EFS | embryonal Fyn-associated substrate |
| | family member 2 | | |
| CDC42EP5 | CDC42 effector protein 5 | ATP6V1C2 | ATPase H+ transporting V1 subunit |
| | | | C2 |
| CHSY3 | chondroitin sulfate synthase 3 | ELMOD1 | ELMO domain containing 1 |
| CFI | complement factor I | CLEC2B | C-type lectin domain family 2 |
| | | | member B |
| C1QTNF1 | C1q and TNF related 1 | CCND1 | cyclin D1 |
| SLC28A3 | solute carrier family 28 member 3 | MYPN | myopalladin |
| PTPRZ1 | protein tyrosine phosphatase | RARRES3 | #N/A |
| | receptor type Z1 | | |
| GRHL3 | grainyhead like transcription factor | SEMA4A | semaphorin 4A |
| | 3 | | |
| LRRN2 | leucine rich repeat neuronal 2 | SERPINA9 | serpin family A member 9 |
| DSC3 | desmocollin 3 | ANXA13 | annexin A13 |
| SORCS2 | sortilin related VPS10 domain | TRPM8 | transient receptor potential cation |
| | containing receptor 2 | | channel subfamily M member 8 |

### Example 2: Testing of Prioritized Candidate Genes and Pathways for their Effect on NEtD in the PARCB Model

The inventors seek to determine the impact of regulatory (epigenetics, transcription, signaling) events on promoting or inhibiting the transdifferentiation process. Their preliminary data has generated a prioritized list of candidate critical transcription factors (e.g., ASCL2), signaling molecules (e.g., BMX, TEK, AVIL) and physiological processes (e.g., inflammation, angiogenesis, wound healing) implicated in contributing to transdifferentiation. The inventors can test the impact of these mechanisms on promoting or inhibiting transdifferentiation through genetic perturbation and pharmacological inhibition of implicated mechanisms in the PARCB model system.

The inventors propose to test the contribution of prioritized candidate genes to promoting, inhibiting, or altering the NEtD trajectory, with an imbedded goal of identifying therapeutic candidates for blocking NEtD. The inventors can focus on modulating critical transcription factors implicated in NEPC (and SCLC), with a goal of more fully defining the epigenetic and transcriptional circuitry that regulates the small cell neuroendocrine state and substrates in prostate cancer.

Prioritized candidates from the PARCB temporal preliminary data, and comparisons to other cancer transdifferentiation and dedifferentiation mechanisms, as well as from integration of data from normal cell differentiation or reprogramming (iPSC) programs can be tested. The genes upregulated in the PARCB temporal data during the transition stage were rank ordered, and enrichment analysis was performed (FIG. 14). The enriched gene sets included the categories of inflammation (NFκB, JAK/STAT), angiogenesis, and wound healing. Cell type inference analysis (SingleR) also demonstrated the transitional cells were enriched in stem and iPSC programs (not shown). Gene-based analysis revealed additional gene candidates in these and other pathways (FIG. 14).

Experimental procedures: The inventors can start with a pipeline for exogenous expression and knockdown/out. When available, in vivo drugs can be tested (e.g. Nf-kB (Gamble C et al., Br J Pharmacol. 2012; 165 (4): 802-819), Tenascin C (Midwood KS et al., J Cell Commun Signal. 2009 December; 3 (3-4): 287-310), AVIL/Hedgehog (Bariwal J, et al., Med Res Rev. 2019 May; 39 (3): 1137-1204. And Jamieson C. et al., Blood Cancer Discov. 2020 Sep. 1;1 (2): 134-145), BMX (Chen S, et al., Cancer Res. 2018 Sep. 15;78 (18): 5203-5215 and Jarboe JS et al., Recent Patents Anticancer Drug Discov. 2013 September; 8 (3): 228-238)/TEK (Saharinen P. et al., Nat Rev Drug Discov. Nature Publishing Group; 2017 September; 16 (9): 635-661)). Exogenous expression or gene knockdown/out constructs can be incorporated into the PARCB lentiviral cocktail. For example, the NF-κB pathway can be modulated by alterations of both the core (e.g. RELA/p65) and the negative feedback (e.g. IKK complex) proteins in the pathway, expression of a dominant negative forms of the IKK complex proteins (Pasparakis M. et al., Cell Death Differ. Nature Publishing Group; 2006 May; 13 (5): 861-872), and using small molecule inhibitors. Perturbations and inhibitors of the JAK/STAT pathway can be tested as positive controls for impact on transdifferentiation. Tumors with candidate pathways perturbed can be collected at 4-, 6-, 8- and 10-week timepoints, and subjected to Rhapsody platform-based targeted resequencing to determine where they fall on the established PARCB NEtD trajectory. If higher throughput is needed, the PARCB forward genetics systems provides the flexibility to do small scale screens (n=˜ 25 genes).

The inventors have found that many (n=˜ 20 identified thus far) development and differentiation programs lead to an arc-like trajectory when the corresponding transcriptome or epigenome data is analyzed by unbiased PCA. While some details of these programs may be context specific, the inventors hypothesize that others can be shared between multiple programs, such as epigenetic loosening to allow for differentiation (see above). Thus, they can use integrative bioinformatic analysis of arc-like trajectories (transcriptomic, and available epigenetic data (e.g., existing PARCB data)) to identify genes and pathways commonly upregulated in multiple differentiation arcs.

The inventors anticipate that a subset of the tested perturbations will alter the transdifferentiation kinetics or trajectory. In experiments designed to knockdown/knockout a gene involved in promoting NEtD, other related factors may be redundant. When possible, the inventors may target convergence points (bottle neck points) of pathways (e.g. targeting NF-κB/IKK rather than upstream signaling). Additionally, the approach of exogenous expression to promote NEtD will in general be less prone to redundancy effects. While this proposal is focused on in vivo experiments, the inventors can watch for results that suggest ways to build an NEtD in vitro or organoid model system (note the PARCB model involves an organoid step that can be an experimental platform).

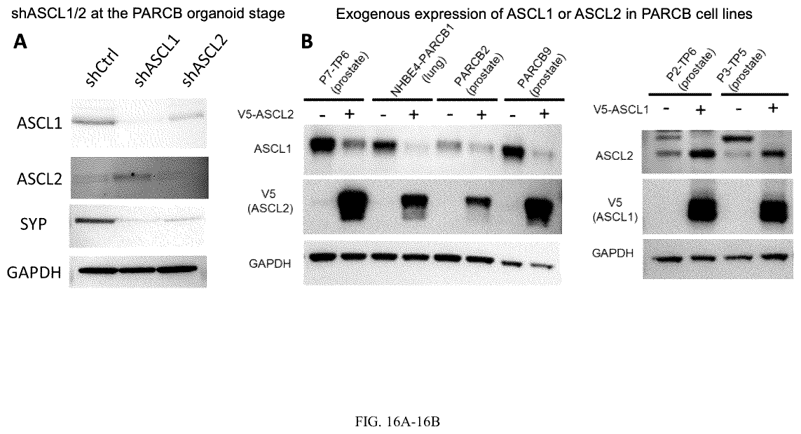

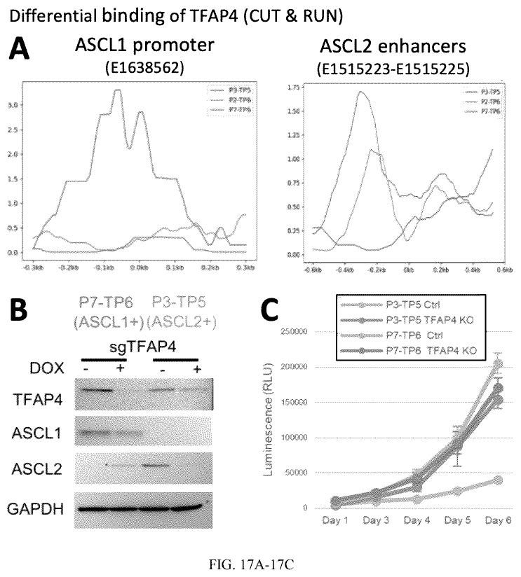

The inventors propose to define the core transcriptional regulatory circuitries that impact neuroendocrine transdifferentiation. Using in vitro experiments, the inventors can define the regulatory circuitry between transdifferentiation factors (FIG. 16-17); and using in vivo experiments, the inventors can test how these factors alter the course of NEtD, as it is critical to understand how these factors impact the trajectory of transdifferentiation, for which ex vivo models do not exist.

The inventors' PARCB temporal trajectory data identified a bifurcation to either a ASCL1-positive or ASCL2-positive endpoint. The ASCL2+state was also POU2F3+. In the PARCB model, ASCL1 and ASCL2 expression levels are mutually exclusive in single cells during NEPC trans-differentiation. This led to two hypotheses: 1) these two factors mutually regulate each other's expression, or 2) they share a common upstream transcription factor that alternates their transcription through regulated differential binding to respective gene regulatory elements. To test the first hypothesis, the inventors expressed V5-tagged ASCL2 in multiple PARCB tumor derived cell lines (lung and prostate) and observed that ASCL1 protein expression was significantly suppressed in these cells (FIG. 16). In contrast, expression of V5-tagged ASCL1 increased ASCL2 expressions both at protein and mRNA levels (FIG. 16). Thus, in the model cells ASCL1 and ASCL2 mutually regulate each other at the protein level, but each in the opposite manner.

To test the second hypothesis of a common regulator, known promoter and enhancer regions of ASCL1 and ASCL2 were first annotated in the PARCB time course ATAC-seq data. An opposing pattern of open and closed chromatin formation was found on both the ASCL1 promoter and the ASCL2 enhancer regions (not shown). A rank list of transcription factors that have matching motifs in the regions was generated to determine potential shared regulators. An extensive literature search of all the factors whose motifs were found in both ASCL1 and ASCL2 regulatory regions, revealed that TFAP4 (a.k.a. AP-4) is known to form different transcription complex to either activate or repress target genes and thus mediate cell fate decisions. The TFAP4 motif was shared in both the ASCL1 promoter (ranked 2^{nd}) and the ASCL2 enhancer region (ranked 6^{th}) in the top shared transcription factor motifs. TFAP4 is expressed across all the SCLC, NEPC patient derived and PARCB tumor derived cell lines tested.

The direct differential binding of TFAP4 to those regulatory regions was confirmed by the CUT&RUN technique, a chromatin immunoprecipitation experiment using TFAP4 antibody in both ASCL1+ and ASCL2+ PARCB tumor-derived cell lines. TFAP4 was found to have higher binding affinity near the ASCL1 promoter in ASCL1+ cell lines than ASCL2+ cell lines (FIG. 17A). In contrast, TFAP4 consistently bound to ASCL2 enhancer regions in ASCL2+ cell lines compared to ASCL1+ cell lines (FIG. 17A). This result supports that TFAP4 regulates transcription of ASCL1 and ASCL2 in a context-specific manner.

To determine whether TFAP4 directly regulates the expression of ASCL1 and ASCL2, the inventors introduced a doxycycline-inducible CRISPR sgRNA targeting TFAP4 in ASCL1+ and ASCL2+ cell lines. Both ASCL1 and ASCL2 expression decreased after the induced TFAP4 knockout by the addition of doxycycline in the respective cell lines, and interestingly, ASCL2 appeared when ASCL1 was suppressed in an ASCL1+ cell line (FIG. 17B). Cell growth assays show a mild decrease in ASCL1+ cell growth, and in contrast a drastic increase in ASCL2+growth upon the knockout of TFAP4 (FIG. 17C). Thus in the transcriptional regulatory circuit studies, the inventors found a reciprocal, non-symmetric regulatory relationship between ASCL1 and ASCL2; and within this circuit, ASCL1 and ASCL2 have a shared positive regulatory factor, TFAP4.

The inventors will seek to further refine this initial draft of the ASCL1, ASCL2 and TFAP4 circuitry. The inventors can include additional transcription factors such as NEUROD1 and POU2F3. The inventors can continue in vitro TF-modulation cell line-based experiments such as those described in FIG. 16-17, to provide first draft circuit diagrams. As an additional approach, the inventors can move the more impactful perturbations into the PARCB in vivo transformation model setting. This additional approach is important for two reasons. First, the in vitro studies require cell lines, which have already committed to their ASCL1 or ASCL2 subtype, while in vivo studies will allow assessment of the impact of the gene perturbation during the transition stage of NEtD. Second, it is recognized in the field of therapy-induced NEtD that in vivo factors are central to the transdifferentiation process.

In vitro experiments can use PARCB model-derived cell lines and lentivirus-mediated genetic engineering. Patient cancer-derived prostate cancer cell lines such as LNCaP C4-2B (adenocarcinoma that can be driven to undergo NEtD) and H660 (NEPC) can also be tested. In vivo experiments can be executed, as described previously, by adding additional genetic perturbations (exogenous expression or knockdown/knockout) to the PARCB cocktail of lentiviruses. In some cases, it may be informative to turn on or off a gene in the NEtD transition stage of the model. In these cases the inventors can use doxycycline inducible promoters. The inventors can measure both transcriptomic (RNAseq, bulk and single cell) and epigenetic (ATAC-seq) changes. They note that it was the combined analysis of transcriptomic (RNA-seq) profiles and epigenomic (ATAC-seq)-based motif analysis that led to the identification of TFAP4 as a member of the NEPC subtype circuitry (FIG. 17).

Many of the prioritized targets are implicated to impact NEPC and/or SCLC. However, much of the data, especially in prostate cancer, on these factors comes from endpoint states (i.e., post-transdifferentiation). The inventors aim to provide results on how these factors impact NEtD in a dynamic fashion during the transition states.

Tumors with candidate TFs perturbed can be collected at 4-, 6-, 8- and 10-week timepoints. In cases where the knockdown or knockout is targeting a gene expressed and potentially required at early timepoints (e.g. during lentiviral infection or organoid culture), the inventors can include an inducible approach, with induction occurring at approximately the 2-4 week mark. Tumors can be profiled by Rhapsody platform-based targeted resequencing to determine where they fall on the established PARCB NEID trajectory.

The inventors anticipate that a subset of the tested perturbations will alter the NEtD trajectory or endpoint states. For example, knockdown/knockout of ASCL1 would be expected to drive cells down the ASCL2 bifurcation pathway, and vice-versa. Their preliminary data supports that TFAP4 positively regulates both ASCL1 and ASCL2, so the outcome of TFAP4 perturbations may be more difficult to predict. The inventors anticipate that this set of experiments will further define the critical transcription factor regulatory circuit of NEPC, and the factors most impactful during the NEtD transition stage. Future experiments can test the degree of similarity in the case of SCLC subtypes and TF circuitry-which could be done using the lung SCLC version of the P ARCB model, or other SCLC models and patient resources.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Nadal, R., Schweizer, M., Kryvenko, O. N., Epstein, J. I., and Eisenberger, M. A. (2014). Small cell carcinoma of the prostate. Nat Rev Urol 11, 213-219. 10.1038/nrurol.2014.21.
2. Pietanza, M. C., Byers, L. A., Minna, J. D., and Rudin, C. M. (2015). Small cell lung cancer: will recent progress lead to improved outcomes? Clin Cancer Res 21, 2244-2255. 10.1158/1078-0432.CCR-14-2958.
3. Yamada, Y., and Beltran, H. (2021). Clinical and Biological Features of Neuroendocrine Prostate Cancer. Curr Oncol Rep 23, 15. 10.1007/s11912-020-01003-9.
4. Balanis, N. G., Sheu, K. M., Esedebe, F. N., Patel, S. J., Smith, B.A., Park, J. W., Alhani, S., Gomperts, B. N., Huang, J., Witte, O. N., and Graeber, T. G. (2019). Pan-cancer Convergence to a Small-Cell Neuroendocrine Phenotype that Shares Susceptibilities with Hematological Malignancies. Cancer Cell 36, 17-34 e17. 10.1016/j.ccell.2019.06.005.
5. Cejas, P., Xie, Y., Font-Tello, A., Lim, K., Syamala, S., Qiu, X., Tewari, A. K., Shah, N., Nguyen, H.M., Patel, R. A., et al. (2021). Subtype heterogeneity and epigenetic convergence in neuroendocrine prostate cancer. Nat Commun 12, 5775. 10.1038/s41467-021-26042-z.
6. Park, J. W., Lee, J. K., Sheu, K. M., Wang, L., Balanis, N. G., Nguyen, K., Smith, B. A., Cheng, C., Tsai, B. L., Cheng, D. H., et al. (2018). Reprogramming normal human epithelial tissues to a common, lethal neuroendocrine cancer lineage. Science 362, 91-95. 10.1126/science.aat5749.
7. Wang, L., Smith, B.A., Balanis, N.G., Tsai, B. L., Nguyen, K., Cheng, M. W., Obusan, M. B., Esedebe, F. N., Patel, S. J., Zhang, H., et al. (2020). A genetically defined disease model reveals that urothelial cells can initiate divergent bladder cancer phenotypes. Proc Natl Acad Sci USA 117, 563-572. 10.1073/pnas. 1915770117.
8. Aggarwal, R., Huang, J., Alumkal, J. J., Zhang, L., Feng, F. Y., Thomas, G. V., Weinstein, A. S., Friedl, V., Zhang, C., Witte, O. N., et al. (2018). Clinical and Genomic Characterization of Treatment-Emergent Small-Cell Neuroendocrine Prostate Cancer: A Multi-institutional Prospective Study. J Clin Oncol 36, 2492-2503. 10.1200/JCO.2017.77.6880.
9. Dicken, H., Hensley, P. J., and Kyprianou, N. (2019). Prostate tumor neuroendocrine differentiation via EMT: The road less traveled. Asian J Urol 6, 82-90. 10.1016/j.ajur.2018.11.001.
10. Beltran, H., Hruszkewycz, A., Scher, H. I., Hildesheim, J., Isaacs, J., Yu, E. Y., Kelly, K., Lin, D., Dicker, A., Arnold, J., et al. (2019). The Role of Lineage Plasticity in Prostate Cancer Therapy Resistance. Clin Cancer Res 25, 6916-6924. 10.1158/1078-0432.CCR-19-1423.
11. Davies, A., Zoubeidi, A., and Selth, L. A. (2020). The epigenetic and transcriptional landscape of neuroendocrine prostate cancer. Endocr Relat Cancer 27, R35-R50. 10.1530/ERC-19-0420.
12. Ku, S. Y., Rosario, S., Wang, Y., Mu, P., Seshadri, M., Goodrich, Z. W., Goodrich, M.M., Labbe, D.P., Gomez, E. C., Wang, J., et al. (2017). Rb1 and Trp53 cooperate to suppress prostate cancer lineage plasticity, metastasis, and antiandrogen resistance. Science 355, 78-83. 10.1126/science.aah4199.
13. Mu, P., Zhang, Z., Benelli, M., Karthaus, W. R., Hoover, E., Chen, C.C., Wongvipat, J., Ku, S.Y., Gao, D., Cao, Z., et al. (2017). SOX2 promotes lineage plasticity and antiandrogen resistance in TP53- and RB1-deficient prostate cancer. Science 355, 84-88. 10.1126/science.aah4307.
14. Adams, E. J., Karthaus, W. R., Hoover, E., Liu, D., Gruet, A., Zhang, Z., Cho, H., DiLoreto, R., Chhangawala, S., Liu, Y., et al. (2019). FOXA1 mutations alter pioneering activity, differentiation and prostate cancer phenotypes. Nature 571, 408-412. 10.1038/s41586-019-1318-9.
15. Parolia, A., Cieslik, M., Chu, S. C., Xiao, L., Ouchi, T., Zhang, Y., Wang, X., Vats, P., Cao, X., Pitchiaya, S., et al. (2019). Distinct structural classes of activating FOXA1 alterations in advanced prostate cancer. Nature 571, 413-418. 10.1038/s41586-019-1347-4.
16. Guo, H., Ci, X., Ahmed, M., Hua, J. T., Soares, F., Lin, D., Puca, L., Vosoughi, A., Xue, H., Li, E., et al. (2019). ONECUT2 is a driver of neuroendocrine prostate cancer. Nat Commun 10, 278. 10.1038/s41467-018-08133-6.
17. Rotinen, M., You, S., Yang, J., Coetzee, S. G., Reis-Sobreiro, M., Huang, W. C., Huang, F., Pan, X., Yanez, A., Hazelett, D. J., et al. (2018). ONECUT2 is a targetable master regulator of lethal prostate cancer that suppresses the androgen axis. Nat Med 24, 1887-1898. 10.1038/s41591-018-0241-1.
18. Ireland, A.S., Micinski, A.M., Kastner, D. W., Guo, B., Wait, S. J., Spainhower, K. B., Conley, C. C., Chen, O. S., Guthrie, M. R., Soltero, D., et al. (2020). MYC Drives Temporal Evolution of Small Cell Lung Cancer Subtypes by Reprogramming Neuroendocrine Fate. Cancer Cell 38, 60-78 e12. 10.1016/j.ccell.2020.05.001.
19. Marjanovic, N. D., Hofree, M., Chan, J. E., Canner, D., Wu, K., Trakala, M., Hartmann, G. G., Smith, O. C., Kim, J. Y., Evans, K. V., et al. (2020). Emergence of a High-Plasticity Cell State during Lung Cancer Evolution. Cancer Cell 38, 229-246 e213. 10.1016/j.ccell.2020.06.012.
20. Garcia-Bellido, A., and Santamaria, P. (1978). Developmental Analysis of the Achaete-Scute System of DROSOPHILA MELANOGASTER. Genetics 88, 469-486. 10.1093/genetics/88.3.469.
21. Garcia-Bellido, A., and de Celis, J. F. (2009). The complex tale of the achaete-scute complex: a paradigmatic case in the analysis of gene organization and function during development. Genetics 182, 631-639. 10.1534/genetics. 109.104083.
22. Borges, M., Linnoila, R.I., van de Velde, H.J., Chen, H., Nelkin, B. D., Mabry, M., Baylin, S. B., and Ball, D. W. (1997). An achaete-scute homologue essential for neuroendocrine differentiation in the lung. Nature 386, 852-855. 10.1038/386852a0.
23. Borromeo, M. D., Savage, T. K., Kollipara, R. K., He, M., Augustyn, A., Osborne, J. K., Girard, L., Minna, J. D., Gazdar, A. F., Cobb, M. H., and Johnson, J. E. (2016). ASCL1 and NEUROD1 Reveal Heterogeneity in Pulmonary Neuroendocrine Tumors and Regulate Distinct Genetic Programs. Cell Rep 16, 1259-1272. 10.1016/j.celrep.2016.06.081.
24. Nouruzi, S., Ganguli, D., Tabrizian, N., Kobelev, M., Sivak, O., Namekawa, T., Thaper, D., Baca, S.C., Freedman, M. L., Aguda, A., Davies, A., and Zoubeidi, A. (2022). ASCL1 activates neuronal stem cell-like lineage programming through remodeling of the chromatin landscape in prostate cancer. Nat Commun 13, 2282. 10.1038/s41467-022-29963-5.
25. Guillemot, F., Nagy, A., Auerbach, A., Rossant, J., and Joyner, A. L. (1994). Essential role of Mash-2 in extraembryonic development. Nature 371, 333-336. 10.1038/371333a0.
26. Kinoshita, M., Li, M. A., Barber, M., Mansfield, W., Dietmann, S., and Smith, A. (2021). Disabling de novo DNA methylation in embryonic stem cells allows an illegitimate fate trajectory. Proc Natl Acad Sci USA 118. 10.1073/pnas.2109475118.
27. Murata, K., Jadhav, U., Madha, S., van Es, J., Dean, J., Cavazza, A., Wucherpfennig, K., Michor, F., Clevers, H., and Shivdasani, R.A. (2020). Ascl2-Dependent Cell Dedifferentiation Drives Regeneration of Ablated Intestinal Stem Cells. Cell Stem Cell 26, 377-390 e376. 10.1016/j.stem.2019.12.011.
28. Stange, D. E., Engel, F., Longerich, T., Koo, B. K., Koch, M., Delhomme, N., Aigner, M., Toedt, G., Schirmacher, P., Lichter, P., Weitz, J., and Radlwimmer, B. (2010). Expression of an ASCL2 related stem cell signature and IGF2 in colorectal cancer liver metastases with 11p15.5 gain. Gut 59, 1236-1244. 10.1136/gut.2009.195701.
29. van der Flier, L. G., van Gijn, M. E., Hatzis, P., Kujala, P., Haegebarth, A., Stange, D. E., Begthel, H., van den Born, M., Guryev, V., Oving, I., et al. (2009). Transcription factor achaete scute-like 2 controls intestinal stem cell fate. Cell 136, 903-912. 10.1016/j.cell.2009.01.031.
30. Zhu, R., Yang, Y., Tian, Y., Bai, J., Zhang, X., Li, X., Peng, Z., He, Y., Chen, L., Pan, Q., et al. (2012). Ascl2 knockdown results in tumor growth arrest by miRNA-302b-related inhibition of colon cancer progenitor cells. PLOS One 7, e32170. 10.1371/journal.pone.0032170.
31. Brady, N. J., Bagadion, A. M., Singh, R., Conteduca, V., Van Emmenis, L., Arceci, E., Pakula, H., Carelli, R., Khani, F., Bakht, M., et al. (2021). Temporal evolution of cellular heterogeneity during the progression to advanced AR-negative prostate cancer. Nat Commun 12, 3372. 10.1038/s41467-021-23780-y.
32. George, J., Lim, J. S., Jang, S.J., Cun, Y., Ozretic, L., Kong, G., Leenders, F., Lu, X., Fernandez-Cuesta, L., Bosco, G., et al. (2015). Comprehensive genomic profiles of small cell lung cancer. Nature 524, 47-53. 10.1038/nature14664.
33. Beltran, H., Prandi, D., Mosquera, J. M., Benelli, M., Puca, L., Cyrta, J., Marotz, C., Giannopoulou, E., Chakravarthi, B. V., Varambally, S., et al. (2016). Divergent clonal evolution of castration-resistant neuroendocrine prostate cancer. Nat Med 22, 298-305. 10.1038/nm.4045.
34. Abida, W., Cyrta, J., Heller, G., Prandi, D., Armenia, J., Coleman, I., Cieslik, M., Benelli, M., Robinson, D., Van Allen, E. M., et al. (2019). Genomic correlates of clinical outcome in advanced prostate cancer. Proc Natl Acad Sci USA 116, 11428-11436. 10.1073/pnas.1902651116.
35. Labrecque, M. P., Coleman, I. M., Brown, L. G., True, L. D., Kollath, L., Lakely, B., Nguyen, H. M., Yang, Y. C., da Costa, R. M. G., Kaipainen, A., et al. (2019). Molecular profiling stratifies diverse phenotypes of treatment-refractory metastatic castration-resistant prostate cancer. J Clin Invest 129, 4492-4505. 10.1172/JCI128212.
36. Sharp, A., Welti, J.C., Lambros, M. B. K., Dolling, D., Rodrigues, D. N., Pope, L., Aversa, C., Figueiredo, I., Fraser, J., Ahmad, Z., et al. (2019). Clinical Utility of Circulating Tumour Cell Androgen Receptor Splice Variant-7 Status in Metastatic Castration-resistant Prostate Cancer. Eur Urol 76, 676-685. 10.1016/j.eururo.2019.04.006.
37. Lambert, S. A., Jolma, A., Campitelli, L. F., Das, P. K., Yin, Y., Albu, M., Chen, X., Taipale, J., Hughes, T. R., and Weirauch, M. T. (2018). The Human Transcription Factors. Cell 175, 598-599. 10.1016/j.cell.2018.09.045.
38. Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nat Methods 10, 1213-1218. 10.1038/nmeth.2688.
39. Richard, A., Boullu, L., Herbach, U., Bonnafoux, A., Morin, V., Vallin, E., Guillemin, A., Papili Gao, N., Gunawan, R., Cosette, J., et al. (2016). Single-Cell-Based Analysis Highlights a Surge in Cell-to-Cell Molecular Variability Preceding Irreversible Commitment in a Differentiation Process. PLOS Biol 14, e1002585. 10.1371/journal.pbio.1002585.
40. Shannon, C. E. (1997). The mathematical theory of communication. 1963. MD Comput 14, 306-317.
41. van Heeringen, S. J., and Veenstra, G. J. (2011). GimmeMotifs: a de novo motif prediction pipeline for ChIP-sequencing experiments. Bioinformatics 27 270-271. 10.1093/bioinformatics/btq636.
42. Smith, B. A., Balanis, N. G., Nanjundiah, A., Sheu, K. M., Tsai, B. L., Zhang, Q., Park, J. W., Thompson, M., Huang, J., Witte, O. N., and Graeber, T. G. (2018). A Human Adult Stem Cell Signature Marks Aggressive Variants across Epithelial Cancers. Cell Rep 24, 3353-3366 e3355. 10.1016/j.celrep.2018.08.062.
43. Han, M., Li, F., Zhang, Y., Dai, P., He, J., Li, Y., Zhu, Y., Zheng, J., Huang, H., Bai, F., and Gao, D. (2022). FOXA2 drives lineage plasticity and KIT pathway activation in neuroendocrine prostate cancer. Cancer Cell 40, 1306-1323 e1308. 10.1016/j.ccell.2022.10.011.
44. Park, J. W., Lee, J. K., Witte, O. N., and Huang, J. (2017). FOXA2 is a sensitive and specific marker for small cell neuroendocrine carcinoma of the prostate. Mod Pathol 30, 1262-1272. 10.1038/modpathol.2017.44.
45. Sreekumar, A., and Saini, S. (2023). Role of transcription factors and chromatin modifiers in driving lineage reprogramming in treatment-induced neuroendocrine prostate cancer. Front Cell Dev Biol 11, 1075707. 10.3389/fcell.2023.1075707.
46. Rudin, C. M., Poirier, J. T., Byers, L. A., Dive, C., Dowlati, A., George, J., Heymach, J. V., Johnson, J. E., Lehman, J. M., MacPherson, D., et al. (2019). Molecular subtypes of small cell lung cancer: a synthesis of human and mouse model data. Nat Rev Cancer 19, 289-297. 10.1038/s41568-019-0133-9.
47. Cheng, S., Prieto-Dominguez, N., Yang, S., Connelly, Z. M., StPierre, S., Rushing, B., Watkins, A., Shi, L., Lakey, M., Baiamonte, L. B., et al. (2020). The expression of YAP1 is increased in high-grade prostatic adenocarcinoma but is reduced in neuroendocrine prostate cancer. Prostate Cancer Prostatic Dis 23, 661-669. 10.1038/s41391-020-0229-z.
48. Aran, D., Looney, A. P., Liu, L., Wu, E., Fong, V., Hsu, A., Chak, S., Naikawadi, R. P., Wolters, P. J., Abate, A. R., Butte, A. J., and Bhattacharya, M. (2019). Reference-based analysis of lung single-cell sequencing reveals a transitional profibrotic macrophage. Nat Immunol 20, 163-172. 10.1038/s41590-018-0276-y.
49. He, M. X., Cuoco, M. S., Crowdis, J., Bosma-Moody, A., Zhang, Z., Bi, K., Kanodia, A., Su, M. J., Ku, S. Y., Garcia, M. M., et al. (2021). Transcriptional mediators of treatment resistance in lethal prostate cancer. Nat Med 27, 426-433. 10.1038/s41591-021-01244-6.
50. Chan, J. M., Zaidi, S., Love, J. R., Zhao, J. L., Setty, M., Wadosky, K. M., Gopalan, A., Choo, Z. N., Persad, S., Choi, J., et al. (2022). Lineage plasticity in prostate cancer depends on JAK/STAT inflammatory signaling. Science 377, 1180-1191. 10.1126/science.abn0478.
51. Dong, B., Miao, J., Wang, Y., Luo, W., Ji, Z., Lai, H., Zhang, M., Cheng, X., Wang, J., Fang, Y., et al. (2020). Single-cell analysis supports a luminal-neuroendocrine transdifferentiation in human prostate cancer. Commun Biol 3, 778. 10.1038/s42003-020-01476-1.
52. Qiu, X., Mao, Q., Tang, Y., Wang, L., Chawla, R., Pliner, H. A., and Trapnell, C. (2017). Reversed graph embedding resolves complex single-cell trajectories. Nat Methods 14, 979-982. 10.1038/nmeth.4402.
53. Bergen, V., Lange, M., Peidli, S., Wolf, F. A., and Theis, F. J. (2020). Generalizing RNA velocity to transient cell states through dynamical modeling. Nat Biotechnol 38, 1408-1414. 10.1038/s41587-020-0591-3.
54. Barker, N., van Es, J. H., Kuipers, J., Kujala, P., van den Born, M., Cozijnsen, M., Haegebarth, A., Korving, J., Begthel, H., Peters, P. J., and Clevers, H. (2007). Identification of stem cells in small intestine and colon by marker gene Lgr5. Nature 449, 1003-1007. 10.1038/nature06196.
55. Margolin, A. A., Nemenman, I., Basso, K., Wiggins, C., Stolovitzky, G., Dalla Favera, R., and Califano, A. (2006). ARACNE: an algorithm for the reconstruction of gene regulatory networks in a mammalian cellular context. BMC Bioinformatics 7 Suppl 1, S7. 10.1186/1471-2105-7-S1-S7.
56. Gay, C.M., Stewart, C. A., Park, E. M., Diao, L., Groves, S. M., Heeke, S., Nabet, B. Y., Fujimoto, J., Solis, L. M., Lu, W., et al. (2021). Patterns of transcription factor programs and immune pathway activation define four major subtypes of SCLC with distinct therapeutic vulnerabilities. Cancer Cell 39, 346-360 e347. 10.1016/j.ccell.2020.12.014.
57. Tang, F., Xu, D., Wang, S., Wong, C. K., Martinez-Fundichely, A., Lee, C. J., Cohen, S., Park, J., Hill, C. E., Eng, K., et al. (2022). Chromatin profiles classify castration-resistant prostate cancers suggesting therapeutic targets. Science 376, eabe1505. 10.1126/science.abe1505.
58. Heinz, S., Benner, C., Spann, N., Bertolino, E., Lin, Y.C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589. 10.1016/j.molcel.2010.05.004.
59. Jackstadt, R., Roh, S., Neumann, J., Jung, P., Hoffmann, R., Horst, D., Berens, C., Bornkamm, G. W., Kirchner, T., Menssen, A., and Hermeking, H. (2013). AP4 is a mediator of epithelial-mesenchymal transition and metastasis in colorectal cancer. J Exp Med 210, 1331-1350. 10.1084/jem.20120812.
60. Kim, M. Y., Jeong, B.C., Lee, J. H., Kee, H. J., Kook, H., Kim, N. S., Kim, Y. H., Kim, J. K., Ahn, K. Y., and Kim, K. K. (2006). A repressor complex, AP4 transcription factor and geminin, negatively regulates expression of target genes in nonneuronal cells. Proc Natl Acad Sci USA 103, 13074-13079. 10.1073/pnas.0601915103.
61. Skene, P.J., Henikoff, J. G., and Henikoff, S. (2018). Targeted in situ genome-wide profiling with high efficiency for low cell numbers. Nat Protoc 13, 1006-1019. 10.1038/nprot.2018.015.
62. Rudin, C.M., Brambilla, E., Faivre-Finn, C., and Sage, J. (2021). Small-cell lung cancer. Nat Rev Dis Primers 7, 3. 10.1038/s41572-020-00235-0.
63. Schaffer, B.E., Park, K. S., Yiu, G., Conklin, J. F., Lin, C., Burkhart, D. L., Karnezis, A. N., Sweet-Cordero, E. A., and Sage, J. (2010). Loss of p130 accelerates tumor development in a mouse model for human small-cell lung carcinoma. Cancer Res 70, 3877-3883. 10.1158/0008-5472.CAN-09-4228.
64. Jia, D., Augert, A., Kim, D.W., Eastwood, E., Wu, N., Ibrahim, A.H., Kim, K. B., Dunn, C. T., Pillai, S. P. S., Gazdar, A. F., et al. (2018). Crebbp Loss Drives Small Cell Lung Cancer and Increases Sensitivity to HDAC Inhibition. Cancer Discov 8, 1422-1437. 10.1158/2159-8290.CD-18-0385.
65. Bishop, J.L., Thaper, D., Vahid, S., Davies, A., Ketola, K., Kuruma, H., Jama, R., Nip, K. M., Angeles, A., Johnson, F., et al. (2017). The Master Neural Transcription Factor BRN2 Is an Androgen Receptor-Suppressed Driver of Neuroendocrine Differentiation in Prostate Cancer. Cancer Discov 7, 54-71. 10.1158/2159-8290.CD-15-1263.
66. Zou, M., Toivanen, R., Mitrofanova, A., Floch, N., Hayati, S., Sun, Y., Le Magnen, C., Chester, D., Mostaghel, E. A., Califano, A., et al. (2017). Transdifferentiation as a Mechanism of Treatment Resistance in a Mouse Model of Castration-Resistant Prostate Cancer. Cancer Discov 7, 736-749. 10.1158/2159-8290.CD-16-1174.
67. Faugeroux, V., Pailler, E., Oulhen, M., Deas, O., Brulle-Soumare, L., Hervieu, C., Marty, V., Alexandrova, K., Andree, K. C., Stoecklein, N. H., et al. (2020). Genetic characterization of a unique neuroendocrine transdifferentiation prostate circulating tumor cell-derived eXplant model. Nat Commun 11, 1884. 10.1038/s41467-020-15426-2.
68. Tsoi, J., Robert, L., Paraiso, K., Galvan, C., Sheu, K. M., Lay, J., Wong, D. J. L., Atefi, M., Shirazi, R., Wang, X., et al. (2018). Multi-stage Differentiation Defines Melanoma Subtypes with Differential Vulnerability to Drug-Induced Iron-Dependent Oxidative Stress. Cancer Cell 33, 890-904 e895. 10.1016/j.ccell.2018.03.017.
69. Deng, Q., Ramskold, D., Reinius, B., and Sandberg, R. (2014). Single-cell RNA-seq reveals dynamic, random monoallelic gene expression in mammalian cells. Science 343, 193-196. 10.1126/science. 1245316.
70. Basili, D., Zhang, J.L., Herbert, J., Kroll, K., Denslow, N. D., Martyniuk, C. J., Falciani, F., and Antczak, P. (2018). In Silico Computational Transcriptomics Reveals Novel Endocrine Disruptors in Largemouth Bass (Micropterus salmoides). Environ Sci Technol 52, 7553-7565. 10.1021/acs.est.8b02805.
71. Jiang, S., Williams, K., Kong, X., Zeng, W., Nguyen, N. V., Ma, X., Tawil, R., Yokomori, K., and Mortazavi, A. (2020). Single-nucleus RNA-seq identifies divergent populations of FSHD2 myotube nuclei. PLOS Genet 16, e1008754. 10.1371/journal.pgen. 1008754.
72. Kassambara, A., Reme, T., Jourdan, M., Fest, T., Hose, D., Tarte, K., and Klein, B. (2015). GenomicScape: an easy-to-use web tool for gene expression data analysis. Application to investigate the molecular events in the differentiation of B cells into plasma cells. PLOS Comput Biol 11, e1004077. 10.1371/journal.pcbi.1004077.
73. O'Meara, C.C., Wamstad, J. A., Gladstone, R. A., Fomovsky, G. M., Butty, V. L., Shrikumar, A., Gannon, J. B., Boyer, L. A., and Lee, R. T. (2015). Transcriptional reversion of cardiac myocyte fate during mammalian cardiac regeneration. Circ Res 116, 804-815. 10.1161/CIRCRESAHA.116.304269.
74. Yan, L., Yang, M., Guo, H., Yang, L., Wu, J., Li, R., Liu, P., Lian, Y., Zheng, X., Yan, J., et al. (2013). Single-cell RNA-Seq profiling of human preimplantation embryos and embryonic stem cells. Nature Structural & Molecular Biology 20, 1131-1139. 10.1038/nsmb.2660.
75. Ebisuya, M., and Briscoe, J. (2018). What does time mean in development? Development 145. 10.1242/dev.164368.
76. Ivakhnitskaia, E., Lin, R. W., Hamada, K., and Chang, C. (2018). Timing of neuronal plasticity in development and aging. Wiley Interdiscip Rev Dev Biol 7. 10.1002/wdev.305.

77 Montavon, T., and Soshnikova, N. (2014). Hox gene regulation and timing in embryogenesis. Semin Cell Dev Biol 34, 76-84. 10.1016/j.semcdb.2014.06.005.
78. Huang, Y.H., Klingbeil, O., He, X. Y., Wu, X. S., Arun, G., Lu, B., Somerville, T. D. D., Milazzo, J. P., Wilkinson, J. E., Demerdash, O. E., et al. (2018). POU2F3 is a master regulator of a tuft cell-like variant of small cell lung cancer. Genes Dev 32, 915-928. 10.1101/gad.314815.118.
79. Castro, D.S., Skowronska-Krawczyk, D., Armant, O., Donaldson, I. J., Parras, C., Hunt, C., Critchley, J.A., Nguyen, L., Gossler, A., Gottgens, B., Matter, J. M., and Guillemot, F. (2006). Proneural bHLH and Brn proteins coregulate a neurogenic program through cooperative binding to a conserved DNA motif. Dev Cell 11, 831-844. 10.1016/j.devcel.2006.10.006.
80. Weindorf, S. C., Taylor, A. S., Kumar-Sinha, C., Robinson, D., Wu, Y. M., Cao, X., Spratt, D. E., Kim, M. M., Lagstein, A., Chinnaiyan, A. M., and Mehra, R. (2019). Metastatic castration resistant prostate cancer with squamous cell, small cell, and sarcomatoid elements-a clinicopathologic and genomic sequencing-based discussion. Med Oncol 36, 27. 10.1007/s12032-019-1250-8.
81. Lachmann, A., Giorgi, F. M., Lopez, G., and Califano, A. (2016). ARACNe-AP: gene network reverse engineering through adaptive partitioning inference of mutual information. Bioinformatics 32, 2233-2235. 10.1093/bioinformatics/btw216.
82. Drost, J., Karthaus, W. R., Gao, D., Driehuis, E., Sawyers, C. L., Chen, Y., and Clevers, H. (2016). Organoid culture systems for prostate epithelial and cancer tissue. Nat Protoc 11, 347-358. 10.1038/nprot.2016.006.
83. Shultz, L. D., Ishikawa, F., and Greiner, D. L. (2007). Humanized mice in translational biomedical research. Nat Rev Immunol 7, 118-130. 10.1038/nri2017.
84. Seiler, C. Y., Park, J. G., Sharma, A., Hunter, P., Surapaneni, P., Sedillo, C., Field, J., Algar, R., Price, A., Steel, J., et al. (2014). DNASU plasmid and PSI: Biology-Materials repositories: resources to accelerate biological research. Nucleic Acids Res 42, D1253-1260. 10.1093/nar/gkt1060.
85. Joung, J., Konermann, S., Gootenberg, J. S., Abudayyeh, O. O., Platt, R.J., Brigham, M. D., Sanjana, N. E., and Zhang, F. (2017). Genome-scale CRISPR-Cas9 knockout and transcriptional activation screening. Nat Protoc 12, 828-863. 10.1038/nprot.2017.016.
86. Tiscornia, G., Singer, O., and Verma, I. M. (2006). Production and purification of lentiviral vectors. Nat Protoc 1, 241-245. 10.1038/nprot.2006.37.
87. Vivian, J., Rao, A. A., Nothaft, F. A., Ketchum, C., Armstrong, J., Novak, A., Pfeil, J., Narkizian, J., Deran, A. D., Musselman-Brown, A., et al. (2017). Toil enables reproducible, open source, big biomedical data analyses. Nat Biotechnol 35, 314-316. 10.1038/nbt.3772.
88. Love, M. I., Huber, W., and Anders, S. (2014). Moderated estimation of fold change and dispersion for RNA-seq data with DESeq2. Genome Biol 15, 550. 10.1186/s13059-014-0550-8.
89. Kuleshov, M. V., Jones, M. R., Rouillard, A. D., Fernandez, N. F., Duan, Q., Wang, Z., Koplev, S., Jenkins, S. L., Jagodnik, K. M., Lachmann, A., et al. (2016). Enrichr: a comprehensive gene set enrichment analysis web server 2016 update. Nucleic Acids Res 44, W90-97. 10.1093/nar/gkw377.
90. Quinlan, A. R., and Hall, I.M. (2010). BEDTools: a flexible suite of utilities for comparing genomic features. Bioinformatics 26, 841-842. 10.1093/bioinformatics/btq033.
91. Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47. 10.1093/nar/gkv007.
92. Zerbino, D. R., Johnson, N., Juettemann, T., Wilder, S. P., and Flicek, P. (2014). WiggleTools: parallel processing of large collections of genome-wide datasets for visualization and statistical analysis. Bioinformatics 30, 1008-1009. 10.1093/bioinformatics/btt737.
93. Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic Acids Res 44, W160-165. 10.1093/nar/gkw257.
94. Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821. 10.1016/j.cell.2019.05.031.
95. Greulich, F., Mechtidou, A., Horn, T., and Uhlenhaut, N. H. (2021). Protocol for using heterologous spike-ins to normalize for technical variation in chromatin immunoprecipitation. STAR Protoc 2, 100609. 10.1016/j.xpro.2021.100609.

What is claimed is:

1. A method for treating cancer in a subject, the method comprising administering an inhibitor of a gene, wherein the gene comprises one or more of tenascin C (TNC), advillin (AVIL), S100A7, PPARG, LOX lysyl oxidase, KLF5, APOBEC2, FOSL1, FOXM1, hedgehog, RELA, p65, IKK complex, JAK, STAT, TFAP4, geminin, OCA-1, OCA-2, Nf-kB, Nf-kB family and associated regulators, angiogenesis regulators such as BMX kinase, TEK kinase, periostin (POSTN), and VEGF family, stress genes, such as the JUN/FOS family, a gene from the S100 family, and cell state regulators such as ASCL1, POU2F3, NEUROD1, ASCL2, and YAP1.

2. A method for treating cancer prostate cancer or lung cancer in a subject, the method comprising administering an inhibitor of a gene, wherein the gene comprises one or more genes from Tables 1-6.

3. A method for inhibiting cancer transdifferentiation in a subject having cancer, the method comprising administering an inhibitor of a gene, wherein the gene comprises one or more of tenascin C (TNC), advillin (AVIL), S100A7, PPARG, LOX lysyl oxidase, KLF5, APOBEC2, FOSL1, FOXM1, hedgehog, RELA, p65, IKK complex, JAK, STAT, TFAP4, geminin, OCA-1, OCA-2, Nf-KB, Nf-kB family and associated regulators, angiogenesis regulators such as BMX kinase, TEK kinase, periostin (POSTN), and VEGF family, stress genes, such as the JUN/FOS family, a gene from the S100 family, and cell state regulators such as ASCL1, POU2F3, NEUROD1, ASCL2, and YAP1.

4. A method for inhibiting cancer transdifferentiation in a subject having cancer, the method comprising administering an inhibitor of a gene, wherein the gene comprises one or more genes from Tables 1-6.

5. The method of any one of claims 1-4, wherein the cancer comprises lung, prostate, basal cell carcinoma, hematopoietic cancer, ovarian cancer, epithelial cancer, sarcomas, small round cell-cancers of childhood, or neuroblastoma.

6. The method of any one of claims 3-5, wherein inhibiting transdifferentiation comprises inhibiting neuroendocrine or small cell transdifferentiation.

7. The method of claim 5 or 6, wherein the prostate cancer comprises prostate adenocarcinoma, castration-resistant prostate cancer, castration-sensitive prostate cancer, or hormone-refractory prostate cancer.

8. The method of claim 5 or 6, wherein the lung cancer comprises non-small cell lung cancer, adenocarcinoma, adenocarcinoma in situ, squamous cell carcinoma, large cell carcinoma, large cell neuroendocrine carcinoma, adenosquamous carcinoma, sarcomatoid carcinoma, or small cell lung cancer.

9. The method of claim 5 or 6, wherein the hematopoietic cancer comprises leukemia or lymphoma.

10. The method of claim 9, wherein the cancer comprises acute lymphoblastic leukemia, acute myeloid leukemia, chronic lymphocytic leukemia, acute monocytic leukemia, Hodgkin's lymphoma, or Non-Hodgkin's lymphoma.

11. The method of any one of claims 1-10, wherein the method comprises administration of an additional agent.

12. The method of any one of claims 1-10, wherein the subject is being treated with or has been prescribed an additional agent or therapy.

13. The method of claim 12, wherein the subject is being treated with an additional agent or therapy and wherein the subject has been determined to be resistant to the additional agent or therapy.

14. The method of any one of claims 1-8, wherein the subject is one that has not been treated with an additional agent or therapy.

15. The method of any one of claims 1-14, wherein the cancer comprises prostate cancer.

16. The method of claim 15, wherein the additional agent comprises one or more of androgen suppression therapy, chemotherapy, immunotherapy, targeted therapy, radiation, or surgery.

17. The method of claim 16, wherein the androgen suppression therapy comprises one or more of leuprolide, goserelin, triptorelin, leuprolide mesylate, degarelix, relugolix, abiraterone, ketoconazole, flutamide, bicalutamide, nilutamide, enzalutamide, apalutamide, or darolutamid.

18. The method of claim 16 or 17, wherein the immunotherapy comprises pembrolizumab.

19. The method of any one of claims 16-18, wherein the targeted therapy comprises rucaparib and/or olaparib.

20. The method of any one of claims 1-14, wherein the cancer comprises lung cancer.

21. The method of claim 20, wherein the additional agent comprises one or more of chemotherapy, immunotherapy, radiation therapy, targeted therapy, or surgery.

22. The method of claim 21, wherein the chemotherapy comprises cisplatin, carboplatin, paclitaxel, albumin-bound paclitaxel, docetaxel, gemcitabine, vinorelbine, etoposide, pemetrexed, or combinations thereof.

23. The method of claim 21 or 23, wherein the immunotherapy comprises nivolumab, atezolizumab, durvalumab, ipilimumab, tremelimumab, or combinations thereof.

24. The method of any one of claims 21-23, wherein the targeted therapy comprises bevacizumab, ramucirumab, sotorasib, adagrasib, erlotinib, afatinib, gefitinib, osimertinib, dacomitinib, amivantamab, mobocertinib, necitumumab, crizotinib, ceritinib, alectinib, brigatinib, lorlatinib, entrectinib, dabrafenib, trametinib, selpercatinib, pralsetinib, capmatinib, tepotinib, trastuzumab deruxtecan, larotrectinib, and combinations thereof.

25. The method of any one of claims 1-24, wherein the inhibitor comprises an inhibitor nucleic acid, inhibitory protein, or inhibitory small molecule.

26. The method of claim 25, wherein the inhibitor is an siRNA, a double stranded RNA, a short hairpin RNA, or an antisense oligonucleotide.

27. The method of claim 25 or 26, wherein the inhibitor is an antibody.

28. The method of any of claims 1-27, wherein the cancer comprises stage I, II, III, or IV cancer.

29. The method of any one of claims 1-28, wherein the cancer comprises metastatic cancer.

30. The method of any one of claims 1-28, wherein the cancer comprises non-metastatic cancer.

31. The method of any one of claims 1-30, wherein the subject is a human.
